(19) United States
(12) Patent Application Publication
(10) Pub. No.: US 2022/0306640 A1
(43) Pub. Date: Sep. 29, 2022

(54) HYDANTOIN DERIVATIVE

(71) Applicant: ONO PHARMACEUTICAL CO., LTD., Osaka (JP)

(72) Inventors: Yota NISHIOKA, Osaka (KR); Masakuni KURONO, Osaka (KR); Rena NISHIZAWA, Osaka (JP); Balachandra BANDODKAR, Beijing (CN); Xuechao GAO, Beijing (CN); Zhilong WAN, Beijing (CN); Ranran LV, Beijing (CN); Kevin DOYLE, Saffron Walden (GB); Michael GOLDSMITH, Saffron Walden (GB)

(73) Assignee: ONO PHARMACEUTICAL CO., LTD., Osaka (JP)

(21) Appl. No.: 17/638,375

(22) PCT Filed: Sep. 4, 2020
(86) PCT No.: PCT/CN2020/113408

§ 371 Date: Feb. 25, 2022

(30) Foreign Application Priority Data
Sep. 6, 2019 (CN) PCT/CN2019/104694
Aug. 5, 2020 (CN) PCT/CN2020/107031

(51) Int. Cl. C07D 487/04; C07D 471/04; C07D 401/14; C07D 403/12; C07D 513/04

(52) U.S. Cl. CPC C07D 487/04; C07D 471/04; C07D 401/14; C07D 403/12; C07D 513/04

(57) ABSTRACT

A compound of formula (I-a):

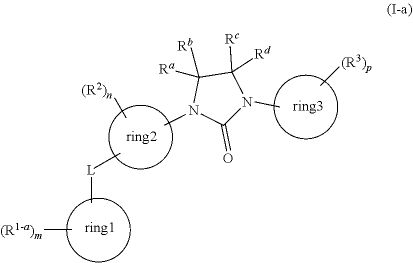

wherein the symbols are defined in the specification, and which has a strong DDR1 inhibitory activity, and can be a therapeutic agent for DDR1-related diseases, for example, a cancer, a kidney disease, a cardiovascular disease, a central nervous system disease, or fibrosis.

The present application claims priority benefit to International Patent Application No. PCT/CN2019/104694, filed on Sep. 6, 2019, and International Patent Application No. PCT/CN2020/107031 filed on Aug. 5, 2020, all of which are incorporated by reference herein in their entirety.

### TECHNICAL FIELD

One embodiment of the present invention relates to compounds represented by the following formula (I-a) or (I):

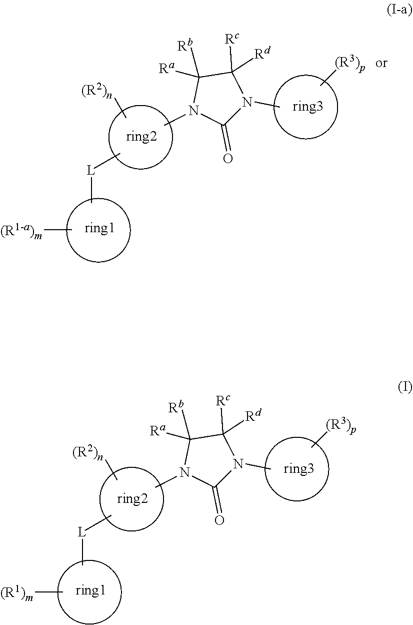

(wherein all the symbols represent the same meanings as described below), and to salts, solvates, and N-oxides thereof, or prodrugs of these (hereinafter, these will be referred to also as “present compounds”).

### BACKGROUND ART

DDR1 (discoidin domain receptor 1) is a receptor tyrosine kinase having a discoidin (DS)-like domain in its extracellular domain. DDR1 is activated upon binding of its ligand, collagen, to the DS-like domain, and, upon activation, transmits the collagen signal to the cell interior. The signal activates signaling pathways such as the p38MAPK pathway and the ERK pathway, and elicits an inflammatory response that increases collagen synthesis. Specifically, the signal is known to cause inflammation, excessive fiber formation, and renal function loss in the kidneys. Overexpression of DDR1 in various types of cancers such as lung cancer, breast cancer, esophageal cancer, head and neck cancer, liver cancer, prostate cancer, lymphoma, and leukemia, and DDR1's involvement in atherosclerosis, Alzheimer's disease, and Parkinson's disease are also known (see International Review of Cell and Molecular Biology, 2014, Vol. 310, p. 39-87 (NPL1); Nephron, 2017, Vol. 137, No. 3, p. 212-220 (NPL2); and Journal of Neuroimmunology, 2017, Vol. 311, p. 1-9 (NPL3)). Involvement of DDR1 in various kidney diseases is particularly well known. For example, knockout of DDR1 is known to increase survival rate and maintain renal functions in an Alport syndrome model mouse (see Matrix Biology, 2010, Vol. 29, issue 5, p. 346-356 (NPL4)). DDR1 is also known to be involved in the pathology of CKD (chronic kidney disease) (see Journal of the American Society of Nephrology, 2006, Vol. 17, issue 12, p. 3374-3381 (NPL5), and Cell Adhesion and Migration, 2018, 12, 4, p. 299-304 (NPL6)). A compound that inhibits the activity of DDR1 would therefore be useful for the treatment of various cancers, kidney diseases, and cardiovascular diseases.

In related art of the present invention, WO2007/018137 (PTL1) reports that compounds represented by the following general formula (A) have an inhibitory effect against a plurality of kinases, including Raf, KDR, and Flt-3.

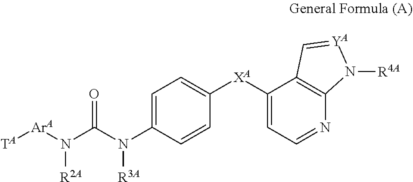

wherein:

Ar^{A} is an arylene linking group selected from

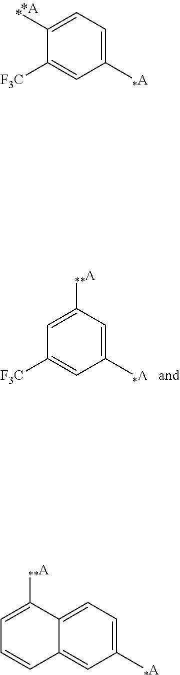

wherein *^{A} represents a nitrogen atom binding site, and **^{A} represents a binding site for T^{A};

T^{A} is —(O)_{nA}—R^{A}, wherein R^{A} is a C1-C6 alkyl group, a C3-C8 cycloalkyl group, a phenyl group, a tetrahydrofuranyl group, or the like, and these may be substituted with 1 to 3 substituents, and n is 0 or 1;

X^{A} is O, S(O)_{mA}, CH₂, C═O, or NR^{1A};

R^{2A}, R^{3A}, and R^{4A} are each independently selected from a hydrogen atom and C1-C3 alkyl (the alkyl may be substituted with 1 to 3 substituents);

R^{2A} and R^{3A} may form a five- or six-membered heterocycle with a urea structure containing a nitrogen atom that binds to these groups, and the heterocycle may be substituted with 1 to 3 substituents independently selected from an oxo group and a hydroxyl group; and

Y^{A} is CH or N.

(It is to be noted that the definitions of these groups are excerpts.)

There is also report that compounds represented by the following general formula (B) have an inhibitory effect against a plurality of kinases, including c-Met, KDR, c-Kit, Flt-3, and Flt-4 (see WO2005/030140 (PTL2), and WO2006/108059 (PTL3)),

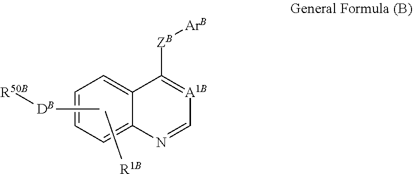

wherein:

R^{1B} is selected from —H, halogen, —OR^{3B}, —NO₂, —NH₂, —NR^{3B}R^{4B}, and optionally substituted lower alkyl;

A^{1B} is selected from ═N—, ═CH—, and ═C(CN)—;

Z^{B} is selected from —S(O)₀₋₂—, —O—, and —NR^{5B}—;

Ar^{B} is a group of the following formula II or III,

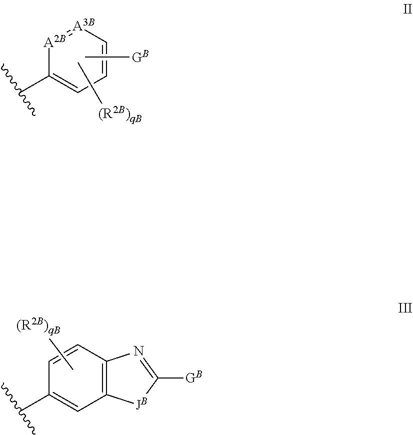

wherein:
R^{2B} is selected from —H, halogen, optionally substituted lower alkyl, and the like;
qB is 0 to 4;
G^{B} is a group -B^{B}-L^{B}-T^{B}, wherein B^{B} is non-existent or selected from —N(R^{13B})—, —N(SO₂R^{13B})—, —O—, —S(O)₀₋₂—, and —C(═O)—; L^{B} is non-existent or selected from carbonyl, sulfone, alkylene, optionally substituted four- to six-membered heterocyclyl containing at least one nitrogen atom, and the like; and T^{B} is —H, optionally substituted alkyl, or the like;
J^{B} is selected from —S(O)₀₋₂—, —O—, and —NR^{15B}—; and
A^{2B} and A^{3B} are each independently selected from ═N—, and ═C(R^{2B})—;

D^{B} is selected from —O—, —S(O)₀₋₂—, and —NR^{15B}—; and

R^{50B} is R^{3B} or follows the following formula IV,

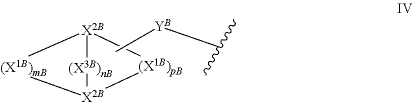

wherein X^{1B}, X^{2B}, and, optionally, X^{3B} represent atoms of a bridged saturated ring system containing at most four ring heteroatoms represented by X^{1B}, X^{2B}, or X^{3B}.

(It is to be noted that the definitions of these groups are excerpts.)

WO2007/012661 (PTL4) reports that compounds represented by the following general formula (C) have antagonistic activity against a melanin-concentrating hormone receptor (MCHR),

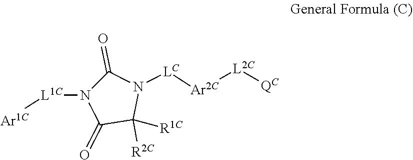

wherein Ar^{1C} represents aryl, heteroaryl, cycloalkyl, or a heterocyclic group that may be substituted with 1 to 5 groups;

L^{1C} represents a single bond, or C1-C6 alkylene, or the like;

R^{1C} and R^{2C} may be the same or different, and represent hydrogen atoms or C1-C6 alkyl groups, wherein R^{1C} or R^{2C} may form a 5- to 7-membered heterocyclic ring with Ar^{2C} or with L^{C} and the hydantoin ring attached thereto;

L^{C} represents a single bond, C1-C6 alkylene, or the like;

Ar^{2C} represents aryl, heteroaryl, or a heterocyclic ring that may be substituted with 1 to 4 groups;

L^{2C} represents C1-C6 alkylene, or the like; and

Q^{C} represents a basic group, or a group represented by NR^{5C}R^{6C}.

(It is to be noted that the definitions of these groups are excerpts.)

WO2019/014304 (PTL5) reports that compounds represented by the following general formula (D) are useful as selective inhibitors of ROCK kinases.

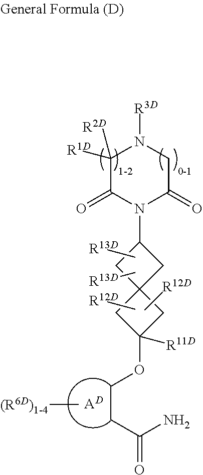

wherein, ring AD is heteroaryl comprising carbon and 1-2 nitrogen atoms;

R^{3D} represents substituted —(CR^{4D}R^{4D})_{nD}-4- to 15-membered heterocycle, or the like.

(It is to be noted that the definitions of these groups are excerpts.)

However, none of the related art teaches or suggests the prominent DDR1 inhibitory activity produced by the present compounds.

### CITATION LIST

### Patent Literatures

[Patent Literature 1] WO2007/018137
[Patent Literature 2] WO2005/030140
[Patent Literature 3] WO2006/108059
[Patent Literature 4] WO2007/012661
[Patent Literature 5] WO2019/014304

### Non-Patent Literatures

[Non-Patent Literature 1] International Review of Cell and Molecular Biology, 2014, Vo. 3 10, p. 39-87
[Non-Patent Literature 2] Nephron, 2017, Vol. 137, No. 3, p. 212-220
[Non-Patent Literature 3] Journal of Neuroimmunology, 2017, Vol. 311, p. 1-9
[Non-Patent Literature 4] Matrix Biology 2010. Vol. 29, issue 5, p. 346-356
[Non-Patent Literature 5] Journal of the American Society of Nephrology, 2006, Vol. 17, issue 12, p. 3374-3381
[Non-Patent Literature 6] Cell Adhesion and Migration, 2018, 12, 4, p. 299-304

### SUMMARY OF INVENTION

### Technical Problem

An object of the present invention is to provide a novel compound having a DDR1 inhibitory effect, and to use the compound for the prevention and/or treatment of DDR1-related disease. The invention is also intended to provide a pharmaceutical composition containing the compound.

### Solution to Problem

In order to achieve the foregoing objects, the present inventors conducted intensive studies to find a compound having a DDR1 inhibitory effect, and found that compounds represented by the following general formula (I-a) have prominent DDR1 inhibitory activity. The present invention was completed on the basis of this finding.

Specifically, the present invention relates to the following, for example.

[1] A compound represented by the following general formula (I-a), or a salt thereof,

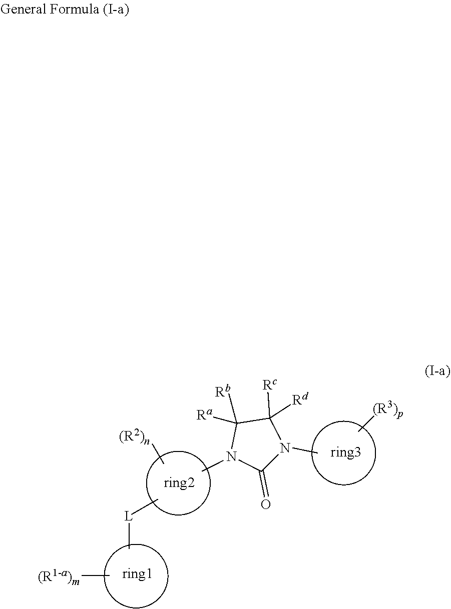

wherein:

ring 1 represents (1) a five- to seven-membered nitrogen-containing monocyclic heterocycle, or (2) an eight- to ten-membered nitrogen-containing bicyclic heterocycle,

R^{1-a} represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—C1-6 alkylene)-NR⁴R⁵, (9) a five- to seven-membered monocyclic heterocyclyl, (10) NR⁴R⁵, (11) C(═O)NR⁴R⁵, (12) a cyano group, (13) a hydroxyl group, (14) an oxo group, (15) a C1-6 alkylsulfonyl group, (16) a Q-(C3-7 monocyclic carbocyclyl) group, or (17) a Q-(three- to seven-membered monocyclic heterocyclyl) group wherein:
Q represents any of NR⁶, O, and optionally oxidized S,
R⁴ and R⁵ each independently represent (1) a hydrogen atom, (2) a C1-6 alkyl group, (3) a C3-6 cycloalkyl group, (4) a three- to six-membered saturated heterocyclyl, (5) C(═O)R⁷, (6) (C1-6 alkylene)-NR⁸R⁹, (7) a phenyl group, or (8) a benzyl group, or R⁴ and R⁵ and the nitrogen atom they are attached thereto together form a five- to seven-membered nitrogen-containing monocyclic heterocycle, wherein, when R⁴ or R⁵ represents a C1-6 alkyl group, one of the carbon atoms in the C1-6 alkyl group may be replaced by NR¹⁰, O, or optionally oxidized S, and, when R⁴ or R⁵ represents (C1-6 alkylene)-NR⁸R⁹, the C1-6 alkylene may be substituted with an oxo group,
R⁶ represents (1) a hydrogen atom, (2) a C1-6 alkyl group, (3) a C3-6 cycloalkyl group, (4) a phenyl group, or (5) a benzyl group,
R⁷ represents (1) a hydrogen atom, (2) a C1-6 alkyl group, (3) a C3-6 cycloalkyl group, (4) a phenyl group, or (5) a benzyl group,
R⁸ and R⁹ each independently represent (1) a hydrogen atom, (2) a C1-6 alkyl group, (3) a C3-6 cycloalkyl group, (4) a phenyl group, or (5) a benzyl group,
R¹⁰ represents (1) a hydrogen atom, (2) a C1-6 alkyl group, (3) a C3-6 cycloalkyl group, (4) a phenyl group, or (5) a benzyl group,

when R^{1-a} represents any of (2) to (11), (15), (16) or (17), R^{1-a} may be substituted with one or more substituents selected from a halogen atom, a C1-6 alkyl group, a C1-6 haloalkyl group, a C1-6 hydroxyalkyl group, a hydroxyl group, a cyano group, and an oxo group,

when m is 2 or more, the plurality of R^{1-a} may be the same or different, and, two R^{1-a} may form a five- to seven-membered monocyclic ring with atoms they are attached thereto,

L represents (1) a bond, (2) CR¹¹R¹², (3) C(═O), (4) O, (5) NR¹³, or (6) optionally oxidized S, wherein R¹¹ and R¹² each independently represent (1) a hydrogen atom or (2) a C1-6 alkyl group, and R¹³ represents (1) a hydrogen atom or (2) a C1-6 alkyl group,

ring 2 represents (1) a C3-7 monocyclic carbocycle, (2) five- to seven-membered nitrogen-containing monocyclic heterocycle, (3) a C5-8 bridged carbocycle,

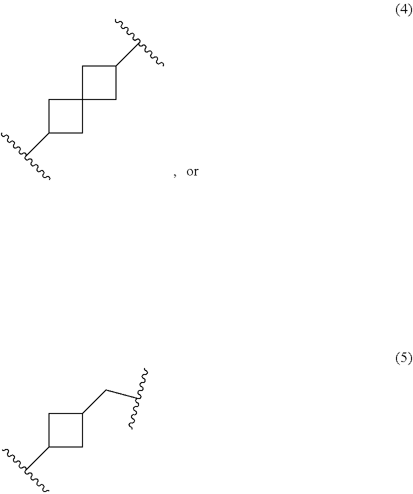 [SMILES: *C1CC2(C1)CC(*)C2 ; *CC1CC(*)C1]

R² represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7)
NR¹⁴R¹⁵, (8) a (C1-6 alkylene)-(five- to seven-membered monocyclic ring) group, (9) a cyano group, (10) a hydroxyl group, or (11) an oxo group, wherein:
   R¹⁴ and R¹⁵ each independently represent (1) a hydrogen atom, (2) a C1-6 alkyl group, (3) a C3-6 cycloalkyl group, (4) a three- to six-membered saturated heterocyclyl, (5) acetyl group, (6) a phenyl group, or (7) a benzyl group, or R¹⁴ and R¹⁵ and the nitrogen atom they are attached thereto together form a five- to seven-membered nitrogen-containing monocyclic heterocycle,
when R¹⁴ or R¹⁵ represent a C1-6 alkyl group, one of the carbon atoms in the C1-6 alkyl group may be replaced by NR¹⁶, O, or optionally oxidized S, and
R¹⁶ represents (1) a hydrogen atom, (2) a C1-6 alkyl group, (3) a C3-6 cycloalkyl group, (4) a phenyl group, or (5) a benzyl group,

when R² represents any of (2) to (8), one of the carbon atoms in R² may be replaced by NR¹⁷, O, or optionally oxidized S, wherein R¹⁷ represents (1) a hydrogen atom, (2) a C1-6 alkyl group, or (3) C(═O)R¹⁸, wherein R¹⁸ represents (1) a NH—(C1-6 alkyl) group, or (2) an O(C1-6 alkyl) group,

when R² represents any of (2) to (8), R² may be substituted with one or more substituents selected from a halogen atom, a C1-6 alkyl group, a C1-6 haloalkyl group, a C1-6 hydroxyalkyl group, a hydroxyl group, a cyano group, and an oxo group,

when n is 2 or more, the plurality of R² may be the same or different, and, two R² may form a C3-7 monocyclic carbocycle with atoms they are attached thereto,

when L representing NR¹³, R^{1-a}, R¹³ and atoms they are attached thereto may form a five- to seven-membered nitrogen-containing monocyclic heterocycle, or when L representing NR¹³, R², R¹³ and atoms they are attached thereto may form a five- to seven-membered nitrogen-containing monocyclic heterocycle,

R^{a} and R^{b} each independently represent (1) a hydrogen atom, (2) a halogen atom, (3) a C1-6 alkyl group, (4) a C1-6 alkoxy group, or (5) a hydroxyl group, or (6) R^{a} and R^{b} together represent an oxo group,

R^{c} and R^{d} each independently represent (1) a hydrogen atom, (2) a halogen atom, (3) a C1-6 alkyl group, (4) a C1-6 alkoxy group, or (5) a hydroxyl group, or (6) R^{c} and R^{d} together represent an oxo group,

when R^{a} and R^{b} represent an oxo group, RC and R^{d} do not represent an oxo group,

ring 3 represents (1) a C5-7 monocyclic carbocycle, or (2) a five- to seven-membered monocyclic heterocycle containing 1 to 4 heteroatoms selected from N, O, and optionally oxidized S,

R³ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) a C1-6 alkylsulfonyl group, (8) NR¹⁹R²⁰, (9) (C1-6 alkylene)-R²¹, (10) O—C1-6 alkylene)-R²¹, (11) 0-R²², (12) a three- to seven-membered monocyclic heterocyclyl, (13) a cyano group, (14) a hydroxyl group, or (15) an oxo group, wherein:
R¹⁹ and R²⁰ each independently represent (1) a hydrogen atom or (2) a C1-6 alkyl group, or, R¹⁹ and R²⁰ and the nitrogen atom they are attached thereto may together form a five- to seven-membered nitrogen-containing monocyclic heterocycle, wherein one of the carbon atoms in the five- to seven-membered nitrogen-containing monocyclic heterocycle may be replaced by NR²³, O, or optionally oxidized S,
R²¹ represents (1) a C3-6 cycloalkyl group, (2) a C1-6 alkylsulfonyl group, or NR¹⁹R²⁰,
R²² represents (1) a C3-6 cycloalkyl group, (2) a three- to six-membered saturated heterocyclyl, or (3) a C1-6 alkylsulfonyl group, wherein the C3-6 cycloalkyl group and the three- to six-membered saturated heterocyclyl may be substituted with C1-6 alkyl groups, and
R²³ represents (1) a hydrogen atom or (2) a C1-6 alkyl group,

when R³ represents any of (2) to (12), R³ may be substituted with one or more substituents selected from a halogen atom, a C1-6 alkyl group, a C1-6 haloalkyl group, a C1-6 hydroxyalkyl group, a hydroxyl group, a cyano group, and an oxo group,

when p is 2 or more, the plurality of R³ may be the same or different,

m represents an integer of 0 to 10,

n represents an integer of 0 to 10, and

p represents an integer of 0 to 10.

[2] The compound or a salt thereof according to item [1], wherein the compound is a compound represented by the following general formula (I),

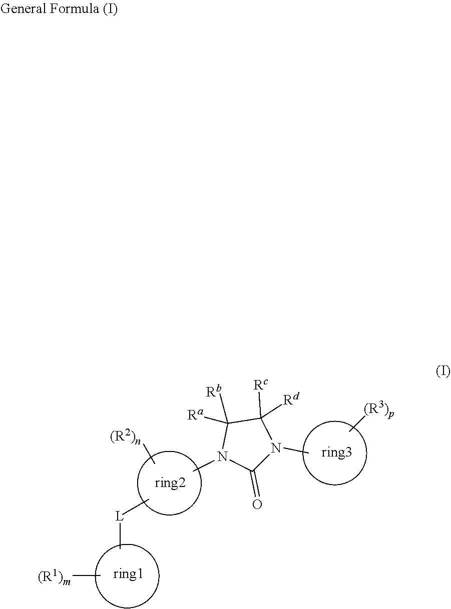

wherein:

ring 1 represents (1) a five- to seven-membered nitrogen-containing monocyclic heterocycle, or (2) an eight- to ten-membered nitrogen-containing bicyclic heterocycle,

R¹ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—(C1-6 alkylene)-NR⁴R⁵, (9) a five- to seven-membered monocyclic heterocyclyl, (10) NR⁴R⁵, (11) C(═O)NR⁴R⁵, (12) a cyano group, (13) a hydroxyl group, or (14) an oxo group, wherein:
Q represents any of NR, O, and optionally oxidized S,
R⁴ and R⁵ each independently represent (1) a hydrogen atom, (2) a C1-6 alkyl group, (3) a C3-6 cycloalkyl group, (4) a three- to six-membered saturated heterocyclyl, (5) C(═O)R⁷, (6) (C1-6 alkylene)-NR⁸R⁹, (7) a phenyl group, or (8) a benzyl group, wherein, when R⁴ or R⁵ represents a C1-6 alkyl group, one of the carbon atoms in the C1-6 alkyl group may be replaced by NR¹, O, or optionally oxidized S, and, when R⁴ and R⁵ represent C1-6 alkyl groups, R⁴ and R⁵ and the nitrogen atom attached thereto may together form a five- to seven-membered nitrogen-containing monocyclic heterocycle, and, when R⁴ or R⁵ represents (C1-6 alkylene)-NR⁸R⁹, the C1-6 alkylene may be substituted with an oxo group,
R⁶ represents (1) a hydrogen atom, (2) a C1-6 alkyl group, (3) a C3-6 cycloalkyl group, (4) a phenyl group, or (5) a benzyl group,
R⁷ represents (1) a hydrogen atom, (2) a C1-6 alkyl group, (3) a C3-6 cycloalkyl group, (4) a phenyl group, or (5) a benzyl group,
R⁸ and R⁹ each independently represent (1) a hydrogen atom, (2) a C1-6 alkyl group, (3) a C3-6 cycloalkyl group, (4) a phenyl group, or (5) a benzyl group,
R¹⁰ represents (1) a hydrogen atom, (2) a C1-6 alkyl group, (3) a C3-6 cycloalkyl group, (4) a phenyl group, or (5) a benzyl group,

when R¹ represents any of (2) to (11), R¹ may be substituted with one or more substituents selected from a halogen atom, a C1-6 alkyl group, a C1-6 haloalkyl group, a C1-6 hydroxyalkyl group, a hydroxyl group, a cyano group, and an oxo group,

when m is 2 or more, the plurality of R¹ may be the same or different,

L represents (1) a bond, (2) CR¹¹R¹², (3) C(═O), (4) O, (5) NR¹³, or (6) optionally oxidized S, wherein R¹¹ and R¹² each independently represent (1) a hydrogen atom or (2) a C1-6 alkyl group, and R¹³ represents (1) a hydrogen atom or (2) a C1-6 alkyl group,

ring 2 represents (1) a C3-7 monocyclic carbocycle, (2) five- to seven-membered nitrogen-containing monocyclic heterocycle, (3) a C5-8 bridged carbocycle,

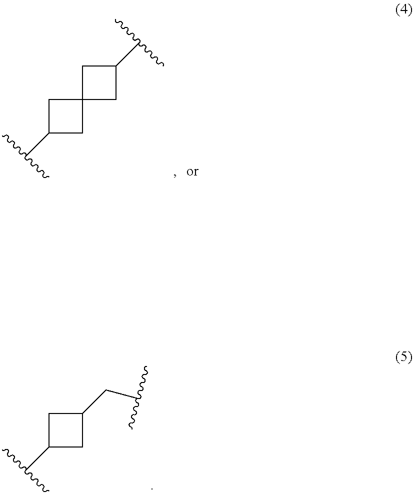 [SMILES: *C1CC2(C1)CC(*)C2 ; *CC1CC(*)C1]

R² represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) NR¹⁴R¹⁵, (8) a (C1-6 alkylene)-(five- to seven-membered monocyclic ring) group, (9) a cyano group, (10) a hydroxyl group, or (11) an oxo group, wherein:
R¹⁴ and R¹⁵ each independently represent (1) a hydrogen atom, (2) a C1-6 alkyl group, (3) a C3-6 cycloalkyl group, (4) a three- to six-membered saturated heterocyclyl, (5) acetyl group, (6) a phenyl group, or (7) a benzyl group,
when R¹⁴ or R¹⁵ represent a C1-6 alkyl group, one of the carbon atoms in the C1-6 alkyl group may be replaced by NR¹⁶, O, or optionally oxidized S,
when R¹⁴ and R¹⁵ represent C1-6 alkyl groups, R¹⁴ and R¹⁵ and the nitrogen atom attached thereto may together form a five- to seven-membered nitrogen-containing monocyclic heterocycle, and
R¹⁶ represents (1) a hydrogen atom, (2) a C1-6 alkyl group, (3) a C3-6 cycloalkyl group, (4) a phenyl group, or (5) a benzyl group,

when R² represents any of (2) to (8), one of the carbon atoms in R² may be replaced NR¹⁷, O, or optionally oxidized S, wherein R¹⁷ represents (1) a hydrogen atom, (2) a C1-6 alkyl group, or (3) C(═O)R¹⁸, wherein R¹⁸ represents (1) a NH—(C1-6 alkyl) group, or (2) an O(C1-6 alkyl) group,

when R² represents any of (2) to (8), R² may be substituted with one or more substituents selected from a halogen atom, a C1-6 alkyl group, a C1-6 haloalkyl group, a C1-6 hydroxyalkyl group, a hydroxyl group, a cyano group, and an oxo group,

when n is 2 or more, the plurality of R² may be the same or different, and, when two R² represent C1-6 alkyl groups, the two R² may form a C3-7 monocyclic carbocycle with atoms constituting the ring 2,

when one of R¹ and R² represents a C1-6 alkyl group with L representing NR¹³ and R¹³ representing a C1-6 alkyl group, one of R¹ and R² may form a five- to seven-membered nitrogen-containing monocyclic heterocycle with NR¹³,

R^{a} and R^{b} each independently represent (1) a hydrogen atom, (2) a halogen atom, (3) a C1-6 alkyl group, (4) a C1-6 alkoxy group, or (5) a hydroxyl group, or (6) R^{a} and R^{b} together represent an oxo group,

R^{c} and R^{d} each independently represent (1) a hydrogen atom, (2) a halogen atom, (3) a C1-6 alkyl group, (4) a C1-6 alkoxy group, or (5) a hydroxyl group, or (6) R^{c} and R^{d} together represent an oxo group,

when R^{a} and R^{b} together represent an oxo group, R^{c} and R^{d} together do not represent an oxo group,

ring 3 represents a C5-7 monocyclic carbocycle, or a five- to seven-membered monocyclic heterocycle containing 1 to 4 heteroatoms selected from N, O, and optionally oxidized S,

R³ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) a C1-6 alkylsulfonyl group, (8) NR¹⁹R²⁰, (9) (C1-6 alkylene)-R²¹, (10) O—(C1-6 alkylene)-R²¹, (11) O—R²², (12) a three- to seven-membered monocyclic heterocyclyl, (13) a cyano group, (14) a hydroxyl group, or (15) an oxo group, wherein:
R¹⁹ and R²⁰ each independently represent (1) a hydrogen atom or (2) a C1-6 alkyl group, and when R¹⁹ and R²⁰ represent C1-6 alkyl groups, R¹⁹ and R²⁰ and the nitrogen atom attached thereto may together form a five- to seven-membered nitrogen-containing monocyclic heterocycle, wherein one of the carbon atoms in the five- to seven-membered nitrogen-containing monocyclic heterocycle may be replaced by NR²³, O, or optionally oxidized S,
R²¹ represents (1) a C3-6 cycloalkyl group, (2) a C1-6 alkylsulfonyl group, or (3) NR¹⁹R²⁰,
R²² represents (1) a C3-6 cycloalkyl group, (2) a three- to six-membered saturated heterocyclyl, or (3) a C1-6 alkylsulfonyl group, wherein the C3-6 cycloalkyl group and the three- to six-membered saturated heterocyclyl may be substituted with C1-6 alkyl groups, and
R²³ represents (1) a hydrogen atom or (2) a C1-6 alkyl group,

when R³ represents any of (2) to (12), R³ may be substituted with one or more substituents selected from a halogen atom, a C1-6 alkyl group, a C1-6 haloalkyl group, a C1-6 hydroxyalkyl group, a hydroxyl group, a cyano group, and an oxo group,

when p is 2 or more, the plurality of R³ may be the same or different,

m represents an integer of 0 to 10,

n represents an integer of 0 to 10, and

p represents an integer of 0 to 10.

[3] The compound or a salt thereof according to item [1] or [2], wherein the compound represented by general formula (I-a) or (I) is a compound represented by the following general formula (I-A), (I-B), or (I-C),

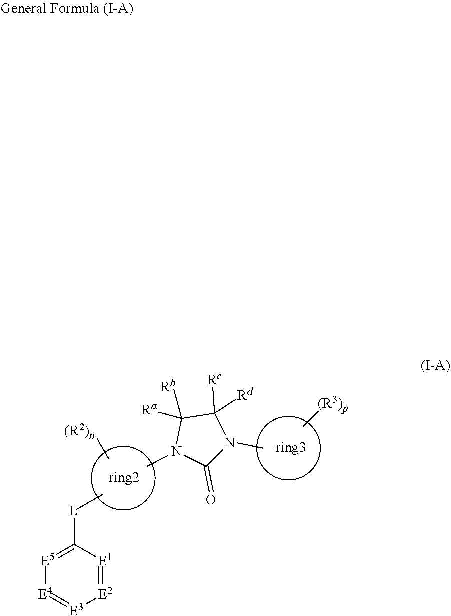

wherein:

E¹, E², E³, E⁴, and E⁵ each independently represent (1) CH, (2) CR¹, or (3) N, wherein at least one of E¹, E², E³, E⁴, and E⁵ represent N, and

other symbols represent the same meanings as described in item [1] or [2] above,

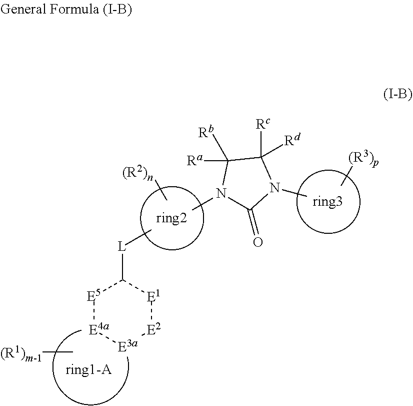

wherein:

E^{3a} and E^{4a} each independently represent (1) C or (2) N, wherein at least one of E¹, E², E^{3a}, E^{4a}, and E⁵ represent N,

ring 1-A, including E^{3a} and E^{4a}, represent a five- to seven-membered monocyclic ring group,

the broken-line bonds represent aromatic bonds,

m−1 represents an integer of 0 to 5, and

other symbols represent the same meanings as described in item [1], [2] or in general formula (I-A) above,

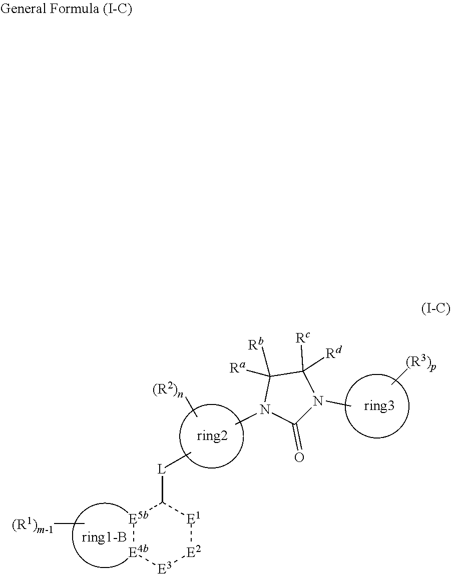

wherein:

E^{4b} and E^{5b} each independently represent (1) C or (2) N, wherein at least one of E¹, E², E³, E^{4b}, and E^{5b} represent N,

ring 1-B, including E^{4b} and E^{5b}, represents a five- to seven-membered monocyclic ring group, and

other symbols represent the same meanings as described in item [1], [2] or in general formulae (I-A) and (I-B) above.

[4] The compound or a salt thereof according to any one of item [1] to [3], wherein the moiety

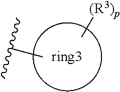

is

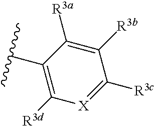

wherein:

X represents CR^{3e} or N,

R^{3a}, R^{3c}, R^{3d}, and R^{3e} each independently represent (1) a hydrogen atom, (2) a halogen atom, (3) a C1-6 alkyl group, (4) a C1-6 alkoxy group, (5) a cyano group, or (6) a hydroxyl group, and, when R^{3a}, R^{3c}, R^{3d}, or R^{3e} represents a C1-6 alkyl group or a C1-6 alkoxy group, the C1-6 alkyl group or a C1-6 alkoxy group may be substituted with 1 to 5 substituents selected from a halogen atom and a hydroxyl group,

R^{3b} represents (1) a C1-6 alkyl group, (2) a C1-6 alkoxy group, (3) a cyano group, or (4) a hydroxyl group, and, when R^{3b} represents a C1-6 alkyl group or a C1-6 alkoxy group, the C1-6 alkyl group or a C1-6 alkoxy group may be substituted with 1 to 5 substituents selected from a halogen atom and a hydroxyl group.

[5] The compound or a salt thereof according to any one of items [1] to [4], wherein the moiety

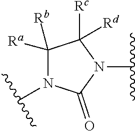

is

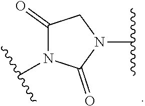 [SMILES: *N1CC(=O)N(*)C1=O]

[6] The compound or a salt thereof according to any one of items [1] to [5], wherein the moiety

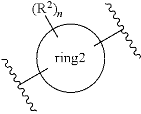

is selected from

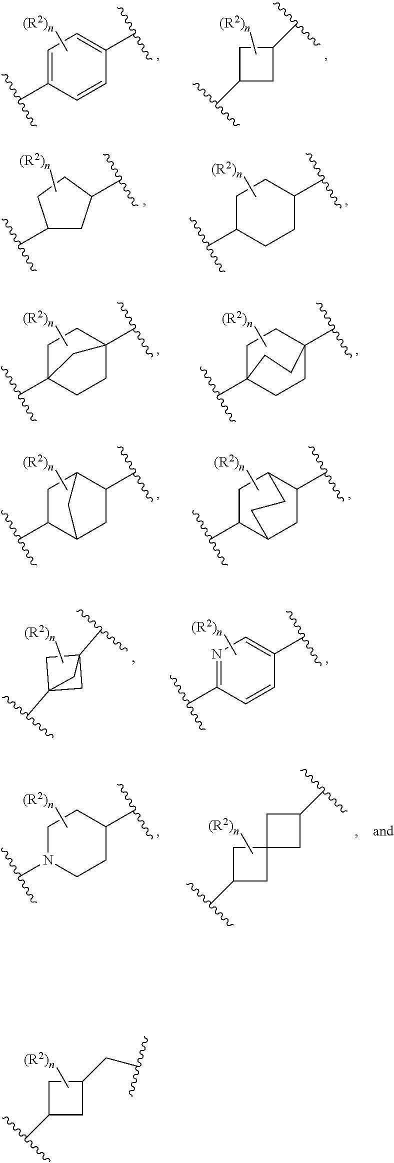

wherein all the symbols represent the same meanings as described above.

[7] The compound or a salt thereof according to item [1] or [2], wherein the compound is a compound represented by the following general formula (I-1),

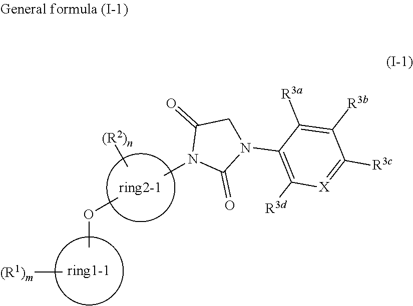

wherein the moiety

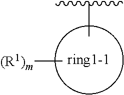

represents

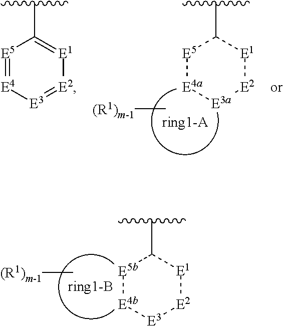

wherein all the symbols represent the same meanings as described in item [3], and the moiety

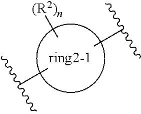

represents any one of the following

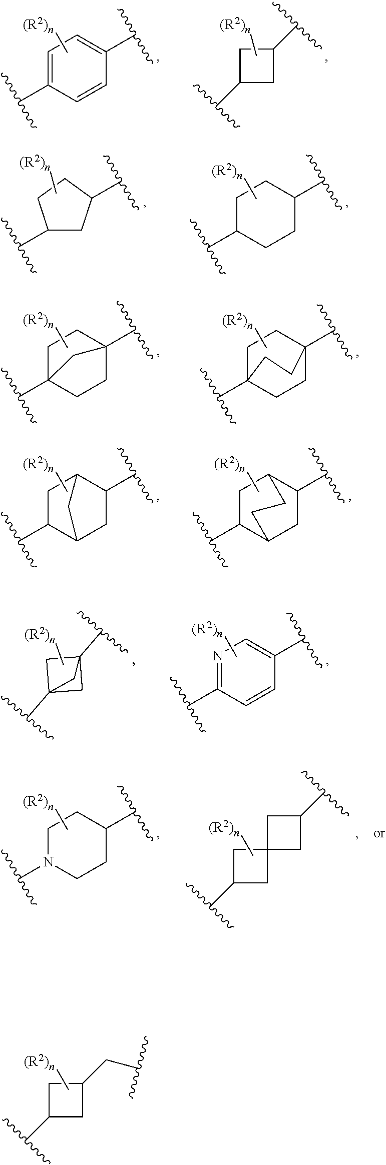

wherein all the symbols represent the same meanings as described in item [1] or [2], and other symbols represent the same meanings as described in items [1] to [6].

[8] The compound or a salt thereof according to item [1], wherein the compound is a compound represented by the following general formula (I-3),

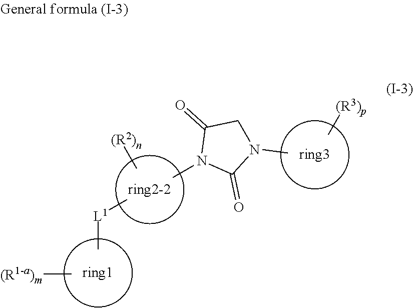

wherein L¹ represents (1) CR¹¹R¹², (2) C(═O), (3) O, (4) NR¹³, or (5) optionally oxidized S, wherein R¹¹ and R¹² each independently represent (1) a hydrogen atom or (2) a C1-6 alkyl group, and R¹³ represents (1) a hydrogen atom or (2) a C1-6 alkyl group, ring 2-2 represents (1) a C3-7 monocyclic carbocycle, (2) five- to seven-membered nitrogen-containing monocyclic heterocycle, (3) a C5-8 bridged carbocycle, or

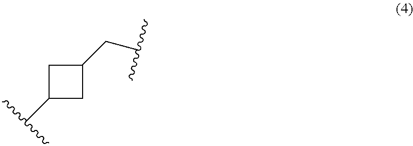 [SMILES: *CC1CC(*)C1]

and other symbols represent the same meanings as described in item [1].

[9] The compound or a salt thereof according to item [8], wherein the moiety

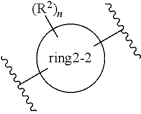

is selected from

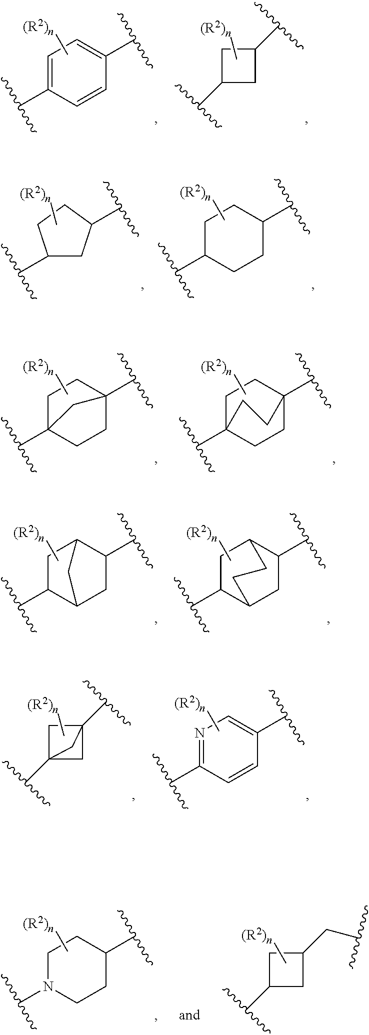

wherein all the symbols represent the same meanings as described in item [1].

[10] The compound or a salt thereof according to items [1] or [9], wherein the compound is a compound represented by the following general formula (I-4),

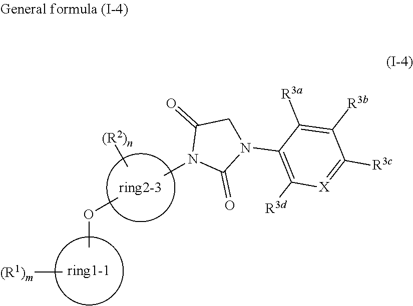

the moiety

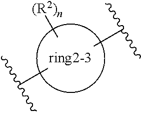

represents any one of the following

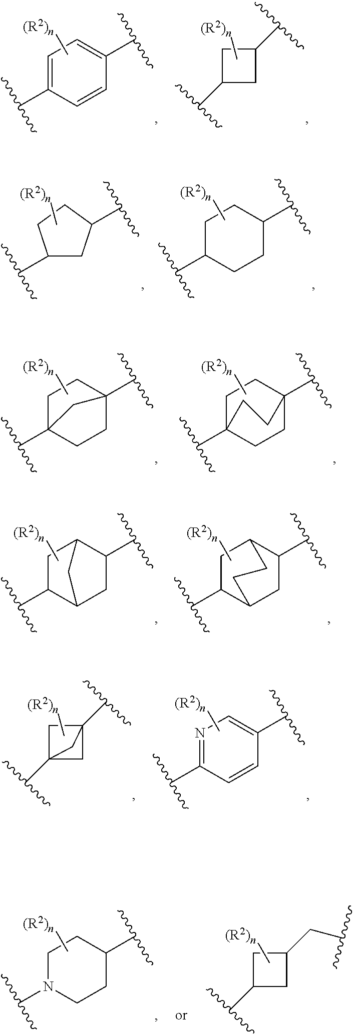

wherein all the symbols represent the same meanings as described in item [1], and other symbols represent the same meanings as described in item [7].

[11] The compound or a salt thereof according to item [1], wherein the compound is a compound represented by the following general formula (I-5),

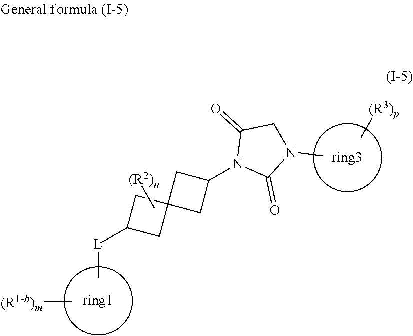

wherein R^{1-b} represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—C1-6 alkylene)-NR⁴R⁵, (9) a five- to seven-membered monocyclic heterocyclyl, (10) NR⁴R⁵, (11) a cyano group, (12) a hydroxyl group, (13) an oxo group, (14) a C1-6 alkylsulfonyl group, (15) a Q-(C3-7 monocyclic carbocyclyl) group, or (16) a Q-(three- to seven-membered monocyclic heterocyclyl) group,

when R^{1-b} represents any of (2) to (10), (14), (15) or (16), R^{1-b} may be substituted with one or more substituents selected from a halogen atom, a C1-6 alkyl group, a C1-6 haloalkyl group, a C1-6 hydroxyalkyl group, a hydroxyl group, a cyano group, and an oxo group,

when m is 2 or more, the plurality of R^{1-b} may be the same or different, and, two R^{1-b} may form a five- to seven-membered monocyclic ring with atoms they are attached thereto, and other symbols represent the same meanings as described in item [1].

[12] The compound or a salt thereof according to item [7] or [10], wherein the moiety

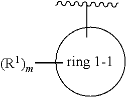

is

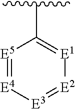

wherein all the symbols represent the same meanings as described in item [3].

[13] The compound or a salt thereof according to item [7] or [10], wherein the moiety

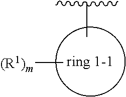

is

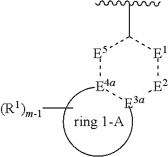

wherein all the symbols represent the same meanings as described in item [3].

[14] The compound or a salt thereof according to item [7] or [10], wherein the moiety

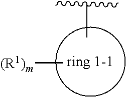

is

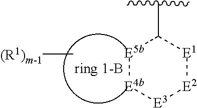

wherein all the symbols represent the same meanings as described in item [3].

[15] The compound or a salt thereof according to item [7], wherein the compound is a compound represented by the following general formula (I-1),

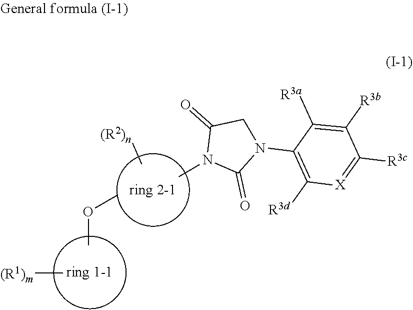

wherein the moiety

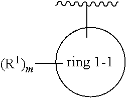

represents

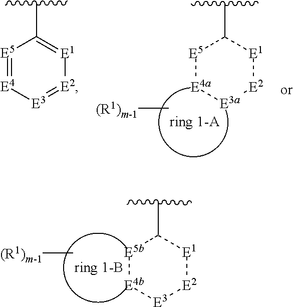

wherein

E¹, E², E³, E⁴, and E⁵ each independently represent (1) CH, (2) CR¹, or (3) N, wherein at least one of E, E², E³, E⁴, and E⁵ represent N, and

E^{3a} and E^{4a} each independently represent (1) C or (2) N, wherein at least one of E, E², E^{3a}, E^{4a}, and E⁵ represent N,

ring 1-A, including E^{3a} and E^{4a}, represent a five- to seven-membered monocyclic ring group,

E^{4b} and E^{5b} each independently represent (1) C or (2) N, wherein at least one of E¹, E², E³, E^{4b}, and E^{5b} represent N,

ring 1-B, including E^{4b} and E^{5b}, represents a five- to seven-membered monocyclic ring group, and

the broken-line bonds represent aromatic bonds,

R¹ represents (1) a halogen atom, (2) a C1-6 alkyl group, (3) a C1-6 alkoxy group, or (10) NR⁴R⁵,

when R¹ represents any of (2) to (3), R¹ may be substituted with one hydroxyl group,
R⁴ and R each independently represent (1) a hydrogen atom, or (2) a C1-6 alkyl group,

m−1 represents an integer of 0 to 5, and

when m is 2 or more, the plurality of R¹ may be the same or different,

the moiety

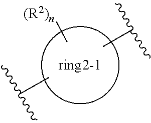

represents any one of the following

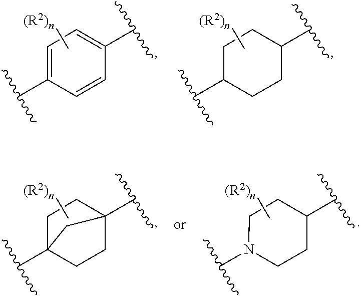

R² represents (2) a C1-6 alkyl group, or (4) a phenyl,

n represents an integer of 0 to 5,

when n is 2 or more, the plurality of R² may be the same or different,

X represents CR^{3e} or N,

R^{3a}, R^{3bc}, R^{3d}, and R^{3e} each independently represent (1) a hydrogen atom, or (2) a halogen atom,

R^{3b} represents (1) a C1-6 alkyl group, (2) a C1-6 alkoxy group, or (7) a C1-6 alkylsulfonyl group, and, when R^{3b} represents a C1-6 alkyl group or a C1-6 alkoxy group, the C1-6 alkyl group or a C1-6 alkoxy group may be substituted with 1 to 5 halogen atom.

[16] The compound or a salt thereof according to item [7], wherein the compound is

a compound represented by the following general formula (I-1),

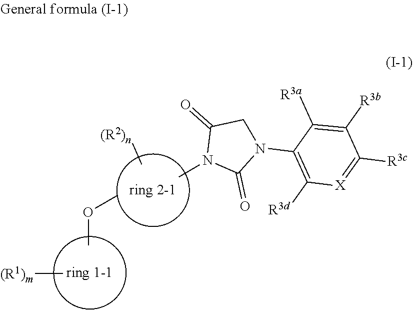

wherein the moiety

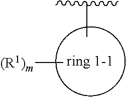

represents

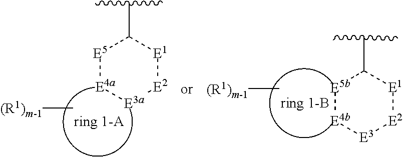

wherein

E¹, E², E³, and E⁵ each independently represent (1) CH, or (3) N, and

E^{3a} and E⁴a each independently represent C, wherein at least one of E¹, E², and E⁵ represent N,

ring 1-A, including E^{3a} and E^{4a}, represent a five-membered monocyclic ring group,

E^{4b} and E^{5b} each independently represent C, wherein at least one of E, E², E³ represent N,

ring 1-B, including E^{4b} and E^{5b}, represents a five-membered monocyclic ring group, and

the broken-line bonds represent aromatic bonds,

R¹ represents (1) a halogen atom, (2) a C1-6 alkyl group, or (3) a C1-6 alkoxy group,

when R¹ represents any of (2) to (3), R¹ may be substituted with one hydroxyl group,
m−1 represents an integer of 0 to 5, and

when m is 2 or more, the plurality of R¹ may be the same or different,

the moiety

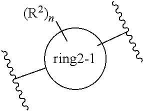

represents any one of the following

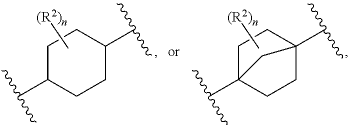

R² represents H,

n represents 0,

X represents N,

R^{3a}, R^{3c}, and R^{3d} each independently represent a hydrogen atom,

R^{3b} represents (1) a C1-6 alkyl group, and, the C1-6 alkyl group may be substituted with 1 to 5 halogen atom.

[17] The compound or a salt thereof according to item [7], wherein the compound is a compound represented by the following general formula (I-1),

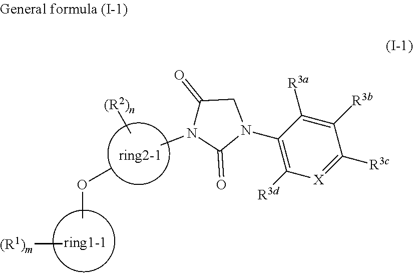

wherein the moiety

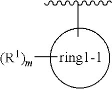

represents

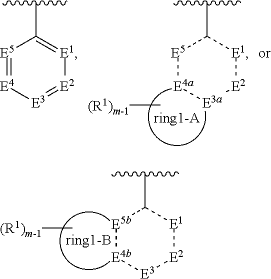

wherein

E¹, E², E³, E⁴, and E⁵ each independently represent (1) CH, (2) CR^{I}, or (3) N, wherein at least one of E¹, E², E³, E⁴, and E⁵ represent N, and

E^{3a} and E^{4a} each independently represent (1) C or (2) N, wherein at least one of E¹, E², E^{3a}, E^{4a}, and E⁵ represent N,

ring 1-A, including E^{3a} and E^{4a}, represent a five- to seven-membered monocyclic ring group,

E^{4b} and E^{5b} each independently represent (1) C or (2) N, wherein at least one of E¹, E², E³, E^{4b}, and E^{5b} represent N,

ring 1-B, including E^{4b} and E^{5b}, represents a five- to seven-membered monocyclic ring group, and

the broken-line bonds represent aromatic bonds,

R¹ represents (3) a C1-6 alkoxy group, or (10) NR⁴R⁵,
R⁴ and R⁵ each independently represent (1) a hydrogen atom, or (2) a C1-6 alkyl group,

m−1 represents an integer of 0 to 5, and

when m is 2 or more, the plurality of R¹ may be the same or different,

the moiety

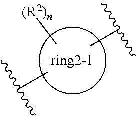

represents any one of the following

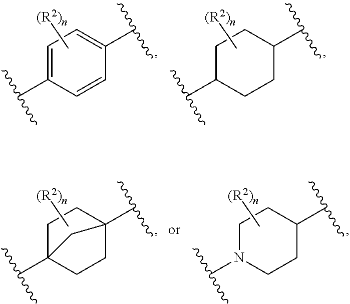

R² represents (2) a C1-6 alkyl group, or (4) a phenyl,

n represents an integer of 0 to 10,

when n is 2 or more, the plurality of R² may be the same or different,

X represents CR^{3e} or N,

R^{3a}, R^{3c}, R^{3d}, and R^{3e} each independently represent (1) a hydrogen atom, or (2) a halogen atom,

R^{3b} represents (1) a C1-6 alkyl group, or (2) a C1-6 alkoxy group, and, when R^{3b} represents a C1-6 alkyl group or a C1-6 alkoxy group, the C1-6 alkyl group or a C1-6 alkoxy group may be substituted with 1 to 5 halogen atom.

[18] The compound or a salt thereof according to any one of items [1] to [17], wherein the compound is (1) 3-{4-[(2-amino-5-pyrimidinyl)oxy]phenyl}-1-[5-(trifluoromethyl)-3-pyridinyl]-2,4-imidazolidinedione,
(2) 3-[4-(1H-pyrazolo[3,4-b]pyridin-5-yloxy)phenyl]-1-[5-(trifluoromethyl)-3-pyridinyl]-2,4-imidazolidinedione,
(3) 1-[3-(methylsulfonyl)-5-(trifluoromethyl)phenyl]-3-[4-(1H-pyrazolo[3,4-b]pyridin-5-yloxy)phenyl]-2,4-imidazolidinedione,
(4) 3-{4-[(2-amino-4-pyrimidinyl)oxy]-2-methylphenyl}-1-[5-(trifluoromethyl)-3-pyridinyl]-2,4-imidazolidinedione,
(5) 3-{trans-4-[(7-methoxy-4-quinazolinyl)oxy]cyclohexyl}-1-[5-(trifluoromethyl)-3-pyridinyl]-2,4-imidazolidinedione,
(6) 1-[3-fluoro-5-(trifluoromethyl)phenyl]-3-[trans-4-(thieno[3,2-b]pyridin-6-yloxy)cyclohexyl]-2,4-imidazolidinedione,
(7) 3-{3-isopropyl-4-[(2-oxo-2,3-dihydro-1H-imidazo[4,5-b]pyridin-7-yl)oxy]phenyl}-1-[5-(trifluoromethyl)-3-pyridinyl]-2,4-imidazolidinedione,
(8) 3-[trans-4-(7H-pyrrolo[2,3-d]pyrimidin-4-yloxy)cyclohexyl]-1-[5-(trifluoromethyl)-3-pyridinyl]-2,4-imidazolidinedione,
(9) 3-[3-methyl-4-(pyrazolo[1,5-a]pyrimidin-7-yloxy)phenyl]-1-[5-(trifluoromethyl)-3-pyridinyl]-2,4-imidazolidinedione,
(10) 3-[4-(7H-pyrrolo[2,3-d]pyrimidin-4-yloxy)bicyclo[2.2.1]hept-1-yl]-1-[5-(trifluoromethyl)-3-pyridinyl]-2,4-imidazolidinedione,
(11) 3-{4-[(2-amino-4-pyrimidinyl)oxy]-2-methylphenyl}-1-[4-fluoro-3-(trifluoromethoxy)phenyl]-2,4-imidazolidinedione,
(12) 3-{4-[(2-amino-4-pyrimidinyl)oxy]-2-methylphenyl}-1-[3-fluoro-5-(trifluoromethyl)phenyl]-2,4-imidazolidinedione,
(13) 3-{5-[(2-amino-4-pyrimidinyl)oxy]-2-biphenylyl}-1-[5-(trifluoromethyl)-3-pyridinyl]-2,4-imidazolidinedione,
(14) 3-[3-ethyl-4-(1H-pyrrolo[2,3-b]pyridin-4-yloxy)phenyl]-1-[5-(trifluoromethyl)-3-pyridinyl]-2,4-imidazolidinedione,
(15) 1-{4-[(2-amino-4-pyrimidinyl)oxy]-3-ethylphenyl}-3-[3-(trifluoromethyl)phenyl]-2-imidazolidinone,
(16) 3-{1-[(7-methoxy-4-quinolinyl)methyl]-4-piperidinyl}-1-[5-(trifluoromethyl)-3-pyridinyl]-2,4-imidazolidinedione, or
(17) 3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione.

[19] The compound or a salt thereof according to any one of items [1] to [17], wherein the compound is (1) 3-(trans-4-{[3-(2-hydroxyethoxy)-1H-pyrazolo[3,4-b]pyridin-5-yl]oxy}cyclohexyl)-1-[5-(trifluoromethyl)-3-pyridinyl]-2,4-imidazolidinedione,
(2) 3-(4-{[6-(2-hydroxy-2-propanyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione,
(3) 3-{4-[(5-fluoro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(4) 3-{4-[(5-chloro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(5) 5-{2,4-dioxo-3-[4-(7H-pyrrolo[2,3-d]pyrimidin-4-yloxy)bicyclo[2.2.1]hept-1-yl]-1-imidazolidinyl}nicotinonitrile,
(6) 3-{2,4-dioxo-3-[4-(7H-pyrrolo[2,3-d]pyrimidin-4-yloxy)bicyclo[2.2.1]hept-1-yl]-1-imidazolidinyl}benzonitrile, or
(7) 3-[4-(1H-pyrazolo[3,4-b]pyridin-4-yloxy)bicyclo[2.2.1]hept-1-yl]-1-[5-(trifluoromethyl)-3-pyridinyl]-2,4-imidazolidinedione.

[20] A pharmaceutical composition comprising the compound of any one of items [1] to [19], or a salt thereof.

[21] The pharmaceutical composition according to item [20], which comprises a DDR1 inhibitor.

[22] The pharmaceutical composition according to item [20], which is a preventive and/or therapeutic agent for a DDR1-related disease.

[23] The pharmaceutical composition according to item [22], wherein the DDR1-related disease is a cancer, a kidney disease, a cardiovascular disease, a central nervous system disease, or fibrosis.

[24] The pharmaceutical composition according to item [22], wherein the DDR1-related disease is a cancer, and the cancer is lung cancer, breast cancer, esophageal cancer, head and neck cancer, liver cancer, prostate cancer, lymphoma, leukemia, melanoma, pancreas cancer, neuroblastoma, glioma, colon cancer, renal cell carcinoma, thyroid cancer, stomach cancer, bladder cancer, ovarian cancer, endometrial cancer, brain tumor, or sarcoma.

[25] The pharmaceutical composition according to item [22], wherein the DDR1-related disease is a kidney disease, and the kidney disease is acute kidney failure, chronic kidney failure, renal fibrosis, diabetic nephropathy, membranoproliferative glomerulonephritis, mesangial proliferative nephritis, interstitial nephritis, lupus nephritis, amyloid kidney, pyelonephritis, crescentic glomerulonephritis, glomerular nephritis, membranous nephropathy, IgA nephropathy, focal glomerulosclerosis, hypertensive nephrosclerosis, Alport syndrome, Goodpasture's syndrome, or nephrotic nephritis.

[26] A method for preventing and/or treating a DDR1-related disease, comprising administering an effective amount of the compound of any one of items [1] to [19] or a salt thereof to a mammal.

[27] A compound of any one of items [1] to [19] or a salt thereof for use in prevention and/or treatment of a DDR1-related disease.

[28] Use of a compound of any one of items [1] to [19] or a salt thereof for production of a preventive and/or therapeutic agent for a DDR1-related disease.

### Advantageous Effects of Invention

The present compound has prominent DDR1 inhibitory activity, has a DDR1-selective inhibitory activity to a specific kinase, and a pharmaceutical composition containing the present compound can be used for the prevention and/or treatment of a DDR1-related disease.

### DESCRIPTION OF EMBODIMENTS

The present invention is described below in detail.

In the present invention, examples of the halogen atom include a fluorine atom, a chlorine atom, a bromine atom, and an iodine atom.

In the present invention, examples of the C1-6 alkyl group include linear or branched C1-6 alkyl chains, including, for example, methyl, ethyl, propyl, isopropyl, butyl, isobutyl, sec-butyl, tert-butyl, pentyl, hexyl, and isomers thereof.

In the present invention, examples of the C1-6 haloalkyl group include C1-6 alkyl group substituted with at least one halogen atom, including, for example, fluoromethyl, chloromethyl, bromomethyl, iodomethyl, difluoromethyl, trifluoromethyl, 1-fluoroethyl, 2-fluoroethyl, 2-chloroethyl, pentafluoroethyl, 1-fluoropropyl, 2-chloropropyl, 3-fluoropropyl, 3-chloropropyl, 4,4,4-trifluorobutyl, and 4-bromobutyl.

In the present invention, examples of the C1-6 alkoxy group include linear or branched alkyl chains, including, for example, methoxy, ethoxy, propoxy, isopropoxy, butoxy, isobutoxy, sec-butoxy, tert-butoxy, pentoxy, hexoxy, and isomers thereof.

In the present invention, examples of the C1-6 alkylene include linear or branched C1-6 alkyl chains, including, for example, methylene, ethylene, propylene, isopropylene, butylene, isobutylene, sec-butylene, tert-butylene, pentylene, hexylene, and isomers thereof.

In the present invention, examples of the C2-6 alkenyl group include vinyl, propenyl, butenyl, pentenyl, hexenyl, and isomers thereof.

In the present invention, examples of the C2-6 alkynyl group include ethynyl, propynyl, butynyl, pentynyl, hexynyl, and isomers thereof.

In the present invention, examples of the C1-6 alkylsulfonyl group include methylsulfonyl, ethylsulfonyl, propylsulfonyl, isopropylsulfonyl, butylsulfonyl, isobutylsulfonyl, sec-butylsulfonyl, tert-butylsulfonyl, pentylsulfonyl, hexylsulfonyl, and isomers thereof.

In the present invention, examples of the C1-6 hydroxyalkyl group include C1-6 alkyl substituted with at least one hydroxyl group, including, for example, hydroxymethyl, 1-hydroxyethyl, 2-hydroxyethyl, 1-hydroxypropyl, 2-hydroxypropyl, 3-hydroxypropyl, 1-hydroxybutyl, 2-hydroxybutyl, 3-hydroxybutyl, 4-hydroxybutyl, hydroxypentyl, hydroxyhexyl, and isomers thereof.

In the present invention, examples of the C3-6 cycloalkyl group include cyclopropyl, cyclobutyl, cyclopentyl, and cyclohexyl.

In the present invention, examples of the three- to six-membered saturated heterocycle include aziridine, oxirane, thiirane, azetidine, oxetane, thiethane, pyrrolidine, tetrahydrofuran, tetrahydrothiophene, piperidine, tetrahydropyran, tetrahydrothiopyran, imidazolidine, pyrazolidine, tetrahydrooxazole, tetrahydroisoxazole, tetrahydrothiazole, tetrahydroisothiazole, dioxolan, dithiolane, morpholine, thiomorpholine, perhydropyrimidine, and perhydropyridazine rings.

In the present invention, examples of the C3-7 monocyclic carbocycle include cyclopropane, cyclobutane, cyclopentane, cyclohexane, cycloheptane, cyclobutene, cyclopentene, cyclohexene, cycloheptene, cyclobutadiene, cyclopentadiene, cyclohexadiene, cycloheptadiene, and benzene rings.

In the present invention, examples of the three- to seven-membered monocyclic heterocycle include aziridine, oxirane, thiirane, azetidine, oxetane, thiethane, pyrrole, oxazole, isoxazole, thiazole, isothiazole, pyrroline, pyrrolidine, dihydrooxazole, tetrahydrooxazole, dihydroisoxazole, tetrahydroisoxazole, dihydrothiazole, tetrahydrothiazole, dihydroisothiazole, tetrahydroisothiazole, imidazole, pyrazole, furazan, oxadiazole, thiadiazole, imidazoline, imidazolidine, pyrazoline, pyrazolidine, dihydrofurazan, tetrahydrofurazan, dihydrooxadiazole, tetrahydrooxadiazole, dihydrothiadiazole, tetrahydrothiadiazole, triazole, triazoline, triazolidine, tetrazole, tetrazoline, tetrazolidine, furan, dihydrofuran, tetrahydrofuran, dioxolan, thiophene, dihydrothiophene, tetrahydrothiophene, dithiolane, pyridine, oxazine, thiazine, dihydropyridine, tetrahydropyridine, piperidine, dihydrooxazine, tetrahydrooxazine, dihydrothiazine, tetrahydrothiazine, morpholine, thiomorpholine, pyrazine, pyrimidine, pyridazine, oxadiazine, thiadiazine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydrooxadiazine, tetrahydrooxadiazine, dihydrothiadiazine, tetrahydrothiadiazine, pyran, dihydropyran, tetrahydropyran, oxathiane, dioxane, thiopyran, dihydrothiopyran, tetrahydrothiopyran, dithiane, azepine, diazepine, oxepin, thiepine, oxazepine, oxadiazepine, thiazepine, thiadiazepine, dihydroazepine, tetrahydroazepine, perhydroazepine, dihydrodiazepine, tetrahydrodiazepine, perhydrodiazepine, dihydrooxepin, tetrahydrooxepin, perhydrooxepin, dihydrothiepine, tetrahydrothiepine, perhydrothiepine, dihydrooxazepine, tetrahydrooxazepine, perhydrooxazepine, dihydrooxadiazepine, tetrahydrooxadiazepine, perhydrooxadiazepine, dihydrothiazepine, tetrahydrothiazepine, perhydrothiazepine, dihydrothiadiazepine, tetrahydrothiadiazepine, and perhydrothiadiazepine rings.

In the present invention, examples of the five- to seven-membered nitrogen-containing monocyclic heterocycle include pyrrole, oxazole, isoxazole, thiazole, isothiazole, pyrroline, pyrrolidine, dihydrooxazole, tetrahydrooxazole, dihydroisoxazole, tetrahydroisoxazole, dihydrothiazole, tetrahydrothiazole, dihydroisothiazole, tetrahydroisothiazole, imidazole, pyrazole, furazan, oxadiazole, thiadiazole, imidazoline, imidazolidine, pyrazoline, pyrazolidine, dihydrofurazan, tetrahydrofurazan, dihydrooxadiazole, tetrahydrooxadiazole, dihydrothiadiazole, tetrahydrothiadiazole, triazole, triazoline, triazolidine, tetrazole, tetrazoline, tetrazolidine, pyridine, oxazine, thiazine, dihydropyridine, tetrahydropyridine, piperidine, dihydrooxazine, tetrahydrooxazine, dihydrothiazine, tetrahydrothiazine, morpholine, thiomorpholine, pyrazine, pyrimidine, pyridazine, oxadiazine, thiadiazine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydrooxadiazine, tetrahydrooxadiazine, dihydrothiadiazine, tetrahydrothiadiazine, azepine, diazepine, oxazepine, oxadiazepine, thiazepine, thiadiazepine, dihydroazepine, tetrahydroazepine, perhydroazepine, dihydrodiazepine, tetrahydrodiazepine, perhydrodiazepine, dihydrooxazepine, tetrahydrooxazepine, perhydrooxazepine, dihydrooxadiazepine, tetrahydrooxadiazepine, perhydrooxadiazepine, dihydrothiazepine, tetrahydrothiazepine, perhydrothiazepine, dihydrothiadiazepine, tetrahydrothiadiazepine, and perhydrothiadiazepine rings.

In the present invention, the five- to seven-membered monocyclic ring group means C5-7 monocyclic carbocycle or five- to seven-membered monocyclic heterocycle.

In the present invention, examples of the C5-7 monocyclic carbocycle include cyclopentane, cyclohexane, cycloheptane, cyclopentene, cyclohexene, cycloheptene, cyclopentadiene, cyclohexadiene, cycloheptadiene, and benzene rings.

In the present invention, examples of the five- to seven-membered monocyclic heterocycle or the five- to seven-membered monocyclic heterocycle containing 1 to 4 heteroatoms selected from N, O, and optionally oxidized S include pyrrole, oxazole, isoxazole, thiazole, isothiazole, pyrroline, pyrrolidine, dihydrooxazole, tetrahydrooxazole, dihydroisoxazole, tetrahydroisoxazole, dihydrothiazole, tetrahydrothiazole, dihydroisothiazole, tetrahydroisothiazole, imidazole, pyrazole, furazan, oxadiazole, thiadiazole, imidazoline, imidazolidine, pyrazoline, pyrazolidine, dihydrofurazan, tetrahydrofurazan, dihydrooxadiazole, tetrahydrooxadiazole, dihydrothiadiazole, tetrahydrothiadiazole, triazole, triazoline, triazolidine, tetrazole, tetrazoline, tetrazolidine, furan, dihydrofuran, tetrahydrofuran, dioxolan, thiophene, dihydrothiophene, tetrahydrothiophene, dithiolane, pyridine, oxazine, thiazine, dihydropyridine, tetrahydropyridine, piperidine, dihydrooxazine, tetrahydrooxazine, dihydrothiazine, tetrahydrothiazine, morpholine, thiomorpholine, pyrazine, pyrimidine, pyridazine, oxadiazine, thiadiazine, dihydropyrazine, tetrahydropyrazine, piperazine, dihydropyrimidine, tetrahydropyrimidine, perhydropyrimidine, dihydropyridazine, tetrahydropyridazine, perhydropyridazine, dihydrooxadiazine, tetrahydrooxadiazine, dihydrothiadiazine, tetrahydrothiadiazine, pyran, dihydropyran, tetrahydropyran, oxathiane, dioxane, thiopyran, dihydrothiopyran, tetrahydrothiopyran, dithiane, azepine, diazepine, oxepin, thiepine, oxazepine, oxadiazepine, thiazepine, thiadiazepine, dihydroazepine, tetrahydroazepine, perhydroazepine, dihydrodiazepine, tetrahydrodiazepine, perhydrodiazepine, dihydrooxepin, tetrahydrooxepin, perhydrooxepin, dihydrothiepine, tetrahydrothiepine, perhydrothiepine, dihydrooxazepine, tetrahydrooxazepine, perhydrooxazepine, dihydrooxadiazepine, tetrahydrooxadiazepine, perhydrooxadiazepine, dihydrothiazepine, tetrahydrothiazepine, perhydrothiazepine, dihydrothiadiazepine, tetrahydrothiadiazepine, and perhydrothiadiazepine rings.

In the present invention, examples of the eight- to ten-membered nitrogen-containing bicyclic heterocycle include thienopyrazole, thienoimidazole, pyrazolothiazole, indole, isoindole, indolizine, indazole, purine, pteridin, benzooxazole, benzothiazole, benzoimidazole, imidazopyridine, imidazopyrimidine, imidazopyridazine, thienopyridine, pyrrolopyridine, pyrrolopyrimidine, pyrrolopyridazine, pyrazolopyridine, pyrazolopyrimidine, pyridopyrazine, benzofurazan, benzothiadiazole, benzotriazole, indoline, isoindoline, dihydroimidazopyridine, dihydropyrrolopyridine, dihydroindazole, perhydroindazole, dihydrobenzooxazole, perhydrobenzooxazole, dihydrobenzothiazole, perhydrobenzothiazole, dihydrobenzoimidazole, perhydrobenzoimidazole, dihydropyridopyrazine, tetrahydropyridopyrazine, pyridooxazine, dihydropyridooxazine, quinoline, isoquinoline, quinolizine, phthalazine, pteridin, naphthyridine, quinoxaline, quinazoline, cinnoline, dihydroquinoline, tetrahydroquinoline, perhydroquinoline, dihydroisoquinoline, tetrahydroisoquinoline, perhydroisoquinoline, dihydropteridin, tetrahydropteridin, dihydrophthalazine, tetrahydrophthalazine, perhydrophthalazine, dihydronaphthyridine, tetrahydronaphthyridine, perhydronaphthyridine, dihydroquinoxaline, tetrahydroquinoxaline, perhydroquinoxaline, dihydroquinazoline, tetrahydroquinazoline, perhydroquinazoline, dihydrocinnoline, tetrahydrocinnoline, perhydrocinnoline, dihydrobenzooxazine, dihydrobenzothiazine, and pyrazinomorpholine rings.

In the present invention, examples of the C5-8 bridged carbocycle include bicyclo[1.1.1]pentane, bicyclo[2.1.1]hexane, bicyclo[2.1.1]hexene, bicyclo[2.2.1]heptane, bicyclo[2.2.1]heptene, bicyclo[3.1.1]heptane, bicyclo[3.1.1]heptene, bicyclo[2.2.2]octane, bicyclo[2.2.2]octene, bicyclo[3.2.1]octane, and bicyclo[3.2.1]octene rings.

In the present invention, the ring 1 or ring 1-1 is preferably

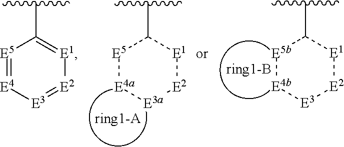

wherein all the symbols represent the same meanings as described herein.

More preferably, the ring 1 or ring 1-1 is

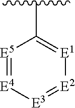

wherein all the symbols represent the same meanings as described herein.

Even more preferably, the ring 1 or ring 1-1 is

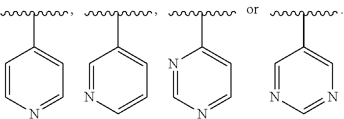

In the present invention, preferred as

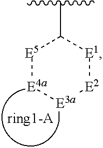

is any of the ring structures represented by the following formulae:

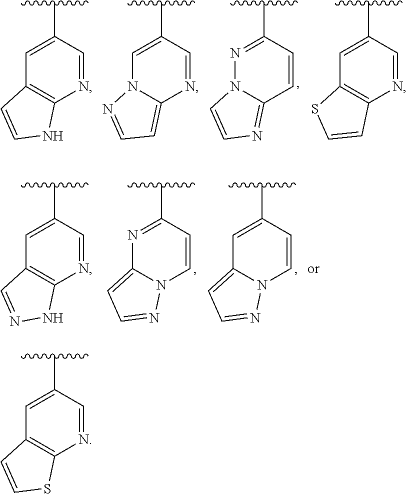

In the present invention, preferred as

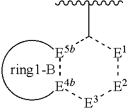

is any of the ring structures represented by the following formulae.

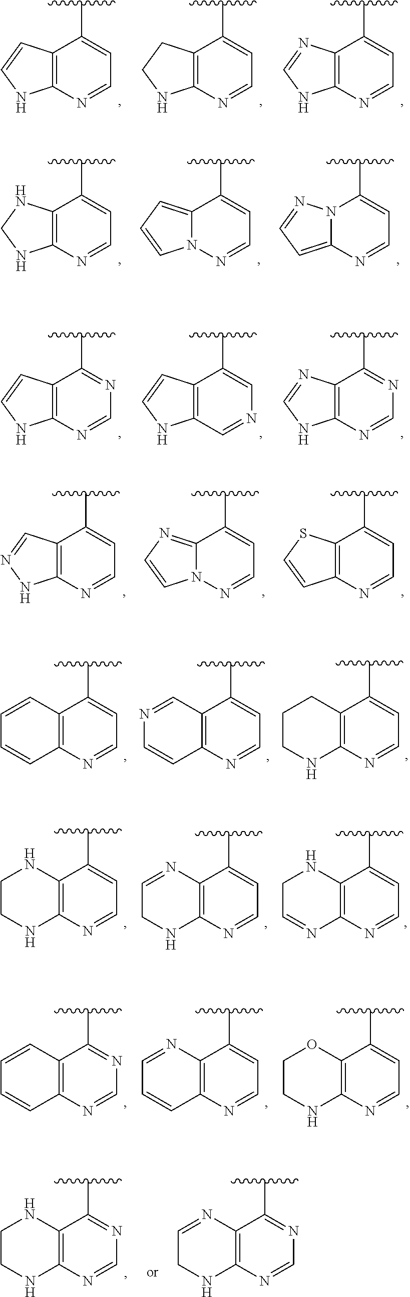

In the present invention, more preferred as

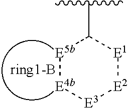

is any of the ring structures represented by the following formulae.

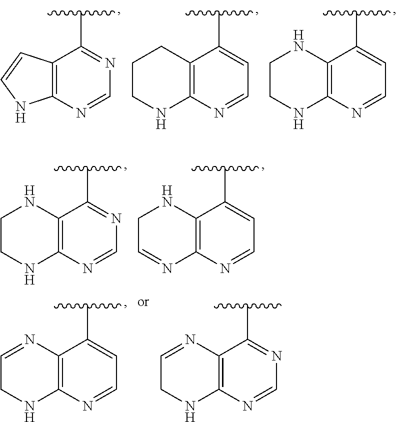

In the present invention, R^{1-a} is preferably a halogen atom, an optionally substituted C1-6 alkyl group, an optionally substituted C1-6 alkoxy group, an optionally substituted C3-7 monocyclic carbocyclyl, an optionally substituted five- to seven-membered monocyclic heterocyclyl, NR⁴R⁵, C(═O)NR⁴R⁵, a cyano group, a hydroxyl group, or an oxo group. More preferably, R^{1-a} is a halogen atom, an optionally (preferably halogen) substituted C1-6 alkyl group, an optionally (preferably halogen) substituted C1-6 alkoxy group, NR⁴R⁵, or an oxo group.

In the present invention, R¹ is preferably a halogen atom, an optionally substituted C1-6 alkyl group, an optionally substituted C1-6 alkoxy group, an optionally substituted C3-7 monocyclic carbocyclyl, an optionally substituted five- to seven-membered monocyclic heterocyclyl, NR⁴R⁵, C(═O)NR⁴R⁵, a cyano group, a hydroxyl group, or an oxo group. More preferably, R¹ is a halogen atom, an optionally (preferably halogen) substituted C1-6 alkyl group, an optionally (preferably halogen) substituted C1-6 alkoxy group, NR⁴R⁵, or an oxo group.

In the present invention, R⁴ and R⁵ are each preferably hydrogen atom or an optionally (preferably halogen) substituted C1-6 alkyl group. More preferably, at least one of R⁴ and R⁵ is hydrogen atom, further preferably, both of R⁴ and R⁵ are hydrogen atoms.

In the present invention, L is preferably CR¹¹R¹², O, or NR¹³, more preferably O.

In the present invention, L¹ is preferably CR¹¹R¹², O, or NR¹³, more preferably O.

In the present invention, at least one of R¹¹ and R¹² is preferably hydrogen atom, more preferably both of R¹¹ and R¹² are hydrogen atoms.

In the present invention, R¹³ is preferably hydrogen atom.

In the present invention, preferred as

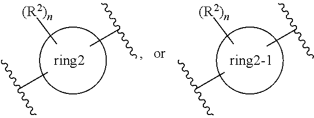

is any of the ring structures represented by the following formulae:

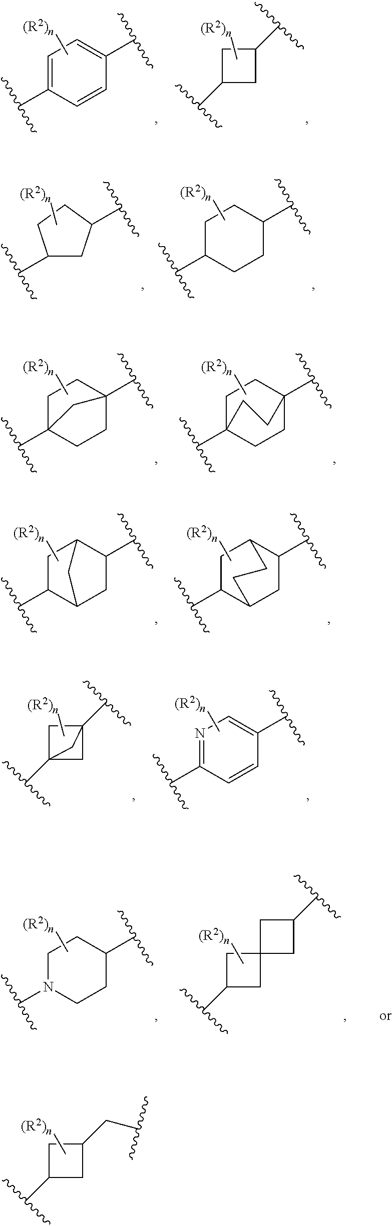

wherein all the symbols represent the same meanings as described herein.

In the present invention, more preferred as,

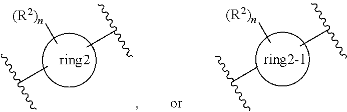

is any of the ring structures represented by the following formulae:

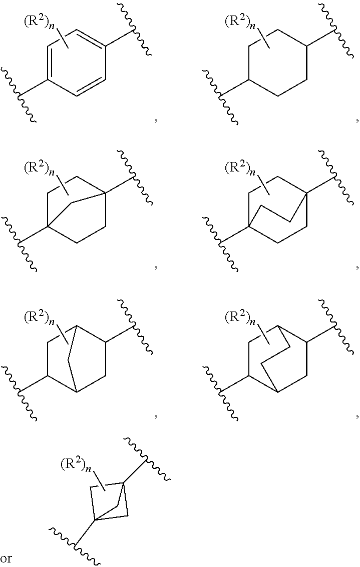

wherein all the symbols represent the same meanings as described herein.

In the present invention, preferred as

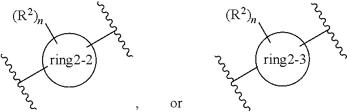

is any of the ring structures represented by the following formulae:

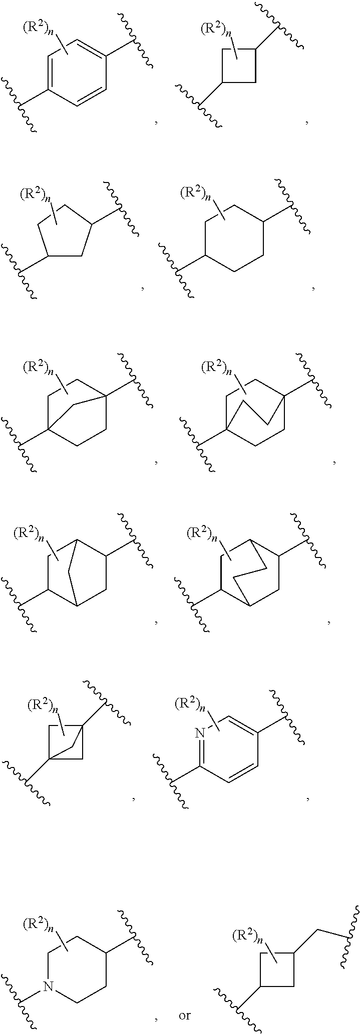

wherein all the symbols represent the same meanings as described herein.

In the present invention, more preferred as

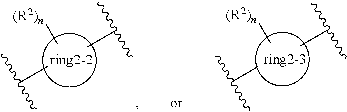

is any of the ring structures represented by the following formulae:

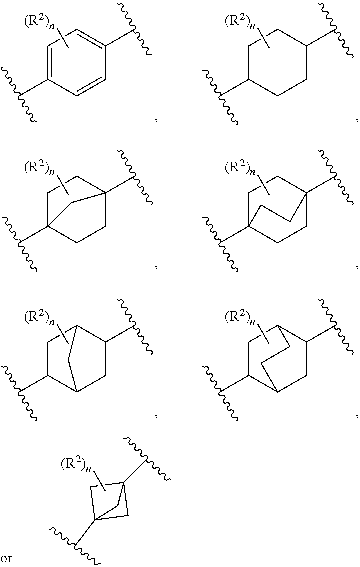

wherein all the symbols represent the same meanings as described herein.

In the present invention, R² is preferably a halogen atom, an optionally (preferably halogen) substituted C1-6 alkyl group, an optionally (preferably halogen) substituted C1-6 alkoxy group, an optionally (preferably halogen) substituted C2-6 alkenyl group, or an optionally (preferably halogen) substituted C2-6 alkynyl group. More preferably, R² is a halogen atom, an optionally (preferably halogen) substituted C1-6 alkyl group, or an optionally (preferably halogen) substituted C1-6 alkoxy group.

In the present invention, preferred as

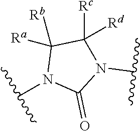

is

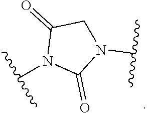 [SMILES: *N1CC(=O)N(*)C1=O]

In the present invention, preferred as

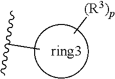

is

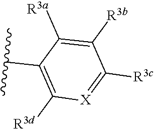

wherein all the symbols represent the same meanings as described herein.

In the present invention, R³ is preferably a halogen atom, an optionally (preferably halogen) substituted C1-6 alkyl group, an optionally (preferably halogen) substituted C1-6 alkoxy group, an optionally (preferably halogen) substituted C3-7 monocyclic carbocyclyl, a cyano group, a hydroxyl group, or an oxo group. More preferably, R³ is a halogen atom, an optionally (preferably halogen) substituted C1-6 alkyl group, or an optionally (preferably halogen) substituted C1-6 alkoxy group.

In the present invention, R^{3a}, R^{3c}, R^{3d}, and R³, are each preferably hydrogen atom or halogen atom.

In the present invention, R^{3b} is preferably an optionally (preferably halogen) substituted C1-6 alkyl group or an optionally (preferably halogen) substituted C1-6 alkoxy group.

In the present invention, m is preferably 0-5, more preferably 0-3.

In the present invention, n is preferably 0-5, more preferably 0-3.

In the present invention, p is preferably 0-5, more preferably 0-3.

In the present invention, the compound represented by general formula (I-a) or (1) is preferably a compound represented by the following general formula (I-A), (I-B), or (I-C):

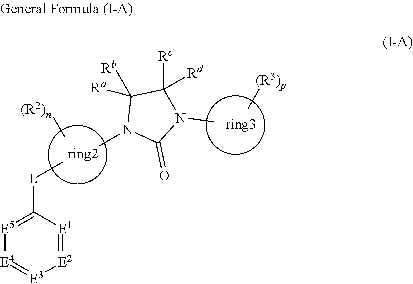

wherein all the symbols represent the same meanings as described herein.

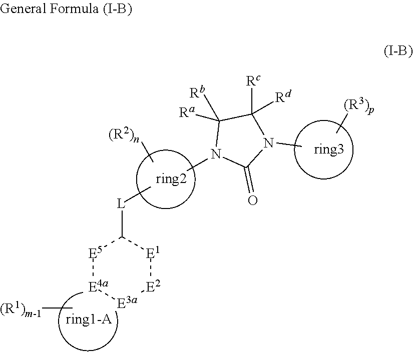

wherein all the symbols represent the same meanings as described herein.

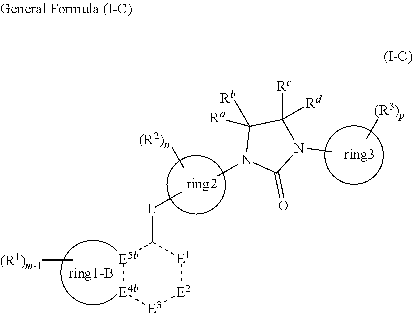

wherein all the symbols represent the same meanings as described herein.

The compound represented by general formula (I-A) is preferably a compound represented by the following general formula (I-A-1):

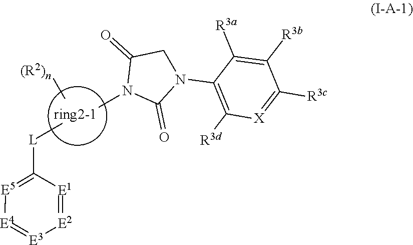

wherein all the symbols represent the same meanings as described herein.

The compound represented by general formula (I-A) is more preferably a compound represented by the following general formula (I-A-2):

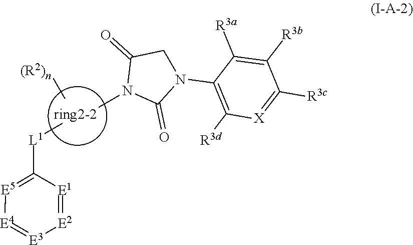

wherein all the symbols represent the same meanings as described herein.

The compound represented by general formula (I-B) is preferably a compound represented by the following general formula (I-B-1):

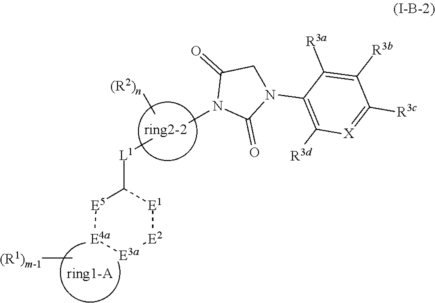

wherein all the symbols represent the same meanings as described herein.

The compound represented by general formula (I-B) is more preferably a compound represented by the following general formula (I-B-2):

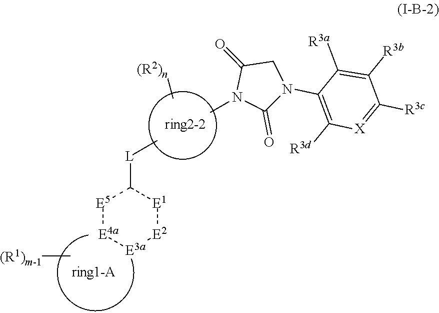

wherein all the symbols represent the same meanings as described herein.

The compound represented by general formula (I-C) is preferably a compound represented by the following general formula (I-C-1):

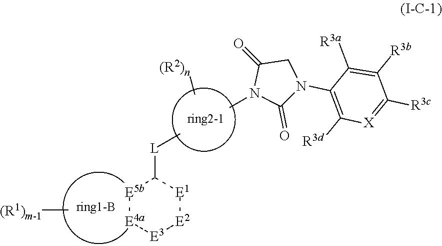

wherein all the symbols represent the same meanings as described herein.

The compound represented by general formula (I-C) is more preferably a compound represented by the following general formula (I-C-2):

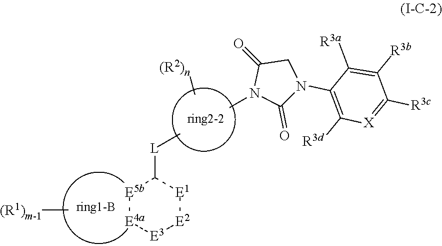

wherein all the symbols represent the same meanings as described herein.

In the present invention, the compound represented by general formula (I-a) or (I) is more preferably a compound represented by the following general formula (I-1):

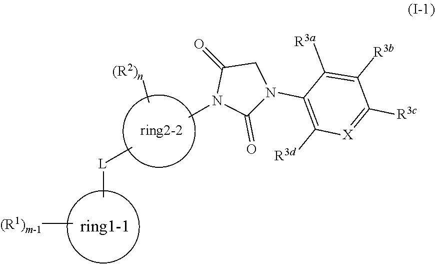

wherein all the symbols represent the same meanings as described herein.

Even more preferably, the compound represented by general formula (I-a) or (I) is a compound represented by the following general formula (I-2):

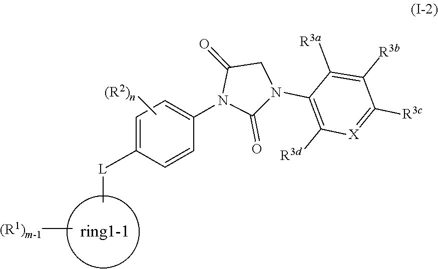

wherein all the symbols represent the same meanings as described herein.

In other embodiment, the compound represented by general formula (I-a) is preferably a compound represented by the following general formula (I-3):

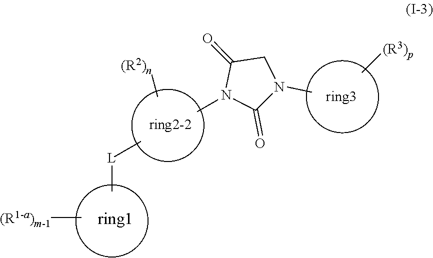

wherein all the symbols represent the same meanings as described herein.

In the present invention, the compound represented by general formula (I-a) or (I) is more preferably a compound represented by the following general formula (I-4):

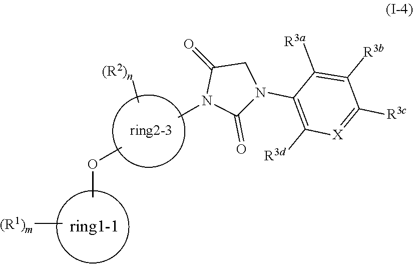

wherein all the symbols represent the same meanings as described herein.

In other embodiment, the compound represented by general formula (I-a) is

preferably a compound represented by the following general formula (I-5):

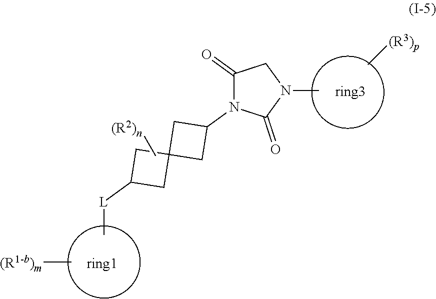

wherein all the symbols represent the same meanings as described herein.

In the present invention, the compound or a salt thereof is a compound represented by the following general formula (I-1),

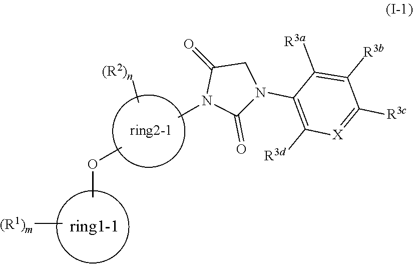

wherein the moiety

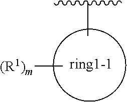

represents

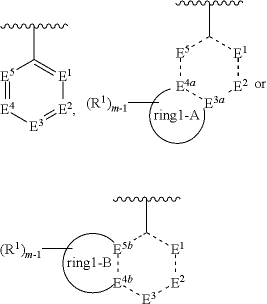

wherein

E¹, E², E³, E⁴, and E⁵ each independently represent (1) CH, (2) CR¹, or (3) N, wherein at least one of E¹, E², E³, E⁴, and E⁵ represent N, and

E^{3a} and E^{4a} each independently represent (1) C or (2) N, wherein at least one of E¹, E², E^{3a}, E^{4a}, and E⁵ represent N,

ring 1-A, including E^{3a} and E^{4a}, represent a five- to seven-membered monocyclic ring group,

E^{4b} and E^{5b} each independently represent (1) C or (2) N, wherein at least one of E, E², E³, E⁴b, and E⁵b represent N,

ring 1-B, including E^{4b} and E^{5b}, represents a five- to seven-membered monocyclic ring group, and

the broken-line bonds represent aromatic bonds,

R¹ represents (1) a halogen atom, (2) a C1-6 alkyl group, (3) a C1-6 alkoxy group, or (10) NR⁴R⁵,

when R¹ represents any of (2) to (3), R¹ may be substituted with one hydroxyl group,
R⁴ and R⁵ each independently represent (1) a hydrogen atom, or (2) a C1-6 alkyl group,

m−1 represents an integer of 0 to 5, and

when m is 2 or more, the plurality of R¹ may be the same or different,

the moiety

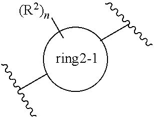

represents any one of the following

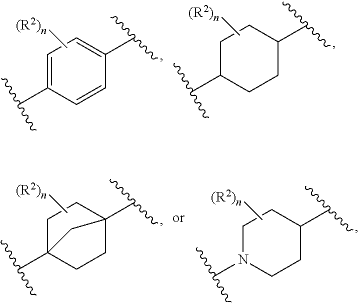

R² represents (2) a C1-6 alkyl group, or (4) a phenyl,

n represents an integer of 0 to 5,

when n is 2 or more, the plurality of R² may be the same or different,

X represents CR^{3e} or N,

R^{3a}, R^{3c}, R^{3d}, and R^{3e} each independently represent (1) a hydrogen atom, or (2) a halogen atom,

R^{3b} represents (1) a C1-6 alkyl group, (2) a C1-6 alkoxy group, or (7) a C1-6 alkylsulfonyl group, and, when R^{3b} represents a C1-6 alkyl group or a C1-6 alkoxy group, the C1-6 alkyl group or a C1-6 alkoxy group may be substituted with 1 to 5 halogen atom.

In the present invention, the compound or a salt thereof is a compound represented by the following general formula (I-1),

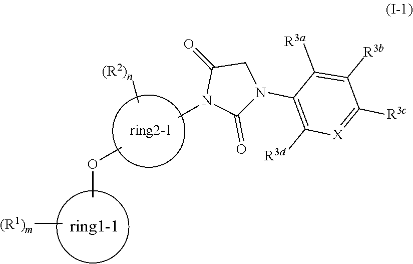

wherein the moiety

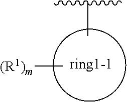

represents

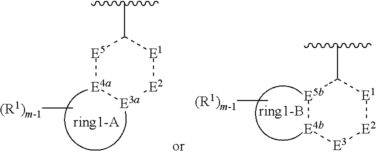

wherein

E¹, E², E³, and E⁵ each independently represent (1) CH, or (3) N, and

E^{3a} and E^{4a} each independently represent C, wherein at least one of E¹, E², and E⁵ represent N,

ring 1-A, including E^{3a} and E^{4a}, represent a five-membered monocyclic ring group, E^{4b} and E^{5b} each independently represent C, wherein at least one of E¹, E², E3 represent N,

ring 1-B, including E^{4b} and E^{5b}, represents a five-membered monocyclic ring group, and

the broken-line bonds represent aromatic bonds,

R¹ represents (1) a halogen atom, (2) a C1-6 alkyl group, or (3) a C1-6 alkoxy group,

when R¹ represents any of (2) to (3), R¹ may be substituted with one hydroxyl group,
m−1 represents an integer of 0 to 5, and

when m is 2 or more, the plurality of R¹ may be the same or different,

the moiety

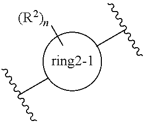

represents any one of the following

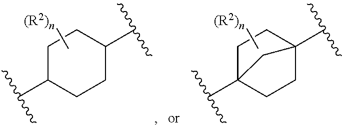

R² represents H,

n represents 0,

X represents N,

R³, R^{3c}, and R^{3d} each independently represent a hydrogen atom,

R^{3b} represents (1) a C1-6 alkyl group, and, the C1-6 alkyl group may be substituted with 1 to 5 halogen atom.

In the present invention, the compound or a salt thereof is a compound represented by the following general formula (I-1),

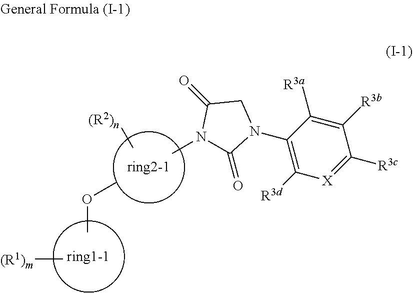

wherein the moiety

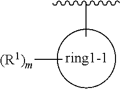

represents

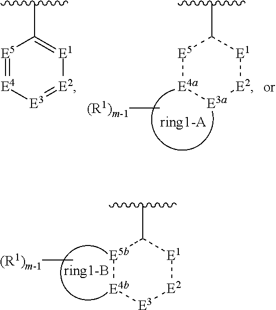

wherein

E¹, E², E³, E⁴, and E⁵ each independently represent (1) CH, (2) CR¹, or (3) N, wherein at least one of E, E², E³, E⁴, and E⁵ represent N, and

E^{3a} and E^{4a} each independently represent (1) C or (2) N, wherein at least one of E¹, E², E^{3a}, E^{4a}, and E⁵ represent N,

ring 1-A, including E^{3a} and E^{4a}, represent a five- to seven-membered monocyclic ring group,

E^{4b} and E^{5b} each independently represent (1) C or (2) N, wherein at least one of E¹, E², E³, E^{4b}, and E^{5b} represent N,

ring 1-B, including E⁴b and E^{5b}, represents a five- to seven-membered monocyclic ring group, and

the broken-line bonds represent aromatic bonds,

R¹ represents (3) a C1-6 alkoxy group, or (10) NR⁴R⁵,
R⁴ and R⁵ each independently represent (1) a hydrogen atom, or (2) a C1-6 alkyl group,

m−1 represents an integer of 0 to 5, and

when m is 2 or more, the plurality of R¹ may be the same or different,

the moiety

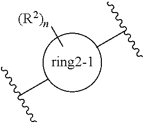

represents any one of the following

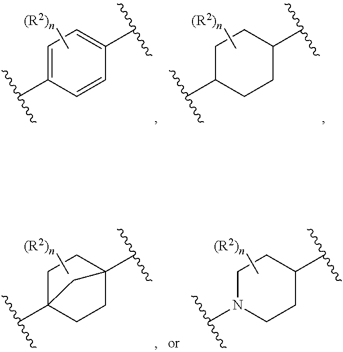

R² represents (2) a C1-6 alkyl group, or (4) a phenyl,

n represents an integer of 0 to 10,

when n is 2 or more, the plurality of R² may be the same or different,

X represents CR^{3e} or N,

R^{3a}, R^{3c}, R^{3d}, and R^{3e} each independently represent (1) a hydrogen atom, or (2) a halogen atom,

R^{3b} represents (1) a C1-6 alkyl group, or (2) a C1-6 alkoxy group, and, when R^{3b} represents a C1-6 alkyl group or a C1-6 alkoxy group, the C1-6 alkyl group or a C1-6 alkoxy group may be substituted with 1 to 5 halogen atom.

The definitions of the above-mentioned preferred groups (single or any combination thereof) are also applied to the general formula (I-a), (I), (I-A), (I-B), (I-C), (I-A-1), (I-A-2), (I-B-1), (I-B-2), (I-C-1), (I-C-2), (I-1), (I-2), (I-3), (I-4) or (I-5).

In the present invention, a compound of general formula (I-a), (I), (I-A), (I-B), (I-C), (I-A-1), (I-A-2), (I-B-1), (I-B-2), (I-C-1), (I-C-2), (I-1), (I-2), (I-3), (I-4), or (I-5) including a combination of the above-listed preferred groups is preferred.

Preferred as the compounds for use in the present invention are the compounds presented in Examples, and the following (1) to (17) are more preferable: (1) 3-{4-[(2-amino-5-pyrimidinyl)oxy]phenyl}-1-[5-(trifluoromethyl)-3-pyridinyl]-2,4-imidazolidinedione,
(2) 3-[4-(1H-pyrazolo[3,4-b]pyridin-5-yloxy)phenyl]-1-[5-(trifluoromethyl)-3-pyridinyl]-2,4-imidazolidinedione,
(3) 1-[3-(methylsulfonyl)-5-(trifluoromethyl)phenyl]-3-[4-(1H-pyrazolo[3,4-b]pyridin-5-yloxy)phenyl]-2,4-imidazolidinedione,
(4) 3-{4-[(2-amino-4-pyrimidinyl)oxy]-2-methylphenyl}-1-[5-(trifluoromethyl)-3-pyridinyl]-2,4-imidazolidinedione,
(5) 3-{trans-4-[(7-methoxy-4-quinazolinyl)oxy]cyclohexyl}-1-[5-(trifluoromethyl)-3-pyridinyl]-2,4-imidazolidinedione,
(6) 1-[3-fluoro-5-(trifluoromethyl)phenyl]-3-[trans-4-(thieno[3,2-b]pyridin-6-yloxy)cyclohexyl]-2,4-imidazolidinedione,
(7) 3-{3-isopropyl-4-[(2-oxo-2,3-dihydro-1H-imidazo[4,5-b]pyridin-7-yl)oxy]phenyl}-1-[5-(trifluoromethyl)-3-pyridinyl]-2,4-imidazolidinedione,
(8) 3-[trans-4-(7H-pyrrolo[2,3-d]pyrimidin-4-yloxy)cyclohexyl]-1-[5-(trifluoromethyl)-3-pyridinyl]-2,4-imidazolidinedione,
(9) 3-[3-methyl-4-(pyrazolo[1,5-a]pyrimidin-7-yloxy)phenyl]-1-[5-(trifluoromethyl)-3-pyridinyl]-2,4-imidazolidinedione,
(10) 3-[4-(7H-pyrrolo[2,3-d]pyrimidin-4-yloxy)bicyclo[2.2.1]hept-1-yl]-1-[5-(trifluoromethyl)-3-pyridinyl]-2,4-imidazolidinedione,
(11) 3-{4-[(2-amino-4-pyrimidinyl)oxy]-2-methylphenyl}-1-[4-fluoro-3-(trifluoromethoxy)phenyl]-2,4-imidazolidinedione,
(12) 3-{4-[(2-amino-4-pyrimidinyl)oxy]-2-methylphenyl}-1-[3-fluoro-5-(trifluoromethyl)phenyl]-2,4-imidazolidinedione,
(13) 3-{5-[(2-amino-4-pyrimidinyl)oxy]-2-biphenylyl}-1-[5-(trifluoromethyl)-3-pyridinyl]-2,4-imidazolidinedione,
(14) 3-[3-ethyl-4-(1H-pyrrolo[2,3-b]pyridin-4-yloxy)phenyl]-1-[5-(trifluoromethyl)-3-pyridinyl]-2,4-imidazolidinedione,
(15) 1-{4-[(2-amino-4-pyrimidinyl)oxy]-3-ethylphenyl}-3-[3-(trifluoromethyl)phenyl]-2-imidazolidinone,
(16) 3-{1-[(7-methoxy-4-quinolinyl)methyl]-4-piperidinyl}-1-[5-(trifluoromethyl)-3-pyridinyl]-2,4-imidazolidinedione, or
(17) 3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione or salt thereof.

In the present invention, the following (1) to (7) are more preferable: (1) 3-(trans-4-{[3-(2-hydroxyethoxy)-1H-pyrazolo[3,4-b]pyridin-5-yl]oxy}cyclohexyl)-1-[5-(trifluoromethyl)-3-pyridinyl]-2,4-imidazolidinedione,
(2) 3-(4-{[6-(2-hydroxy-2-propanyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione,
(3) 3-{4-[(5-fluoro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(4) 3-{4-[(5-chloro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(5) 5-{2,4-dioxo-3-[4-(7H-pyrrolo[2,3-d]pyrimidin-4-yloxy)bicyclo[2.2.1]hept-1-yl]-1-imidazolidinyl}nicotinonitrile,
(6) 3-{2,4-dioxo-3-[4-(7H-pyrrolo[2,3-d]pyrimidin-4-yloxy)bicyclo[2.2.1]hept-1-yl]-1-imidazolidinyl}benzonitrile, or
(7) 3-[4-(1H-pyrazolo[3,4-b]pyridin-4-yloxy)bicyclo[2.2.1]hept-1-yl]-1-[5-(trifluoromethyl)-3-pyridinyl]-2,4-imidazolidinedione.

Unless otherwise specifically mentioned, all isomers are included in the present invention. For example, alkyl, alkenyl, alkynyl, and alkoxy may be straight chain or branched. Moreover, all isomers due to double bond, ring and fused ring (E-, Z-, cis- and trans-forms), isomers due to the presence of asymmetric carbon(s) etc. (R-, S-, a- and P-configuration, enantiomer and diastereomer), optically active substances having optical rotation (D-, L-, d- and I-forms), polar compounds by chromatographic separation (more polar compounds and less polar compounds), equilibrium compounds, rotational isomers, a mixture thereof in any proportion and a racemic mixture are included in the present invention.

In the present invention, unless it is explicitly stated otherwise, as apparent to a person skilled in the art, a symbol:

[symbol]

represents that a substituent is bound to a far side of a paper plane (i.e. α-configuration), [symbol]
represents that a substituent is bound to a near side of a paper plane (i.e. β-configuration), and [symbol]
represents α-configuration, β-configuration or an arbitrary mixture thereof.

The compound represented by the general formula (I-a) is converted into a corresponding salt by the known method. As a salt, a pharmaceutically acceptable salt is preferable. Examples of a suitable salt include acid addition salts (e.g. inorganic acid salts such as hydrochloride, hydrobromide, hydroiodide, sulfate, phosphate and nitrate, organic acid salts such as acetate, lactate, tartrate, benzoate, citrate, methanesulfonate, ethanesulfonate, benzenesulfonate, toluenesulfonate, isethionate, glucuronate, gluconate, etc.), salts of an alkali metal (potassium, sodium etc.), salts of an alkaline earth metal (calcium, magnesium etc.), ammonium salts or salts of a pharmaceutically acceptable organic amine (e.g. tetramethylammonium, triethylamine, methylamine, dimethylamine, cyclopentylamine, benzylamine, phenethylamine, piperidine, monoethanolamine, diethanolamine, tris(hydroxymethyl)aminomethane, lysine, arginine, N-methyl-D-glucamine etc.) etc.

The compound represented by the general formula (I-a) and a salt thereof may be present in an unsolvated form, or in a solvated form with a pharmaceutically acceptable solvent such as water or ethanol.

The compound represented by the general formula (I-a) and a salt thereof can be also converted into a solvate. It is preferable that the solvate is low-toxic and water-soluble. Examples of a suitable solvate include solvates with water, or an alcoholic solvent (e.g. ethanol etc.).

The compound of the present invention can be converted into an N-oxide by known methods. The N-oxide is the compound wherein nitrogen of the compound represented by formula (I-a) is oxidized.

And, the prodrug of the compound represented by the general formula (I-a) refers to a compound which is converted into the compound represented by the general formula (I-a) by a reaction with an enzyme or gastric acid etc. in a living body. Specifically, examples include, when the compound represented by the general formula (I-a) has an amino group, compounds in which the amino group is eicosanoylated, alanylated, pentylaminocarbonized, (5-methyl-2-oxo-1,3-dioxolene-4-yl)methoxycarbonylated, tetrahydrofuranylated, pyrrolidylmethylated, pivaloyloxymethylated, acetoxymethylated, or tert-butylated, when the compound represented by the general formula (I-a) has a hydroxy group, compounds in which the hydroxy group is acetylated, palmitoylated, propanoylated, pivaloylated, succinylated, fumarylated, alanylated, or dimethylaminomethylcarbonylated and, when the compound represented by the general formula (I-a) has a carboxy group, compounds in which the carboxy group is ethylesterified, phenylesterified, carboxymethylesterified, dimethylaminomethylesterified, pivaloyloxymethylesterified, ethoxycarbonyloxyethylesterified, phthalidylesterified, (5-methyl-2-oxo-1,3-dioxolene-4-yl)methylesterified, cyclohexyloxycarbonylethylesterified, or methylamidated, and these compounds can be produced by the known method. And, the prodrug of the compound represented by the general formula (I-a) may be any of a hydrate and a non-hydrate. Alternatively, the prodrug of the compound represented by the general formula (I-a) may be a compound which is changed into the compound represented by the general formula (I-a) under the physiological condition, as described in “Development of Medicaments”, vol. 7 “Molecular Design”, p. 163-198, published by HirokawaShoten in 1990.

The compound represented by the general formula (I-a) can form a cocrystal with an appropriate cocrystal former. As the cocrystal, pharmaceutically acceptable cocrystal that is formed with a pharmaceutically acceptable cocrystal former is preferable. The cocrystal is typically defined as a crystal that is formed of two or more different molecules by intermolecular interaction that is different from ionic bond. Furthermore, the cocrystal may be a composite of a neutral molecule and a salt. The cocrystal can be prepared by a well-known method, for example, melting crystallization, recrystallization from a solvent, or physically pulverizing the components together. Appropriate cocrystal formers include ones described in WO2006/007448.

In the present invention, all mentioning about the present compounds are inclusive of compounds represented by general formula (I-a), salts thereof, N-oxides thereof, solvates (for example, hydrates) thereof, and cocrystals thereof, including N-oxides, solvates (for example, hydrates), and cocrystals of salts of compounds represented by general formula (I-a). Further, each atom constituting the compound represented by the general formula (I-a) may be substituted with an isotope thereof (e.g. ²H, ³H, ¹¹C, ¹³C, ¹⁴C, ¹³N, ¹⁵N, ¹⁵O, ¹⁷O, ¹⁸O, ³⁵S, ¹⁸F, ³⁶Cl, ¹²³I, ¹²⁵I etc.) etc. [Process for producing compound of the present invention]

The compound of the present invention can be produced by the well-known methods, for example, the method described in Comprehensive Organic Transformations: A Guide to Functional Group Preparations, 3rd Edition (Richard C. Larock, John Wiley & Sons Inc, 2018), the methods described in Examples, or the like, with appropriate modification and in combination thereof.

In each reaction in the present specification, a starting material can be used as a salt thereof. The order in which each reaction is carried out can be appropriately changed depending on the introduced substituent, protecting group, or reaction conditions.

Of the compound represented by the general formula (I-a), a compound represented by the general formula (I-X) can be produced by the process represented by the following reaction scheme 1:

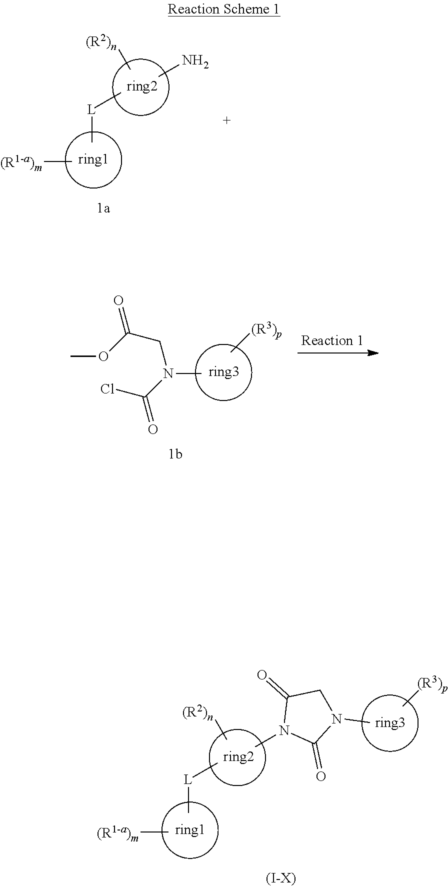

(wherein, all the symbols represent the same meanings as described above)

In the reaction scheme 1, the reaction 1 is known, typically, it can be performed by heating in an organic solvent (e.g. N,N-dimethylformamide, dimethyl sulfoxide, chloroform, dichloromethane, diethyl ether, tetrahydrofuran, methyl t-butyl ether etc.) and in the presence of a base (e.g. pyridine, triethylamine, dimethylaniline, dimethylaminopyridine, diisopropylethylamine etc.).

Examples of aniline compound represented by general formula 1a can be produced by the reaction scheme 2.

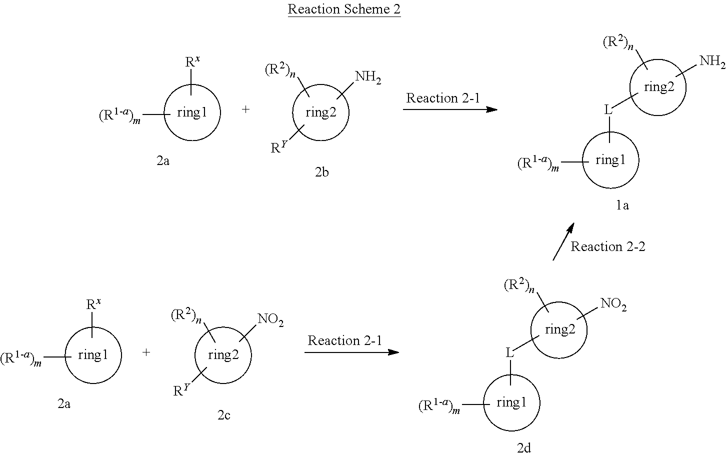

(wherein the reaction scheme 2, Rx and R^{Y} represent -L-R^{L} or R^{z},
when Rx represents -L-R^{L}, R^{Y} represents R^{Z}, or when Rx represents R^{Z}, R^{Y} represents -L-R^{L},
R^{L} represents hydrogen atom or halogen atom,
R^{Z} represents halogen atom, nitro group, methanesulfonyloxy group, p-toluenesulfonyloxy group, or trifluoromethanesulfonyloxy group,
and the other symbols represent the same meanings as described above)

In the reaction scheme 2, the reaction 2-1 is known, and can be carried out by subjecting the compound to a nucleophilic substitution reaction or a coupling reactions using transition metal catalyst.

The nucleophilic substitution reaction is known, and is carried out, for example, in an organic solvent (N,N-dimethylformamide, dimethyl sulfoxide, chloroform, dichloromethane, diethyl ether, tetrahydrofuran, methyl t-butyl ether etc.), with hydroxide of alkaline metal (sodium hydroxide, potassium hydroxide, lithium hydroxide, etc.), hydroxide of alkaline earth metal (barium hydroxide, calcium hydroxide, etc.), or carbonate (sodium carbonate or potassium carbonate, etc.), or an aqueous solution thereof or a mixture thereof at 0 to 100° C.

The coupling reactions using transition metal catalyst is known, for example, Ullmann ether synthesis reaction, Buchwald coupling reaction and the like.

The Ullmann ether synthesis reaction is carried out, for example, in an organic solvent (benzene, toluene, N,N-dimethylformamide, 1,4-dioxane, tetrahydrofuran, methanol, acetonitrile, dimethoxyethane, acetone, etc.) in the presence of a phosphine ligand (triphenylphosphine, 4,5-bis(diphenylphosphino)-9,9-dimethyl-9H-xanthene (Xanthophos) etc.), a catalyst (copper iodide etc.), and a base (sodium ethylate, sodium hydroxide, potassium hydroxide, triethylamine, sodium carbonate, sodium bicarbonate, potassium carbonate, cesium carbonate, thallium carbonate, tripotassium phosphate, cesium fluoride, barium hydroxide, tetrabutylammonium fluoride, etc.) or an aqueous solution thereof or a mixture thereof at room temperature to 130° C.

The Buchwald coupling reaction is carried out, for example, in an organic solvent (benzene, toluene, N,N-dimethylformamide, 1,4-dioxane, tetrahydrofuran, methanol, acetonitrile, dimethoxyethane, acetone, etc.) in the presence of a phosphine ligand (2-dicyclohexylphosphino-2′,6′-dimethoxybiphenyl (SPhos), 2-dicyclohexylphosphino-2′,4′,6′-triisopropylbiphenyl (XPhos), tri-tert-butylphosphine, etc.), a catalyst (tetrakis(triphenylphosphine)palladium(0), bis(triphenylphosphine)palladium(II) dichloride, palladium(II) acetate, palladium black, [1,1′-bis(diphenylphosphino)ferrocene]dichloropalladium(II) dichloromethane adduct, allylpalladium(II) chloride dimer, etc.), and a base (sodium ethoxide, sodium hydroxide, potassium hydroxide, triethylamine, sodium carbonate, sodium bicarbonate, potassium carbonate, cesium carbonate, thallium carbonate, tripotassium phosphate, cesium fluoride, barium hydroxide, tetrabutylammonium fluoride, etc.) or an aqueous solution thereof or a mixture thereof at room temperature to 130° C.

In the reaction scheme 2, the reaction 2-2 is known, and can be carried out by subjecting the compound to a reduction reaction of the nitro group. The reduction reaction of a nitro group is well known, and can be carried out by, for example, the following method.

(1) The reduction reaction is carried out in, for example, a solvent [ethers (tetrahydrofuran, dioxane, dimethoxyethane, diethyl ether, etc.), alcohols (methanol, ethanol, and the like), benzenes (benzene, toluene, and the like), ketones (acetone, methyl ethyl ketone, and the like), nitrites (acetonitrile, and the like), amides (dimethylformamide, and the like), water, ethyl acetate, acetic acid or a mixture solvent of two or more thereof] in the presence of a hydrogeneration catalyst (palladium-carbon, palladium black, palladium, palladium hydroxide, platinum dioxide, platinum-carbon, nickel, Raney-nickel, ruthenium chloride, etc.), in the presence or absence of acids (hydrochloric acid, sulfuric acid, hypochlorous acid, boric acid, tetrafluoroboric acid, acetic acid, p-toluenesulfonic acid, oxalic acid, trifluoroacetic acid, formic acid, and the like), at normal pressure or reduced pressure under a hydrogen atmosphere, in the presence of formic acid ammonium or in the presence of hydrazine, at 0 to 200° C.

(2) The reaction is carried out, for example, in a water-miscible solvent (ethanol, methanol, tetrahydrofuran, etc.) in the presence or absence of an acid (hydrochloric acid, hydrobromic acid, ammonium chloride, acetic acid, ammonium formate, etc.) using a metal reagent (zinc, iron, tin, tin chloride, iron chloride, samarium, indium, sodium borohydride-Nickel chloride, etc.) at 0 to 150° C.

Examples of compound which is a hydantoin precursor represented by general formula 1b can be produced by the reaction scheme 3.

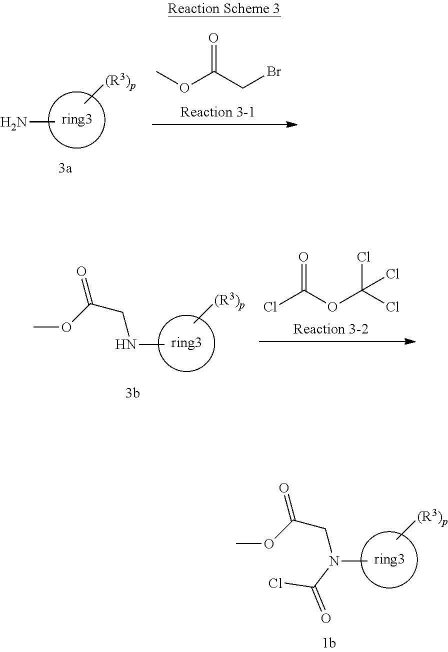

(wherein, all the symbols represent the same meanings as described above)

In the reaction scheme 3, the reaction 3-1 is known, and is carried out, for example, in an organic solvent (N,N-dimethylformamide, dimethyl sulfoxide, chloroform, dichloromethane, diethyl ether, tetrahydrofuran, methyl t-butyl ether etc.), with hydroxide of alkaline metal (sodium hydroxide, potassium hydroxide, lithium hydroxide, etc.), hydroxide of alkaline earth metal (barium hydroxide, calcium hydroxide, etc.), or carbonate (sodium carbonate or potassium carbonate, etc.) or an aqueous solution thereof or a mixture thereof at 0 to 100° C.

In the reaction scheme 3, the reaction 3-2 is known, and is carried out, for example, in an organic solvent (N,N-dimethylformamide, dimethyl sulfoxide, chloroform, dichloromethane, diethyl ether, tetrahydrofuran, methyl t-butyl ether etc.), and in the presence of a base (pyridine, triethylamine, dimethylaniline, N,N-dimethylaminopyridine, diisopropylethylamine etc.), at 0 to 100° C.

The compound represented by the general formula (I-X) can also be produced by the process represented by the following reaction scheme 4:

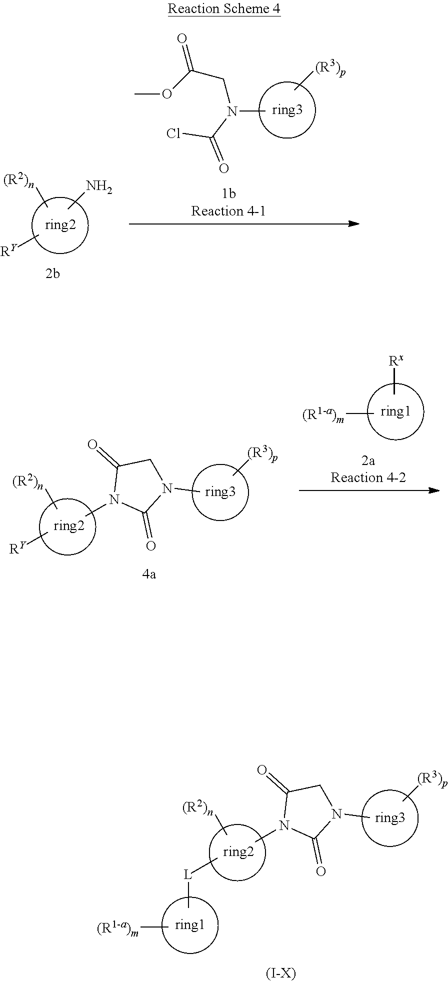

(wherein, all the symbols represent the same meanings as described above)

In the reaction scheme 4, the reaction 4-1 is known and the compound represented by the general formula 4a can be produced by subjecting the compound represented by the general formula 2b and the compound represented by the general formula 1b to the same method as in the above-mentioned reaction 1.

The reaction 4-2 is known and the compound represented by the general formula (I-X) can be produced by subjecting the compound represented by the general formula 4a and the compound represented by the general formula 2a to the same method as in the above-mentioned reaction 2-1.

Of the compound represented by the general formula (I-a), a compound represented by the general formula (I-Y) can be produced by the process represented by the following reaction scheme 5:

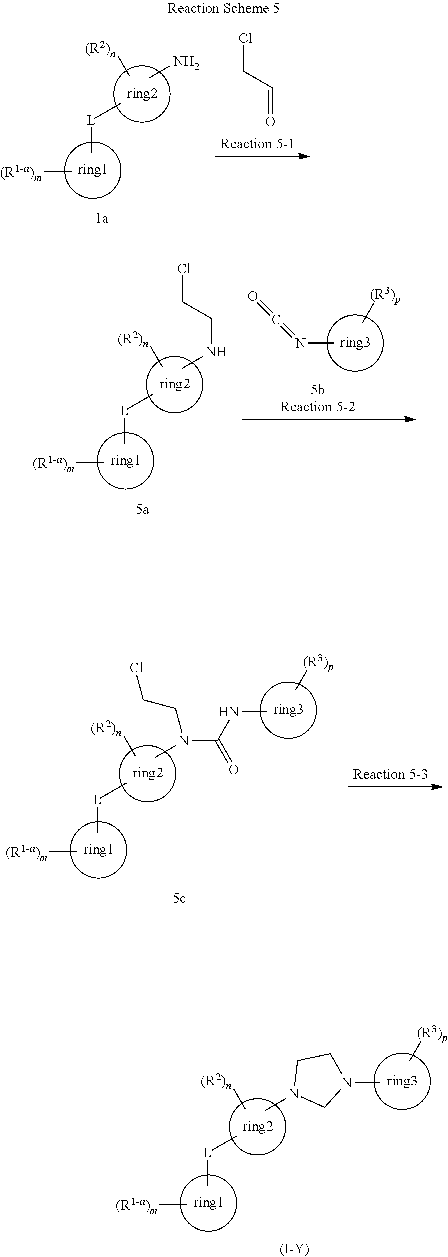

(wherein, all the symbols represent the same meanings as described above)

In the reaction scheme 5, the reaction 5-1 is known and the compound represented by the general formula 5a can be produced by subjecting the compound represented by the general formula L a to a reductive amination reaction. The reductive amination reaction is well-known, and can be performed, for example, by a reaction at 0 to 100° C. in a mixed solvent of an inert organic solvent (dimethylformamide, dimethyl sulfoxide, chloroform, methylene chloride, dichloroethane, diethyl ether, tetrahydrofuran, acetonitrile etc.) and acetic acid in the presence of a reducing agent (sodium triacetoxyborohydride, sodium cyanoborohydride etc.).

The reaction 5-2 is known, and is carried out, for example, in an organic solvent (N,N-dimethylformamide, dimethyl sulfoxide, chloroform, dichloromethane, diethyl ether, tetrahydrofuran, methyl t-butyl ether etc.), and in the presence or absence of a base (pyridine, triethylamine, dimethylaniline, dimethylaminopyridine, diisopropylethylamine etc.), at 0 to 100° C.

The reaction 5-3 is known, and is carried out, for example, in an organic solvent (tetrahydrofuran, dichloromethane, chloroform, benzene, toluene, xylene, hexane, heptane, cyclohexane, diethyl ether, 1,4-dioxane, acetone, ethyl methyl ketone, acetonitrile, dimethyl sulfoxide, N,N-dimethylformamide, N,N-dimethylacetamide, ethyl acetate etc.), and in the presence of a base (potassium tert-butoxide, potassium hydroxide, sodium hydride etc.), at 0 to 100° C.

The compound represented by the general formula (I-a) can be produced by the process represented by the following reaction scheme 6:

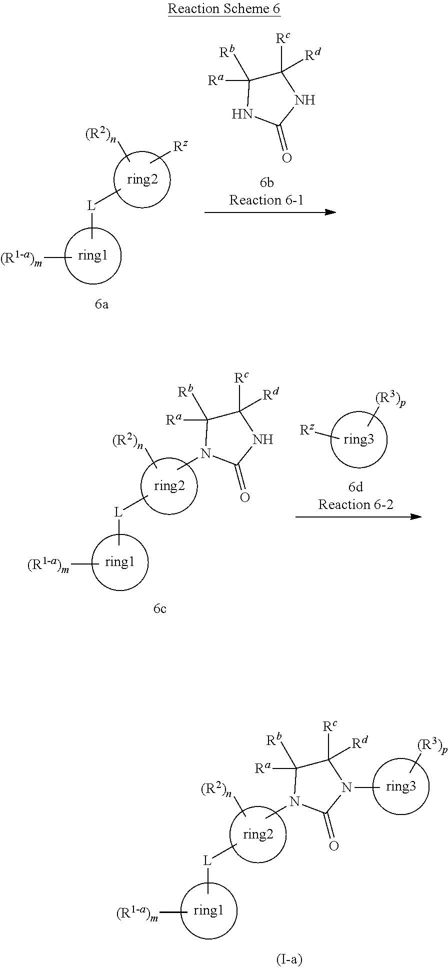

(wherein, all the symbols represent the same meanings as described above)

In the reaction scheme 6, the reaction 6-1 is known and the compound represented by the general formula 6c can be produced by subjecting the compound represented by the general formula 6a to the same method as in the above-mentioned reaction 2-1.

The reaction 6-2 is known and the compound represented by the general formula (I-a) can be produced by subjecting the compound represented by the general formula 6e to the same method as in the above-mentioned reaction 2-1.

Of the present compound, compounds other than those shown above can be produced by the known method, for example, the method described in Comprehensive Organic Transformations: A Guide to Functional Group Preparations, 3rd Edition (Richard C. Larock, John Wiley & Sons Inc, 2018) and the like, or by using a combination of a method in which a part of the known method is modified and the like.

In each reaction in the present specification, a protective group can be introduced to the compound in the above reaction schemes as necessary.

Examples of a protective group for a carboxyl group include methyl, ethyl, tert-butyl, trichloroethyl, benzyl (Bn), phenacyl, p-methoxybenzyl, trityl, 2-chlorotrityl and the like.

Examples of a protective group for an amino group include a benzyloxycarbonyl group, a tert-butoxycarbonyl group, an allyloxycarbonyl (Alloc) group, a 1-methyl-1-(4-biphenyl)ethoxycarbonyl (Bpoc) group, a trifluoroacetyl group, a 9-fluororenylmethoxycarbonyl group, a benzyl (Bn) group, a p-methoxybenzyl group, a benzyloxymethyl (BOM) group, a 2-(trimethylsilyl)ethoxymethyl (SEM) group, and the like. Examples of a protective group for a hydroxyl group include a methyl group, a trityl group, a methoxymethyl (MOM) group, a 1-ethoxyethyl (EE) group, a methoxyethoxymethyl (MEM) group, a 2-tetrahydropyranyl (THP) group, a trimethylsilyl (TMS) group, a triethylsilyl (TES) group, a t-butyldimethylsilyl (TBDMS) group, a t-butyldiphenylsilyl (TBDPS) group, an acetyl (Ac) group, a pivaloyl group, a benzoyl group, a benzyl (Bn) group, a p-methoxybenzyl group, an allyloxycarbonyl (Alloc) group, a 2,2,2-trichloroethoxycarbonyl (Troc) group, and the like.

A method of introducing a protective group can be performed by the method described in T. W. Greene, Protective Groups in Organic Synthesis, Wiley, New York, 5th Edition, 2014, for example, in introduction of a protective group such as a tert-butoxycarbonyl group, a benzyloxycarbonyl group, a fluorenylcarbonyl group, a trityl group, an o-nitrobenzenesulfenyl group etc., the introduction can be performed by a reaction at −50 to 100° C. in a solvent such as dichloromethane, chloroform, 1,2-dichloroethane, tetrahydrofuran, dioxane, toluene, ethyl acetate or water using di-tert-butyl dicarbonate, benzyloxycarbonyl chloride, fluorenylcarbonyl chloride, trityl chloride, o-nitrobenzenesulfenyl chloride or the like, respectively. Thereupon, if necessary, introduction can be performed using a base such as amines such as triethylamine, diisopropylethylamine and the like, organic acid salts such as sodium 2-ethylhexanoate and potassium 2-ethylhexanoate, or inorganic bases such as sodium hydroxide and potassium carbonate.

In each reaction in the present specification, when a protecting group is present in each of the compounds represented by general formulae, a deprotection reaction can be performed, as necessary.

A deprotection reaction of the protective group is known, and can be carried out by the methods mentioned below. Examples thereof include: (1) deprotection reactions by alkaline hydrolysis, (2) deprotection reaction in acidic conditions, (3) deprotection reaction by hydrogenolysis, (4) deprotection reaction of a silyl group, (5) deprotection reaction using metal, (6) deprotection reaction using a metal complex, and the like. These methods are specifically described:

(1) The deprotection reaction by alkaline hydrolysis condition is carried out, for example, in an organic solvent (for example, methanol, tetrahydrofuran, dioxane, etc.) with hydroxide of alkaline metal (for example, sodium hydroxide, potassium hydroxide, lithium hydroxide, etc.), hydroxide of alkaline earth metal (for example, barium hydroxide, calcium hydroxide, and the like), or carbonate (for example, sodium carbonate or potassium carbonate, and the like), or an aqueous solution thereof or a mixture thereof at 0 to 40° C.

(2) The deprotection reaction in acidic conditions is carried out, for example, in an organic solvent (for example, dichloromethane, chloroform, dioxane, ethyl acetate, methanol, isopropyl alcohol, tetrahydrofuran, anisole, etc.), organic acid (for example, acetic acid, trifluoroacetic acid, methanesulfonic acid, p-tosyl acid, etc.), or inorganic acid (for example, hydrochloric acid, sulfuric acid, etc.), or a mixture thereof (for example, hydrogen bromide/acetic acid, etc.) in the presence or absence of 2,2,2-trifluoroethanol at 0 to 100° C.

(3) The deprotection reaction by hydrogenolysis is carried out, for example, in a solvent (for example, ethers (tetrahydrofuran, dioxane, dimethoxyethane, diethyl ether, etc.), alcohols (for example, methanol, ethanol, and the like), benzenes (for example, benzene, toluene, etc.), ketones (for example, acetone, methyl ethyl ketone, and the like), nitriles (for example, acetonitrile, and the like), amides (for example, N,N-dimethylformamide, and the like), water, ethyl acetate, acetic acid, or a mixture of two or more thereof, etc.) in the presence of a catalyst (for example, palladium-carbon, palladium black, palladium hydroxide-carbon, platinum oxide, Raney nickel, etc.) under hydrogen atmosphere at normal pressure or elevated pressure, or in the presence of ammonium formate at 0 to 200° C.

(4) The deprotection reaction of a silyl group is carried out, for example, in a water-miscible organic solvent (for example, tetrahydrofuran, acetonitrile, and the like), by using tetrabutylammonium fluoride at 0 to 40° C. The reaction is also carried out, for example, in organic acid (for example, acetic acid, trifluoroacetic acid, methanesulfonic acid, p-tosyl acid, etc.), or in inorganic acid (for example, hydrochloric acid, sulfuric acid, and the like) or a mixture thereof (for example, hydrogen bromide/acetic acid, and the like) at −10 to 10° C.

(5) The deprotection reaction using a metal is carried out, for example, in an acidic solvent (for example, acetic acid, a buffer of pH 4.2 to 7.2, a mixed solution of the solution and an organic solvent such as tetrahydrofuran, etc.) in the presence of powder zinc, if necessary, with an ultrasonic wave applied at 0 to 40° C.

(6) The deprotection reaction using a metal complex is carried out, for example, in an organic solvent (for example, dichloromethane, N,N-dimethylformamide, tetrahydrofuran, ethyl acetate, acetonitrile, dioxane, ethanol, etc.), water or a mixed solvent thereof in the presence of a trap reagent (for example, tributyltin hydride, triethylsilane, dimedone, morpholine, diethylamine, pyrrolidine, etc.), an organic acid (for example, acetic acid, formic acid, 2-ethylhexanic acid, etc.) and/or in the presence of an organic acid salt (for example, sodium 2-ethylhexanate, potassium 2-ethylhexanate, and the like) in the presence or absence of a phosphine reagent (for example, triphenylphosphine, and the like) using a metal complex (for example, tetrakis(triphenylphosphine)palladium(O), dichlorobis(triphenylphosphine)palladium (II), palladium acetate (II), chlorotris(triphenylphosphine)rhodium (I), etc.) at 0 to 40° C.

In addition to the above-mentioned methods, the deprotection reaction can be carried out by the method described in for example, T. W. Greene, Protective Groups in Organic Synthesis, Wiley, New York, 5th Edition, 2014.

In each reaction in the present specification, compounds used as starting material, represented by general formula 2a, 2b, 2c, 3a, 5b, 6a, 6b and 6d, respectively, are well known or can be produced by well-known methods.

In each reaction in the present specification, a reaction accompanying heating can be performed using a water bath, an oil bath, a sand bath or a microwave as apparent to a person skilled in the art.

In each reaction in the present specification, conveniently, a solid phase-supporting reagent supported by a high-molecular polymer (e.g. polystyrene, polyacrylamide, polypropylene, polyethylene glycol etc.) may be used.

In each reaction in the present specification, the reaction products can be purified by conventional purification methods, for example, by distillation at normal or reduced pressure, by high performance liquid chromatography using silica gel or magnesium silicate, thin layer chromatography, ion-exchange resin, scavenger resin, or column chromatography, washing, recrystallization, or the like. The purification may be done after each reaction or after several reactions.

### [Toxicity]

Since toxicity of the present compound is low, it can be used safely as a medicament.

### [Application to Medicaments]

The compound of the present invention has a potent and selective DDR1 inhibitory activity. The compound of the present invention therefore, can be used by a single substance or a combination with the other drugs for the prevention and/or treatment of a disease which is related to the DDR1 receptor. Example of the disease which is related to the DDR1 receptor include cancers, kidney diseases, cardiovascular diseases, central nervous system diseases and fibrosis, and the like.

As used herein, “cancer” includes, for example, tumor developed from a mutation of a normal cell. Malignant tumor can develop from any organ or tissue of the entire body. Unless specifically distinguished, “cancer” is used synonymously with “malignant tumor”. In the present invention, cancers include, for example, lung cancer, breast cancer, esophageal cancer, head and neck cancer, liver cancer, prostate cancer, lymphoma, leukemia, melanoma, pancreatic cancer, neuroblastoma, glioma, colon cancer, renal cell cancer, thyroid cancer, stomach cancer, bladder cancer, ovarian cancer, endometrial cancer, brain cancer, sarcoma, and the like.

In the present invention, kidney diseases include, for example, acute kidney failure, chronic kidney failure, renal fibrosis, diabetic nephropathy, membranoproliferative glomerulonephritis, mesangial proliferative nephritis, interstitial nephritis, lupus nephritis, amyloid kidney, pyelonephritis, meningogenic glomerulonephritis, membranous nephropathy, IgA nephropathy, focal glomerulosclerosis, hypertensive nephrosclerosis, Alport syndrome, Goodpasture's syndrome, Nephrotic nephritis, and the like.

In the present invention, cardiovascular diseases include, for example, atherosclerosis, thrombotic vascular disease, thrombotic microangiopathy, and the like.

In the present invention, central nervous system diseases include, for example, Alzheimer's disease, Parkinson's disease, and the like.

In the present invention, fibrosis includes, for example, pulmonary fibrosis, hepatic fibrosis, myelofibrosis, and the like.

In order to use the present compound for the purpose of preventing and/or treating the above-mentioned diseases, the said compound which works as an active ingredient, usually formulated with a pharmaceutically acceptable carrier such as various additives or solvents, is systemically or locally administered in an oral or parenteral form. Wherein a pharmaceutically acceptable carrier means a substance other than an active ingredient, which is generally used in the formulation of a pharmaceutical product. Pharmaceutically acceptable carriers are preferred which do not exhibit pharmacological effects at the dosage of the formulation, are harmless, and do not interfere with the therapeutic effect of the active ingredient. Pharmaceutically acceptable carriers can also be used for purposes such as increasing the usefulness of the active ingredient and drug product, facilitating formulation, stabilizing quality, or improving usability.

Specifically, it is appropriate to select the materials listed in the “Japanese Pharmaceutical Excipients Directory” published by YAKUJI NIPPO, Ltd. (edited by the International Pharmaceutical Excipients Council Japan) or other publications, depending on the purpose.

A dose of the present compound is different depending on an age, a weight, symptom, therapeutic effect, an administration method, a treatment time and the like, but usually, the present compound is orally administered in a range of 1 μg to 1 g per once per adult, once to several times a day, or parenterally administered in a range of 0.1 μg to 300 mg per once per adult, once to several times a day, or intravenously continuously administered in a range of 1 hour to 24 hours a day.

Of course, as described above, since a dose varies depending on a variety of conditions, the dose is sufficient at a dose smaller than the aforementioned dose in some cases, or administration beyond the range is required in some cases.

The compound of the present invention is usually administered systemically or locally, by oral or parenteral administration. Examples of oral agents include liquid medicines for internal use (for example, elixirs, syrups, pharmaceutically acceptable water-based agents, suspensions, and emulsions), and solid medicine for internal use (for example, tablets (including sublingual tablets and orally disintegrating tablets), pills, capsules (including hard capsules, soft capsules, gelatin capsules, and microcapsules), powders, granules, and lozenges). Examples of parenteral agents include liquid medicines (for example, injection agents (subcutaneous injection agents, intravenous injection agents, intramuscular injection agents, intraperitoneal injection agents, and drip agents, and the like), eye drops (for example, aqueous eye drops (aqueous eye drops, aqueous eye drop suspensions, viscous eye drops, and solubilized eye drops, etc.), and nonaqueous eye drops (for example, nonaqueous eye drops and nonaqueous eye drop suspensions), and the like), agents for external use (for example, ointments (ophthalmic ointments, and the like)), and ear drops, and the like. These formulations may be controlled release agents such as rapid release formulations, sustained release formulations, and the like. These formulations can be produced by well-known methods, for example, by the methods described in The Japanese Pharmacopoeia.

Liquid medicines for internal use as the oral agent can be produced by, for example, dissolving or suspending an active ingredient in a generally used diluent (for example, purified water, ethanol, or mixture liquid thereof, or the like). The liquid medicine may include a wetting agent, a suspension agent, a sweetening agent, a flavoring material, an aromatic substance, a preservative, a buffer agent, and the like.

Solid medicines for internal use as the oral agent are formulated by, for example, mixing the active ingredient with, for example, a vehicle (for example, lactose, mannitol, glucose, microcrystalline cellulose, starch, and the like), a binder (for example, hydroxypropyl cellulose, polyvinylpyrrolidone, magnesium metasilicate aluminate, and the like), a disintegrant (for example, sodium carboxymethylcellulose, and the like), a lubricant (for example, magnesium stearate, and the like), a stabilizer, a dissolution adjuvant (for example, glutamic acid, aspartic acid, and the like), and the like, and formulating according to standard methods. As necessary, coating may be carried out with a coating agent (for example, sugar, gelatin, hydroxypropyl cellulose, hydroxypropyl methyl cellulose phthalate, and the like), and coating of two or more layers may be employed.

Agents for external use as parenteral agents are produced by well-known methods or generally used prescriptions. For example, an ointment may be produced by incorporation or melting of an active ingredient into base material. The ointment base material is selected from well-known material or generally used material. For example, a single material or a mixture of two or more of materials are selected from higher fatty acids and higher fatty acid esters (for example, adipic acid, myristic acid, palmitic acid, stearic acid, oleic acid, adipate esters, myristate esters, palmitate esters, stearate esters, oleate esters, and the like), waxes (for example, beeswax, spermaceti, ceresin, and the like), surfactants (for example, polyoxyethylene alkyl ether phosphate esters, and the like), higher alcohols (for example, cetanol, stearyl alcohol, etostearyl alcohol, and the like), silicone oils (for example, dimethylpolysiloxane, and the like), hydrocarbons (for example, hydrophilic petrolatum, white petrolatum, purified lanolin, liquid paraffin, and the like), glycols (for example, ethylene glycol, diethylene glycol, propylene glycol, polyethylene glycol, macrogol, and the like), plant oils (for example, castor oil, olive oil, sesame oil, turpentine oil, and the like), animal oils (for example, mink oil, egg yolk oil, squalane, squalene, and the like), water, absorption promoters, and anti-irritants. Furthermore, a humectant, preservative, stabilizer, antioxidant, fragrance, and the like, may be included.

The injection agents as parenteral agents include solutions, suspensions, emulsions and solid injection agents to be dissolved or suspended in a solvent before use. The injection agent is used by, for example, dissolving, suspending or emulsifying an active ingredient in a solvent. Examples of the solvent include distilled water for injection, physiological saline, vegetable oils, and alcohols such as propylene glycol, polyethylene glycol, ethanol, and mixtures thereof. Furthermore, the injection agent may contain a stabilizer, a dissolution aid (glutamic acid, aspartic acid, and Polysorbate 80 (registered trademark), etc.), a suspending agent, an emulsifying agent, a soothing agent, a buffer, a preservative, and the like. Such an injection agent is produced by sterilizing at the final step or employing an aseptic process. Furthermore, it is also possible to employ an aseptic solid product such as a freeze-dried product produced and sterilized or dissolved in aseptic distilled water for injection or other solvent before use.

The compound of the present invention may be administered as a combination drug in combination with other drugs in order to accomplish the following purposes:

1) to supplement and/or enhance the preventive and/or therapeutic effect of the compound;
2) to improve the kinetics, improvement of absorption, and reduction of the dose of the compound; and/or
3) to eliminate the side effects of the compound.

A combination drug of the compound of the present invention and other drugs may be administered in the form of a compounding agent including these components mixed into one formulation, or may be administered in separate formulations. Administration as separate formulations includes simultaneous administration and administration at different times. In the administration at different times, the compound of the present invention may be administered before the other drug. Alternatively, the other drug may be administered before the compound of the present invention. The method for the administration of these drugs may be the same as each other or different from each other.

The other drugs for supplementing and/or enhancing the preventive and/or therapeutic effect of the compound of the present invention against cancer include, for example, alkylating agents, antimetabolites, anticancer antibiotics, plant formulations, hormones, platinum compounds, topoisomerase inhibitors, kinase inhibitors, anti-CD20 antibodies, anti-HER2 antibodies, anti-EGFR antibodies, anti-VEGF antibodies, proteasome inhibitors, HDAC inhibitors, immunomodulators, and the like.

Examples of the alkylating agents include cyclophosphamide, ifosfamide, dacarbazine, temozolomide, nimustine hydrochloride, ranimustine, bendamustine, thiotepa carboquone, and the like.

Examples of the antimetabolites include methotrexate, pemetrexed, fluorouracil, tegafur, tegafur uracil, tegafur gimestat otastat potassium, doxifluridine, capecitabine, cytarabine, gemcitabine hydrochloride, fludarabine, nelarabine, carmofur procarbazine hydrochloride, and the like.

Examples of the anticancer antibiotics include mitomycin C, doxorubicin hydrochloride, aclarubicin hydrochloride, pirarubicin hydrochloride, epirubicin, chromomycin A3, bleomycin, peplomycin sulfate, therarubicin, and the like.

Examples of the plant formulations include irinotecan hydrochloride, etoposide, vincristine sulfate, vinblastine sulfate, vindesine sulfate, vinorelbine tartrate, docetaxel hydrate, eribulin mesylate, paclitaxel, and the like.

Examples of the hormones include estramustine phosphate sodium, flutamide, bicalutamide, goserelin acetate, leuprorelin acetate, tamoxifen citrate, toremifene citrate, anastrozole, letrozole, exemestane, mepitiostane, medroxyprogesterone acetate, epitiostanol, fosfestrol, fadrozole hydrochloride hydrate, abiraterone, fulvestrant, aminoglutethimide, and the like.

Examples of the platinum compounds include carboplatin, cisplatin, nedaplatin, and oxaliplatin, and the like.

Examples of the topoisomerase inhibitor include topotecan, sobuzoxane, and the like.

Examples of the kinase inhibitors include EGFR inhibitors including erlotinib, gefitinib and afatinib; HER2 inhibitors including lapatinib; BCR-ABL inhibitors including imatinib; ALK inhibitors including crizotinib; multikinase inhibitors including regorafenib, dasatinib, and the like.

Examples of the anti-CD20 antibodies include rituximab, ibritumomab, ibritumomab tiuxetan, and ocrelizumab.

Examples of the HER2 antibodies include trastuzumab, trastuzumab emtancin, pertuzumab, and the like.

Examples of the anti-EGFR antibodies include cetuximab, panitumumab, and the like.

Examples of the anti-VEGF antibodies include bevacizumab, and the like.

Examples of the proteasome inhibitors include bortezomib, and the like.

Examples of the HDAC inhibitors include vorinostat and the like.

Examples of the immunomodulators include thalidomide, lenalidomide, pomalidomide, and the like.

The other drugs for supplementing and/or enhancing the preventive and/or therapeutic effect of the compound of the present invention against renal diseases include, for example, calcium antagonists, renin angiotensin inhibitors, beta blockers, diuretics, antiplatelet agents, anticoagulants, dyslipidemic agents, active vitamin D, phosphorus adsorbents, potassium adsorbents, oral adsorbents, hyperuricemia agents, erythropoietin formulations, sodium bicarbonate, adrenocortical steroids, immunosuppressive agents, and the like.

Examples of the calcium antagonists include azelnidipine, amlodipine besylate, alanidipine, efonidipine hydrochloride ethanol adduct, cilnidipine, diltiazem hydrochloride, nicardipine hydrochloride, nisoldipine, nitrendipine, nifedipine, nilvadipine, balnidipine hydrochloride, felodipine, benidipine hydrochloride, manidipine hydrochloride, and the like.

Examples of the renin angiotensin inhibitors include azilsartan, irbesartan, olmesartan, olmesartan medoxomil, candesartan, candesartan cilexetil, telmisartan, valsartan, losartan potassium, aliskiren fumarate, alacepril, imidapril hydrochloride, enalapril maleate, captopril, quinapril hydrochloride, cilazapril, temocapril hydrochloride, delapril hydrochloride, trandolapril, benazepril hydrochloride, perindopril erbumin, lisinopril hydrate, and the like.

Examples of the beta blockers include acebutolol hydrochloride, atenolol, carteolol hydrochloride, celiprolol hydrochloride, nadolol, nipradilol, bisoprolol, pindolol, propranolol hydrochloride, betaxolol hydrochloride, metoprolol tartrate, and the like.

Examples of the diuretics include acetazolamide, azosemide, isosorbide, indapamide, potassium canrenoate, spironolactone, torasemide, triamterene, trichlormethiazide, tripamide, tolvaptan, furosemide, bumetanide, benzylhydrochlorothiazide, meticrane, mefruside, mozavaptan hydrochloride, D-mannitol, and the like.

Examples of the antiplatelet agents include aspirin, ticlopidine, clopidogrel, sarpogrelate, cilostazol, dipyridamole, ticagrelor, beraprost sodium, ethyl icosapentate, prasugrel, and the like.

Examples of the anticoagulants include warfarin, dabigatran, riveroxaban, apixaban, edoxaban, and the like.

Examples of the dyslipidemic agents include simvastatin, pravastatin, atorvastatin, pitavastatin, clofibrate, fenofibrate, clinofibrate, pemafibrate, cholestyramine, nicotinic acid, probucol, ezetimibe, lomitapide mesylate, evolocumab, alirocumab, ethyl icosapentate, dextran sulfate sodium sulfur 5, polyene phosphatidylcholine, elastase ES, omega-3 acid ethyl esters, gamma-oryzanol, and the like.

Examples of the active vitamin D include calcipotriol, tacalcitol, calcitriol, alfacalcidol, falecalcitriol, eldecalcitol, maxacalcitol, and the like.

Examples of the phosphorus adsorbents include ferric citrate hydrate, sucroferric oxyhydroxide, sevelamer hydrochloride, lanthanum carbonate hydrate, precipitated calcium carbonate, bixalomer, and the like.

Examples of the potassium adsorbents include calcium polystyrene sulfonate, and the like.

Examples of the oral adsorbents include spherical charcoal, and the like.

Examples of the hyperuricemia agents include allopurinol, febuxostat, topiroxostat, probenecid, benzbromarone, bucolome, and the like.

Examples of the adrenocortical steroids include hydrocortisone, prednisolone, methylprednisolone, triamcinolone, dexamethasone, betamethasone, and the like.

Examples of the immunosuppressive agents include cyclosporine, mizoribine, cyclophosphamide, azathioprine, tacrolimus, and the like.

The other drugs for supplementing and/or enhancing the preventive and/or therapeutic effect of the compound of the present invention against central nervous system diseases include, for example, anti-Alzheimer's disease drugs, anti-Parkinson's drugs, and the like.

Examples of the anti-Alzheimer's disease drugs include donepezil, rivastigmine, galantamine, memantine, and the like.

Examples of the anti-Parkinson's drugs include trihexyphenidyl hydrochloride, biperiden hydrochloride, lactate biperidene, mazaticol hydrochloride hydrate, piroheptine hydrochloride, levodopa, amantadine hydrochloride, bromocriptine mesilate, pergolide mesilate, ropinirole hydrochloride, pramipexole hydrochloride hydrate, cabergoline, apomorphine hydrochloride hydrate, rotigotine, selegiline hydrochloride, rasagiline mesilate, entacapone, stratifin, talipexole hydrochloride, zonisamide, and the like.

Other drug may be administered by combining arbitrary two or more kinds at an appropriate proportion.

A dose of the aforementioned other drug can be appropriately selected based on a dose which is clinically used. And, a compounding ratio of the present compound and other drug can be appropriately selected depending on an age and a weight of a subject to be administered, an administration method, an administration time, a target disease, symptom, a combination and the like. For example, other drug may be used at 0.01 to 100 parts by mass based on 1 part by mass of the present compound. Other drug may be administered by combining arbitrary two or more kinds at an appropriate proportion. And, the aforementioned drug includes not only drugs which have been found out up to now, but also drugs which will be found out from now on.

Unless it otherwise defines, all the technical terms, scientific terms and abbreviated words which are used in this specification have the same meaning as the meaning generally understood by a person skilled in the art.

An entire content of all patent documents and non-patent documents or reference documents which are explicitly cited in the present specification is cited herein as a part of the present specification.

### EXAMPLES

The present invention will be described in details by referring to Examples hereinbelow, but the present invention is not limited to Examples.

Concerning chromatographic separation and TLC, a solvent in parentheses corresponds to an eluting solvent or a developing solvent employed and a ratio is expressed by volume ratio.

Concerning NMR, a solvent in parentheses corresponds to a solvent used for the measurement.

As used herein, when enantiomers or diastereomers were isolated in stereochemically enriched form with unknown stereochemistry, they were assigned by their respective chiral HPLC retention times.

A compound name used in the present specification is given by using a computer program ACD/Name (registered trademark) of Advanced Chemistry Development which generally denominates a compound according to the IUPAC nomenclature or by denomination according to the IUPAC nomenclature.

LC-MS/ELSD was performed by any of the following conditions in Table 1.

TABLE 1

| | | | Column | | Total | |
|---|---|---|---|---|---|---|
| | | | Temp | | Flow | |
| Method | Instrument | Column | (deg.) | Gradient | (mL/min) | PDA total |
| 1 | Shimadzu | CORTECS | 40 | A) 0.09% formic | 0.8 | 190-400 nm |
| | LCMS-2020 | C18+ | | acid (FA), Water | | |
| | | 50 mm. 2.1 | | B) 0.1% FA, MeCN | | |
| | | mm. 2.7 μm | | A/B = 95/5→ | | |
| | | | | 5/95 (2.00 min) | | |
| 2 | Shimadzu | Shim-pack | 40 | A) 0.05% trifluoro | 1.2 | 190-400 nm |
| | LCMS-2020 | XR-ODS | | acetic acid (TFA), Water | | |
| | | 50 mm. 3.0 | | B) 0.05% TFA, MeCN | | |
| | | mm. 2.2 μm | | A/B = 95/5→ | | |
| | | | | 0/100 (2.00 min) | | |
| 3 | Shimadzu | Kinetex EVO | 40 | A) 5 mmolNH₄HCO₃, | 1.2 | 190-400 nm |
| | LCMS-2020 | 50 mm. 3.0 | | Water | | |
| | | mm. 2.6 μm | | B) MeCN | | |
| | | | | A/B = 90/10→ | | |
| | | | | 5/95 (2.00 min) | | |
| 4 | Shimadzu | Shim-pack | 40 | A) 0.05% TFA, Water | 1.5 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 40/60 (3.00 min)→ | | |
| | | | | 5/95 (3.30 min) | | |
| 4-1 | Shimadzu | Shim-pack | 40 | A) 0.05% TFA, Water | 1.5 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 50/50 (2.90 min)→ | | |
| | | | | 5/95 (3.30 min) | | |
| 5 | Shimadzu | Ascentis | 40 | A) 0.05% TFA, Water | 1.5 | 190-400 nm |
| | LCMS-2020 | Express Cl8 | | B) 0.05% TFA, MeCN | | |
| | | 50 mm. 3.0 | | A/B = 95/5→ | | |
| | | mm. 2.7 μm | | 5/95 (2.00 min) | | |
| 6 | Shimadzu | Shim-pack | 40 | A) 0.05% TFA, Water | 1.2 | 190-400 nm |
| | LCMS- | XR-ODS, | | B) 0.05% TFA, MeCN | | |
| | 2010EV | 50 mm. 3.0 | | A/B = 70/30→ | | |
| | | mm. 2.2 μm | | 40/60 (4.1 min)→ | | |
| | | | | 5/95 (4.40 min) | | |
| 7 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 40/60 (3.00 min)→ | | |
| | | | | 5/95 (3.40 min) | | |
| 8 | Shimadzu | Kinetex XB- | 40 | A) 0.09% FA, Water | 1.2 | 190-400 nm |
| | LCMS-2020 | C18, | | B) 0.1% FA, MeCN | | |
| | | 50 mm. 2.1 | | A/B = 95/5→ | | |
| | | mm. 2.6 μm | | 5/95 (2.00 min) | | |
| 9 | Shimadzu | Shim-pack | 40 | A) 0.05% TFA, Water | 1.5 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 5/95 (2.00 min) | | |
| 10 | Shimadzu | Xselect CSH | 40 | A) 0.1% FA, Water | 1.2 | 190-400 nm |
| | LCMS-2020 | C18, | | B) 0.1% FA, MeCN | | |
| | | 50 mm. 3.0 | | A/B = 95/5→ | | |
| | | mm. 2.5 μm | | 5/95 (2.10 min) | | |
| 11 | Shimadzu | Kinetex | 40 | A) 5 mmolNH₄HCO₃, | 1.2 | 190-400 nm |
| | LCMS-2020 | 2.6 um EVO | | Water | | |
| | | C18 100A, | | B) MeCN | | |
| | | 50 mm. 3.0 | | A/B = 90/10→ | | |
| | | mm. 2.6 μm | | 30/70 (3.50 min)→ | | |
| | | | | 5/95 (4.00 min) | | |
| 11-2 | Shimadzu | Kinetex | 40 | A) 5 mmolNH₄HCO₃, | 1.2 | 190-400 nm |
| | LCMS-2020 | 2.6 um EVO | | Water | | |
| | | C18 100A, | | B) MeCN | | |
| | | 50 mm. 3.0 | | A/B = 90/10→50/50 (3.00 | | |
| | | mm. 2.6 μm | | min)→5/95 (4.00 min) | | |
| 12 | Shimadzu | Kinetex | 40 | A) 5 mmolNH₄HCO₃, | 1.2 | 190-400 nm |
| | LCMS-2020 | 2.6 um EVO | | Water | | |
| | | C18 100A, | | B) MeCN | | |
| | | 50 mm. 3.0 | | A/B = 90/10→5/95 (2.10 | | |
| | | mm. 2.6 μm | | min) | | |
| 13 | Shimadzu | Kinetex@ | 40 | A) 5 mmolNH₄HCO₃, | 1.2 | 190-400 nm |
| | LCMS-2020 | 2.6 um EVO | | Water | | |
| | | C18 100A, | | B) MeCN | | |
| | | 50 mm. 3.0 | | A/B = 90/10→5/95 (2.00 | | |
| | | mm. 2.6 μm | | min) | | |
| 14 | Shimadzu | Titan C18, | 40 | A) 0.05% TFA, Water | 0.7 | 190-400 nm |
| | LCMS-2020 | 50 mm. 2.1 | | B) 0.05% TFA, MeCN | | |
| | | mm. 1.9 μm | | A/B = 95/5→ | | |
| | | | | 5/95 (2.00 min) | | |
| 15 | Shimadzu | Poroshell | 40 | A) 5 mmolNH₄HCO₃, | 1.0 | 190-400 nm |
| | LCMS-2020 | HPH-C18, | | Water | | |
| | | 50 mm. 3.0 | | B) MeCN | | |
| | | mm. 2.7 μm | | A/B = 95/5→ | | |
| | | | | 30/70 (3.00 min)→ | | |
| | | | | 5/95 (3.20 min) | | |
| 16 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 40/60 (3.00 min)→ | | |
| | | | | 5/95 (3.30 min) | | |
| 16-1 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 60/40 (2.90 min)→ | | |
| | | | | 5/95 (3.30 min) | | |
| 16-2 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 50/50 (2.90 min)→ | | |
| | | | | 5/95 (3.30 min) | | |
| 16-3 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 30/70 (3.00 min)→ | | |
| | | | | 5/95 (3.30 min) | | |
| 17 | Shimadzu | Poroshell | 40 | A) 5 mmolNH₄HCO₃, | 1.0 | 190-400 nm |
| | LCMS-2020 | HPH-C18, | | Water | | |
| | | 50 mm. 3.0 | | B) MeCN | | |
| | | mm. 2.7 μm | | A/B = 95/5→ | | |
| | | | | 5/95 (2.00 min) | | |
| 18 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 80/20→ | | |
| | | mm. 2.2 μm | | 40/60 (2.70 min)→ | | |
| | | | | 5/95 (3.00 min) | | |
| 19 | Shimadzu | CORTECS | 40 | A) 0.09% FA, Water | 0.8 | 190-400 nm |
| | LCMS-2020 | C18+ | | B) 0.1% FA, MeCN | | |
| | | 50 mm. 2.1 | | A/B = 95/5→ | | |
| | | mm. 2.7 μm | | 60/40 (1.00 min)→ | | |
| | | | | 5/95 (1.50 min) | | |
| 20 | Shimadzu | Kinetex XB- | 40 | A) 0.1% FA, Water | 1.2 | 190-400 nm |
| | LCMS-2020 | C18, | | B) 0.1% FA, MeCN | | |
| | | 50 mm. 3.0 | | A/B = 70/30→ | | |
| | | mm. 2.6 μm | | 5/95 (3.50 min) | | |
| 21 | Shimadzu | Kinetex XB- | 40 | A) 0.1% FA, Water | 1.2 | 190-400 nm |
| | LCMS-2020 | C18, | | B) 0.1% FA, MeCN | | |
| | | 50 mm. 3.0 | | A/B = 95/5→ | | |
| | | mm. 2.6 μm | | 40/60 (3.50 min)→ | | |
| | | | | 10/90 (4.50 min) | | |
| 22 | Shimadzu | Kinetex EVO | 40 | A) 5 mmolNH₄HCO₃, | 1.0 | 190-400 nm |
| | LCMS-2020 | 50 mm. 3.0 | | Water | | |
| | | mm. 2.6 μm | | B) MeCN | | |
| | | | | A/B = 95/5→ | | |
| | | | | 5/95 (2.00 min) | | |
| 23 | Shimadzu | Kinetex XB- | 40 | A) 0.1% FA, Water | 1.2 | 190-400 nm |
| | LCMS-2020 | C18, | | B) 0.1% FA, MeCN | | |
| | | 50 mm. 3.0 | | A/B = 95/5→ | | |
| | | mm. 2.6 μm | | 0/100 (2.00 min) | | |
| 24 | Shimadzu | Xselect CSH | 40 | A) 0.1% FA, Water | 1.2 | 190-400 nm |
| | LCMS-2020 | C18, | | B) 0.1% FA, MeCN | | |
| | | 50 mm. 3.0 | | A/B = 95/5→ | | |
| | | mm. 2.5 μm | | 0/100 (2.00 min) | | |
| 25 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 5/95 (4.10 min) | | |
| 26 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 40/60 (3.80 min)→ | | |
| | | | | 0/100 (4.10 min) | | |
| 27 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 40/60 (3.00 min)→ | | |
| | | | | 0/100 (4.00 min) | | |
| 28 | Shimadzu | CORTECS | 40 | A) 0.1% FA, Water | 1.0 | 190-400 nm |
| | LCMS-2020 | C18+ | | B) 0.1% FA, MeCN | | |
| | | 50 mm. 2.1 | | A/B = 90/10→ | | |
| | | mm. 2.7 μm | | 50/50 (3.00 min)→ | | |
| | | | | 0/100 (4.00 min) | | |
| 29 | Shimadzu | CORTECS | 40 | A) 0.1% FA, Water | 1.0 | 190-400 nm |
| | LCMS-2020 | C18+ | | B) 0.1% FA, MeCN | | |
| | | 50 mm. 2.1 | | A/B = 90/10→ | | |
| | | mm. 2.7 μm | | 30/70 (3.60 min)→ | | |
| | | | | 0/100 (4.30 min) | | |
| 30 | Shimadzu | Ascentis | 40 | A) 0.1% FA, Water | 1.2 | 190-400 nm |
| | LCMS-2020 | Express C18, | | B) 0.1% FA, MeCN | | |
| | | 50 mm. 3.0 | | A/B = 95/5→ | | |
| | | mm. 2.7 μm | | 0/100 (2.00 min) | | |
| 31 | Shimadzu | Kinetex XB- | 40 | A) 0.05% TFA, Water | 1.2 | 190-400 nm |
| | LCMS-2020 | C18 100A, | | B) 0.05% TFA, MeCN | | |
| | | 50 mm. 3.0 | | A/B = 95/5→ | | |
| | | mm. 2.6 μm | | 40/60 (4.30 min)→ | | |
| | | | | 0/100 (5.30 min) | | |
| 32 | Shimadzu | CORTECS | 40 | A) 0.09% FA, Water | 0.8 | 190-400 nm |
| | LCMS-2020 | C18+ | | B) 0.1% FA, MeCN | | |
| | | 50 mm. 2.1 | | A/B = 95/5→ | | |
| | | mm. 2.7 μm | | 40/60 (3.25 min)→ | | |
| | | | | 5/95 (3.80 min) | | |
| 33 | Shimadzu | Kinetex | 40 | A) 0.04% NH₄OH, | 1.2 | 190-400 nm |
| | LCMS-2020 | 2.6 um EVO | | Water | | |
| | | C18 100A, | | B) MeCN | | |
| | | 50 mm. 3.0 | | A/B = 90/10→ | | |
| | | mm. 3.0 μm | | 5/95 (2.10 min) | | |
| 34 | Shimadzu | Titank C18 | 40 | A) 5 mmolNH₄HCO₃, | 1.2 | 190-400 nm |
| | LCMS-2020 | 50 mm. 3.0 | | Water | | |
| | | mm. 3.0 μm | | B) MeCN | | |
| | | | | A/B = 90/10→ | | |
| | | | | 5/95 (2.10 min) | | |
| 35 | Shimadzu | Ascentis | 40 | A) 0.1% FA, Water | 1.2 | 190-400 nm |
| | LCMS-2020 | Express C18, | | B) 0.1% FA, MeCN | | |
| | | 50 mm. 3.0 | | A/B = 90/10→ | | |
| | | mm. 2.7 μm | | 0/100 (4.10 min) | | |
| 36 | Shimadzu | CORTECS | 40 | A) 0.1% FA, Water | 1.0 | 190-400 nm |
| | LCMS-2020 | C18 + MVK | | B) 0.05% FA, MeCN | | |
| | | 50 mm. 2.1 | | A/B = 90/10→ | | |
| | | mm. 2.7 μm | | 0/100 (2.00 min) | | |
| 37 | Shimadzu | Kinetex | 40 | A) 5 mmolNH₄HCO₃, | 1.2 | 190-400 nm |
| | LCMS-2020 | 2.6 um EVO | | Water | | |
| | | C18 100A, | | B) MeCN | | |
| | | 50 mm. 3.0 | | A/B = 70/30→ | | |
| | | mm. 2.6 μm | | 5/95 (4.30 min) | | |
| 38 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 5/95 (2.00 min) | | |
| 39 | Shimadzu | Xselect CSH | 40 | A) 0.1% FA, Water | 1.2 | 190-400 nm |
| | LCMS-2020 | C18, | | B) 0.1% FA, MeCN | | |
| | | 50 mm. 3.0 | | A/B = 95/5→ | | |
| | | mm. 2.5 μm | | 40/60 (3.50 min)→ | | |
| | | | | 10/90 (4.50 min) | | |
| 40 | Shimadzu | CORTECS | 40 | A) 0.1% FA, Water | 1.0 | 190-400 nm |
| | LCMS-2020 | C18+ | | B) 0.1% FA, MeCN | | |
| | | 50 mm. 2.1 | | A/B = 90/10→ | | |
| | | mm. 2.7 μm | | 60/40 (3.00 min)→ | | |
| | | | | 0/100 (4.20 min) | | |
| 41 | Shimadzu | Titank C18 | 40 | A) 5 mmolNH₄HCO₃, | 1.2 | 190-400 nm |
| | LCMS-2020 | 50 mm. 3.0 | | Water | | |
| | | mm. 3.0 μm | | B) MeCN | | |
| | | | | A/B = 90/10→ | | |
| | | | | 50/50 (3.00 min)→ | | |
| | | | | 5/95 (4.00 min) | | |
| 42 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 50/50 (2.60 min)→ | | |
| | | | | 0/100 (3.80 min) | | |
| 43 | Shimadzu | Kinetex XB- | 40 | A) 0.05% TFA, Water | 1.2 | 190-400 nm |
| | LCMS-2020 | C18 100 A, | | B) 0.05% TFA, MeCN | | |
| | | 50 mm. 3.0 | | A/B = 95/5→ | | |
| | | mm. 2.6 μm | | 0/100 (2.10 min) | | |
| 44 | Shimadzu | Ascentis | 40 | A) 0.09% FA, Water | 1.5 | 190-400 nm |
| | LCMS-2020 | Express C18, | | B) 0.1% FA, MeCN | | |
| | | 50 mm. 3.0 | | A/B = 95/5→ | | |
| | | mm. 2.7 μm | | 5/95 (2.00 min) | | |
| 45 | Shimadzu | Accucore | 40 | A) 0.1% FA, Water | 1.0 | 190-400 nm |
| | LCMS-2020 | C18, | | B) 0.1% FA, MeCN | | |
| | | 50 mm. 2.1 | | A/B = 70/30→ | | |
| | | mm. 2.6 μm | | 30/70 (3.50 min)→ | | |
| | | | | 5/95 (4.00 min) | | |
| 46 | Shimadzu | HALO C18, | 40 | A) 0.1% FA, Water | 1.5 | 190-400 nm |
| | LCMS-2020 | 30 mm. 3.0 | | B) 0.1% FA, MeCN | | |
| | | mm. 2.0 μm | | A/B = 95/5→ | | |
| | | | | 5/95 (2.50 min) | | |
| 47 | Shimadzu | Shim-pack | 40 | A) 0.05% TFA, Water | 1.5 | 190-400 nm |
| | LCMS-2020 | XR-ODS | | B) 0.05% TFA, MeCN | | |
| | | 50 mm. 3.0 | | A/B = 70/30→ | | |
| | | mm. 2.2 μm | | 30/70 (2.85 min)→ | | |
| | | | | 5/95 (3.20 min) | | |
| 48 | Shimadzu | Kinetex XB- | 40 | A) 0.05% TFA, Water | 1.0 | 190-400 nm |
| | LCMS-2020 | C18 100A, | | B) 0.05% TFA, MeCN | | |
| | | 30 mm. 2.1 | | A/B = 95/5→ | | |
| | | mm. 1.7 μm | | 0/100 (1.50 min) | | |
| 49 | Shimadzu | Kinetex XB- | 40 | A) 0.05% TFA, Water | 1.0 | 190-400 nm |
| | LCMS-2020 | C18 100A, | | B) 0.05% TFA, MeCN | | |
| | | 50 mm. 3.0 | | A/B = 95/5→ | | |
| | | mm. 2.6 μm | | 0/100 (2.20 min) | | |
| 50 | Shimadzu | Accucore | 40 | A) 0.1% FA, Water | 1.0 | 190-400 nm |
| | LCMS-2020 | C18 , | | B) 0.1% FA, MeCN | | |
| | | 50 mm. 2.1 | | A/B = 95/5→ | | |
| | | mm. 2.6 μm | | 50/50 (3.50 min)→ | | |
| | | | | 5/95 (4.00 min) | | |
| 51 | Shimadzu | CORTECS | 40 | A) 0.1% FA, Water | 1.0 | 190-400 nm |
| | LCMS-2020 | C18+ | | B) 0.1% FA, MeCN | | |
| | | 50 mm. 2.1 | | A/B = 100/5→ | | |
| | | mm. 2.7 μm | | 0/100 (2.00 min) | | |
| 52 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 100/0→ | | |
| | | mm. 2.7 μm | | 60/40 (2.50 min)→ | | |
| | | | | 95/95 (3.20 min) | | |
| 53 | Shimadzu | Titank C18 | 40 | A) 5 mmolNH₄HCO₃, | 0.8 | 190-400 nm |
| | LCMS-2020 | 50 mm. 3.0 | | Water | | |
| | | mm. 3.0 μm | | B) MeCN | | |
| | | | | A/B = 70/30→ | | |
| | | | | 5/95 (3.50 min) | | |
| 54 | Shimadzu | ACE Excel 3 | 40 | A) 5 mmolNH₄HCO₃, | 1.2 | 190-400 nm |
| | LCMS-2020 | SuperC18, | | Water | | |
| | | 50 mm. 3.0 | | B) MeCN | | |
| | | mm. 3.0 μm | | A/B = 90/10→ | | |
| | | | | 5/95 (2.10 min) | | |
| 55 | Shimadzu | Shim-pack | 40 | A) 0.05% TFA, Water | 1.0 | 190-400 nm |
| | LCMS-2020 | XR-ODS | | B) 0.05% TFA, MeCN | | |
| | | 50 mm. 3.0 | | A/B = 90/10→ | | |
| | | mm. 2.2 μm | | 5/95 (2.00 min) | | |
| 56 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 0/100 (2.10 min) | | |
| 57 | Shimadzu | HALO C18, | 40 | A) 0.05% TFA, Water | 1.5 | 190-400 nm |
| | LCMS-2020 | 30 mm. 3.0 | | B) 0.05% TFA, MeCN | | |
| | | mm. 2.0 μm | | A/B = 95/5→ | | |
| | | | | 50/50 (1.80 min)→ | | |
| | | | | 5/95 (2.50 min) | | |
| 57-1 | Shimadzu | HALO C18, | 40 | A) 0.05% TFA, Water | 1.5 | 190-400 nm |
| | LCMS-2020 | 30 mm. 3.0 | | B) 0.05% TFA, MeCN | | |
| | | mm. 2.0 μm | | A/B = 95/5→ | | |
| | | | | 5/95 (2.50 min) | | |
| 58 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 70/30→ | | |
| | | mm. 2.7 μm | | 30/70 (2.85 min)→ | | |
| | | | | 95/5 (3.20 min) | | |
| 59 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 100/0→ | | |
| | | mm. 2.7 μm | | 5/95 (2.00 min) | | |
| 60 | Shimadzu | Accucore C18, | 40 | A) 0.1% FA, Water | 1.0 | 190-400 nm |
| | LCMS-2020 | 50 mm. 2.1 | | B) 0.1% FA, MeCN | | |
| | | mm. 2.6 μm | | A/B = 95/5→ | | |
| | | | | 5/95 (3.00 min) | | |
| 61 | Shimadzu | Shim-pack | 40 | A) 0.05% TFA, Water | 1.0 | 190-400 nm |
| | LCMS-2020 | XR-ODS | | B) 0.05% TFA, MeCN | | |
| | | 50 mm. 3.0 | | A/B = 90/10→ | | |
| | | mm. 2.2 μm | | 30/70 (1.90 min)→ | | |
| | | | | 5/95 (2.10 min) | | |
| 62 | Shimadzu | Kinetex | 40 | A) 0.1% FA, Water | 1.0 | 190-400 nm |
| | LCMS-2020 | 1.7 umC18, | | B) 0.1% FA, MeCN | | |
| | | 30 mm. 2.1 | | A/B = 95/5→ | | |
| | | mm. | | 5/95 (1.20 min) | | |
| 63 | Shimadzu | Kinetex EVO | 40 | A) 0.09% FA, Water | 1.5 | 190-400 nm |
| | LCMS-2020 | C18, | | B) 0.1% FA, MeCN | | |
| | | 50 mm. 3.0 | | A/B = 95/5→ | | |
| | | mm. 2.6 μm | | 70/30 (2.60 min)→ | | |
| | | | | 5/95 (3.70 min) | | |
| 63-1 | Shimadzu | Kinetex | 40 | A) 0.09% FA, Water | 1.5 | 190-400 nm |
| | LCMS-2020 | EVO C18, | | B) 0.1% FA, MeCN | | |
| | | 50 mm. 3.0 | | A/B = 95/5→ | | |
| | | mm. 2.6 μm | | 50/50 (3.00 min)→ | | |
| | | | | 5/95 (3.70 min) | | |
| 64 | Shimadzu | Ascentis | 40 | A) 0.09% FA, Water | 1.5 | 190-400 nm |
| | LCMS-2020 | Express C18, | | B) 0.1% FA, MeCN | | |
| | | 50 mm. 3.0 | | A/B = 95/5→ | | |
| | | mm. 2.7 μm | | 40/60 (3.25 min)→ | | |
| | | | | 5/95 (3.70 min) | | |
| 65 | Shimadzu | Kinetex | 40 | A) 0.1% FA, Water | 1.0 | 190-400 nm |
| | LCMS-2020 | 1.7 umC18, | | B) 0.1% FA, MeCN | | |
| | | 30 mm. 2.1 | | A/B = 95/5→ | | |
| | | mm. | | 0/100 (2.20 min) | | |
| 66 | Shimadzu | Shim-pack | 40 | A) 0.05% TFA, Water | 0.8 | 190-400 nm |
| | LCMS-2020 | XR-ODS | | B) 0.05% TFA, MeCN | | |
| | | 50 mm. 3.0 | | A/B = 95/5→ | | |
| | | mm. 2.2 μm | | 40/60 (3.25 min)→ | | |
| | | | | 5/95 (3.80 min) | | |
| 67 | Shimadzu | Poroshell | 40 | A) 5 mmolNH₄HCO₃, | 1.2 | 190-400 nm |
| | LCMS-2020 | HPH-C18, | | Water | | |
| | | 50 mm. 3.0 | | B) MeCN | | |
| | | mm. 2.7 μm | | A/B = 95/5→ | | |
| | | | | 5/95 (2.00 min) | | |
| 68 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 95/5→40/60 (3.25 | | |
| | | mm. 2.2 μm | | min)→5/95 (3.80 min) | | |
| 69 | Shimadzu | Shim-pack | 40 | A) 0.05% TFA, Water | 1.2 | 190-400 nm |
| | LCMS-2020 | XR-ODS | | B) 0.05% TFA, MeCN | | |
| | | 50 mm. 3.0 | | A/B = 40/60→ | | |
| | | mm. 2.2 μm | | 5/95 (2.00 min) | | |
| 70 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 30/70 (3.60 min)→ | | |
| | | | | 5/95 (4.20 min) | | |
| 71 | Shimadzu | Ascentis | 40 | A) 0.09% FA, Water | 1.5 | 190-400 nm |
| | LCMS-2020 | Express C18, | | B) 0.1% FA, MeCN | | |
| | | 50 mm. 3.0 | | A/B = 95/5→ | | |
| | | mm. 2.7 μm | | 40/60 (3.10 min)→ | | |
| | | | | 5/95 (3.65 min) | | |
| 72 | Shimadzu | Ascentis | 40 | A) 0.05% TFA, Water | 1.2 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 50/50 (3.25 min)→ | | |
| | | | | 5/95 (3.80 min) | | |
| 73 | Shimadzu | Kinetex EVO | 40 | A) 5 mmolNH₄HCO₃, | 1.0 | 190-400 nm |
| | LCMS-2020 | 50 mm. 3.0 | | Water | | |
| | | mm. 2.6 μm | | B) MeCN | | |
| | | | | A/B = 95/5→ | | |
| | | | | 5/95 (3.20 min) | | |
| 74 | Varian 1200 | Luna C18, | | A) 0.1% TFA, Water | 1.0 | 220 nm |
| | LC/MS | 150 mm, 4.6 | | B) 0.1% TFA, MeCN | | (detection ) |
| | | mm | | A/B = 90/10→ | | |
| | | | | 5/95 (20 min) | | |
| 75 | Varian 1200 | Luna C18, | | A) 0.1% TFA, Water | 1.0 | 220 nm |
| | LC/MS | 150 mm, 4.6 | | B) 0.1% TFA, MeCN | | (detection ) |
| | | mm | | A/B = 90/10→ | | |
| | | | | 5/95 (40 min) | | |
| 76 | UPLC + | Acquity | 40 | A) 0.1% FA, Water | 0.5 | 210-400 nm |
| | Waters DAD + | UPLC HSS | | B) 0.1% FA, MeCN | | |
| | Waters | C18 1.8 um | | A/B = 95/5→ | | |
| | SQD2, single | 100 × 2.1 mm. | | 0/100 (3.5 min) | | |
| | quadrapole | | | | | |
| | UPLC-MS | | | | | |
| 77 | UPLC + | Acquity | 40 | A) 10 mmolNH₄HCO₃, | 0.5 | 210-400 nm |
| | Waters DAD + | UPLC BEH | | Water | | |
| | Waters | Shield RP18 | | B) MeCN | | |
| | SQD2, single | 1.7 um 100 × | | A/B = 95/5→ | | |
| | quadrapole | 2.1 mm. | | 100/0 (3.50 min) | | |
| | UPLC-MS | | | | | |
| 78 | UPLC + | Acquity | 40 | A) 0.1% FA, Water | 0.4 | 210-400 nm |
| | Waters DAD + | UPLC HSS | | B) 0.1% FA, MeCN | | |
| | Waters | C18 1.8 um | | A/B = 95/5→ | | |
| | SQD2, single | 100 × 2.1 mm. | | 5/95 (6.0 min) | | |
| | quadrapole | | | | | |
| | UPLC-MS | | | | | |
| 79 | UPLC + | Acquity | 40 | A) 10 mmolNH₄HCO₃, | 0.4 | 210-400 nm |
| | Waters DAD + | UPLC BEH | | Water | | |
| | Waters | Shield RP18 | | B) MeCN | | |
| | SQD2, single | 1.7 um 100 × | | A/B = 95/5→ | | |
| | quadrapole | 2.1 mm | | 5/95 (6.0 min) | | |
| | UPLC-MS | | | | | |
| 80 | UPLC + | ACE-AR | 40 | A) 0.1% FA, Water | 0.5 | 210-400 nm |
| | Waters DAD + | ACE excel | | B) 0.1% FA, MeCN | | |
| | Waters | 2 um C18-AR | | A/B = 95/5→ | | |
| | SQD2, single | | | 0/100 (3.5 min) | | |
| | quadrapole | | | | | |
| | UPLC-MS | | | | | |
| 81 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 5/95 (1.70 min) | | |
| 82 | Varian 1200 | Luna C18, | | A) 0.1% TFA, Water | 1.0 | 220 nm |
| | LC/MS | 150 mm, 4.6 | | B) 0.1% TFA, MeCN | | (dete ction ) |
| | | mm | | A/B = 90/10→ | | |
| | | | | 5/95 (20 min) | | |
| 83 | Waters | YMC Triart | 30 | A) 0.1% TFA, Water | 1.0 | 190-500 nm |
| | Acquity | C18 | | B) 0.1% TFA, MeCN | | |
| | UPLC I- | 2.0 mm × 30 | | A/B = 95/5→ | | |
| | Class system | mm, 1.9 μm | | 5/95 (1.2 min) | | |
| 84 | Shimadzu | Ascentis | 40 | A) 0.05% TFA, Water | 1.5 | 190-400 nm |
| | LCMS-2020 | Express C18 | | B) 0.05% TFA, MeCN | | |
| | | 50 mm. 3.0 | | A/B = 95/5→ | | |
| | | mm. 2.7 μm | | 0/100 (2.00 min) | | |
| 84-1 | Shimadzu | Ascentis | 40 | A) 0.05% TFA, Water | 1.2 | 190-400 nm |
| | LCMS-2020 | Express C18 | | B) 0.05% TFA, MeCN | | |
| | | 50 mm. 3.0 | | A/B = 95/5→ | | |
| | | mm. 2.7 μm | | 0/100 (2.00 min) | | |
| 85 | Shimadzu | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 70/30→ | | |
| | | mm. 2.7 μm | | 40/60 (2.70 min)→ | | |
| | | | | 5/95 (3.20 min) | | |
| 86 | Shimadzu. | Ascentis | 40 | A) 0.05% TFA, Water | 1.5 | 190-400 nm |
| | LCMS-2020 | Express C18, | | B) 0.05% TFA, MeCN | | |
| | | 50 mm. 3.0 | | A/B = 80/20→ | | |
| | | mm. 2.7 μm | | 50/50 (2.90 min)→ | | |
| | | | | 5/95 (3.30 min) | | |
| 87 | Shimadzu | Ascentis | 40 | A) 0.05% TFA, Water | 1.2 | 190-400 nm |
| | LCMS-2020 | Express C18, | | B) 0.05% TFA, MeCN | | |
| | | 50 mm. 3.0 | | A/B = 95/5→ | | |
| | | mm. 2.7 μm | | 30/70 (3.25 min)→ | | |
| | | | | 5/95 (3.80 min) | | |
| 88 | Shimadzu | Luna Omega | 40 | A) 0.09% TFA, Water | 1.5 | 190-400 nm |
| | LCMS-2020 | 50 mm. 3.0 | | B) 0.1% TFA, MeCN | | |
| | | mm. 3 μm | | A/B = 95/5→ | | |
| | | | | 0/100 (2.00 min) | | |
| 89 | Shimadzu | Luna Omega | 40 | A) 0.09% TFA, Water | 1.5 | 190-400 nm |
| | LCMS-2020 | 50 mm. 3.0 | | B) 0.1% TFA, MeCN | | |
| | | mm. 3 μm | | A/B = 95/5→ | | |
| | | | | 70/40 (2.80 min)→ | | |
| | | | | 5/95 (3.60 min) | | |
| 90 | Shimadzu | Luna Omega | 40 | A) 0.09% TFA, Water | 1.5 | 190-400 nm |
| | LCMS-2020 | 50 mm. 3.0 | | B) 0.1% TFA, MeCN | | |
| | | mm. 3 μm | | A/B = 95/5→ | | |
| | | | | 0/100 (1.30 min) | | |
| 91 | Shimadzu | XBridge | 40 | A) 5 mmolNH₄HCO₃, | 1.2 | 190-400 nm |
| | LCMS-2020 | BEH C18 | | Water | | |
| | | 50 mm. 3.0 | | B) MeCN | | |
| | | mm. 2.5 μm | | A/B = 95/5→ | | |
| | | | | 5/95 (2.00 min) | | |
| 92 | Shimadzu | Gemini NM- | 40 | A) 5 mmolNH₄HCO₃, | 1.5 | 190-400 nm |
| | LCMS-2020 | C18 | | Water | | |
| | | 50 mm. 3.0 | | B) MeCN | | |
| | | mm. 3 μm | | A/B = 95/5→ | | |
| | | | | 5/95 (2.00 min) | | |
| 93 | Shimadzu | Poroshell | 40 | A) 0.04% NH₃H₂O, | 1.2 | 190-400 nm |
| | LCMS-2020 | HPH-C18, | | Water | | |
| | | 50 mm. 3.0 | | B) MeCN | | |
| | | mm. 2.7 μm | | A/B = 95/5→ | | |
| | | | | 5/95 (2.00 min) | | |
| 94 | Shimadzu | YMC Triart | 40 | A) 0.04% NH₃H₂O, | 1.2 | 190-400 nm |
| | LCMS-2020 | C18 | | Water | | |
| | | 30 mm × 2.0 | | B) MeCN | | |
| | | mm, 3.0 μm | | A/B = 95/5→ | | |
| | | | | 5/95 (2.00 min) | | |

### Reference Example 1: tert-butyl N-tert-butoxycarbonyl-N-[5-(trifluoromethyl)-3-pyridyl]carbamate

To a stirred solution of 5-(trifluoromethyl)pyridin-3-amine (CAS No. 112110-07-3, 30 g) in pyridine (180 mL) was added N,N-dimethylaminopyridine (678 mg) and di-tert-butyl dicarbonate (101 g) at the room temperature under N₂. The resulting mixture was stirred overnight at 50° C. under N₂. The resulting mixture was diluted with ethyl acetate, washed with saturated brine solution, dried over anhydrous Na₂SO₄. After filtration, the filtrate was concentrated under reduced pressure. The residue was purified by silica gel column chromatography, eluted with (petroleum ether:ethyl acetate=1:1) to afford the title compound (40 g).

### Reference Example 2: tert-butyl N-[5-(trifluoromethyl)-3-pyridyl]carbamate

To a stirred solution of the compound prepared in Reference Example 1 (8.3 g) in MeOH (25 mL) was added NaOMe in MeOH (52 mL, 22.91 mmol) at 0° C. under N₂. The resulting mixture was stirred for 2 hours at room temperature under N₂. The filtrate was concentrated under reduced pressure. The resulting mixture was diluted with ethyl acetate, washed with saturated brine solution and dried over anhydrous Na₂SO₄. The crude product was used in the next step directly without further purification.

ESI-MS m/z: 263.20 [M+H]+.

### Reference Example 3: methyl 2-[tert-butoxycarbonyl-[5-(trifluoromethyl)-3-pyridyl]amino]acetate

To a stirred solution of the compound prepared in Reference Example 2 (5.8 g) in MeCN (180 mL) was added Cs₂CO₃ (22 g) and methyl 2-bromoacetate (3.3 mL) at 0° C. under N₂. The resulting mixture was stirred overnight at 50° C. under N₂. The reaction was monitored by TLC. The resulting mixture was diluted with ethyl acetate, washed with saturated brine solution and dried over anhydrous Na₂SO₄. After filtration, the filtrate was concentrated under reduced pressure. The crude product was used in the next step directly without further purification.

ESI-MS m/z: 335.30 [M+H]+.

### Reference Example 4: methyl 2-[[5-(trifluoromethyl)-3-pyridyl]amino]acetate

To a stirred solution of the compound prepared in Reference Example 3 (5.9 g) in dichloromethane (DCM) (50 mL) was added TFA (10 mL) at 0° C. The resulting mixture was stirred 2 h at room temperature. Evaporated the solvent and diluted with ethyl acetate. The PH was adjusted to 8 with NaHCO₃ (aqueous), washed with saturated brine solution and dried over anhydrous Na₂SO₄. After filtration, the filtrate was concentrated under reduced pressure. The crude product was used in the next step directly without further purification.

ESI-MS m/z: 235.20 [M+H]+.

### Reference Example 5: methyl 2-[chlorocarbonyl-[5-(trifluoromethyl)-3-pyridyl]amino]acetate

To a stirred solution of the compound prepared in Reference Example 4 (10 g) in DCM (250 mL) was added triethylamine (22 mL) and trichloromethyl carbonochloridate (7.7 mL) under N₂. The resulting mixture was refluxed for 4 h under N₂. The resulting mixture was cooled to room temperature and concentrated under reduced pressure. The residue was purified by silica gel column chromatography, eluted with (petroleum ether:ethyl acetate=1:1) to afford the title compound (7.3 g) having the following physical property values.

ESI-MS m/z: 297.1 [M+H]+.

### Reference Example 6: 5-(4-nitrophenoxy)pyrimidin-2-amine

To a stirred solution of 2-aminopyrimidin-5-ol (CAS No. 143489-45-6, 444 mg) and K₂CO₃ (1105 mg) in DMSO (10 mL) were added 1-fluoro-4-nitro-benzene (CAS No. 350-46-9, 564 mg) under nitrogen atmosphere. The resulting mixture was stirred for 2 h at room temperature under nitrogen atmosphere. The resulting mixture was diluted with ethyl acetate. The combined organic layer was washed with H₂O, brine, and dried over anhydrous sodium bicarbonate. After filtration, the filtrate was concentrated under reduced pressure. The residue was purified by silica gel column chromatography, DCM/MeOH (10:1) to afford the title compound (735 mg) having the following physical property values.

ESI-MS m/z: 233.2 [M+H]+.

### Reference Example 7: 5-(4-aminophenoxy)pyrimidin-2-amine

To a stirred solution of the compound prepared in Reference Example 6 (735 mg) in ethanol (30 mL) were added NH₄Cl (847 mg) in 6.0 mL of H₂O. To the above mixture was added Fe (1414 mg) at 80° C., the resulting mixture was stirred for 2 hr at 80° C. under nitrogen atmosphere. The reaction mixture was filtered through a pad of celite (trademark) and the filtrete was collected and concentrated under reduced pressure. The residue was partitioned between ethyl acetate and water. The combined organic layers were washed with water and brine, dried over anhydrous Na₂SO₄, filtered and concentrated under vacuum. The residue was purified by silica gel column chromatography, DCM/MeOH (9:1) to afford the title compound (540 mg) having the following physical property values.

MS(ESI, Pos.): 203.20 (M+H)+;

¹H NMR (400 MHz, DMSO-d6): δ 8.02, 6.76-6.68, 6.58-6.50, 6.44, 4.88.

### Example 1: 3-{4-[(2-amino-5-primidinyl)oxy]phenyl}-1-[5-(trifluoromethyl)-3-pyridinyl]-2,4-imidazolidinedione

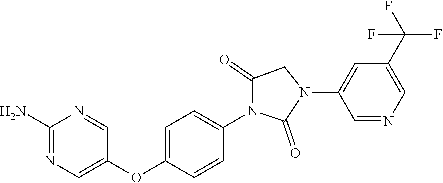 [SMILES: Nc1ncc(Oc2ccc(N3C(=O)CN(c4cncc(C(F)(F)F)c4)C3=O)cc2)cn1]

To a solution of the compound prepared in Reference Example 7 (100 mg) in THE (8 mL), was added N,N-diisopropylethylamine (DIEA) (0.26 mL) and the compound prepared in Reference Example 5 (220 mg) under nitrogen atmosphere. The resulting solution was refluxed at 80° C. for 2 h. The mixture was concentrated under vacuum. The crude product was purified by Prep-HPLC with the following conditions (Column: Xselect CSH OBD Column (trademark) 30*150 mm 5 um, n; Mobile Phase A: Water, Mobile Phase B: MeCN; Flow rate: 60 mL/min; Gradient: 29% B to 47% B in 7 min; 254/220 nm; RT: 6.85) to afford the title compound (48 mg) having the following physical property values.

¹H NMR (400 MHz, DMSO-d6): δ 9.17, 8.77, 8.54-8.48, 8.22, 7.43-7.35, 7.13-7.05, 6.70, 4.74;

LCMS: m/z 431 [M+H]+;

HPLC retention time: 1.167 min (method 5).

### Example 1(1)˜1(40)

A procedure for a purpose similar to that for Reference Example 6→Reference Example 7→Example 1 was carried out by using a corresponding alcohol compound instead of 2-aminopyrimidin-5-ol, a corresponding halide compound instead of 1-fluoro-4-nitro-benzene, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 1(1): 1-[3-(3-pyridinyl)-5-(trifluoromethyl)phenyl]-3-[4-(thieno[3,2-b]pyridin-7-yloxy)phenyl]-2,4-imidazolidinedione

¹H NMR (500 MHz, CDCl₃): δ 8.89, 8.69, 8.56, 8.19, 7.93, 7.86, 7.81, 7.67, 7.60, 7.43, 7.34, 6.71, 4.63;

MS (ESI, Pos.): 547 (M+H)+;

HPLC retention time: 8.03 min (Method 74).

### Example 1(2): 3-[3-ethyl-4-(1H-pyrrolo[2,3-b]pyridin-5-yloxy)phenyl]-1-[3-(trifluoromethyl)phenyl]-2,4-imidazolidinedione

¹H NMR (400 MHz, DMSO-d₆): δ 11.73, 8.17, 8.09, 7.89, 7.69-7.65, 7.54-7.50, 7.36, 7.19, 6.80, 6.44, 4.68, 2.78, 1.25;

LCMS: m/z 481 [M+H]+;

HPLC retention time: 1.654 min (method 5).

### Example 1(3): formic acid—3-[3-ethyl-4-(1H-pyrazol-4-yloxy)phenyl]-1-[5-(trifluoromethyl)-3-pyridinyl]-2,4-imidazolidinedione (1:1)

¹H NMR (300 MHz, DMSO-d₆): δ 9.17, 8.77, 8.53, 8.15, 7.63, 7.30, 7.19, 6.93, 4.75, 2.74, 1.23;

LCMS: m/z 432 [M+H]+;

HPLC retention time: 2.777 min (method 4).

### Example 1(4): 3-[3-ethyl-4-(pyrazolo[1,5-a]pyridin-3-yloxy)phenyl]-1-[5-(trifluoromethyl)-3-pyridinyl]-2,4-imidazolidinedione

¹H NMR (300 MHz, DMSO-d6): δ 9.18, 8.78, 8.67, 8.53, 8.10, 7.45-7.35, 7.23-7.13, 6.95-6.79, 4.75, 2.90, 1.32;

LCMS: m/z 482 [M+H]+;

HPLC retention time: 1.563 min (method 5).

### Example 1(5): 3-[3-bromo-4-(1H-pyrazol-4-yloxy)phenyl]-1-[5-(trifluoromethyl)-3-pyridinyl]-2,4-imidazolidinedione

¹H NMR (400 MHz, DMSO-d₆): δ 9.17, 8.78, 8.51, 7.94-7.72, 7.40, 7.07, 4.75;

LCMS: m/z 482 [M+H]+;

HPLC retention time: 1.565 min (method 1).

### Example 1(6): 3-[4-(1H-pyrrolo[2,3-b]pyridin-5-yloxy)phenyl]-1-[5-(trifluoromethyl)-3-pyridinyl]-2,4-imidazolidinedione

¹H NMR (400 MHz, DMSO-d₆): δ 11.79, 9.17, 8.77, 8.51, 8.12, 7.79, 7.56, 7.40-7.36, 7.09-7.05, 6.46, 4.74;

LCMS: m/z 495 [M+MeCN+H]+;

HPLC retention time: 3.340 min (method 39).

### Example 1(7): 3-[3-bromo-4-(1H-pyrrolo[2,3-b]pyridin-5-yloxy)phenyl]-1-[3-(trifluoromethyl)phenyl]-2,4-imidazolidinedione

¹H NMR (400 MHz, DMSO-d₆): δ 11.81, 8.20-8.12, 7.89, 7.87-7.78, 7.76, 7.69-7.51, 7.38, 6.97, 6.47, 4.68;

LCMS: m/z 531 [M+H]+;

HPLC retention time: 1.650 min (method 5).

### Example 1(8): 3-[4-(1H-pyrrolo[2,3-b]pyridin-5-yloxy)-3-vinylphenyl]-1-[3-(trifluoromethyl)phenyl]-2,4-imidazolidinedione

¹H NMR (400 MHz, DMSO-d₆): δ 11.76, 8.17-8.10, 7.90, 7.77-7.65, 7.55-7.51, 7.27, 7.12, 6.87, 6.45, 5.90, 5.44, 4.70;

LCMS: m/z 479 [M+H]+;

HPLC retention time: 1.639 min (method 5).

### Example 1(9): 3-[3-methyl-4-(1H-pyrrolo[2,3-b]pyridin-5-yloxy)phenyl]-1-[3-(trifluoromethyl)phenyl]-2,4-imidazolidinedione

¹H NMR (400 MHz, DMSO-d₆): δ 11.75, 8.17, 8.10, 7.88, 7.70-7.66, 7.55-7.51, 7.35, 7.19-7.16, 6.80, 6.45, 4.69, 2.33;

LCMS: m/z 467 [M+H]+;

HPLC retention time: 1.826 min (method 1).

### Example 1(10): 3-[3-fluoro-4-(1H-pyrazolo[2,3-b]pyridin-5-yloxy)phenyl]-1-[3-(trifluoromethyl)phenyl]-2,4-imidazolidinedione

¹H NMR (300 MHz, DMSO-d₆): δ 11.82, 8.18, 7.90, 7.78, 7.69, 7.58-7.49, 7.24, 7.10, 6.47, 4.70;

LCMS: m/z 471 [M+H]+;

HPLC retention time: 2.971 min (method 16).

### Example 1(11): 3-[4-(1H-pyrazolo[3,4-b]pyridin-5-yloxy)phenyl]-1-[5-(trifluoromethyl)-3-pyridinyl]-2,4-imidazolidinedione

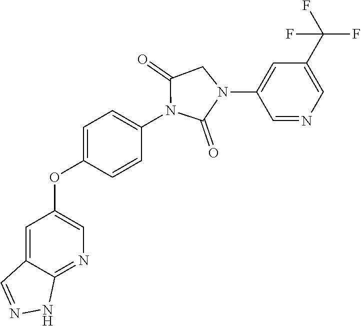 [SMILES: O=C1CN(c2cncc(C(F)(F)F)c2)C(=O)N1c1ccc(Oc2cnc3[nH]ncc3c2)cc1]

¹H NMR (400 MHz, DMSO-d₆): δ 13.78, 9.18, 8.78, 8.52-8.47, 8.14, 8.04, 7.44-7.40, 7.17-7.14, 4.75;

LCMS: m/z 455 [M+H]+;

HPLC retention time: 1.327 min (method 5).

### Example 1(12): 3-[4-(thieno[3,2-b]pyridin-6-yloxy)phenyl]-1-[5-(trifluoromethyl)-3-pyridinyl]-2,4-imidazolidinedione

¹H NMR (400 MHz, DMSO-d₆): δ 9.18, 8.78, 8.59-8.52, 8.35, 8.11, 7.59, 7.48-7.44, 7.24-7.20, 4.76;

LCMS: m/z 471 [M+H]+;

HPLC retention time: 0.969 min (method 62).

### Example 1(13): 3-[4-(thieno[2,3-b]pyridin-5-yloxy)phenyl]-1-[5-(trifluoromethyl)-3-pyridinyl]-2,4-imidazolidinedione

¹H NMR (400 MHz, DMSO-d₆): δ 9.18, 8.77, 8.52, 8.04, 7.98, 7.47-7.44, 7.25-7.21, 4.76;

LCMS: m/z 471 [M+H]+;

HPLC retention time: 1.620 min (method 5).

### Example 1(14): 3-{4-[(2-amino-5-pyrimidinyl)oxy]phenyl}-1-[3-(trifluoromethyl)phenyl]-2,4-imidazolidinedione

¹H NMR (400 MHz, DMSO-d₆): δ 8.22-8.16, 7.90, 7.69, 7.53, 7.41-7.37, 7.10-7.06, 6.70, 4.68;

LCMS: m/z 430 [M+H]+;

HPLC retention time: 1.376 min (method 5).

### Example 1(15): 3-{4-[(2-amino-5-pyrimidinyl)oxy]phenyl}-1-[3-(difluoromethyl)phenyl]-2,4-imidazolidinedione

¹H NMR (400 MHz, DMSO-d₆): δ 8.22, 8.00, 7.76, 7.58, 7.40-7.35, 7.09-7.06, 6.70, 4.64;

LCMS: m/z 412 [M+H]+;

HPLC retention time: 1.569 min (method 55).

### Example 1(16): 3-{4-[(2-amino-5-pyrimidinyl)oxy]phenyl}-1-[3-fluoro-5-(trifluoromethyl)phenyl]-2,4-imidazolidinedione

¹H NMR (400 MHz, DMSO-d₆): δ 8.22, 8.02, 7.83, 7.47, 7.40-7.36, 7.11-7.07, 6.70, 4.68;

LCMS: m/z 448 [M+H]+;

HPLC retention time: 1.760 min (method 55).

### Example 1(17): 3-{4-[(2-amino-5-pyrimidinyl)oxy]phenyl}-1-[4-fluoro-3-(trifluoromethyl)phenyl]-2,4-imidazolidinedione

¹H NMR (400 MHz, DMSO-d₆): δ 8.22, 8.14, 7.95, 7.62, 7.40-7.36, 7.10-7.06, 6.70, 4.68;

LCMS: m/z 448 [M+H]+;

HPLC retention time: 1.362 min (method 57-1).

### Example 1(18): 3-{4-[(2-amino-5-pyrimidinyl)oxy]phenyl}-1-[4-fluoro-3-(trifluoromethoxy)phenyl]-2,4-imidazolidinedione

¹H NMR (300 MHz, DMSO-d₆): δ 8.22, 8.05-8.02, 7.65-7.57, 7.40-7.35, 7.10-7.05, 6.71, 4.63;

LCMS: m/z 464 [M+H]+;

HPLC retention time: 3.020 min (method 64).

### Example 1(19): 3-{4-[(2-amino-5-pyrimidinyl)oxy]phenyl}-1-[3-(difluoromethoxy)phenyl]-2,4-imidazolidinedione

¹H NMR (400 MHz, DMSO-d₆): δ 8.22, 7.62, 7.52-7.45, 7.39-7.36, 7.27, 7.10-7.06, 6.98, 6.70, 4.61;

LCMS: m/z 428 [M+H]+;

HPLC retention time: 1.387 min (method 44).

### Example 1(20): 3-[2-fluoro-4-(1H-pyrazolo[3,4-b]pyridin-5-yloxy)phenyl]-1-[5-(trifluoromethyl)-3-pyridinyl]-2,4-imidazolidinedione

¹H NMR (400 MHz, DMSO-d₆): δ 13.83, 9.17, 8.79, 8.51-8.49, 8.16-8.13, 7.49, 7.18, 6.97, 4.90-4.84;

LCMS: m/z 473 [M+H]+;

HPLC retention time: 2.595 min (method 71).

### Example 1(21): 3-[2-methyl-4-(1H-pyrazolo[3,4-b]pyridin-5-yloxy)phenyl]-1-[5-(trifluoromethyl)-3-pyridinyl]-2,4-imidazolidinedione

¹H NMR (400 MHz, DMSO-d₆): δ 13.77, 9.17, 8.78, 8.51-8.46, 8.14, 8.03, 7.31, 7.03, 6.95, 4.87, 4.75, 2.16;

LCMS: m/z 469 [M+H]+;

HPLC retention time: 1.608 min (method 38).

### Example 1(22): 3-[2-chloro-4-(1H-pyrazolo[3,4-b]pyridin-5-yloxy)phenyl]-1-[5-(trifluoromethyl)-3-pyridinyl]-2,4-imidazolidinedione

¹H NMR (400 MHz, DMSO-d₆): δ 13.81, 9.17, 8.79, 8.51-8.49, 8.16-8.13, 7.54, 7.36, 7.12, 4.99, 4.81;

LCMS: m/z 489 [M+H]+;

HPLC retention time: 2.904 min (method 16-2).

### Example 1(23): 3-[2-methoxy-4-(1H-pyrazolo[3,4-b]pyridin-5-yloxy)phenyl]-1-[5-(trifluoromethyl)-3-pyridinyl]-2,4-imidazolidinedione

¹H NMR (400 MHz, DMSO-d₆): δ 13.78, 9.15, 8.77, 8.49-8.48, 8.15-8.05, 7.29, 6.97, 6.57, 4.93, 4.77, 3.76;

LCMS: m/z 485 [M+H]+;

HPLC retention time: 1.444 min (method 44).

### Example 1(24): 3-[2-(difluoromethoxy)-4-(1H-pyrazolo[3,4-b]pyridin-5-yloxy)phenyl]-1-[5-(trifluoromethyl)-3-pyridinyl]-2,4-imidazolidinedione

¹H NMR (400 MHz, DMSO-d₆): δ 13.83, 9.17, 8.79, 8.51-8.49, 8.17-8.12, 7.50-7.06, 6.97, 4.98, 4.80;

LCMS: m/z 521 [M+H]+;

HPLC retention time: 1.647 min (method 38).

### Example 1(25): 3-{4-[(3-hydroxy-1H-pyrazolo[3,4-b]pyridin-5-yl)oxy]phenyl}-1-[5-(trifluoromethyl)-3-pyridinyl]-2,4-imidazolidinedione

¹H NMR (400 MHz, DMSO-d₆): δ 12.28, 10.84, 9.18, 8.78, 8.52, 8.39, 7.85, 7.42, 7.14, 4.76;

LCMS: m/z 471 [M+H]+;

HPLC retention time: 1.409 min (method 38).

### Example 1(26): 3-{4-[(3-methoxy-1H-pyrazolo[3,4-b]pyridin-5-yl)oxy]phenyl}-1-[5-(trifluoromethyl)-3-pyridinyl]-2,4-imidazolidinedione

¹H NMR (400 MHz, DMSO-d₆): δ 12.71, 9.17, 8.78, 8.52, 8.43, 7.90, 7.41, 7.12, 4.75, 4.00;

LCMS: m/z 485 [M+H]+;

HPLC retention time: 1.399 min (method 5).

### Example 1(27): 3-[4-({3-[3-(methylsulfonyl)propoxy]-1H-pyrazolo[3,4-b]pyridin-5-yl}oxy)phenyl]-1-[5-(trifluoromethyl)-3-pyridinyl]-2,4-imidazolidinedione

¹H NMR (400 MHz, DMSO-d₆): δ 12.74, 9.18, 8.78, 8.52, 8.45, 7.93, 7.43-7.39, 7.15-7.09, 4.75, 4.44, 3.35-3.31, 3.01, 2.26-2.19;

LCMS: m/z 591 [M+H]+;

HPLC retention time: 3.052 min (method 72).

### Example 1(28): 1-[3-(methylsulfonyl)-5-(trifluoromethyl)phenyl]-3-[4-(1H-pyrazolo[3,4-b]pyridin-5-yloxy)phenyl]-2,4-imidazolidinedione

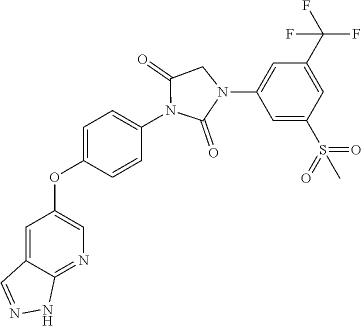 [SMILES: CS(=O)(=O)c1cc(N2CC(=O)N(c3ccc(Oc4cnc5[nH]ncc5c4)cc3)C2=O)cc(C(F)(F)F)c1]

¹H NMR (400 MHz, DMSO-d₆): δ 13.78, 8.53, 8.48, 8.35, 8.14, 8.04-8.01, 7.45-7.41, 7.17-7.13, 4.79, 3.38;

LCMS: m/z 532 [M+H]+;

HPLC retention time: 2.678 min (method 71).

### Example 1(29): 1-{3-[3-(methylsulfonyl)propoxy]-5-(trifluoromethyl)phenyl}-3-[4-(1H-pyrazolo[3,4-b]pyridin-5-yloxy)phenyl]-2,4-imidazolidinedione

¹H NMR (400 MHz, DMSO-d₆): δ 13.78, 8.48, 8.14, 8.03, 7.76, 7.47, 7.43-7.40, 7.15-7.13, 7.07, 4.67, 4.21, 3.30, 3.03, 2.20-2.16;

LCMS: m/z 590 [M+H]+;

HPLC retention time: 2.767 min (method 71).

### Example 1(30): 3-{4-[(3-amino-1H-pyrazolo[3,4-b]pyridin-5-yl)oxy]phenyl}-1-[5-(trifluoromethyl)-3-pyridinyl]-2,4-imidazolidinedione

¹HNMR (400 MHz, DMSO-d₆): δ 12.18-12.08, 9.17, 8.77, 8.51, 8.32, 7.95, 7.43-7.39, 7.14-7.07, 5.59, 4.75;

LCMS: m/z 470 [M+H]+;

HPLC retention time: 1.184 min (method 5).

### Example 1(31): 3-[4-(1H-pyrrolo[2,3-b]pvridin-5-yloxy)phenyl]-1-[3-(trifluoromethyl)phenyl]-2,4-imidazolidinedione

¹HNMR (400 MHz, DMSO-d₆): δ 11.80, 8.12, 7.82, 7.79, 7.70, 7.55, 7.39, 7.07, 6.46, 4.67;

LCMS: m/z 494 [M+H]+;

HPLC retention time: 1.904 min (method 10).

### Example 1(32): 3-[4-(1H-pyrrolo[2,3-b]pyridin-4-yloxy)phenyl]-1-[3-(trifluoromethyl)phenyl]-2,4-imidazolidinedione

¹H NMR (500 MHz, DMSO-d₆): δ 11.79, 8.19, 8.15, 7.89, 7.69, 7.54-7.50, 7.40-7.39, 7.33-7.31, 6.56, 6.21-6.20, 4.71;

LCMS: m/z 453 [M+H]+;

HPLC retention time: 10.91 min (method 74).

### Example 1(33): 3-{4-[(7-hydroxy-6-methoxy-4-quinazolinyl)oxy]-2-methoxyphenyl}-1-[3-(trifluoromethyl)phenyl]-2,4-imidazolidinedione

¹H NMR (400 MHz, DMSO-d₆): δ 10.94, 8.60, 8.16, 7.92, 7.69, 7.59-7.53, 7.45, 7.26, 7.05, 4.93, 4.84, 4.01, 3.78;

LCMS: m/z 541 [M+H]+;

HPLC retention time: 1.688 min (method 2).

### Example 1(34): N—[5-(4-{2,5-dioxo-3-[5-(trifluoromethyl)-3-pyridinyl]-1-imidazolidinyl}phenoxy)-2-pyridinyl]acetamide

¹H NMR (400 MHz, DMSO-d₆): δ 10.57, 9.17, 8.77, 8.52, 8.20-8.13, 7.62, 7.44-7.40, 7.16-7.12, 4.75, 2.09;

LCMS: m/z 472 [M+H]+;

HPLC retention time: 1.492 min (method 60).

### Example 1(35)

### 3-[4-(3H-imidazo[4,5-b]pyridin-6-yloxy)phenyl]-1-[5-(trifluoromethyl)-3-pyridinyl]-2,4-imidazolidinedione

¹H NMR (300 Hz, DMSO-d₆) δ 9.21-9.14, 8.80-8.74, 8.55-8.48, 8.32, 7.87-7.82, 7.46-7.36, 7.19-7.10, 4.76;

LCMS: m/z 455 [M+H]+;

HPLC retention time: 1.072 min (method 5).

### Example 1(36)

### 3-[4-(1H-pyrazolo[4,3-b]pyridin-6-yloxy)phenyl]-1-[5-(trifluoromethyl)-3-pyridinyl]-2,4-imidazolidinedione

¹H NMR (400 MHz, DMSO-d₆) δ ppm: 13.24, 9.19, 8.79, 8.53, 8.45, 8.32, 7.61, 7.49, 7.27,

LCMS: m/z 455 [M+H]+;

HPLC retention time: 1.254 min (method 84).

### Example 1(37)

### 1-{3-[2-(methylsulfonyl)ethoxy]-5-(trifluoromethyl)phenyl}-3-[4-(1H-pyrazolo[3,4-b]pyridin-5-yloxy)phenyl]-2,4-imidazolidinedione

¹H NMR (400 MHz, DMSO-d₆) δ13.78, 8.47, 8.14, 8.03, 7.82, 7.48, 7.42, 7.14, 4.69, 4.48, 3.67, 3.11;

LCMS: m/z 576 [M+H]+;

HPLC retention time: 1.707 min (method 9).

### Example 1(38)

### 3-{4-[(5-amino-2-pyrazinyl)oxy]phenyl}-1-[5-(trifluoromethyl)-3-pyridinyl]-2,4-imidazolidinedione

¹H NMR (300 MHz, DMSO-d₆) δ 9.20-9.15, 8.81-8.76, 8.54-8.50, 7.91, 7.60, 7.43-7.36, 7.17-7.10, 6.32, 4.76;

LCMS: m/z 431 [M+H]+;

HPLC retention time: 2.396 min (method 16-2).

### Example 1(39)

### 3-{4-[(6,7-dimethoxy-3-quinolinyl)oxy]phenyl}-1-[5-(trifluoromethyl)-3-pyridinyl]-2,4-imidazolidinedione

¹H NMR (400 MHz, DMSO-d₆) δ 9.18, 8.78, 8.61, 8.55-8.49, 7.83, 7.50-7.43, 7.41, 7.37, 7.28-7.21, 4.77, 3.93, 3.88;

LCMS: m/z 525 [M+H]+;

HPLC retention time: 1.199 min (method 84).

### Example 1(40)

### 3-{4-[(3-amino-1H-pyrazolo[3,4-b]pyridin-5-yl)oxy]-2,6-dimethylphenyl}-1-[5-(trifluoromethyl)-3-pyridinyl]-2,4-imidazolidinedione trifluoroacetate (1:1

¹H NMR (300 MHz, DMSO-d₆) δ 12.07, 9.18, 8.79, 8.56-8.47, 8.29, 7.94, 6.83, 5.58, 4.94, 2.12;

LCMS: m/z 498 [M+H]+;

HPLC retention time: 1.212 min (method 84).

### Reference Example 8: 4-(4-amino-3-methylphenoxy)-2-pyrimidineamine

To a stirred solution of 4-amino-3-methylphenol (CAS No. 2835-99-6, 3.9 mL) in DMF (200 mL) was added NaH (0.97 g) at 0° C. under N₂. The resulting mixture was stirred for 1 hour at room temperature under N₂. To above mixture was added 2-amino-4-chloropyrimidine (CAS No. 3993-78-0, 5.3 g) at 0° C. under N₂. The resulting mixture was stirred for 2 hours at 110° C. under N₂. The reaction mixture was quenched with H₂O and diluted with ethyl acetate. The combined organic layers were washed with water, brine and dried over anhydrous sodium sulfate. After filtration, the filtrate was concentrated under reduced pressure. The residue was purified by silica gel column chromatography, eluted with ethyl acetate/petroleum ether (1:1) to afford the title compound (5.2 g) having the following physical property values.

ESI-MS m/z: 217.1 [M+H]+;

¹H NMR (300 Hz, DMSO-d₆): δ 8.02, 6.71, 6.71-6.56, 6.52, 5.92, 4.77, 2.04.

### Example 2: 3-{4-[(2-amino-4-pyrimidinyl)oxy]-2-methylphenyl}-1-[5-(trifluoromethyl)-3-pyridinyl]-2,4-imidazolidinedione

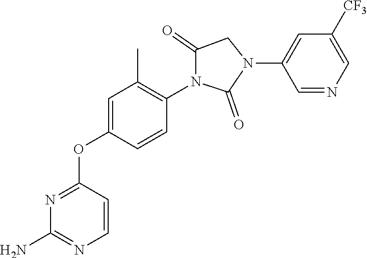 [SMILES: Cc1cc(Oc2ccnc(N)n2)ccc1N1C(=O)CN(c2cncc(C(F)(F)F)c2)C1=O]

To a stirred solution of the compound prepared in Reference Example 8 (2.0 g) in THF (20 mL) was added DIEA (20 mL) and the compound prepared in Reference Example 5 (4.1 g) at 0° C. under N₂. The resulting mixture was stirred for 1 h at 70° C. under N₂. The reaction was concentrated to dryness and the residue was diluted with water and extracted with ethyl acetate. The combined organic layer was washed with water, brine, dried over sodium sulfate, filtered and concentrated. The obtained residue was purified by silica gel column chromatography (petroleum ether:ethyl acetate=1:2) to give 3.0 g crude product. The mixture was diluted with DCM. The precipitation was filtered and washed with DCM. The solid was dried under reduced pressure to obtain the title compound (2.3 g) having the following physical property values.

¹H NMR (400 Hz, DMSO-d₆): δ 9.18, 8.79, 8.52, 8.15, 7.38, 7.23, 7.17, 6.74, 6.22, 4.89, 4.77, 2.20;

LCMS: m/z 445 [M+H]+;

HPLC retention time: 1.213 min (method 1).

### Example 2(1)˜2 (165)

A procedure for a purpose similar to that for Reference Example 8→Example 2 was carried out by using a corresponding halide compound instead of 2-amino-4-chloropyrimidine, a corresponding alcohol compound instead of 4-amino-3-methylphenol, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 2(1): 3-{4-[(6,7-dimethoxy-4-quinolinyl)oxy]phenyl}-1-{3-[(1,1-dioxido-4-thiomorpholinyl)methyl]-5-(trifluoromethyl)phenyl}-2,4-imidazolidinedione

¹H NMR (500 Hz, DMSO-d₆): δ 8.54, 7.79, 7.56, 7.50, 7.47, 7.44-7.41, 7.09, 6.61, 4.71, 4.21, 3.96, 3.93, 3.12-3.08, 3.07-3.03, 2.96;

ESI MS m/z 701 [M+H]+

HPLC retention time: 14.13 min. (Method 75).

### Example 2(2): 2-methyl-2-propanyl 3-(2-[(6,7-dimethoxy-4-quinolinyl)oxy]-5-{2,5-dioxo-3-[5-(trifluoromethyl)-3-pyridinyl]-1-imidazolidinyl}phenyl)-1-azetidinecarboxylate

¹H NMR (300 MHz, CD₃OD): δ 9.15, 8.68, 8.60, 8.46-8.42, 7.77-7.70, 7.65-7.62, 7.58-7.53, 7.38-7.32, 6.57-6.55, 4.72, 4.22-4.19, 4.16-4.12, 4.10-3.89, 1.40-1.28;

LCMS: m/z 680 [M+H]+;

HPLC retention time: 1.333 min (method 5).

### Example 2(3): 3-{3-(difluoromethoxy)-4-[(6,7-dimethoxy-4-quinolinyl)oxy]phenyl}-1-[5-(trifluoromethyl)-3-pyridinyl]-2,4-imidazolidinedione trifluoroacetate (1:1)

¹H NMR (400 MHz, CD₃OD): δ 9.17, 8.72, 8.61, 7.87, 7.73-7.66, 7.48, 7.13-6.76, 4.76, 4.14, 4.10;

LCMS: m/z 591 [M+H]+;

HPLC retention time: 1.253 min (method 5).

### Example 2(4): 3-{4-[(6,7-dimethoxy-4-quinolinyl)oxy]-3-(trifluoromethoxy)phenyl}-1-[5-(trifluoromethyl)-3-pyridinyl]-2,4-imidazolidinedione trifluoroacetate (1:1

¹H NMR (400 MHz, CD₃OD): δ 9.19, 8.75, 8.62, 7.92, 7.86-7.79, 7.51, 7.07, 4.81, 4.16, 4.11;

LCMS: m/z 609 [M+H]+;

HPLC retention time: 1.65 min (method 38).

### Example 2(5): 3-[3-isopropyl-4-({7-[3-(methylsulfonyl)propoxy]-4-quinolinyl}oxy)phenyl]-1-[3-(trifluoromethyl)phenyl]-2,4-imidazolidinedione trifluoroacetate (1:1)

¹H NMR (400 MHz, CD₃OD) δ 8.83, 8.63, 8.22, 7.89-7.86, 7.68-7.62, 7.56-7.42, 6.97, 4.70, 4.46, 3.42, 3.13-3.10, 3.07, 2.48-2.41, 1.28;

LCMS: m/z 642 [M+H]+;

HPLC retention time: 3.160 min (method 7).

### Example 2(6): 3-[3-isopropyl-4-({7-[3-(methylsulfonyl)propoxy]-4-quinolinyl}oxy)phenyl]-1-[1-methyl-2-oxo-5-(trifluoromethyl)-1,2-dihydro-3-pyridinyl]-2,4-imidazolidinedione

¹H NMR (300 MHz, CDCl₃): δ 8.62, 8.32, 8.03, 7.75, 7.53, 7.49, 7.39, 7.30-7.25, 7.20, 6.48, 4.83, 4.33, 3.71, 3.40-3.29, 3.18, 3.01, 2.48, 1.26;

LCMS: m/z 673 [M+H]+;

HPLC retention time: 1.286 min (method 8).

### Example 2(7): 3-[3-isopropyl-4-({7-[3-(methylsulfonyl)propoxy]-4-quinolinyl}oxy)phenyl]-1-[5-(trifluoromethyl)-3-pyridinyl]-2,4-imidazolidinedione trifluoroacetate (1:1)

¹H NMR (300 MHz, CD₃OD): δ 9.18, 8.83, 8.70, 8.66-8.61, 7.73-7.72, 7.68-7.64, 7.58-7.52, 7.46-7.44, 6.98, 4.89, 4.46, 3.44-3.42, 3.29-3.05, 2.48, 1.26;

LCMS: m/z 643 [M+H]+;

HPLC retention time: 1.641 min (method 3).

### Example 2(8): 3-{trans-4-[(7-methoxy-4-quinazolinyl)oxy]cyclohexyl}-1-[2-methoxy-5-(trifluoromethyl)-3-pyridinyl]-2,4-imidazolidinedione

¹H NMR (400 MHz, CD₃OD): δ 8.66, 8.44, 8.18, 8.08, 7.31-7.22, 5.38, 4.37, 4.18, 4.11, 3.96, 2.54-2.38, 1.99-1.90, 1.81-1.69;

LCMS: m/z 532 [M+H]+;

HPLC retention time: 2.011 min (method 10).

### Example 2(9): formic acid—3-{trans-4-[(7-methoxy-4-quinolinyl)oxy]cyclohexyl}-1-[5-(trifluoromethyl)-3-pyridinyl]-2,4-imidazolidinedione (1:1)

¹H NMR (300 MHz, DMSO-d₆): δ 9.10, 8.74, 8.61, 8.49, 8.01, 7.30, 7.19-7.15, 7.06, 4.71-4.64, 4.58, 4.10-3.99, 3.90, 2.41-2.25, 1.85-1.73, 1.70-1.59;

LCMS: m/z 501 [M+H]+;

HPLC retention time: 1.728 min (method 3).

### Example 2(10): formic acid—3-{trans-4-[(6,7-dimethoxy-4-quinolinyl)oxy]cyclohexyl}-1-[5-(trifluoromethyl)-3-pyridinyl]-2,4-imidazolidinedione (1:1)

¹H NMR (400 MHz, DMSO-d₆): δ 9.11, 8.74, 8.50, 7.32, 7.09, 4.69, 4.58, 4.07, 3.90, 2.49-2.28, 1.85, 1.72;

LCMS: m/z 531 [M+H]+;

HPLC retention time: 2.870 min (method 11).

### Example 2(11): formic acid—3-{trans-4-[(7-methoxy-4-uinolinyl)oxy]cyclohexyl}-1-[3-(trifluoromethyl)phenyl]-2,4-imidazolidinedione (1:1)

¹H NMR (400 MHz, DMSO-d₆): δ 9.02, 8.25, 8.17, 7.80-7.78, 7.71-7.63, 7.48-7.43, 5.06, 4.53, 4.17-4.06, 3.99, 2.54-2.46, 2.39-2.31, 1.87-1.72;

LCMS: m/z 500 [M+H]+;

HPLC retention time: 1.510 min (method 9).

### Example 2(12): formic acid—3-{trans-4-[(6,7-dimethoxy-4-quinolinyl)oxy]cyclohexyl}-1-[3-(trifluoromethyl)phenyl]-2,4-imidazolidinedione (1:1)

¹H NMR (400 MHz, CD₃OD): δ 8.68, 8.16, 7.78-7.76, 7.61-7.60, 7.47, 7.35, 5.05-5.02, 4.49, 4.24-4.19, 4.5, 2.65-2.52, 2.52-2.43, 2.03-1.85;

LCMS: m/z 530 [M+H]+;

HPLC retention time: 1.835 min (method 12).

### Example 2(13): formic acid—3-{4-[(6,7-dimethoxy-4-uinolinyl)oxy]-3-isopropylphenyl}-1-[2-fluoro-5-(trifluoromethyl)phenyl]-2,4-imidazolidinedione (1:1)

¹H NMR (400 MHz, DMSO-d₆): δ 8.51, 8.22-8.18, 7.81-7.79, 7.66-7.63, 7.56, 7.43-7.40, 7.39-7.30, 6.46, 4.69, 3.96, 3.16-3.09, 1.20;

LCMS: m/z 584 [M+H]+;

HPLC retention time: 1.854 min (method 14).

### Example 2(14): 3-{4-[(6,7-dimethoxy-4-quinazolinyl)oxy]bicyclo[2.2.2]oct-1-yl}-1-[5-(trifluoromethyl)-3-pyridinyl]-2,4-imidazolidinedione

¹H NMR (400 MHz, DMSO-d₆): δ 9.04, 8.73, 8.59, 8.48, 7.26, 4.48, 3.98-3.95, 2.61-2.55, 2.54-2.48;

LCMS: m/z 558 [M+H]+;

HPLC retention time: 1.351 min (method 5).

### Example 2(15): 3-{4-[(6,7-dimethoxy-4-quinazolinyl)oxy]bicyclo[2.2.2]oct-1-yl}-1-[3-(trifluoromethyl)phenyl]-2,4-imidazolidinedione

¹H NMR (400 MHz, CD₃OD): δ 8.55, 8.11, 7.75-7.73, 7.59-7.53, 7.43-7.41, 7.34, 7.19, 4.29, 3.99-3.95, 2.66-2.63, 2.58-2.53;

LCMS: m/z 557 [M+H]+;

HPLC retention time: 2.889 min (method 16).

### Example 2(16): 3-{3-[(6,7-dimethoxy-4-quinazolinyl)oxy]bicyclo[1.1.1]pent-1-yl}-1-[5-(trifluoromethyl)-3-pyridinyl]-2,4-imidazolidinedione

¹H NMR (400 MHz, CD₃OD): δ 9.09, 8.66, 8.56, 7.42, 7.28, 4.55, 4.03-4.01, 3.04;

LCMS: m/z 516 [M+H]+;

HPLC retention time: 1.428 min (method 9).

### Example 2(17): 3-{3-[(6,7-dimethoxy-4-quinazolinyl)oxy]bicyclo[1.1.1]pent-1-yl}-1-[3-(trifluoromethyl)phenyl]-2,4-imidazolidinedione

¹H NMR (300 MHz, CDCl₃): δ 8.68, 7.92, 7.75-7.72, 7.60-7.56, 7.53-7.50, 7.45-7.26, 4.34, 4.04, 3.04;

LCMS: m/z 515 [M+H]+;

HPLC retention time: 1.906 min (method 1).

### Example 2(18): 3-{trans-4-[(7-methoxy-4-quinazolinyl)oxy]cyclohexyl}-1-[3-(trifluoromethyl)phenyl]-2,4-imidazolidinedione

¹H NMR (400 MHz, DMSO-d₆): δ 8.72, 8.14, 8.02, 7.81, 7.66, 7.47, 7.30-7.25, 5.32-5.25, 4.52, 4.07-4.01, 3.94, 2.39-2.29, 2.07-1.83, 1.80-1.67;

LCMS: m/z 501 [M+H]+;

HPLC retention time: 1.507 min (method 5).

### Example 2(19): 3-{trans-4-[(7-methoxy-4-quinazolinyl)oxy]cyclohexyl}-1-[5-(trifluoromethyl)-3-pyridinyl]-2,4-imidazolidinedione

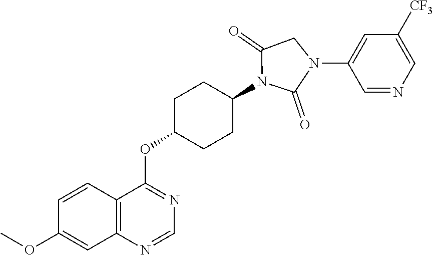 [SMILES: COc1ccc2c(O[C@H]3CC[C@H](N4C(=O)CN(c5cncc(C(F)(F)F)c5)C4=O)CC3)ncnc2c1]

¹H NMR (300 MHz, CDCl₃): δ 8.88, 8.71-8.69, 8.46, 8.05, 7.26, 7.06, 5.45-5.36, 4.36, 4.25-4.15, 3.95, 2.60-2.40, 1.91-1.73;

LCMS: m/z 502 [M+H]+;

HPLC retention time: 1.810 min (method 1).

### Example 2(20): 1-[2-hydroxy-5-(trifluoromethyl)-3-pyridinyl]-3-{trans-4-[(7-methoxy-4-guinazolinyl)oxy]cyclohexyl}-2,4-imidazolidinedione

1H NMR (400 MHz, CD₃OD): δ 8.68, 8.08, 8.02, 7.85, 7.25-7.22, 5.41, 4.47, 4.16, 3.96, 2.58-2.40, 1.94, 1.80-1.75;

LCMS: m/z 518 [M+H]+;

HPLC retention time: 1.566 min (method 1).

### Example 2(21): 3{-(trans-4-[(6,7-dimethoxy-4-guinazolinyl)oxy]cyclohexyl}-1-[3-(trifluoromethyl)phenyl]-2,4-imidazolidinedione

¹H NMR (400 MHz, DMSO-d₆): δ 8.64, 8.15, 7.81, 7.64, 7.49, 7.31, 5.29, 4.53, 4.04, 3.94, 2.33, 1.85, 1.71;

LCMS: m/z 531 [M+H]+;

HPLC retention time: 1.902 min (method 10).

### Example 2(22): 3-{trans-4-[(6,7-dimethoxy-4-quinazolinyl)oxy]cyclohexyl}-1-[5-(trifluoromethyl)-3-pyridinyl]-2,4-imidazolidinedione

¹H NMR (400 MHz, DMSO-d₆): δ 9.10, 8.75, 8.64, 8.49, 7.30, 5.32-5.25, 4.59, 4.10-4.03, 3.94, 2.34, 1.86, 1.76-1.66;

LCMS: m/z 532 [M+H]+;

HPLC retention time: 1.525 min (method 23).

### Example 2(23): 3-{trans-3-[(6,7-dimethoxy-4-guinazolinyl)oxy]cyclopentyl}-1-[3-(trifluoromethyl)phenyl]-2,4-imidazolidinedione

¹H NMR (300 MHz, CDCl₃): δ 8.67, 7.89, 7.77-7.74, 7.61-7.50, 7.44-7.42, 7.32, 7.267, 5.96-5.94, 5.00, 4.33, 4.05, 2.80-2.55, 2.36-2.24, 2.14-2.01;

LCMS: m/z 517 [M+H]+;

HPLC retention time: 1.961 min (method 17).

### Example 2(24): 3-{trans-3-[(6,7-dimethoxy-4-quinazolinyl)oxy]cyclohexyl}-1-[3-(trifluoromethyl)phenyl]-2,4-imidazolidinedione

¹H NMR (400 MHz, DMSO-d₆): δ 8.63, 8.15, 7.78, 7.64, 7.48, 7.31, 5.37, 4.50, 4.17-4.11, 3.94, 2.45-2.35, 2.22, 2.11, 1.92, 1.76, 1.54;

LCMS: m/z 531 [M+H]+;

HPLC retention time: 1.466 min (method 5).

### Example 2(25): 3-{3-ethyl-4-[(7-methoxy-4-quinazolinyl)oxy]phenyl}-1-[3-(trifluoromethoxy)phenyl]-2,4-imidazolidinedione

¹H NMR (400 MHz, DMSO-d₆): δ 8.66, 8.35-8.33, 7.90, 7.67-7.56, 7.45-7.35, 7.18, 4.67, 3.99, 2.54-2.49, 1.12;

LCMS: m/z 539 [M+H]+;

HPLC retention time: 1.755 min (method 5).

### Example 2(26): 3-{4-[(6,7-dimethoxy-4-quinolinyl)oxy]-3-ethyl-2-methylphenyl}-1-[5-(trifluoromethyl)-3-pyridinyl]-2,4-imidazolidinedione

¹H NMR (400 MHz, DMSO-d₆): δ 9.19, 8.79, 8.52, 7.55, 7.43, 7.34, 7.19, 6.47, 4.93-4.76, 3.96, 2.66, 2.22, 1.09;

LCMS: m/z 567 [M+H]+;

HPLC retention time: 1.379 min (method 1).

### Example 2(27): 3-{4-[(6,7-dimethoxy-4-quinolinyl)oxy]-3-fluoro-5-isopropylphenyl}-1-[5-(trifluoromethyl)-3-pyridinyl]-2,4-imidazolidinedione

¹H NMR (400 MHz, CD₃OD): δ 9.16, 8.69, 8.60, 8.45, 7.70, 7.50-7.49, 7.42-7.40, 6.53-6.51, 4.73, 4.03, 3.23-3.16, 1.26;

LCMS: m/z 585 [M+H]+;

HPLC retention time: 1.430 min (method 9).

### Example 2(28): 3-{4-[(7-methoxy-4-quinazolinyl)oxy]-3-methylphenyl}-1-[5-(trifluoromethyl)-3-pyridinyl]-2,4-imidazolidinedione

¹H NMR (400 MHz, DMSO-d₆): δ 9.20, 8.79, 8.66, 8.54, 8.35-8.33, 7.44-7.36, 4.80, 3.99, 2.16;

LCMS: m/z 510 [M+H]+;

HPLC retention time: 1.455 min (method 5).

### Example 2(29): 3-{3-(difluoromethoxy)-4-[(7-methoxy-4-quinazolinyl)oxy]phenyl}-1-[5-(trifluoromethyl)-3-pyridinyl]-2,4-imidazolidinedione

¹H NMR (400 MHz, DMSO-d₆): δ 9.21, 8.80, 8.68, 8.55, 8.32-8.20, 7.71, 7.55-7.41, 7.33-6.97, 4.81, 4.00;

LCMS: m/z 562 [M+H]+;

HPLC retention time: 1.667 min (method 30).

### Example 2(30): 3-{4-[(6,7-dimethoxy-4-quinolinyl)oxy]-2-methylcyclohexyl}-1-[5-(trifluoromethyl)-3-pyridinyl]-2,4-imidazolidinedione

¹H NMR (400 MHz, DMSO-d₆): δ 9.10, 8.75, 8.52-8.50, 7.35, 7.30, 7.03, 4.94, 4.60, 4.21, 3.91, 2.37-2.33, 2.18-2.14, 1.89-1.81, 1.65-1.62, 1.14;

LCMS: m/z 545 [M+H]+;

HPLC retention time: 3.306 min (method 31).

### Example 2(31): 3-{trans-3-[(6,7-dimethoxy-4-quinazolinyl)oxy]cyclohexyl}-1-[3-(trifluoromethyl)phenyl]-2,4-imidazolidinedione

¹H NMR (400 MHz, DMSO-d₆): δ 8.69, 8.15, 7.79, 7.66, 7.49, 7.34, 7.30, 5.41, 4.50, 4.18, 3.95, 2.35, 2.22, 2.10, 1.92, 1.76, 1.54;

LCMS: m/z 531 [M+H]+;

HPLC retention time: 1.559 min (method 9).

### Example 2(32): 3-{6-[(6,7-dimethoxy-4-quinolinyl)oxy]-3-pyridinyl}-1-[5-(trifluoromethyl)-3-pyridinyl]-2,4-imidazolidinedione

¹H NMR (400 MHz, CDCl₃): δ 8.96, 8.75, 8.69, 8.48, 8.43, 7.97, 7.55, 7.36, 7.29, 7.06, 4.63, 4.07, 3.97;

LCMS: m/z 526 [M+H]+;

HPLC retention time: 1.140 min (method 5).

### Example 2(33): 3-{4-[(6,7-dimethoxy-4-uinolinyl)oxy]-3-ethyl-2-fluorophenyl}-1-[5-(trifluoromethyl)-3-pyridinyl]-2,4-imidazolidinedione trifluoroacetate (1:1)

¹H NMR (400 MHz, DMSO-d₆): δ 9.21, 8.83, 8.53, 7.76, 7.62-7.58, 7.41, 6.93, 4.96-4.90, 4.05, 2.70, 1.14;

LCMS: m/z 571 [M+H]+;

HPLC retention time: 2.398 min (method 32).

### Example 2(34): 3-{4-[(6,7-dimethoxy-4-quinolinyl)oxy]phenyl}-1-[5-(trifluoromethyl)-3-pyridinyl]-2,4-imidazolidinedione

¹H NMR (300 MHz, DMSO-d₆): δ 9.20, 8.79, 8.62, 8.54, 7.61-7.58, 7.55, 6.69, 4.79, 3.95, 3.98;

LCMS: m/z 525 [M+H]+;

HPLC retention time: 1.284 min (method 9).

### Example 2(35): 3-{5-bromo-6-[(7-oxo-5,6,7,8-tetrahydro-1,8-naphthyridin-4-yl)oxy]-3-pyridinyl-}-[5-(trifluoromethyl)-3-pyridinyl]-2,4-imidazolidinedione

¹H NMR (400 MHz, DMSO-d₆): δ 10.65, 9.20, 8.81, 8.52, 8.38, 8.25, 8.16, 6.92, 4.83, 2.77, 2.53;

LCMS: m/z 562 [M+H]+;

HPLC retention time: 2.539 min (method 28).

### Example 2(36): 3-{3-bromo-4-[(6,7-dimethoxy-4-quinolinyl)oxy]-5-fluorophenyl}-1-[5-(trifluoromethyl)-3-pyridinyl]-2,4-imidazolidinedione

¹H NMR (400 MHz, DMSO-d₆): δ 9.21, 8.82, 8.55-8.51, 7.87-7.86, 7.76-7.73, 7.59, 7.45, 6.48-6.47, 4.84, 3.97;

LCMS: m/z 621 [M+H]+;

HPLC retention time: 1.277 min (method 5).

### Example 2(37): 3-{4-[(6,7-dimethoxy-4-quinolinyl)oxy]-3-fluoro-5-isopropenylphenyl}-1-[5-(trifluoromethyl)-3-pyridinyl]-2,4-imidazolidinedione

¹H NMR (400 MHz, DMSO-d₆): δ 9.21, 8.81, 8.56, 8.49, 7.60-7.55, 7.47-7.46, 7.42, 6.40-6.39, 5.24, 5.19, 4.82, 3.96, 3.98, 2.01;

LCMS: m/z 583 [M+H]+;

HPLC retention time: 1.317 min (method 5).

### Example 2(38): 3-{3-bromo-4-[(6,7-dimethoxy-4-quinolinyl)oxy]-2-methylphenyl}-1-[5-(trifluoromethyl)-3-pyridinyl]-2,4-imidazolidinedione

¹H NMR (400 MHz, DMSO-d₆): δ 9.20, 8.80, 8.58-8.54, 7.58-7.55, 7.52-7.45, 6.52-6.50, 4.92, 4.77, 3.98, 3.96, 2.33;

LCMS: m/z 617 [M+H]+;

HPLC retention time: 1.376 min (method 9).

### Example 2(39): 3-{3-ethyl-5-fluoro-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.55, 9.19, 8.80, 8.54, 7.99, 7.49-7.38, 6.16, 4.80, 3.05, 2.62-2.59, 1.14;

LCMS: m/z 530 [M+H]+;

HPLC retention time: 1.356 min (method 5).

### Example 2(40): 3-{3-methyl-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.54, 9.18, 8.78, 8.53, 8.00, 7.44, 7.34, 7.20, 6.23, 4.77, 2.97, 2.56, 2.20;

LCMS: m/z 498 [M+H]+;

HPLC retention time: 1.558 min (method 17).

### Example 2(41): 3-{3-(difluoromethoxy)-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.58, 9.19, 8.79, 8.53, 8.03, 7.54, 7.44, 7.40-7.04, 6.36, 4.78, 2.95, 2.55;

LCMS: m/z 550 [M+H]+;

HPLC retention time: 2.564 min (method 35).

### Example 2(42): 3-{3-ethyl-4-[(7-oxo-5,6,7,8-tetrahydro-1,8-naphthyridin-4-yl)oxy]phenyl}-1-[3-(trifluoromethoxy)phenyl]-2,4-imidazolidinedione trifluoroacetate (1:1)

¹H NMR (400 MHz, DMSO-d₆): δ 10.57, 8.01, 7.90, 7.62-7.55, 7.45, 7.34, 7.21-7.17, 6.27, 4.66, 2.99, 2.67-2.56, 1.16;

LCMS: m/z 527 [M+H]+;

HPLC retention time: 1.642 min (method 9).

### Example 2(43): 3-{4-[(6,7-dimethoxy-4-uinolinyl)oxy]-2,3-difluorophenyl}-1-[5-(trifluoromethyl)-3-pyridinyl]-2,4-imidazolidinedione trifluoroacetate (1:1)

¹H NMR (400 MHz, DMSO-d₆): δ 9.21, 8.82, 8.52, 7.71, 7.63-7.56, 7.12, 4.93, 4.02, 4.03;

LCMS: m/z 561 [M+H]+;

HPLC retention time: 1.647 min (method 33).

### Example 2(44): 3-{4-[(6,7-dimethoxy-4-quinolinyl)oxy-3-fluoro-2-methoxy]phenyl}-1-[5-(trifluoromethyl)-3-pyridinyl]-2,4-imidazolidinedione trifluoroacetate (1:1)

¹H NMR (400 MHz, DMSO-d₆): δ 9.20, 8.84-8.80, 8.52, 7.74, 7.59, 7.50-7.44, 7.07, 5.01, 4.86, 4.03, 4.04, 3.96;

LCMS: m/z 573 [M+H]+;

HPLC retention time: 0.959 min (method 36).

### Example 2(45): formic acid—3-{4-[(6,7-dimethoxy-4-quinolinyl)oxy]-2,3-dimethylphenyl}-1-[5-(trifluoromethyl)-3-pyridinyl]-2,4-imidazolidinedione (1:1)

¹H NMR (400 MHz, DMSO-d₆): δ 9.20, 8.80, 8.56, 8.51, 7.58, 7.44, 7.36, 7.23, 6.35, 4.95, 4.82, 3.97, 2.19-2.16;

LCMS: m/z 553 [M+H]+;

HPLC retention time: 1.339 min (method 9).

### Example 2(46): formic acid—1-{4-[(6,7-dimethoxy-4-quinolinyl)oxy]phenyl}-3-[5-(trifluoromethyl)-3-pyridinyl]-2,4-imidazolidinedione (1:1)

¹H NMR (400 MHz, DMSO-d₆): δ 9.09, 9.03, 8.62, 8.37, 7.91-7.88, 7.62, 7.46-7.44, 6.61, 4.77, 4.00, 3.98;

LCMS: m/z 525 [M+H]+;

HPLC retention time: 1.198 min (method 5).

### Example 2(47): 3-{3-bromo-4-[(6,7-dimethoxy-4-quinolinyl)oxy]-2-fluorophenyl}-1-[5-(trifluoromethyl)-3-pyridinyl]-2,4-imidazolidinedione

¹H NMR (400 MHz, DMSO-d₆): δ 9.20, 8.82, 8.58, 8.53, 7.72-7.68, 7.50, 7.46-7.43, 6.64, 5.01-4.82, 3.97, 3.95;

LCMS: m/z 621 [M+H]+;

HPLC retention time: 2.592 min (method 16-2).

### Example 2(48): 3-{6-[(7-oxo-5,6,7,8-tetrahydro-1,8-naphthyridin-4-yl)oxy]-3-pyridinyl}-1-[5-(trifluoromethyl)-3-pyridinyl]-2,4-imidazolidinedione

¹H NMR (300 MHz, DMSO-d₆): δ 10.60, 9.19, 8.79, 8.51, 8.25, 8.12, 8.00, 7.36, 6.82, 4.79, 2.77, 2.50;

LCMS: m/z 485 [M+H]+;

HPLC retention time: 1.161 min (method 5).

### Example 2(49): 3-{4-[(6,7-dimethoxy-4-quinolinyl)oxy]-2-fluoro-3-vinylphenyl}-1-[5-(trifluoromethyl)-3-pyridinyl]-2,4-imidazolidinedione

¹H NMR (400 MHz, DMSO-d₆): δ 9.20, 8.81, 8.60, 8.53, 7.61-7.57, 7.48, 7.32, 6.73-6.61, 6.03, 5.62, 4.96-4.84, 3.98, 3.97;

LCMS: m/z 569 [M+MeCN]+HPLC retention time: 1.376 min (method 9).

### Example 2(50): 3-{2-ethyl-4-[(7-methoxy-4-quinazolinyl)oxy]phenyl}-1-[5-(trifluoromethyl)-3-pyridinyl]-2,4-imidazolidinedione

¹H NMR (400 MHz, DMSO-d₆): δ 9.19, 8.79, 8.69, 8.53, 8.31-8.28, 7.46-7.38, 7.33, 4.94, 4.77, 3.99, 2.59-2.53, 1.13;

LCMS: m/z 546 [M+Na]+

HPLC retention time: 2.949 min (method 16).

### Example 2(51): 3-{2-ethyl-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (300 MHz, DMSO-d₆): δ 10.58, 9.17, 8.78, 8.51, 8.06, 7.40, 7.20, 7.09, 6.46, 4.94-4.73, 2.92, 2.56-2.49, 1.10;

LCMS: m/z 512 [M+H]+;

HPLC retention time: 1.567 min (method 1).

### Example 2(52): 3-{3-bromo-5-fluoro-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.59, 9.20, 8.81, 8.54, 8.02, 7.81-7.69, 6.24, 4.83, 3.06, 2.62;

LCMS: m/z 580 [M+H]+;

HPLC retention time: 1.338 min (method 5).

### Example 2(53): 3-{2-ethyl-4-[(7-methoxy-4-quinazolinyl)oxy]phenyl}-1-[3-(trifluoromethyl)phenyl]-2,4-imidazolidinedione

¹H NMR (400 MHz, DMSO-d₆): δ 8.70, 8.31-8.29, 8.18, 7.93, 7.70, 7.55-7.53, 7.46-7.32, 4.90, 4.73, 4.00, 2.57, 1.14;

LCMS: m/z 523 [M+H]+;

HPLC retention time: 1.697 min (method 5).

### Example 2(54): 3-{3-ethyl-4-[(7-methoxy-4-quinazolinyl)oxy]-2-methylphenyl}-1-[5-(trifluoromethyl)-3-pyridinyl]-2,4-imidazolidinedione trifluoroacetate (1:1)

¹H NMR (300 MHz, DMSO-d₆): δ 9.20, 8.80, 8.69, 8.55, 8.37-8.32, 7.45-7.42, 7.37-7.26, 4.92, 4.80, 4.00, 2.63-2.52, 2.20, 1.06;

LCMS: m/z 538 [M+H]+;

HPLC retention time: 1.956 min (method 58).

### Example 2(55): 3-{3-ethyl-4-[(7-methoxy-4-quinazolinyl)oxy]-2-methylphenyl}-1-[3-(trifluoromethyl)phenyl]-2,4-imidazolidinedione

¹H NMR (400 MHz, DMSO-d₆): δ 8.68, 8.36-8.33, 8.20, 7.92, 7.70, 7.54, 7.44-7.41, 7.36-7.26, 4.87, 4.73, 4.00, 2.59, 2.21, 1.09;

LCMS: m/z 537 [M+H]+;

HPLC retention time: 2.976 min (method 16-3).

### Example 2(56): 3-(3-{-ethyl-4-[(7-methoxy-4-quinazolinyl)oxy]phenyl}-2,4-dioxo-1-imidazolidinyl)benzonitrile

¹H NMR (300 MHz, DMSO-d₆): δ 8.67, 8.36, 8.14, 7.68-7.65, 7.46-7.35, 4.70, 4.00, 2.56, 1.12;

LCMS: m/z 480 [M+H]+;

HPLC retention time: 1.676 min (method 1).

### Example 2(57): 5-(3-{2-ethyl-4-[(7-methoxy-4-quinazolinyl)oxy]phenyl}-2,4-dioxo-1-imidazolidinyl)nicotinonitrile

¹H NMR (400 MHz, DMSO-d₆): δ 9.30, 8.84, 8.70, 8.54, 8.31-8.28, 7.46-7.33, 4.88, 4.74, 3.99, 2.57, 1.13;

LCMS: m/z 481 [M+H]+;

HPLC retention time: 1.688 min (method 1).

### Example 2(58): 3-(3-{2-ethyl-4-[(7-methoxy-4-quinazolinyl)oxy]phenyl}-2,4-dioxo-1-imidazolidinyl)benzonitrile

¹H NMR (400 MHz, DMSO-d₆): δ 8.69, 8.30, 8.10, 7.69-7.63, 7.45-7.39, 7.33, 4.84, 4.69, 3.99, 2.54, 1.12;

LCMS: m/z 480 [M+H]+;

HPLC retention time: 1.503 min (method 5).

### Example 2(59): 3-{4-[(7-methoxy-4-quinazolinyl)oxy]bicyclo[2.2.1]hept-1-yl}-1-[5-(trifluoromethyl)-3-pyridinyl]-2,4-imidazolidinedione

¹H NMR (400 MHz, DMSO-d₆): δ 9.07, 8.73-8.70, 8.49, 8.05, 7.28-7.25, 4.56, 3.94, 2.88, 2.50-2.42, 2.17-2.04;

LCMS: m/z 514 [M+H]+;

HPLC retention time: 2.601 min (method 16).

### Example 2(60): 3-{5-[(7-methoxy-4-quinazolinyl)oxy]bicyclo[2.2.1]hept-2-Yl}-1-[5-(trifluoromethyl)-3-pyridinyl]-2,4-imidazolidinedione

¹H NMR (400 MHz, DMSO-d₆): δ 9.08, 8.73-8.71, 8.48, 8.05, 7.29-7.24, 5.22, 4.54, 3.94, 2.62, 2.47-2.41, 2.18, 2.02-1.97, 1.78-1.66;

LCMS: m/z 514 [M+H]+;

HPLC retention time: 1.858 min (method 1).

### Example 2(61): 3-{5-[(7-methoxy-4-quinazolinyl)oxy]bicyclo[2.2.1]hept-2-yl}-1-[5-(trifluoromethyl)-3-pyridinyl]-2,4-imidazolidinedione

¹H NMR (400 MHz, DMSO-d₆): δ 9.08, 8.73-8.71, 8.48, 8.05, 7.29-7.24, 5.20, 4.54, 3.98-3.93, 2.65, 2.49-2.40, 2.18, 2.01-1.97, 1.78-1.66;

LCMS: m/z 514 [M+H]+;

HPLC retention time: 1.862 min (method 1).

### Example 2(62): 3-{5-[(7-methoxy-4-quinazolinyl)oxy]bicyclo[2.2.1]hept-2-yl}-1-[5-(trifluoromethyl)-3-pyridinyl]-2,4-imidazolidinedione

¹H NMR (400 MHz, DMSO-d₆): δ 9.08, 8.71, 8.47, 8.04, 7.28-7.24, 5.27, 4.54, 4.07-4.03, 3.93, 2.78, 2.50, 2.32, 2.27-2.12, 1.99, 1.76-1.66;

LCMS: m/z 514 [M+H]+;

HPLC retention time: 1.873 min (method 1).

### Example 2(63): 3-{5-[(7-methoxy-4-quinazolinyl)oxy]bicyclo[2.2.1]hept-2-yl-}-[5-(trifluoromethyl)-3-pyridinyl]-2,4-imidazolidinedione

¹H NMR (400 MHz, DMSO-d₆): δ 9.08, 8.71, 8.47, 8.04, 7.28-7.24, 5.27, 4.54, 4.07-4.03, 3.93, 2.78, 2.50, 2.32, 2.27-2.12, 1.99, 1.76-1.66;

LCMS: m/z 514 [M+H]+;

HPLC retention time: 1.872 min (method 1).

### Example 2(64): 3-{4-[(6,7-dimethoxy-4-quinazolinyl)oxy]phenyl}-1-[3-(hydroxymethyl)phenyl]-2,4-imidazolidinedione

¹H NMR (300 MHz, DMSO-d₆): δ 8.59, 7.68-7.38, 7.14, 5.27, 4.65, 4.53, 3.99;

LCMS: m/z 487 [M+H]+;

HPLC retention time: 1.397 min (method 67).

### Example 2(65): 3-{4-[(7-methoxy-4-quinazolinyl)oxy]-2-methylphenyl}-1-[5-(trifluoromethyl)-3-pyridinyl]-2,4-imidazolidinedione

¹H NMR (300 MHz, DMSO-d₆): δ 9.21, 8.80, 8.70, 8.55, 8.31-8.28, 7.47-7.39, 7.33, 4.93, 4.80, 3.99, 2.24;

LCMS: m/z 510 [M+H]+;

HPLC retention time: 1.731 min (method 38).

### Example 2(66): 3-{2-(difluoromethoxy)-4-[(7-methoxy-4-quinazolinyl)oxy]phenyl}-1-[5-(trifluoromethyl)-3-pyridinyl]-2,4-imidazolidinedione

¹H NMR (400 MHz, DMSO-d₆): δ 9.20, 8.81, 8.52, 8.46, 8.16-8.13, 7.74-7.72, 7.65, 7.27, 7.21, 5.05, 4.87, 3.95;

LCMS: m/z 562 [M+H]+;

HPLC retention time: 1.489 min (method 5).

### Example 2(67): 3-{4-[(7-methoxy-4-quinazolinyl)oxy]-2-(1-pyrrolidinyl)phenyl}-1-[5-(trifluoromethyl)-3-pyridinyl]-2,4-imidazolidinedione

¹H NMR (400 MHz, DMSO-d₆): δ 9.19, 8.78, 8.68, 8.53, 8.28-8.25, 7.39-7.37, 7.23, 6.73-6.66, 4.96, 4.77, 3.98, 3.18, 1.84-1.83;

LCMS: m/z 565 [M+H]+;

HPLC retention time: 1.596 min (method 57-1).

### Example 2(68): 3-[2-methyl-4-({7-[3-(4-morpholinyl)propoxy]-4-quinazolinyl}oxy)phenyl]-1-[5-(trifluoromethyl)-3-pyridinyl]-2,4-imidazolidinedione

¹H NMR (400 MHz, DMSO-d₆): δ 9.21, 8.80, 8.54, 8.41, 8.13, 7.61, 7.53, 7.20-7.18, 4.93, 4.81, 4.22, 3.60, 3.31, 2.39, 2.28, 1.96;

LCMS: m/z 623 [M+H]+;

HPLC retention time: 1.348 min (method 38).

### Example 2(69): 3-{4-[(6,7-dimethoxy-4-quinolinyl)oxy]-2-ethylphenyl}-1-[5-(trifluoromethyl)-3-pyridinyl]-2,4-imidazolidinedione

¹H NMR (400 MHz, DMSO-d₆): δ 9.18, 8.80-8.78, 8.55, 8.54-8.52, 7.50, 7.47-7.45, 7.43, 7.36, 7.26-7.22, 6.64, 4.95-4.75, 3.96, 3.93, 2.56, 1.12;

MS (ESI+): m/z 553 (M+H)+;

HPLC retention time: 3.37 min (method 77).

### Example 2(70): 3-{4-[(6,7-dimethoxy-4-quinolinyl)oxy]-3-(1-hydroxyethyl)phenyl}-1-[5-(trifluoromethyl)-3-pyridinyl]-2,4-imidazolidinedione

¹H NMR (400 MHz, DMSO-d₆): δ 9.20-9.18, 8.80-8.78, 8.56-8.52, 7.78-7.77, 7.54-7.54, 7.44-7.40, 7.32-7.29, 6.53-6.51, 5.41-5.39, 4.96-4.90, 4.78, 3.96, 3.94, 1.34;

MS: m/z 569 (M+H)+;

HPLC retention time: 3.25 min (method 78).

### Example 2(71): 3-{4-[(6,7-dimethoxy-4-quinolinyl)oxy]-3-(1-hydroxyethyl)phenyl}-1-[5-(trifluoromethyl)-3-pyridinyl]-2,4-imidazolidinedione

¹H NMR (400 MHz, DMSO-d₆): δ 9.20-9.18, 8.80-8.78, 8.56-8.52, 7.78-7.77, 7.55-7.54, 7.44-7.40, 7.32-7.29, 6.52, 5.40, 4.96-4.90, 4.78, 3.96, 3.94, 1.34;

MS: m/z 569 (M+H)+;

HPLC retention time: 3.27 min (method 78).

### Example 2(72): 3-{4-[(6,7-dimethoxy-4-quinolinyl)oxy]-3-isopropylphenyl}-1-[3-(trifluoromethoxy)phenyl]-2,4-imidazolidinedione

¹H NMR (400 MHz, DMSO-d₆): δ 8.51, 7.91, 7.65-7.61, 7.60-7.57, 7.57-7.54, 7.43, 7.41-7.37, 7.31, 7.19-7.16, 6.45, 4.67, 3.96, 3.95, 3.17-3.07, 1.21-1.18;

MS: m/z 582 (M+H)+;

HPLC retention time: 3.68 min (method 77).

### Example 2(73): 1-(3-cyclopropylphenyl)-3-{4-[(6,7-dimethoxy-4-quinazolinyl)oxy]-3-isopropylphenyl}-2,4-imidazolidinedione

¹H NMR (400 MHz, DMSO-d₆): δ 8.57, 7.62, 7.50-7.47, 7.42, 7.42-7.41, 7.39-7.33, 7.32-7.27, 6.90-6.87, 4.64, 4.00, 3.99, 3.06-2.99, 1.99-1.91, 1.16, 1.01-0.95, 0.73-0.68;

MS: m/z 539 (M+H)+;

HPLC retention time: 3.64 min (method 77).

### Example 2(74): 3-{4-[(6,7-dimethoxy-4-quinolinyl)oxy]-3-ethylphenyl}-1-[5-(trifluoromethyl)-3-pyridinyl]-2,4-imidazolidinedione

¹H NMR (400 MHz, DMSO-d₆): δ 9.19, 8.80-8.78, 8.57-8.54, 8.51, 7.56, 7.52, 7.43-7.39, 7.36-7.33, 6.44, 4.79, 3.96, 3.95, 2.60, 1.16;

MS: m/z 553 (M+H)+;

HPLC retention time: 3.36 min (method 77).

### Example 2(75): 3-{4-[(6,7-dimethoxy-4-quinolinyl)oxy]-3-ethylphenyl}-1-[3-(trifluoromethoxy)phenyl]-2,4-imidazolidinedione

¹H NMR (400 MHz, DMSO-d₆): δ 8.56, 7.96, 7.67-7.60, 7.57-7.55, 7.48, 7.47-7.43, 7.40-7.37, 7.24-7.21, 6.49, 4.72, 4.01, 2.65, 1.21;

MS: m/z 568 (M+H)+;

HPLC retention time: 3.59 min (method 77).

### Example 2(76): 3-{4-[(6,7-dimethoxy-4-quinolinyl)oxy]-3-ethylphenyl}-1-[3-(trifluoromethyl)phenyl]-2,4-imidazolidinedione

¹H NMR (400 MHz, DMSO-d₆): δ 8.51, 8.22-8.20, 7.92-7.88, 7.72-7.66, 7.57, 7.55-7.51, 7.43-7.39, 7.35-7.32, 6.43, 4.72, 3.96, 3.95, 2.60, 1.16;

MS: m/z 552 (M+H)+;

HPLC retention time: 3.54 min (method 77).

### Example 2(77): 3-{4-[(6,7-dimethoxy-4-quinolinyl)oxy]-3-isopropylphenyl}-1-[5-(trifluoromethyl)-3-pyridinyl]-2,4-imidazolidinedione

¹H NMR (400 MHz, DMSO-d₆): δ 9.26-9.24, 8.85, 8.62-8.60, 8.57, 7.63-7.61, 7.49-7.43, 7.40-7.36, 6.51, 4.85, 4.03-3.99, 3.22-3.15, 1.26;

MS: m/z 567 (M+H)+;

HPLC retention time: 2.94 min (method 76).

### Example 2(78): 3-{4-[(6,7-dimethoxy-4-quinolinyl)oxy]-3-isopropylphenyl}-1-[3-(trifluoromethyl)phenyl]-2,4-imidazolidinedione

¹H NMR (400 MHz, DMSO-d₆): δ 8.57, 8.27-8.25, 7.99-7.95, 7.78-7.72, 7.63-7.61, 7.60-7.58, 7.49, 7.47-7.44, 7.39-7.36, 6.51, 4.79-4.77, 4.03, 4.01, 3.24-3.13, 1.27-1.24;

MS: m/z 566 (M+H)+;

HPLC retention time: 3.24 min (method 80).

### Example 2(79): 3-{4-[(6,7-dimethoxy-4-quinolinyl)oxy]-3-isopropylphenyl}-1-[2-(trifluoromethyl)-4-pyridinyl]-2,4-imidazolidinedione

¹H NMR (400 MHz, DMSO-d₆): δ 8.74, 8.52, 8.25, 7.92-7.89, 7.56-7.55, 7.43, 7.41-7.37, 7.33, 6.46, 4.73, 3.96, 3.95, 3.17-3.09, 1.20;

MS: m/z 567 (M+H)+;

HPLC retention time: 2.97 min (method 76).

### Example 2(80): 3-[3-isopropyl-4-({6-methoxy-7-[3-(4-morpholinyl)propoxy]-4-quinolinyl}oxy)phenyl]-1-[3-(trifluoromethyl)phenyl]-2,4-imidazolidinedione

¹H NMR (400 MHz, DMSO-d₆): δ 8.52, 8.22, 7.95-7.91, 7.71, 7.59-7.57, 7.44, 7.41, 7.33, 6.46, 4.74, 4.23, 3.97-3.96, 3.61, 3.18-3.10, 2.50-2.48, 2.45-2.38, 2.04-1.96, 1.21;

MS: m/z 679 (M+H)+;

HPLC retention time: 2.75 min (method 76).

### Example 2(81):3-[3-isopropyl-4-({6-methoxy-7-[3-(4-morpholinyl)propoxy]-4-quinolinyl}oxy)phenyl]-1-[5-(trifluoromethyl)-3-pyridinyl]-2,4-imidazolidinedione

¹H NMR (400 MHz, DMSO-d₆): δ 9.21, 8.82-8.80, 8.58, 8.52, 7.59-7.57, 7.44-7.40, 7.34, 6.46, 4.81, 4.23, 3.96, 3.61, 3.19-3.10, 2.50-2.46, 2.45-2.38, 2.05-1.96, 1.22;

MS: m/z 680 (M+H)+;

HPLC retention time: 2.63 min (method 76).

### Example 2(82): 3-[3-isopropyl-4-({6-methoxy-7-[3-(methylsulfonyl)propoxy]-4-quinolinyl}oxy)phenyl]-1-[5-(trifluoromethyl)-3-pyridinyl]-2,4-imidazolidinedione

¹H NMR (400 MHz, DMSO-d₆): δ 9.22-9.20, 8.81-8.79, 8.77, 8.56, 7.77, 7.65-7.63, 7.56, 7.49-7.47, 6.77, 4.81, 4.40-4.35, 4.04, 3.38-3.32, 3.13-3.04, 2.36-2.28, 1.22-1.19;

MS (ESI+): m/z 673 (M+H)+;

MS: m/z 673 (M+H)+;

HPLC retention time: 3.41 min (method 77).

### Example 2(83): 3-{4-[(6,7-dimethoxy-4-quinolinyl)oxy]-3-isopropylphenyl}-1-[1-methyl-2-oxo-5-(trifluoromethyl)-1,2-dihydro-3-pyridinyl]-2,4-imidazolidinedione

¹H NMR (400 MHz, DMSO-d₆): δ 8.52, 8.49-8.47, 8.03, 7.59-7.57, 7.44, 7.43-7.40, 7.34-7.31, 6.46, 4.65, 3.98, 3.96, 3.61, 3.17-3.09, 1.21;

MS: m/z 597 (M+H)+;

HPLC retention time: 3.42 min (method 77).

### Example 2(84): 3-{4-[(6,7-dimethoxy-4-quinazolinyl)oxy]-3-isopropylphenyl}-1-[5-(trifluoromethyl)-3-pyridinyl]-2,4-imidazolidinedione

¹H NMR (400 MHz, DMSO-d₆): δ 9.20, 8.79, 8.58, 8.55, 7.62, 7.51, 7.43, 7.41-7.35, 4.79, 4.00, 3.08-3.00, 1.16;

MS: m/z 568 (M+H)+;

HPLC retention time: 3.53 min (method 76).

### Example 2(85): 3-{3-isopropyl-4-[(7-methoxy-4-quinolinyl)oxy]phenyl}-1-[5-(trifluoromethyl)-3-pyridinyl]-2,4-imidazolidinedione

¹H NMR (400 MHz, DMSO-d₆): δ 9.22, 8.81, 8.66, 8.57, 8.29, 7.59, 7.46, 7.44-7.40, 7.38-7.33, 6.46, 4.81, 3.97, 3.15-3.07, 1.20;

MS: m/z 537 (M+H)+;

HPLC retention time: 3.10 min (method 76).

### Example 2(86): 3-{4-[(6,7-dimethoxy-4-quinazolinyl)oxy]-3-isopropylphenyl}-1-[3-(trifluoromethyl)phenyl]-2,4-imidazolidinedione

¹H NMR (400 MHz, DMSO-d₆): δ 8.59, 8.21, 7.93, 7.71, 7.64, 7.57-7.53, 7.52, 7.44, 7.40-7.38, 4.74, 4.02, 3.09-3.01, 1.18;

MS: m/z 567 (M+H)+;

HPLC retention time: 3.69 min (method 76).

### Example 2(87):3-{3-isopropyl-4-[(6-methoxy-4-quinolinyl)oxy]phenyl}-1-[5-(trifluoromethyl)-3-pyridinyl]-2,4-imidazolidinedione

¹H NMR (400 MHz, DMSO-d₆): δ 9.22, 8.81, 8.60, 8.57, 8.00, 7.64, 7.60, 7.52, 7.45-7.41, 7.37, 6.59, 4.81, 3.96, 3.19-3.10, 1.22;

MS: m/z 537 (M+H)+.

HPLC retention time: 3.15 min (method 76).

### Example 2(88): 3-{3-isopropyl-4-[(6-methoxy-4-guinazolinyl)oxy]phenyl}-1-[3-(trifluoromethyl)phenyl]-2,4-imidazolidinedione

¹H NMR (400 MHz, DMSO-d₆): δ 8.64, 8.20, 7.98, 7.91, 7.73-7.69, 7.55-7.51, 7.44-7.36, 4.73, 3.99, 3.08-3.00, 1.17;

MS: m/z 537 (M+H)+;

HPLC retention time: 3.77 min (method 76).

### Example 2(89): 3-{trans-3-[(6,7-dimethoxy-4-quinolinyl)oxy]cyclobutyl}-1-[3-(trifluoromethyl)phenyl]-2,4-imidazolidinedione

¹H NMR (400 MHz, CDCl₃): δ 8.55, 7.91-7.90, 7.79-7.75, 7.55, 7.46-7.40, 6.47, 5.34-5.28, 5.18-5.08, 4.36, 4.07, 4.04, 3.42-3.33, 2.82-2.74;

MS: m/z 502 (M+H)+;

HPLC retention time: 3.44 min (method 77).

### Example 2(90): 3-[3-isopropyl-4-(4-quinazolinyloxy)phenyl]-1-[5-(trifluoromethyl)-3-pyridinyl]-2,4-imidazolidinedione

¹H NMR (400 MHz, DMSO-d₆): δ 9.22, 8.81, 8.78, 8.57, 8.48, 8.13-8.05, 7.89-7.84, 7.54, 7.47, 7.40, 4.82, 3.09-3.01, 1.18;

MS: m/z 508 (M+H)+;

HPLC retention time: 3.55 min (method 77).

### Example 2(91): 3-[3-isopropyl-4-(4-quinazolinyloxy)phenyl]-1-[3-(trifluoromethyl)phenyl]-2,4-imidazolidinedione

¹H NMR (400 MHz, DMSO-6): δ 8.77, 8.47, 8.19, 8.11-8.03, 7.92, 7.87-7.82, 7.70, 7.55-7.51, 7.46-7.36, 4.73, 3.07-2.99, 1.16;

MS: m/z 507 (M+H)+;

HPLC retention time: 3.70 min (method 77).

### Example 2(92): 3-{-isopropyl-4-[(7-methoxy-4-quinazolinyl)oxy]phenyl}-1-[3-(trifluoromethyl)phenyl]-2,4-imidazolidinedione

¹H NMR (400 MHz, DMSO-d₆): δ 8.69, 8.38-8.35, 8.21, 7.95-7.91, 7.71, 7.56-7.51, 7.46-7.41, 7.40-7.36, 4.74, 4.01, 3.07-2.99, 1.17;

MS (ESI+): m/z 537 (M+H)+;

HPLC retention time: 3.76 min (method 77).

### Example 2(93): 3-{3-isopropyl-4-[(7-methoxy-4-quinazolinyl)oxy]phenyl}-1-[5-(trifluoromethyl)-3-pyridinyl]-2,4-imidazolidinedione

¹H NMR (400 MHz, DMSO): δ 9.22, 8.81, 8.69, 8.57, 8.38-8.35, 7.52, 7.45-7.42, 7.38, 4.81, 4.01, 3.07-2.99, 1.17;

MS: m/z 538 (M+H)+;

HPLC retention time: 3.60 min (method 77).

### Example 2(94): 4-[4-(3-{4-[(4-ethyl-1-piperazinyl)methyl]-3-(trifluoromethyl)phenyl}-2,5-dioxo-1-imidazolidinyl)-2-isopropylphenoxy]-N-methyl-2-pyridinecarboxamide

¹H NMR (400 MHz, DMSO-d₆): δ 8.84-8.78, 8.57, 8.20, 7.87-7.78, 7.57, 7.43-7.39, 7.31, 7.21, 4.72, 3.61, 3.13-3.02, 2.82-2.80, 2.47-2.30, 1.19-1.16, 1.00;

MS: m/z 639 (M+H)+;

HPLC retention time: 3.61 min (method 77).

### Example 2(95): 3-{4-[(6,7-dimethoxy-4-quinolinyl)oxy]-3-(1-hydroxyethyl)phenyl}-1-[5-(trifluoromethyl)-3-pyridinyl]-2,4-imidazolidinedione

¹H NMR (400 MHz, DMSO-d₆): δ 9.20-9.18, 8.80-8.78, 8.55, 8.54-8.52, 7.78, 7.54, 7.44-7.40, 7.32-7.29, 6.52, 5.40, 4.97-4.90, 4.78, 3.96, 3.94, 1.34;

MS: m/z 569 (M+H)+;

HPLC retention time: 3.14 min (method 77).

### Example 2(96): 4-[2-isopropyl-4-(3-{4-[(4-methyl-1-piperazinyl)methyl]-3-(trifluoromethyl)phenyl}-2,5-dioxo-1-imidazolidinyl)phenoxy]-N-methyl-2-pyridinecarboxamide

¹H NMR (400 MHz, DMSO-d₆): δ 8.81, 8.57, 8.21, 7.88-7.78, 7.57, 7.43-7.39, 7.31, 7.21, 4.72, 3.65, 3.13-3.02, 2.82-2.79, 2.78-2.57, 2.46-2.36, 1.17;

MS: m/z 591, 625 (M+H)+;

HPLC retention time: 2.88 min (method 76).

### Example 2(97): 4-(4-{2,5-dioxo-3-[4-(1-piperazinylmethyl)-3-(trifluoromethyl)phenyl]-1-imidazolidinyl}-2-isopropylphenoxy)-N-methyl-2-pyridinecarboxamide

¹H NMR (400 MHz, DMSO-d₆): δ 8.83-8.78, 8.57, 8.21-8.19, 7.88-7.80, 7.57, 7.43-7.39, 7.31, 7.21, 4.73-4.71, 3.60, 3.13-3.02, 2.82-2.77, 2.41-2.35, 1.17. NH not observed;

MS (ESI+): m/z 611 (M+H)+;

HPLC retention time: 2.85 min (method 76).

### Example 2(98): 3-{4-[(7-hydroxy-6-methoxy-4-quinazolinyl)oxy]-3-isopropylphenyl}-1-[5-(trifluoromethyl)-3-pyridinyl]-2,4-imidazolidinedione

¹H NMR (400 MHz, DMSO-d₆): δ 10.81, 9.21, 8.81, 8.57, 8.51, 7.62, 7.51, 7.39-7.37, 7.27, 4.81, 4.02, 3.09-3.02, 1.18;

MS: m/z 554 (M+H)+;

HPLC retention time: 3.15 min (method 77).

### Example 2(99): 3-[3-isopropyl-4-(4-quinolinyloxy)phenyl]-1-[5-(trifluoromethyl)-3-pyridinyl]-2,4-imidazolidinedione

¹H NMR (400 MHz, DMSO-d₆): δ 9.22, 8.81, 8.75, 8.57, 8.41, 8.09, 7.90-7.86, 7.73, 7.60, 7.46-7.38, 6.61, 4.81, 3.16-3.08, 1.21;

MS: m/z 507 (M+H)+;

HPLC retention time: 3.05 min (method 76).

### Example 2(100): 3-[3-isopropyl-4-(4-quinolinyloxy)phenyl]-1-[3-(trifluoromethyl)phenyl]-2,4-imidazolidinedione

¹H NMR (400 MHz, DMSO-d₆): δ 8.75, 8.41, 8.22, 8.08, 7.95-7.86, 7.75-7.68, 7.60, 7.55, 7.45-7.37, 6.60, 4.74, 3.16-3.08, 1.21;

MS: m/z 506 (M+H)+;

HPLC retention time: 3.73 min (method 77).

### Example 2(101): 3-[3-isopropyl-4-({6-methoxy-7-[3-(methylsulfonyl)propoxy]-4-quinazolinyl}oxy)phenyl]-1-[5-(trifluoromethyl)-3-pyridinyl]-2,4-imidazolidinedione

¹H NMR (400 MHz, DMSO-d₆): δ 9.22, 8.81, 8.60, 8.57, 7.66, 7.52, 7.46, 7.43-7.36, 4.81, 4.37, 4.03, 3.08, 2.35-2.25, 1.18;

MS: m/z 674 (M+H)+;

HPLC retention time: 3.30 min (method 77).

### Example 2(102): 3-{4-[(6,7-dimethoxy-4-quinazolinyl)oxy]-3-isopropylphenyl}-1-[3-(trifluoromethoxy)phenyl]-2,4-imidazolidinedione

¹H NMR (400 MHz, DMSO-d₆): δ 8.58, 7.90, 7.65-7.61, 7.60-7.56, 7.49, 7.43, 7.40-7.34, 7.19-7.16, 4.67, 4.01, 4.00, 3.09-2.98, 1.16;

MS: m/z 583 (M+H)+;

HPLC retention time: 3.64 min (method 77).

### Example 2(103): 1-(5-cyclopropyl-3-pyridinyl)-3-{4-[(6,7-dimethoxy-4-quinolinyl)oxy]-3-isopropylphenyl}-2,4-imidazolidinedione

¹H NMR (400 MHz, DMSO-d₆): δ 8.74, 8.51, 8.22, 7.72-7.70, 7.56-7.54, 7.43, 7.40-7.37, 7.31, 6.44, 4.69, 3.96, 3.95, 3.16-3.08, 2.05-1.97, 1.21-1.18, 1.08-1.02, 0.81-0.76;

MS: m/z 539 (M+H)+;

HPLC retention time: 3.36 min (method 77).

### Example 2(104): 3-{3-isopropyl-4-[(7-methoxy-1,6-naphthyridin-4-yl)oxy]phenyl}-1-[3-(trifluoromethyl phenyl]-2,4-imidazolidinedione

¹H NMR (400 MHz, DMSO-d₆): δ 9.54, 8.81, 8.22, 7.93, 7.71, 7.61, 7.55, 7.46, 7.27, 6.46, 4.74, 4.05, 3.16-3.08, 1.20;

MS: m/z 537 (M+H)+;

HPLC retention time: 3.67 min (method 77).

### Example 2(105): 3-{3-isopropyl-4-[(7-methoxy-1,6-naphthyridin-4-yl)oxy]phenyl}-1-[5-(trifluoromethyl)-3-pyridinyl]-2,4-imidazolidinedione

¹H NMR (400 MHz, DMSO-d₆) δ 9.54, 9.22, 8.82-8.80, 8.57, 7.61, 7.46, 7.27, 6.46, 4.81, 4.05, 3.16-3.08, 1.20;

MS: m/z 538 (M+H)+;

HPLC retention time: 3.33 min (method 76).

### Example 2(106): 1-(5-cyclopropyl-3-pyridinyl)-3-{4-[(6,7-dimethoxy-4-quinazolinyl)oxy]-3-isopropylphenyl}-2,4-imidazolidinedione

¹H NMR (400 MHz, DMSO-d₆): δ 8.75, 8.59-8.56, 8.23-8.22, 7.70, 7.63-7.60, 7.49, 7.42, 7.39-7.33, 4.69, 4.03-3.97, 3.05-3.01, 2.04-1.99, 1.20-1.13, 1.06-1.04, 0.82-0.79;

MS: m/z 540 (M+H)+;

HPLC retention time: 3.36 min (method 77).

### Example 2(107): 3-{3-isopropyl-4-[(7-methoxy-4-quinolinyl)oxy]phenyl}-1-[1-methyl-2-oxo-5-(trifluoromethyl)-1,2-dihydro-3-pyridinyl]-2,4-imidazolidinedione

¹H NMR (400 MHz, DMSO-d₆): δ 8.63, 8.48-8.46, 8.27, 8.01, 7.57, 7.44, 7.42-7.38, 7.35-7.31, 6.43, 4.63, 3.95, 3.31, 3.13-3.02, 1.17;

MS: m/z 567 (M+H)+;

HPLC retention time: 3.06 min (method 80).

### Example 2(108): 3-{4-[(6,7-dimethoxy-4-quinolinyl)oxy]-2-methylphenyl}-1-[5-(trifluoromethyl)-3-pyridinyl]-2,4-imidazolidinedione

¹H NMR (400 MHz, DMSO-d₆): δ 9.22, 8.84-8.81, 8.56, 7.73, 7.60-7.57, 7.49, 7.42, 6.93, 4.94, 4.82, 4.06, 4.04, 2.29;

MS: m/z 539 (M+H)+;

HPLC retention time: 3.29 min (method 77).

### Example 2(109): 3-{3-sec-butyl-4-[(6,7-dimethoxy-4-quinolinyl)oxy]phenyl}-1-[5-(trifluoromethyl)-3-pyridinyl]-2,4-imidazolidinedione

¹H NMR (400 MHz, CDCl₃): δ 8.95, 8.75-8.73, 8.57-8.54, 8.51, 7.57, 7.47-7.45, 7.35, 7.22, 6.46, 4.62, 4.07, 4.06, 3.01-2.91, 1.92-1.92, 1.71-1.55, 1.23, 0.85;

MS: m/z 581 (M+H)+;

HPLC retention time: 2.99 min (method 76).

### Example 2(110): 3-{3-isopropyl-4-[(6-methoxy-1,5-naphthyridin-4-yl)oxy]phenyl}-1-[3-(trifluoromethyl)phenyl]-2,4-imidazolidinedione

¹H NMR (400 MHz, DMSO-d₆): δ 8.70, 8.32, 8.21, 7.92, 7.70, 7.55-7.51, 7.33-7.29, 7.16-7.09, 4.72, 3.86, 3.31-3.23, 1.27;

MS: m/z 537 (M+H)+;

HPLC retention time: 3.73 min (method 77).

### Example 2(111): 3-{3-isopropyl-4-[(6-methoxy-1,5-naphthyridin-4-yl)oxy]phenyl}-1-[5-(trifluoromethyl)-3-pyridinyl]-2,4-imidazolidinedione

¹H NMR (400 MHz, DMSO-d₆): δ 9.20, 8.80, 8.70, 8.56, 8.32, 7.52, 7.33-7.29, 7.16-7.10, 4.79, 3.85, 3.31-3.24, 1.27;

MS: m/z 538 (M+H)+;

HPLC retention time: 3.49 min (method 76).

### Example 2(112): 4-(4-{2,5-dioxo-3-[3-(3-pyridinyl)-5-(trifluoromethyl)phenyl]-1-imidazolidinyl}phenoxy)-2-pyridinecarboxamide

¹H NMR (500 MHz, CDCl₃): δ 8.89, 8.70-8.68, 8.45, 8.20, 7.94-7.93, 7.92-7.82, 7.67, 7.58, 7.43, 7.24, 7.04, 5.54, 4.61;

MS: m/z 534 (M+H)+;

HPLC retention time: 9.63 min (method 82).

### Example 2(113): 3-{4-[(6,7-dimethoxy-4-quinolinyl)oxy]phenyl}-1-[3-(trifluoromethyl)phenyl]-2,4-imidazolidinedione

¹H NMR (400 MHz, DMSO-d₆): δ 8.54, 8.20, 7.90, 7.69, 7.59-7.50, 7.51, 7.45-7.40, 6.61, 4.72, 3.96, 3.93;

MS: m/z 524 (M+H)+;

HPLC retention time: 1.424 min (method 9).

### Example 2(114): 3-{4-[(2-anilino-4-pyridinyl)oxy]phenyl}-1-[3-(trifluoromethyl)phenyl]-2,4-imidazolidinedione

¹H NMR (300 MHz, CDCl₃): δ 4.53, 6.31-6.40, 6.44, 6.54-6.78, 6.96-7.09, 7.14-7.27, 7.27-7.37, 7.41-7.51, 7.52-7.65, 7.73-7.85, 7.89-7.99, 8.02-8.17; TLC Rf: 0.50 (AcOEt/n-Hexane=1/1).

### Example 2(115): 3-[trans-4-(1H-pyrrolo[2,3-b]pyridin-5-yloxy)cyclohexyl]-1-[5-(trifluoromethyl)-3-pyridinyl]-2,4-imidazolidinedione

¹H NMR (400 MHz, DMSO-d₆): δ 11.48, 9.10, 8.73, 8.48, 8.00, 7.60, 7.42, 6.35, 4.58-4.56, 4.02, 2.60-2.54, 2.08-2.04, 1.67-1.51;

LCMS: m/z 460 [M+H]+;

HPLC retention time: 3.086 min (method 26).

### Example 2(116): 3-[trans-4-(1H-pyrrolo[2,3-b]pyridin-5-yloxy)cyclohexyl]-1-[3-(trifluoromethyl)phenyl]-2,4-imidazolidinedione

¹H NMR (300 MHz, DMSO-d₆): δ 11.50, 8.13, 8.00, 7.82, 7.67-7.60, 7.50-7.41, 6.36, 4.56-4.53, 4.05-3.97, 2.62-2.54, 2.08-2.04, 1.68-1.63;

LCMS: m/z 459 [M+H]+;

HPLC retention time: 1.608 min (method 30).

### Example 2(117): 3-{trans-4-[(2-amino-5-pyrimidinyl)oxy]cyclohexyl}-1-[3-(trifluoromethyl)phenyl]-2,4-imidazolidinedione

¹H NMR (400 MHz, DMSO-d₆): δ 8.13, 8.07, 7.87-7.77, 7.65, 7.49, 6.26, 4.52, 4.37, 4.11-3.89, 2.46, 2.01-1.99, 1.71-1.42;

LCMS: m/z 436 [M+H]+;

HPLC retention time: 1.258 min (method 57-1).

### Example 2(118): 3-[trans-4-(1H-pyrazolo[3,4-b]pyridin-5-yloxy)cyclohexyl]-1-[3-(trifluoromethyl)phenyl]-2,4-imidazolidinedione

¹H NMR (400 MHz, DMSO-d₆): δ 8.26, 8.14, 8.00, 7.87, 7.80, 7.64, 7.48, 4.51, 4.39-4.32, 4.03-3.95, 2.37-2.30, 2.28-2.20, 1.84-1.75, 1.59-1.41;

LCMS: m/z 460 [M+H]+;

HPLC retention time: 1.781 min (method 38).

### Example 2(119): 3-[trans-4-(1H-pyrazolo[3,4-b]pyridin-5-yloxy)cyclohexyl]-1-[5-(trifluoromethyl)-3-pyridinyl]-2,4-imidazolidinedione

¹H NMR (400 MHz, DMSO-d₆): δ 13.48, 9.09, 8.74, 8.48, 8.26, 8.00, 7.87, 4.57, 4.39-4.34, 4.03-3.97, 2.36-2.20, 1.80-1.77, 1.55-1.47;

LCMS: m/z 461 [M+H]+;

HPLC retention time: 1.592 min (method 38).

### Example 2(120):1-[4-fluoro-3-(trifluoromethyl)phenyl]-3-[trans-4-(1H-pyrazolo[3,4-b]pyridin-5-yloxy)cyclohexyl]-2,4-imidazolidinedione

¹H NMR (400 MHz, DMSO-d₆): δ 13.48, 8.26, 8.14, 8.00, 7.88-7.87, 7.59, 4.51, 4.42-4.32, 4.04-3.93, 2.39-2.11, 1.82-1.69, 1.58-1.41;

LCMS: m/z 478 [M+H]+;

HPLC retention time: 1.796 min (method 38).

### Example 2(121): 1-[3-fluoro-5-(trifluoromethyl)phenyl]-3-[trans-4-(1H-pyrazolo[3,4-b]pyridin-5-yloxy)cyclohexyl]-2,4-imidazolidinedione

¹H NMR (400 MHz, DMSO-d₆): δ 13.48, 8.26, 8.00, 7.87, 7.75, 7.45, 4.50, 4.38-4.33, 4.04-3.94, 2.35-2.20, 1.89-1.71, 1.58-1.41;

LCMS: m/z 478 [M+H]+;

HPLC retention time: 0.682 min (method 69).

### Example 2(122): 3-[trans-4-(thieno[3,2-b]pyridin-6-yloxy)cyclohexyl]-1-[3-(trifluoromethyl)phenyl]-2,4-imidazolidinedione

¹H NMR (300 MHz, DMSO-d₆): δ 8.37, 8.28, 8.16, 7.88, 7.78, 7.65, 7.50-7.44, 4.52-4.49, 4.07-3.94, 2.38-2.21, 1.81-1.77, 1.56-1.51;

LCMS: m/z 476 [M+H]+;

HPLC retention time: 1.538 min (method 5).

### Example 2(123): 3-[trans-4-(thieno[3,2-b]pyridin-6-yloxy)cyclohexyl]-1-[5-(trifluoromethyl)-3-pyridinyl]-2,4-imidazolidinedione

¹H NMR (300 MHz, DMSO-d₆): δ 9.10, 8.75, 8.49, 8.37, 8.28, 7.89, 7.46, 4.58, 4.51-4.44, 4.06-3.98, 2.42-2.22, 1.82-1.78, 1.60-1.52;

LCMS: m/z 477 [M+H]+;

HPLC retention time: 1.377 min (method 5).

### Example 2(124): 1-[4-fluoro-3-(trifluoromethyl)phenyl]-3-[trans-4-(thieno[3,2-b]pyridin-6-yloxy)cyclohexyl]-2,4-imidazolidinedione

¹H NMR (400 MHz, DMSO-d₆): δ 8.36, 8.28, 8.16-8.13, 7.89-7.84, 7.63, 7.46, 4.52-4.47, 3.99, 2.37-2.34, 2.24-2.22, 1.80-1.77, 1.55-1.52;

LCMS: m/z 494 [M+H]+;

HPLC retention time: 1.552 min (method 5).

### Example 2(125): 1-[3-fluoro-5-(trifluoromethyl)phenyl]-3-[trans-4-(thieno[3,2-b]pvridin-6-yloxy)cyclohexyl]-2,4-imidazolidinedione

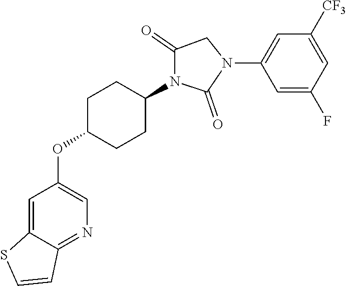 [SMILES: O=C1CN(c2cc(F)cc(C(F)(F)F)c2)C(=O)N1[C@H]1CC[C@H](Oc2cnc3ccsc3c2)CC1]

¹H NMR (300 MHz, DMSO-d₆): δ 8.36, 8.27, 8.00, 7.89, 7.78-7.71, 7.46, 4.55-4.44, 4.02, 2.39-2.18, 1.86-1.74, 1.62-1.45;

LCMS: m/z 494 [M+H]+;

HPLC retention time: 3.071 min (method 70).

### Example 2(126): 3-(trans-3-{[2-(1-methyl-1H-pyrazol-4-yl)-4-pyridinyl]oxy}cyclobutyl)-1-[3-(1-piperazinylmethyl)-5-(trifluoromethyl)phenyl]-2,4-imidazolidinedione

¹H NMR (400 MHz, CDCl₃): δ 8.37, 7.92-7.90, 7.81-7.79, 7.73-7.71, 7.42-7.41, 6.88, 6.56-6.54, 5.20-5.13, 5.09-5.00, 4.34, 3.95, 3.55, 3.32-3.24, 2.93-2.89, 2.68-2.60, 2.47-2.40. NH peak not observed;

MS: m/z 570 (M+H)+;

HPLC retention time: 2.39 min (method 76).

### Example 2(127): 3-(trans-3-{[2-(1-methyl-1H-pyrazol-4-yl)-4-pyridinyl]oxy}cyclobutyl)-1-[4-(1-piperazinylmethyl)-3-(trifluoromethyl)phenyl]-2,4-imidazolidinedione

¹H NMR (400 MHz, CDCl₃): δ 8.37, 7.91-7.90, 7.87-7.83, 7.73-7.69, 6.88, 6.56-6.53, 5.20-5.12, 5.09-4.99, 4.32, 3.95, 3.63, 3.32-3.23, 2.96-2.90, 2.68-2.59, 2.50-2.43. NH peak not observed;

MS: m/z 570 (M+H)+;

HPLC retention time: 2.47 min (method 78).

### Example 2(128): 3-{[trans-3-(1H-pyrrolo[2,3-b]pyridin-4-yloxy)cyclobutyl]methyl}-1-[5-(trifluoromethyl)-3-pyridinyl]-2,4-imidazolidinedione trifluoroacetate (1:1)

¹H NMR (300 MHz, DMSO-d₆): δ1.35-1.95, 2.08-2.45, 3.82-4.19, 4.36-4.86, 6.16-6.45, 6.57-6.87, 7.09-7.34, 7.87-8.22, 8.36-8.56, 8.60-8.89, 8.93-9.19, 11.13-11.73;

MS: m/z 446 (M+H)+;

HPLC retention time: 0.76 min (method 83).

### Example 2(129): 3-(trans-3-{[2-(1-methyl-1H-pyrazol-4-yl)-4-pyridinyl]oxy}cyclobutyl)-1-[3-(trifluoromethoxy)phenyl]-2,4-imidazolidinedione

¹H NMR (400 MHz, DMSO-d₆): δ 8.31, 8.27, 7.99, 7.85-7.84, 7.54, 7.15-7.12, 7.12-7.10, 6.70-6.66, 5.16-5.12, 4.85-4.76, 4.48, 3.88-3.86, 3.26-3.18, 2.48-2.43;

MS: m/z 488 (M+H)+;

HPLC retention time: 2.87 min (method 76).

### Example 2(130): 3-{trans-3-[(6,7-dimethoxy-4-quinolinyl)oxy]cyclobutyl}-1-[5-(trifluoromethyl)-3-pyridinyl]-2,4-imidazolidinedione

¹H NMR (400 MHz, CDCl₃): δ 8.90, 8.72-8.71, 8.55, 8.48-8.46, 7.41, 6.46, 5.33-5.27, 5.19-5.09, 4.41, 4.07, 4.04, 3.41-3.32, 2.83-2.75;

MS: m/z 503 (M+H)+;

HPLC retention time: 3.22 min (method 77).

### Example 2(131): 3-[trans-3-(4-quinolinyloxy)cyclobutyl]-1-[3-(trifluoromethyl)phenyl]-2,4-imidazolidinedione

¹H NMR (400 MHz, CDCl₃): δ 8.73, 8.28-8.24, 8.05, 7.90, 7.79-7.75, 7.75-7.70, 7.58-7.52, 7.46-7.43, 6.55, 5.36-5.30, 5.16-5.07, 4.35, 3.42-3.33, 2.82-2.74;

MS: m/z 442 (M+H)+;

HPLC retention time: 3.53 min (method 77).

### Example 2(132): 3-[trans-3-(4-quinolinyloxy)cyclobutyl]-1-[5-(trifluoromethyl)-3-pyridinyl]-2,4-imidazolidinedione

¹H NMR (400 MHz, CDCl₃): δ 8.89, 8.74, 8.72-8.71, 8.48-8.46, 8.28-8.24, 8.07-8.03, 7.75-7.70, 7.57-7.52, 6.54, 5.35-5.29, 5.17-5.08, 4.40, 3.41-3.33, 2.83-2.75;

MS: m/z 443 (M+H)+;

HPLC retention time: 3.34 min (method 77).

### Example 2(133): 3-{4-[(6,7-dimethoxy-4-quinolinyl)oxy]phenyl}-1-{3-[2-(1-piperidinyl)ethoxy]-5-(trifluoromethyl)phenyl}-2,4-imidazolidinedione

¹H NMR (500 MHz, CDCl₃): δ 8.54, 7.58-7.53, 7.52, 7.45, 7.40, 7.32, 7.00, 6.61, 4.52, 4.23-4.18, 4.06, 4.05, 2.87-2.80, 2.62-2.51, 1.69-1.61, 1.51-1.43;

MS: m/z 651 (M+H)+;

HPLC retention time: 8.82 min (method 82).

### Example 2(134): 3-{4-[(6,7-dimethoxy-4-quinolinyloxy]phenyl}-1-{3-[2-(4-morpholinyl)ethoxy]-5-(trifluoromethyl)phenyl}-2,4-imidazolidinedione

¹H NMR (500 MHz, CDCl₃): δ 8.54, 7.60, 7.56, 7.52, 7.49, 7.38, 7.32, 7.01, 6.62, 4.53, 4.20, 4.07, 4.05, 3.76, 2.85, 2.63-2.59;

MS: m/z 653 (M+H)+;

HPLC retention time: 8.34 min (method 82).

### Example 2(135): 3-{4-[(6,7-dimethoxy-4-quinolinyl)oxy]phenyl}-1-{3-[2-(1-pyrrolidinyl)ethoxy]-5-(trifluoromethyl)phenyl}-2,4-imidazolidinedione

¹H NMR (500 MHz, CDCl₃): δ 8.55, 7.58, 7.55, 7.52, 7.46-7.43, 7.32, 7.01, 6.61, 4.53, 4.26, 4.06, 4.05, 3.04, 2.77, 1.89;

MS: m/z 637 (M+H)+;

HPLC retention time: 8.64 min (method 82).

### Example 2(136): 3-{4-[(6,7-dimethoxy-4-quinolinyl)oxy]phenyl}-1-{3-[2-(4-methyl-1-piperazinyl)ethoxy]-5-(trifluoromethyl)phenyl}-2,4-imidazolidinedione

¹H NMR (500 MHz, CDCl₃): δ 8.55, 7.58, 7.55, 7.52, 7.45, 7.39, 7.32, 7.00, 6.61, 4.53, 4.17, 4.06, 4.05, 2.87, 2.78-2.67, 2.67-2.50, 2.38;

MS: m/z 656 (M+H)+;

HPLC retention time: 7.80 min (method 82).

### Example 2(137): 3-{4-[(6,7-dimethoxy-4-quinolinyl)oxy]phenyl}-1-{3-[2-(1-piperazinyl)ethoxy]-5-(trifluoromethyl)phenyl}-2,4-imidazolidinedione

¹H NMR (500 MHz, CDCl₃): δ 8.54, 7.59, 7.55, 7.52, 7.45, 4.38, 7.32, 7.00, 6.60, 4.53, 4.17, 4.06, 4.05, 3.01, 2.85, 2.69-2.63;

MS: m/z 652 (M+H)+;

HPLC retention time: 7.61 min (method 82).

### Example 2(138): 3-{-4-[(6,7-dimethoxy-4-quinolinyl)oxy]phenyl}-1-{3-[2-(dimethylamino)ethoxy]-5-(trifluoromethyl)phenyl}-2,4-imidazolidinedione

¹H NMR (500 MHz, CDCl₃): δ 8.54, 7.57-7.53, 7.52, 7.47-7.44, 7.32, 7.01, 6.61, 4.52, 4.16, 4.06, 4.05, 2.80, 2.39;

MS: m/z 611 (M+H)+;

HPLC retention time: 8.28 min (method 82).

### Example 2(139): 3-{4-[(6,7-dimethoxy-4-quinolinyl)oxy]phenyl}-1-{3-[2-(1,1-dioxido-4-thiomorpholinyl)ethoxy]-5-(trifluoromethyl)phenyl}-2,4-imidazolidinedione

¹H NMR (500 MHz, DMSO-d₆): δ 8.54, 7.79, 7.56, 7.50, 7.47, 7.44-7.41, 7.09, 6.61, 4.71, 4.21, 3.96, 3.93, 3.12-3.08, 3.07-3.03, 2.96;

MS: m/z 701 (M+H)+;

HPLC retention time: 14.13 min (method 75).

### Example 2(140): 1-[3-(2-aminoethoxy)-5-(trifluoromethyl)phenyl]-3-{4-[(6,7-dimethoxy-4-quinolinyl)oxy]phenyl}-2,4-imidazolidinedione

¹H NMR (500 MHz, DMSO-d₆): δ 8.54, 7.76, 7.57, 7.50, 7.48, 7.44-7.41, 7.06, 6.61, 4.71, 4.04, 3.96, 3.93, 2.91;

MS: m/z 583 (M+H)+;

HPLC retention time: 8.05 min (method 82).

### Example 2(141): 3-{3-isopropyl-4-[(7-oxo-5,6,7,8-tetrahydro-4-pteridinyl)oxy]phenyl}-1-[3-(trifluoromethyl)phenyl]-2,4-imidazolidinedione

¹H NMR (400 MHz, DMSO-d₆): δ 11.15, 8.18, 7.94-7.89, 7.80, 7.68, 7.51, 7.45, 7.29-7.27, 7.21, 6.21, 4.70, 3.94, 3.12-3.02, 1.15;

LCMS: m/z 527 [M+H]+;

HPLC retention time: 3.282 min (method 6).

### Example 2(142): 3-{4-[(2,3-diamino-4-pyridinyl)oxy]-3-ethylphenyl}-1-[3-(trifluoromethoxy)phenyl]-2,4-imidazolidinedione

¹H NMR (400 MHz, DMSO-d₆): δ 7.88, 7.61-7.54, 7.35, 7.29-7.19, 7.19-7.13, 6.92, 5.96, 5.69, 4.64, 4.56, 2.67, 1.19;

LCMS: m/z 488 [M+H]+;

HPLC retention time: 1.312 min (method 5).

### Example 2(143): 3-{3-ethyl-4-[(2-oxo-1,2-dihydropyrido[2,3-b]pyrazin-8-yl)oxy]phenyl}-1-[3-(trifluoromethoxy)phenyl]-2,4-imidazolidinedione trifluoroacetate (1:1)

¹H NMR (400 MHz, DMSO-d₆): δ 12.75, 8.45, 8.38, 7.91, 7.62-7.55, 7.49, 7.38, 7.31, 7.24-7.14, 6.80, 4.67, 2.63, 1.16;

LCMS: m/z 526 [M+H]+;

HPLC retention time: 4.207 min (method 22).

### Example 2(144): 3-{3-isopropyl-4-[(7-oxo-7,8-dihydro-4-pteridinyl)oxy]phenyl}-1-[3-(trifluoromethyl)phenyl]-2,4-imidazolidinedione

¹H NMR (400 MHz, DMSO-d₆): δ 13.26, 8.52, 8.24-8.19, 7.93, 7.71, 7.55-7.50, 7.37-7.32, 4.71, 3.07-3.00, 1.17;

LCMS: m/z 525 [M+H]+;

HPLC retention time: 1.708 min (method 1).

### Example 2(145):3-{3-isopropyl-4-[(2-oxo-2,3-dihydro-1H-imidazo[4,5-b]pyridin-7-yl)oxy]phenyl}-1-[3-(trifluoromethyl)phenyl]-2,4-imidazolidinedione trifluoroacetate (1:1)

¹H NMR (300 MHz, DMSO-d₆): δ 11.43, 11.34, 8.20, 7.95-7.85, 7.80, 7.69, 7.58-7.47, 7.32, 7.18, 6.33, 4.71, 3.26-3.15, 1.20;

LCMS: m/z 512 [M+H]+;

HPLC retention time: 1.474 min (method 5).

### Example 2(146): 3-{3-ethyl-4-[(3-oxo-3,4-dihydropyrido[2,3-b]pyrazin-8-yl)oxy]phenyl}-1-[3-(trifluoromethoxy)phenyl]-2,4-imidazolidinedione trifluoroacetate (1:1)

¹H NMR (400 MHz, DMSO-d₆): δ 12.96, 8.38, 8.22, 7.90, 7.62, 7.57, 7.50, 7.37, 7.28, 7.23-7.13, 6.52, 4.67, 2.59, 1.15;

LCMS: m/z 526 [M+H]+;

HPLC retention time: 1.647 min (method 17).

### Example 2(147): 3-{3-isopropyl-4-[(6-oxo-5,6,7,8-tetrahydro-4-pteridinyl)oxy]phenyl}-1-[3-(trifluoromethyl)phenyl]-2,4-imidazolidinedione

¹H NMR (400 MHz, D₂O+DMSO-d₆): δ 10.5, 8.17, 7.89-7.87, 7.77, 7.67, 7.53, 7.39, 7.26-7.23, 7.14-7.12, 4.69, 4.05, 3.16-3.14, 1.18;

LCMS: m/z 527 [M+H]+;

HPLC retention time: 3.282 min (method 2).

### Example 2(148): 3-{3-ethyl-4-[(2-oxo-1,2-dihydropyrido[2,3-b]pyrazin-8-yl)oxy]phenyl}-1-[5-(trifluoromethyl)-3-pyridinyl]-2,4-imidazolidinedione trifluoroacetate (1:1)

¹H NMR (400 MHz, DMSO-d₆): δ 12.76, 9.19, 8.79, 8.54-8.38, 7.50, 7.39-7.30, 6.81, 4.79, 2.67, 1.16;

LCMS: m/z 511 [M+H]+;

HPLC retention time: 2.456 min (method 25).

### Example 2(149): 3-{3-isopropyl-4-[(2-oxo-2,3-dihydro-1H-imidazo[4,5-b]pyridin-7-yl)oxy]phenyl}-1-[5-(trifluoromethyl)-3-pyridinyl]-2,4-imidazolidinedione

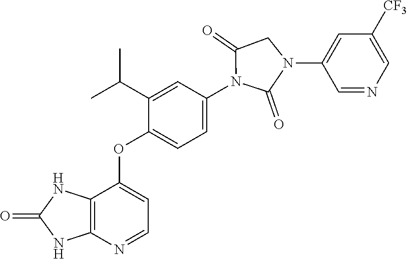 [SMILES: CC(C)c1cc(N2C(=O)CN(c3cncc(C(F)(F)F)c3)C2=O)ccc1Oc1ccnc2[nH]c(=O)[nH]c12]

¹H NMR (400 MHz, DMSO-d₆): δ 11.42, 11.32, 9.18, 8.78, 8.53, 7.80, 7.49, 7.33, 7.18, 6.33, 4.77, 3.23-3.17, 1.21;

LCMS: m/z 513 [M+H]+;

HPLC retention time: 3.254 min (method 26).

### Example 2(150): 3-{3-ethyl-4-[(7-oxo-5,6,7,8-tetrahydro-4-pteridinyl)oxy]phenyl}-1-[5-(trifluoromethyl)-3-pyridinyl]-2,4-imidazolidinedione

¹H NMR (400 MHz, DMSO-d₆): δ 11.17, 9.18, 8.78, 8.53, 7.79, 7.39-7.36, 7.32, 7.30-7.23, 6.21, 4.79, 3.94, 2.57-2.49, 1.13;

LCMS: m/z 514 [M+H]+;

HPLC retention time: 1.316 min (method 5).

### Example 2(151): 3-{3-ethyl-4-[(7-oxo-7,8-dihydro-4-pteridinyl)oxy]phenyl}-1-[5-(trifluoromethyl)-3-pyridinyl]-2,4-imidazolidinedione

¹H NMR (400 MHz, DMSO-d₆): δ 13.29, 9.20, 8.79, 8.54-8.52, 8.25, 7.45, 7.36, 4.79, 2.56-2.49, 1.12;

LCMS: m/z 512 [M+H]+;

HPLC retention time: 1.443 min (method 9).

### Example 2(152): 3-{4-[(6-amino-4-pyrimidinyl)oxy]-3-ethylphenyl}-1-[5-(trifluoromethyl)-3-pyridinyl]-2,4-imidazolidinedione trifluoroacetate (1:1)

¹H NMR (400 MHz, CD₃OD): δ 9.14, 8.68, 8.59, 8.35-8.32, 7.54, 7.44, 7.29, 5.99-5.97, 4.71, 2.64, 1.23;

LCMS: m/z 459 [M+H]+;

HPLC retention time: 1.462 min (method 1).

### Example 2(153): 3-{4-[(2-amino-5-chloro-4-pyridinyl)oxy]-3-ethylphenyl}-1-[5-(trifluoromethyl)-3-pyridinyl]-2,4-imidazolidinedione

¹H NMR (400 MHz, DMSO-d₆): δ 9.10, 8.74, 8.49, 8.18, 7.52, 7.45-7.36, 6.01, 4.73, 2.54, 1.14;

LCMS: m/z 492 [M+H]+;

HPLC retention time: 1.417 min (method 1).

### Example 2(154): 3-{3-methyl-4-[(2-oxo-1,2-dihydropyrido[2,3-b]pyrazin-8-yl)oxy]phenyl}-1-[5-(trifluoromethyl)-3-pyridinyl]-2,4-imidazolidinedione

¹H NMR (400 MHz, DMSO-d₆): δ 9.20, 8.79, 8.54, 8.41-8.30, 7.48-7.39, 6.81, 4.79, 2.26;

LCMS: m/z 497 [M+H]+;

HPLC retention time: 1.225 min (method 22).

### Example 2(155): 3-{3-(difluoromethoxy)-4-[(3-oxo-3,4-dihydropyrido[2,3-b]pyrazin-8-yl)oxy]phenyl}-1-[5-(trifluoromethyl)-3-pyridinyl]-2,4-imidazolidinedione

¹H NMR (300 MHz, DMSO-d₆): δ 13.01, 9.21, 8.81-8.80, 8.55, 8.43, 8.22, 7.59, 7.53-7.47, 7.44-7.01, 6.68, 4.80;

LCMS: m/z 549 [M+H]+;

HPLC retention time: 2.278 min (method 11-2).

### Example 2(156): 3-{3-ethyl-4-[(3-oxo-3,4-dihydropyrido[2,3-b]pyrazin-8-yl)oxy]phenyl}-1-[5-(trifluoromethyl)-3-pyridinyl]-2,4-imidazolidinedione trifluoroacetate (1:1)

¹H NMR (400 MHz, DMSO-d₆): δ 12.96, 9.19, 8.78, 8.54, 8.39, 7.51, 7.40-7.38, 7.38, 7.29, 6.52, 4.78, 2.61, 1.16;

LCMS: m/z 552 [M+MeCN+H]+

HPLC retention time: 1.615 min (method 10).

### Example 2(157): 3-{3-isopropyl-4-[(3-oxo-1,2,3,4-tetrahydropyrido[2,3-b]pyrazin-8-yl)oxy]phenyl}-1-[5-(trifluoromethyl)-3-pyridinyl]-2,4-imidazolidinedione

¹H NMR (400 MHz, DMSO-d₆): δ 10.81, 9.19, 8.78, 8.53, 7.55-7.46, 7.30, 7.09, 6.26, 4.78, 3.85, 3.26, 1.21;

LCMS: m/z 527 [M+H]+;

HPLC retention time: 1.495 min (method 23).

### Example 2(158): 3-{3-isopropyl-4-[(2-oxo-1,2,3,4-tetrahydropyrido[2,3-b]pyrazin-8-yl)oxy]phenyl}-1-[5-(trifluoromethyl)-3-pyridinyl]-2,4-imidazolidinedione

¹H NMR (400 MHz, DMSO-d₆): δ 10.39, 9.18, 8.78, 8.53, 7.55, 7.45, 7.29, 7.09, 6.86, 5.94, 4.76, 3.94, 3.28-3.24, 1.19;

LCMS: m/z 527 [M+H]+;

HPLC retention time: 1.149 min (method 30).

### Example 2(159): formic acid—3-{4-[(2,3-diamino-4-pyridinyl)oxy]-3-methylphenyl}-1-[5-(trifluoromethyl)-3-pyridinyl]-2,4-imidazolidinedione (1:1)

¹H NMR (400 MHz, DMSO-d₆): δ 9.17, 8.77, 8.52, 7.34, 7.27-7.22, 6.92, 5.98, 5.68, 4.75, 4.56, 2.32;

LCMS: m/z 459 [M+H]+;

HPLC retention time: 1.053 min (method 5).

### Example 2(160): 3-{4-[(2,3-diamino-4-pyridinyl)oxy]-3-ethylphenyl}-1-[5-(trifluoromethyl)-3-pyridinyl]-2,4-imidazolidinedione

¹H NMR (400 MHz, DMSO-d₆): δ 9.16, 8.77, 8.52, 7.36, 7.26-7.25, 6.92, 5.96, 5.63, 4.75, 4.52, 2.68, 1.19;

LCMS: m/z 473 [M+H]+;

HPLC retention time: 1.192 min (method 10).

### Example 2(161): 3-{4-[(2,3-diamino-4-pyridinyl)oxy]-3-(difluoromethoxy)phenyl}-1-[5-(trifluoromethyl)-3-pyridinyl]-2,4-imidazolidinedione

¹H NMR (400 MHz, DMSO-d₆): δ 9.17, 8.78, 8.52, 7.45, 7.35-7.27, 7.09-7.05, 6.15-5.91, 4.75;

LCMS: m/z 511 [M+H]+;

HPLC retention time: 1.619 min (method 34).

### Example 2(162): 3-{3-(difluoromethoxy)-4-[(2-oxo-1,2-dihydropyrido[2,3-b]pyrazin-8-yl)oxy]phenyl}-1-[5-(trifluoromethyl)-3-pyridinyl]-2,4-imidazolidinedione

¹H NMR (400 MHz, DMSO-d₆): δ 12.69, 9.20, 8.80, 8.54, 8.46-8.42, 7.58, 7.53, 7.51-7.11, 6.99, 4.80;

LCMS: m/z 549 [M+H]+;

HPLC retention time: 3.989 min (method 41).

### Example 2(163): 3-{3-bromo-4-[(2,3-diamino-4-pyridinyl)amino]phenyl}-1-[3-(trifluoromethoxy)phenyl]-2,4-imidazolidinedione

¹H NMR (300 MHz, DMSO-d₆): δ 7.87, 7.70, 7.61-7.53, 7.36-7.33, 7.30-7.28, 7.17-7.10, 6.22, 5.96, 4.62-4.55;

LCMS: m/z 539 [M+H]+;

HPLC retention time: 1.507 min (method 38).

### Example 2(164): 3-{4-[(2,3-diamino-4-pyridinyl)oxy]-3-isopropylphenyl}-1-[3-(trifluoromethyl)phenyl]-2,4-imidazolidinedione

¹H NMR (300 MHz, DMSO-d₆): δ 8.18-8.14, 7.90, 7.66, 7.53-7.50, 7.41, 7.27-7.21, 6.91, 5.94, 5.68, 4.68-4.51, 3.29, 1.21;

LCMS: m/z 486 [M+H]+;

HPLC retention time: 2.950 min (method 68).

### Example 2(165): 3-{4-[(2-oxo-2,3-dihydro-1H-imidazo[4,5-b]pyridin-7-yl)oxy]phenyl}-1-[3-(trifluoromethyl)phenyl]-2,4-imidazolidinedione

¹H NMR (300 MHz, DMSO-d₆): δ 4.69, 6.47-6.57, 7.15-7.34, 7.36-7.58, 7.59-7.73, 7.75-7.98, 8.03-8.26, 11.16-11.31, 11.34-11.63; TLC Rf: 0.45 (AcOEt).

### Reference Example 9: 3-(3-ethyl-4-hydroxy-phenyl)-1-[5-(trifluoromethyl)-3-pyridyl]imidazolidine-2,4-dione

To a solution of 4-amino-2-ethylphenol (CAS No. 178698-88-9, 1640 mg) in THE (30 mL), DIEA (4936 mg) and the compound prepared in Reference Example 5 (4540 mg) was added. The resulting mixture was heated for overnight under reflux. The solvent was evaporated under vacuum, the crude product was purified by column chromatography (Petroleum ether/EtOAc=2:1) to afford the title compound (3200 mg) having the following physical property values.

MS(ESI, Pos.): 365 (M+H)+;

¹H-NMR (DMSO-d₆): δ 9.69, 9.16, 8.76, 8.52, 7.09, 7.03, 6.87, 4.72, 2.57, 1.13.

### Example 3: 3-{4-[(2-amino-4-pyrimidinyl)oxy]-3-ethylphenyl}-1-[5-(trifluoromethyl)-3-pyridyl]imidazolidine-2,4-dione

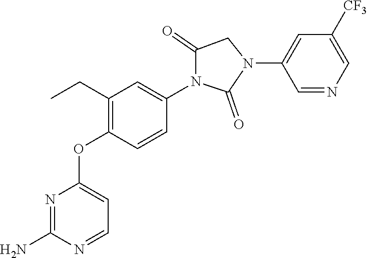 [SMILES: CCc1cc(N2C(=O)CN(c3cncc(C(F)(F)F)c3)C2=O)ccc1Oc1ccnc(N)n1]

To a solution of the compound prepared in Reference Example 9 (150 mg) in toluene (3 mL) was added 2-amino-4-chloropyrimidine (106 mg), XPhos Pd (CAS No. 1028206-56-5, 100 mg), and K₂CO₃ (113 mg). The resulting reaction mixture was stirred at 100° C. overnight. The reaction mixture was allowed to cooled to room temperature then concentrated under reduced pressure. The crude compound was purified by silica gel with elution of petroleum ether/ethyl acetate=1:1 to afford the title compound (40 mg) having the following physical property values.

¹H NMR (400 MHz, DMSO-d₆): δ 9.18, 8.78, 8.53, 8.13, 7.39-7.24, 6.69, 6.22, 4.78, 2.54-2.50, 1.12;

LCMS: m/z 459 [M+H]+;

HPLC retention time: 1.308 min (method 1).

### Example 3(1) 3(245)

A procedure for a purpose similar to that for Reference Example 9→Example 3 was carried out by using a corresponding alcohol compound instead of 4-amino-2-ethylphenol, a corresponding chlorocarbonyl compound instead of the compound prepared in Reference Example 5, and a corresponding halide compound instead of 2-amino-4-chloropyrimidine, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 3(1): 1-[3-(3-pyridinyl)-5-(trifluoromethyl)phenyl]-3-[4-(1H-pyrrolo[2,3-b]pyridin-4-yloxy)phenyl]-2,4-imidazolidinedione

¹H NMR (500 MHz, DMSO-d₆): δ 11.80, 9.04, 8.67-8.66, 8.36, 8.26-8.23, 8.15, 8.11, 7.87, 7.58-7.52, 7.40-7.39, 7.34-7.32, 6.57, 6.21-6.19, 4.8;

LCMS: m/z 529 [M+H]+;

HPLC retention time: 8.19 min (method 82).

### Example 3(2): 3-[3-ethyl-4-(1H-pyrazolo[3,4-b]pyridin-4-yloxy)phenyl]-1-[5-(trifluoromethyl)-3-pyridinyl]-2,4-imidazolidinedione trifluoroacetate (1:1)

¹H NMR (300 MHz, DMSO-d₆): δ 9.17, 8.77, 8.53, 8.37, 7.66, 7.51, 7.39, 6.44, 4.78, 2.60, 1.13;

LCMS: m/z 483 [M+H]+;

HPLC retention time: 1.538 min (method 1).

### Example 3(3): 3-[3-ethyl-4-(3H-imidazo[4,5-b]pyridin-7-yloxy)phenyl]-1-[5-(trifluoromethyl)-3-pyridinyl]-2,4-imidazolidinedione trifluoroacetate (1:1)

¹H NMR (400 MHz, DMSO-d₆): δ 9.19, 8.79, 8.75, 8.54, 8.37, 7.53, 7.42-7.35, 6.63, 4.80, 2.66, 1.15;

LCMS: m/z 483 [M+H]+;

HPLC retention time: 1.298 min (method 1).

### Example 3(4): 3-[3-ethyl-4-(7H-pyrrolo[2,3-d]pyrimidin-4-yloxy)phenyl]-1-[5-(trifluoromethyl)-3-pyridinyl]-2,4-imidazolidinedione trifluoroacetate (1:1)

¹H NMR (400 MHz, DMSO-d₆): δ 12.25, 9.19, 8.78, 8.54, 8.31, 7.50-7.35, 6.56-6.55, 4.79, 2.66, 1.10;

LCMS: m/z 483 [M+H]+;

HPLC retention time: 1.772 min (method 2).

### Example 3(5): 3-[3-ethyl-4-(9H-purin-6-yloxy)phenyl]-1-[5-(trifluoromethyl)-3-pyridinyl]-2,4-imidazolidinedione trifluoroacetate (1:1)

¹H NMR (400 MHz, DMSO-d₆): δ 9.20, 8.79, 8.55, 8.44, 7.46-7.35, 4.80, 2.66, 1.11;

LCMS: m/z 484 [M+H]+;

HPLC retention time: 1.292 min (method 3).

### Example 3(6): 3-{4-[(2-amino-4-pyridinyl)oxy]-3-ethylphenyl}-1-[5-(trifluoromethyl)-3-pyridinyl]-2,4-imidazolidinedione trifluoroacetate (1:1)

¹H NMR (400 MHz, DMSO-d₆): δ 9.19, 8.79, 8.54, 8.16, 7.83, 7.44, 7.35, 7.22, 6.18, 6.03, 5.81, 4.78, 2.56, 1.14;

LCMS: m/z 458 [M+H]+;

HPLC retention time: 1.197 min (method 1).

### Example 3(7): 3-{3-isopropyl-4-[(3-oxo-3,4-dihydropyrido[2,3-b]pyrazin-8-yl)oxy]phenyl}-1-[5-(trifluoromethyl)-3-pyridinyl]-2,4-imidazolidinedione trifluoroacetate (1:1)

¹H NMR (400 MHz, DMSO-d₆): δ 12.97, 9.20, 8.79, 8.55, 8.40, 8.23, 7.56, 7.39, 7.30, 6.54, 4.80, 3.16-3.10, 1.18;

LCMS: m/z 525 [M+H]+;

HPLC retention time: 2.696 min (method 4).

### Example 3(8): 3-[4-(1H-pyrrolo[2,3-b]pyridin-4-yloxy)-3-(trifluoromethoxy)phenyl]-1-[5-(trifluoromethyl)-3-pyridinyl]-2,4-imidazolidinedione

¹H NMR (400 MHz, CD₃OD): δ 9.18, 8.72, 8.62, 8.15, 7.75, 7.63, 7.44, 7.35, 6.61, 6.36, 4.75;

LCMS: m/z 538 [M+H]+;

HPLC retention time: 1.318 min (method 5).

### Example 3(9): 3-[4-(3H-imidazo[4,5-b]pyridin-7-yloxy)-3-isopropylphenyl]-1-[3-(trifluoromethyl)phenyl]-2,4-imidazolidinedione trifluoroacetate (1:1)

¹H NMR (300 MHz, DMSO-d₆): δ 8.72, 8.36, 8.20, 7.95-7.89, 7.72, 7.69-7.66, 7.58-7.55, 7.42-7.32, 6.62, 4.72, 3.23-3.12, 1.21;

LCMS: m/z 496 [M+H]+;

HPLC retention time: 1.402 min (method 9).

### Example 3(10): 3-[4-(7H-pyrrolo[2,3-d]pyrimidin-4-yloxy)bicyclo[2.2.2]oct-1-yl]-1-[5-(trifluoromethyl)-3-pyridinyl]-2,4-imidazolidinedione

¹H NMR (300 MHz, CD₃OD): δ 9.01, 8.61, 8.51, 8.28, 7.18, 6.44, 4.59, 2.64-2.53, 2.49-2.44;

LCMS: m/z 487 [M+H]+;

HPLC retention time: 2.912 min (method 15).

### Example 3(11): 3-[4-(7H-pyrrolo[2,3-d]pyrimidin-4-yloxy)bicyclo[2.2.2]oct-1-yl]-1-[3-(trifluoromethyl)phenyl]-2,4-imidazolidinedione

¹H NMR (400 MHz, DMSO-d₆): δ 11.95, 8.31, 8.13, 7.76-7.73, 7.65-7.61, 7.49-7.47, 7.31-7.29, 6.37, 4.42, 2.61-2.54, 2.52-2.48;

LCMS: m/z 486 [M+H]+;

HPLC retention time: 1.825 min (method 9).

### Example 3(12): 3-(4-{[2-amino-6-(2-methoxyethoxy)-4-pyrimidinyl]oxy}-3-ethylphenyl)-1-[5-(trifluoromethyl)-3-pyridinyl]-2,4-imidazolidinedione

¹H NMR (400 MHz, DMSO-d₆): δ 9.18, 8.79, 8.53, 7.37, 7.31-7.28, 7.22, 6.73, 5.52, 4.77, 4.35-4.33, 3.62-3.60, 3.33-3.27, 2.56-2.50, 1.14-1.11;

LCMS: m/z 533 [M+H]+;

HPLC retention time: 1.728 min (method 17).

### Example 3(13): formic acid—3-{4-[(2-amino-4-pyrimidinyl)oxy]-3-isopropylphenyl}-1-[5-(trifluoromethyl)-3-pyridinyl]-2,4-imidazolidinedione (1:1)

¹H NMR (400 MHz, CD₃OD): δ 9.14, 8.67, 8.60, 8.12-8.11, 7.51, 7.36, 7.21, 6.35, 4.70, 3.13-3.03, 1.23;

LCMS: m/z 473 [M+H]+;

HPLC retention time: 2.026 min (method 18).

### Example 3(14): 3-{3-ethyl-4-[(2-{[2-(methylsulfonyl)ethyl]amino}-4-pyrimidinyl)oxy]phenyl}-1-[5-(trifluoromethyl)-3-pyridinyl]-2,4-imidazolidinedione trifluoroacetate (1:1)

¹H NMR (400 MHz, CD₃OD): δ 9.86, 9.52, 9.27, 8.22, 7.53, 7.44, 7.34, 6.70, 5.36, 4.80, 4.08, 3.17, 2.64, 1.25;

LCMS: m/z 565 [M+H]+;

HPLC retention time: 0.777 min (method 81).

### Example 3(15): 3-[3-ethyl-4-(7H-pyrrolo[2,3-d]pyrimidin-4-yloxy)phenyl]-1-[3-(trifluoromethoxy)phenyl]-2,4-imidazolidinedione

¹H NMR (400 MHz, DMSO-d₆): δ 12.27, 8.31, 7.90, 7.62-7.57, 7.50, 7.42, 7.34, 7.17, 6.56, 4.67, 2.54-2.49, 1.12-1.05;

LCMS: m/z 520 [M+Na]+;

HPLC retention time: 1.570 min (method 5).

### Example 3(16): 3-[trans-4-(7H-pyrrolo[2,3-d]pyrimidin-4-yloxy)cyclohexyl]-1-[3-(trifluoromethyl)phenyl]-2,4-imidazolidinedione

¹H NMR (400 MHz, DMSO-d₆): δ 12.01, 8.36, 8.14, 7.81, 7.65, 7.49, 7.34, 6.45, 5.36-5.13, 4.53, 4.15-3.93, 2.32, 1.83, 1.63;

LCMS: m/z 460 [M+H]+;

HPLC retention time: 1.804 min (method 20).

### Example 3(17): 3-[trans-4-(7H-pyrrolo[2,3-d]pyrimidin-4-yloxy)cyclohexyl]-1-[5-(trifluoromethyl)-3-pyridinyl]-2,4-imidazolidinedione

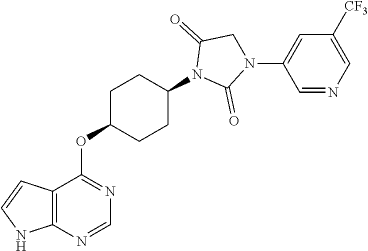 [SMILES: O=C1CN(c2cncc(C(F)(F)F)c2)C(=O)N1[C@H]1CC[C@@H](Oc2ncnc3[nH]ccc23)CC1]

¹H NMR (400 MHz, DMSO-d₆): δ 12.01, 9.10, 8.74, 8.49, 8.36, 7.34, 6.45, 5.28-5.22, 4.58, 4.04-4.03, 2.31, 1.83, 1.64;

LCMS: m/z 461 [M+H]+;

HPLC retention time: 3.063 min (method 21).

### Example 3(18): 3-[trans-3-(7H-pyrrolo[2,3-d]pyrimidin-4-yloxy)cyclopentyl]-1-[3-(trifluoromethyl)phenyl]-2,4-imidazolidinedione

¹H NMR (300 MHz, CDCl₃): δ 10.20, 8.47, 7.90, 7.77, 7.56, 7.45, 7.19, 6.60, 5.94-5.91, 5.00, 4.32, 2.79-2.62, 2.61-2.48, 2.40-2.19, 2.17-2.09;

LCMS: m/z 446 [M+H]+;

HPLC retention time: 1.629 min (method 9).

### Example 3(19): 3-[3-ethyl-4-(3H-imidazo[4,5-b]pyridin-7-yloxy)phenyl]-1-[3-(trifluoromethoxy)phenyl]-2,4-imidazolidinedione

¹H NMR (300 MHz, DMSO-d₆): δ 8.24, 8.13, 7.90, 7.63-7.60, 7.48, 7.35, 7.26, 7.17, 6.50, 4.67, 2.63, 1.17;

LCMS: m/z 498 [M+H]+;

HPLC retention time: 1.269 min (method 5).

### Example 3(20): 3-{4-[(2-amino-4-pyrimidinyl)oxy]-3-ethylphenyl}-1-[3-(trifluoromethoxy)phenyl]-2,4-imidazolidinedione

¹H NMR (400 MHz, DMSO-d₆): δ 8.15-8.13, 7.88, 7.62-7.55, 7.38, 7.37-7.24, 7.18, 6.78, 6.24, 4.65, 2.54-2.51, 1.13;

LCMS: m/z 474 [M+H]+;

HPLC retention time: 1.317 min (method 5).

### Example 3(21): 3-[2-fluoro-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.89, 8.20-8.13, 7.90, 7.69, 7.62-7.53, 7.44, 7.38, 7.15-7.13, 6.69, 6.22, 4.95-4.79;

LCMS: m/z 471 [M+H]+;

HPLC retention time: 1.742 min (method 10).

### Example 3(22): 3-[2-methoxy-4-(1H-pyrrolo[2,3-b]pyridin-4-yloxy)phenyl]-1-[3-(trifluoromethyl)phenyl]-2,4-imidazolidinedione trifluoroacetate (1:1)

¹H NMR (400 MHz, CDCl₃): δ 13.90, 8.07, 8.00, 7.83, 7.58, 7.49, 7.47-7.42, 6.96-6.94, 6.79, 6.60, 4.65, 4.57, 3.86;

LCMS: m/z 483 [M+H]+;

HPLC retention time: 1.582 min (method 10).

### Example 3(23): 3-[2-fluoro-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.89, 9.18, 8.80, 8.51, 8.20, 7.58, 7.45-7.33, 7.40-7.36, 7.18-7.13, 6.70, 6.22, 4.90-4.86;

LCMS: m/z 472 [M+H]+;

HPLC retention time: 1.539 min (method 10).

### Example 3(24): 3-[2-methoxy-4-(1H-pyrrolo[2,3-b]pyridin-4-yloxy)phenyl]-1-[5-(trifluoromethyl)-3-pyridinyl]-2,4-imidazolidinedione trifluoroacetate (1:1)

¹H NMR (400 MHz, CDCl₃): δ 13.83, 8.99, 8.74, 8.56, 8.11, 7.44-7.42, 6.97-6.95, 6.80, 6.61-6.60, 4.70-4.59, 3.87;

LCMS: m/z 484 [M+H]+;

HPLC retention time: 1.414 min (method 10).

### Example 3(25): 3-{3-isopropyl-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.53, 9.18, 8.78, 8.54, 8.01, 7.50, 7.34, 7.17, 6.27, 4.77, 3.13, 2.98, 2.58, 1.19;

LCMS: m/z 567 [M+MeCN+H]+;

HPLC retention time: 1.647 min (method 24).

### Example 3(26): 3-[3-ethyl-4-(pyrazolo[1,5-a]pyrimidin-7-yloxy)phenyl]-1-[5-(trifluoromethyl)-3-pyridinyl]-2,4-imidazolidinedione

¹H NMR (400 MHz, DMSO-d₆): δ 9.19, 9.11, 8.79, 8.54, 8.07, 7.44, 7.36, 6.91, 6.44, 4.79, 2.60-2.52, 1.14;

LCMS: m/z 505 [M+Na]+;

HPLC retention time: 2.730 min (method 27).

### Example 3(27): 3-[3-ethyl-4-(imidazo[1,2-b]pyridazin-8-yloxy)phenyl]-1-[5-(trifluoromethyl)-3-pyridinyl]-2,4-imidazolidinedione

¹H NMR (400 MHz, DMSO-d₆): δ 9.20, 8.79, 8.55, 8.34-8.32, 7.77, 7.54, 7.42, 6.25, 4.79, 2.62, 1.16;

LCMS: m/z 483 [M+H]+;

HPLC retention time: 1.523 min (method 1).

### Example 3(28): 3-{3-ethyl-4-[(6-methoxyimidazo[1,2-b]pyridazin-8-yl)oxy]phenyl}-1-[5-(trifluoromethyl)-3-pyridinyl]-2,4-imidazolidinedione trifluoroacetate (1:1)

¹H NMR (400 MHz, CD₃OD): δ 9.16, 8.69, 8.60, 8.19, 7.91, 7.62, 7.52, 7.43, 6.20, 4.74, 4.01, 2.73, 1.26;

LCMS: m/z 513 [M+H]+;

HPLC retention time: 2.698 min (method 4-1).

### Example 3(29): 3-[3-ethyl-4-(pyrazolo[1,5-a]pyrimidin-5-yloxy)phenyl]-1-[5-(trifluoromethyl)-3-pyridinyl]-2,4-imidazolidinedione

¹H NMR (400 MHz, CD₃OD): δ 9.15, 8.83, 8.68, 8.61, 8.00, 7.50, 7.42, 7.31, 6.79, 6.29, 4.72, 2.66, 1.21;

LCMS: m/z 483 [M+H]+;

HPLC retention time: 1.712 min (method 1).

### Example 3(30): 3-{3-ethyl-4-[(2-methyl-4-pyrimidinyl)oxy]phenyl}-1-[5-(trifluoromethyl)-3-pyridinyl]-2,4-imidazolidinedione

¹H NMR (400 MHz, DMSO-d₆): δ 9.19, 8.79, 8.60, 8.54, 7.44, 7.36-7.31, 6.98, 4.78, 2.53-2.48, 2.45, 1.11;

LCMS: m/z 458 [M+H]+;

HPLC retention time: 1.648 min (method 1).

### Example 3(31): 3-{4-[(2-amino-6-fluoro-4-pyridinyl)oxy]-3-ethylphenyl}-1-[5-(trifluoromethyl)-3-pyridinyl]-2,4-imidazolidinedione

¹H NMR (400 MHz, DMSO-d₆): δ 9.17, 8.78, 8.53, 7.45, 7.36, 7.27, 6.41, 5.80, 5.64, 4.77, 2.53, 1.13;

LCMS: m/z 517 [M+MeCN+H]+;

HPLC retention time: 1.872 min (method 10).

### Example 3(32): 3-{4-[(2-amino-3-fluoro-4-pyridinyl]oxyl-3-ethylphenyl}-1-[5-(trifluoromethyl)-3-pyridinyl]-2,4-imidazolidinedione

¹H NMR (400 MHz, DMSO-d₆): δ 9.19, 8.79, 8.54, 7.73, 7.48, 7.38, 7.28, 6.20, 4.78, 2.63, 1.18;

LCMS: m/z 476 [M+H]+;

HPLC retention time: 1.404 min (method 10).

### Example 3(33): 3-[3-methyl-4-(7H-pyrrolo[2,3-d]pyrimidin-4-yloxy)phenyl]-1-[5-(trifluoromethyl)-3-pyridinyl]-2,4-imidazolidinedione

¹H NMR (400 MHz, DMSO-d₆): δ 12.27, 9.20, 8.79, 8.54, 8.31, 7.51-7.49, 7.42, 7.37-7.32, 6.57-6.56, 4.79, 2.13;

LCMS: m/z 491 [M+Na]+

HPLC retention time: 1.340 min (method 5).

### Example 3(34): formic acid—3-[4-(3H-imidazo[4,5-b]pyridin-7-yloxy)-3-methylphenyl]-1-[5-(trifluoromethyl)-3-pyridinyl]-2,4-imidazolidinedione (1:1)

¹H NMR (400 MHz, DMSO-d₆): δ 13.21, 9.19, 8.79, 8.54, 8.37, 8.22, 7.47, 7.36-7.26, 6.47, 4.79, 2.24;

LCMS: m/z 469 [M+H]+;

HPLC retention time: 1.012 min (method 5).

### Example 3(35): 3-{4-[(2-amino-4-pyrimidinyl)oxy]-3-methylphenyl}-1-[5-(trifluoromethyl)-3-pyridinyl]-2,4-imidazolidinedione

¹H NMR (400 MHz, DMSO-d₆): δ 9.19, 8.78, 8.52, 8.14, 7.37, 7.32-7.25, 6.71, 6.22, 4.77, 2.14;

LCMS: m/z 445 [M+H]+;

HPLC retention time: 1.083 min (method 5).

### Example 3(36): 3-[3-(difluoromethoxy)-4-(7H-pyrrolo[2,3-d]pyrimidin-4-yloxy)phenyl]-1-[5-(trifluoromethyl)-3-pyridinyl]-2,4-imidazolidinedione

¹H NMR (400 MHz, DMSO-d₆): δ 12.31, 9.20, 8.80, 8.54, 8.31, 7.63, 7.54-7.51, 7.45, 7.30-6.93, 6.64, 4.80;

LCMS: m/z 521 [M+H]+;

HPLC retention time: 1.476 min (method 30).

### Example 3(37): 3-[3-(difluoromethoxy)-4-(3H-imidazo[4,5-b]pyridin-7-yloxy)phenyl]-1-[5-(trifluoromethyl)-3-pyridinyl]-2,4-imidazolidinedione

¹H NMR (300 MHz, DMSO-d₆): δ 9.20, 8.80, 8.55, 8.40, 8.27, 7.57, 7.49, 7.41-7.00, 6.67, 4.79;

LCMS: m/z 521 [M+H]+;

HPLC retention time: 1.438 min (method 10).

### Example 3(38): 3-{3-fluoro-4-[(7-oxo-5,6,7,8-tetrahydro-1,8-naphthyridin-4-yl)oxy]-5-vinylphenyl}-1-[5-(trifluoromethyl)-3-pyridinyl]-2,4-imidazolidinedione

¹H NMR (400 MHz, DMSO-d₆): δ 10.56, 9.20, 8.80, 8.54, 7.98, 7.74, 7.55, 6.80, 6.13, 5.95, 5.52, 4.83, 3.06, 2.60;

LCMS: m/z 528 [M+H]+;

HPLC retention time: 1.591 min (method 1).

### Example 3(39): 3-{4-[(2-amino-4-pyrimidinyl)oxy]-3-(difluoromethoxy)phenyl}-1-[5-(trifluoromethyl)-3-pyridinyl]-2,4-imidazolidinedione

¹H NMR (400 MHz, DMSO-d₆): δ 9.20, 8.80, 8.54, 8.17, 7.53, 7.49, 7.44-6.91, 6.79, 6.30, 4.78;

LCMS: m/z 497 [M+H]+;

HPLC retention time: 1.295 min (method 23).

### Example 3(40): 3-{3-ethyl-4-[(3-oxo-3,4-dihydro-2H-pyrido[3,2-b][1,4]oxazin-8-yl)oxy]phenyl}-1-[5-(trifluoromethyl)-3-pyridinyl]-2,4-imidazolidinedione

¹H NMR (400 MHz, DMSO-d₆): δ 11.35, 9.18, 8.79, 8.54, 7.84, 7.43, 7.32, 7.13, 6.48, 4.77, 4.70, 2.66, 1.18;

LCMS: m/z 555 [M+MeCN+H]+;

HPLC retention time: 1.694 min (method 10).

### Example 3(41):3-{3-ethyl-4-[(7-oxo-5,6,7,8-tetrahydro-1,8-naphthyridin-4-yl)oxy]phenyl}-1-[5-(trifluoromethyl)-3-pyridinyl]-2,4-imidazolidinedione

¹H NMR (400 MHz, DMSO-d₆): δ 10.54, 9.18, 8.78, 8.53, 8.01, 7.45, 7.35, 7.19, 6.26, 4.77, 2.99, 2.62-2.55, 1.15;

LCMS: m/z 553 [M+MeCN+H]+;

HPLC retention time: 1.685 min (method 10).

### Example 3(42): 3-{4-[(2-amino-4-pyrimidinyl)oxy]-3-ethylphenyl}-1-(3-isopropoxyphenyl)-2,4-imidazolidinedione

¹H NMR (400 MHz, DMSO-d₆): δ 8.15, 7.38-7.20, 6.76-6.72, 6.24, 4.66-4.60, 2.54, 1.29, 1.13;

LCMS: m/z 448 [M+H]+;

HPLC retention time: 1.288 min (method 5).

### Example 3(43): 3-{4-[(2-amino-4-pyrimidinyl)oxy]-3-ethylphenyl}-1-[3-(difluoromethoxy)phenyl]-2,4-imidazolidinedione

¹H NMR (400 MHz, DMSO-d₆): δ 8.15, 7.63, 7.53-7.46, 7.39, 7.32-7.24, 6.98, 6.78, 6.25, 4.64, 2.67, 1.14;

LCMS: m/z 456 [M+H]+;

HPLC retention time: 1.226 min (method 5).

### Example 3(44): 3-{4-[(2-amino-4-pyrimidinyl)oxy]-3-ethylphenyl}-1-[5-(trifluoromethyl)-1H-pyrazol-3-yl]-2,4-imidazolidinedione

¹H NMR (400 MHz, DMSO-d₆): δ 8.14, 7.37-7.24, 6.72, 6.22, 4.58, 2.55-2.49, 1.12;

LCMS: m/z 448 [M+H]+;

HPLC retention time: 2.450 min (method 16-1).

### Example 3(45): 3-[3-ethyl-4-(pyrazolo[1,5-a]pyridin-5-yloxy)phenyl]-1-[5-(trifluoromethyl)-3-pyridinyl]-2,4-imidazolidinedione

¹H NMR (400 MHz, DMSO-d₆): δ 9.19, 8.78, 8.70, 8.54, 7.94, 7.46, 7.24, 7.21, 6.89, 6.76, 6.47, 4.77, 2.65, 1.18;

LCMS: m/z 482 [M+H]+;

HPLC retention time: 1.738 min (method 33).

### Example 3(46): 3-[3-ethyl-4-(imidazo[1,2-b]pyridazin-6-yloxy)phenyl]-1-[5-(trifluoromethyl)-3-pyridinyl]-2,4-imidazolidinedione

¹H NMR (400 MHz, DMSO-d₆): δ 9.19, 8.79, 8.54, 8.32, 8.22-8.14, 7.64, 7.45-7.40, 7.37, 7.22, 4.78, 2.67, 1.15;

LCMS: m/z 483 [M+H]+;

HPLC retention time: 1.812 min (method 34).

### Example 3(47): 3-[3-methyl-4-(pyrazolo[1,5-a]pyrimidin-7-yloxy)phenyl]-1-[5-(trifluoromethyl)-3-pyridinyl]-2,4-imidazolidinedione

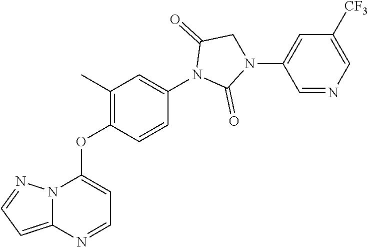 [SMILES: Cc1cc(N2C(=O)CN(c3cncc(C(F)(F)F)c3)C2=O)ccc1Oc1ccnc2ccnn12]

¹H NMR (400 MHz, DMSO-d₆): δ 9.19, 9.10, 8.79, 8.54, 8.07, 7.42-7.33, 6.90, 6.43, 4.79, 2.17;

LCMS: m/z 469 [M+H]+;

HPLC retention time: 2.826 min (method 16-2).

### Example 3(48): 3-[3-(difluoromethoxy)-4-(pyrazolo[1,5-a]pyrimidin-7-yloxy)phenyl]-1-[5-(trifluoromethyl)-3-pyridinyl]-2,4-imidazolidinedione

¹H NMR (400 MHz, DMSO-d₆): δ 9.20-9.11, 8.80, 8.54, 8.09, 7.64, 7.53, 7.48-7.45, 7.33-6.97, 6.93, 6.46, 4.80;

LCMS: m/z 562 [M+MeCN+H]+;

HPLC retention time: 1.392 min (method 36).

### Example 3(49): 3-[3-ethyl-4-(pyrazolo[1,5-a]pyrimidin-7-yloxy)phenyl]-1-[3-(trifluoromethoxy)phenyl]-2,4-imidazolidinedione

¹H NMR (400 MHz, DMSO-d₆): δ 9.11, 8.08, 7.92, 7.64-7.57, 7.44, 7.38-7.34, 7.19-7.17, 6.91, 6.45, 4.69, 2.58-2.53, 1.14;

LCMS: m/z 498 [M+H]+;

HPLC retention time: 2.267 min (method 37).

### Example 3(50): 3-[3-ethyl-4-(4-pyridinyloxy)phenyl]-1-[5-(trifluoromethyl)-3-pyridinyl]-2.4-imidazolidinedione trifluoroacetate (1:1)

¹H NMR (400 MHz, DMSO-d₆): δ 9.21, 8.80-8.74, 8.55, 7.57, 7.48-7.34, 4.80, 2.57, 1.15;

LCMS: m/z 443 [M+H]+;

HPLC retention time: 1.074 min (method 5).

### Example 3(51): 3-[3-ethyl-4-(3-pyridinyloxy)phenyl]-1-[5-(trifluoromethyl)-3-pyridinyl]-2,4-imidazolidinedione

¹H NMR (400 MHz, DMSO-d₆): δ 9.18, 8.79, 8.54, 8.40-8.38, 7.47-7.40, 7.39-7.28, 7.06-7.04, 4.77, 2.67, 1.19;

LCMS: m/z 443 [M+H]+;

HPLC retention time: 1.169 min (method 5).

### Example 3(52): 3-[3-ethyl-4-(4-pyrimidinyloxy)phenyl]-1-[5-(trifluoromethyl)-3-pyridinyl]-2,4-imidazolidinedione

¹H NMR (400 MHz, DMSO-d₆): δ 9.20, 8.79-8.72, 8.54, 7.45, 7.37-7.26, 4.79, 2.53, 1.11;

LCMS: m/z 485 [M+MeCN+H]+;

HPLC retention time: 1.263 min (method 36).

### Example 3(53): 3-[3-ethyl-4-(pyrrolo[1,2-b]pyridazin-4-yloxy)phenyl]-1-[5-(trifluoromethyl)-3-pyridinyl]-2,4-imidazolidinedione

¹H NMR (400 MHz, DMSO-d₆): δ 9.20, 8.80, 8.56, 8.03, 7.92, 7.52, 7.42, 6.87, 6.74, 5.74, 4.80, 2.60, 1.16;

LCMS: m/z 523 [M+MeCN+H]+;

HPLC retention time: 2.070 min (method 10).

### Example 3(54): 3-[7-(1H-pyrrolo[2,3-b]pyridin-4-yloxy)-2,3-dihydro-1H-inden-4-yl]-1-[3-(trifluoromethyl)phenyl]-2,4-imidazolidinedione

¹H NMR (400 MHz, DMSO-d₆): δ 11.87, 8.19, 8.14, 7.90, 7.70, 7.54-7.52, 7.41-7.40, 7.27, 7.10, 6.47, 6.26, 4.80-4.65, 2.89, 2.77, 2.07-1.98;

LCMS: m/z 493 [M+H]+;

HPLC retention time: 2.823 min (method 16-2).

### Example 3(55): formic acid—3-[4-(1H-pyrrolo[2,3-b]pyridin-4-yloxy)-5,6,7,8-tetrahydro-1-naphthalenyl]-1-[3-(trifluoromethyl)phenyl]-2,4-imidazolidinedione (1:1)

¹H NMR (300 MHz, DMSO-d₆): δ 11.82, 8.20, 8.13, 7.89, 7.67, 7.54, 7.40-7.38, 7.28, 7.09, 6.39, 6.26, 4.82, 2.62, 1.68;

LCMS: m/z 507 [M+H]+;

HPLC retention time: 1.385 min (method 5).

### Example 3(56): formic acid—3-[4-(2,3-dihydro-1H-pyrrolo[2,3-b]pyridin-4-yloxy)-3-ethylphenyl]-1-[5-(trifluoromethyl)-3-pyridinyl]-2,4-imidazolidinedione (1:1)

¹H NMR (400 MHz, DMSO-d₆): δ 9.17, 8.78, 8.53, 7.65, 7.40, 7.30, 7.12, 6.53, 5.90, 4.76, 3.51, 2.85, 2.61, 1.16;

LCMS: m/z 484 [M+H]+;

HPLC retention time: 1.629 min (method 38).

### Example 3(57): 3-[3-ethyl-4-(5,6,7,8-tetrahydro-1,8-naphthyridin-4-yloxy)phenyl]-1-[5-(trifluoromethyl)-3-pyridinyl]-2,4-imidazolidinedione

¹H NMR (400 MHz, DMSO-d₆): δ 9.18, 8.78, 8.53, 7.69, 7.40, 7.30, 7.06, 6.54, 5.78, 4.76, 3.27, 2.78, 2.67, 1.82-1.80, 1.18;

LCMS: m/z 498 [M+H]+;

HPLC retention time: 1.366 min (method 9).

### Example 3(58): 3-[3-ethyl-4-(1H-pyrrolo[2,3-c]pyridin-4-yloxy)phenyl]-1-[5-(trifluoromethyl)-3-pyridinyl]-2,4-imidazolidinedione trifluoroacetate (1:1)

¹H NMR (400 MHz, DMSO-d₆): δ 13.06, 9.18, 9.05, 8.78, 8.54, 8.19, 8.02, 7.50, 7.30, 7.13, 6.61, 4.78, 2.75, 1.23;

LCMS: m/z 482 [M+H]+;

HPLC retention time: 5.224 min (method 40).

### Example 3(59): 3-{3-[3-ethyl-4-(1H-pyrrolo[2,3-b]pyridin-4-yloxy)phenyl]-2,4-dioxo-1-imidazolidinyl}benzonitrile

¹H NMR (400 MHz, DMSO-d₆): δ 8.57, 8.14-8.12, 7.69-7.62, 7.46-7.36, 4.70, 4.01, 4.00, 2.68-2.50, 1.13;

LCMS: m/z 510 [M+H]+;

HPLC retention time: 1.369 min (method 5).

### Example 3(60): 3-{4-[(2-amino-4-pyrimidinyl)oxy]-2-isopropenylphenyl}-1-[5-(difluoromethoxy)-3-pyridinyl]-2,4-imidazolidinedione

¹H NMR (400 MHz, DMSO-d₆): δ 8.77, 8.33, 8.17, 8.04, 7.55-7.19, 6.76, 6.24, 5.14, 4.87-4.69, 2.00;

LCMS: m/z 469 [M+H]+;

HPLC retention time: 1.195 min (method 44).

### Example 3(61): 3-[3-bromo-4-(pyrrolo[1,2-b]pyridazin-4-yloxy)phenyl]-1-[5-(trifluoromethyl)-3-pyridinyl]-2,4-imidazolidinedione

¹H NMR (400 MHz, DMSO-d₆): δ 9.21, 8.81, 8.55, 8.06, 7.96-7.94, 7.69-7.62, 6.89, 6.73, 5.79, 4.81;

LCMS: m/z 532 [M+H]+;

HPLC retention time: 2.060 min (method 45).

### Example 3(62): 3-[4-(pyrrolo[1,2-b]pyridazin-4-yloxy)-3-vinylphenyl]-1-[5-(trifluoromethyl)-3-pyridinyl]-2,4-imidazolidinedione

¹H NMR (400 MHz, DMSO-d₆): δ 9.21, 8.80, 8.56, 8.03, 7.93-7.88, 7.53-7.47, 6.88, 6.80-6.72, 5.93-5.89, 5.71, 5.43, 4.82;

LCMS: m/z 521 [M+MeCN+H]+;

HPLC retention time: 1.556 min (method 36).

### Example 3(63): 3-[5-(7H-pyrrolo[2,3-d]pyrimidin-4-yloxy)bicyclo[2.2.1]hept-2-yl]-1-[5-(trifluoromethyl)-3-pyridinyl]-2,4-imidazolidinedione

¹H NMR (400 MHz, DMSO-d₆): δ 12.00, 9.08, 8.74, 8.49, 8.36, 7.34, 6.45, 5.16, 4.60-4.48, 3.98-3.93, 2.74-2.50, 2.47-2.40, 2.27-2.22, 2.17-1.99, 1.84-1.56;

LCMS: m/z 473 [M+H]+;

HPLC retention time: 2.779 min (method 16-2).

### Example 3(64): 3-[5-(7H-pyrrolo[2,3-d]pyrimidin-4-yloxy)bicyclo[2.2.1]hept-2-yl]-1-[5-(trifluoromethyl)-3-pyridinyl]-2,4-imidazolidinedione

¹H NMR (400 MHz, DMSO-d₆): δ 11.98, 9.10, 8.75, 8.49, 8.36, 7.33, 6.45, 5.44, 4.60-4.54, 4.26-4.22, 2.58-2.48, 2.33-2.26, 2.08, 1.98-1.82, 1.80-1.67, 1.65-1.37;

LCMS: m/z 495 [M+Na]+;

HPLC retention time: 1.367 min (method 5).

### Example 3(65): 3-[4-(7H-pyrrolo[2,3-d]pyrimidin-4-yloxy)bicyclo[2.2.1]hept-1-yl]-1-[5-(trifluoromethyl)-3-pyridinyl]-2,4-imidazolidinedione

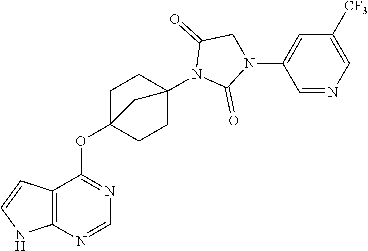 [SMILES: O=C1CN(c2cncc(C(F)(F)F)c2)C(=O)N1C12CCC(Oc3ncnc4[nH]ccc34)(CC1)C2]

¹H NMR (300 MHz, DMSO-d₆): δ 11.99, 9.07, 8.73, 8.49, 8.33, 7.34, 6.45, 4.55, 2.84, 2.51-2.38, 2.15-2.00;

LCMS: m/z 473 [M+H]+;

HPLC retention time: 1.467 min (method 46).

### Example 3(66): 3-[6-(7H-pyrrolo[2,3-d]pyrimidin-4-yloxy)spiro[3.3]hept-2-yl]-1-[5-(trifluoromethyl)-3-pyridinyl]-2,4-imidazolidinedione

¹H NMR (300 MHz, DMSO-d₆): δ 12.01, 9.07, 8.73, 8.47, 8.33, 7.34, 6.45, 5.35, 4.54-4.46, 2.95-2.89, 2.78-2.75, 2.60-2.57, 2.49-2.42, 2.32-2.21;

LCMS: m/z 473 [M+H]+;

HPLC retention time: 1.394 min (method 5).

### Example 3(67): 3-[3-isopropyl-4-(7H-pyrrolo[2,3-d]pyrimidin-4-yloxy)phenyl]-1-[3-(trifluoromethoxy)phenyl]-2,4-imidazolidinedione

¹H NMR (400 MHz, DMSO-d₆): δ 12.28, 8.32, 7.90, 7.64, 7.58, 7.51-7.47, 7.32, 7.18, 6.57, 4.67, 3.05-3.01, 1.14;

LCMS: m/z 512 [M+H]+;

HPLC retention time: 2.516 min (method 47).

### Example 3(68): 3-{4-[(6-amino-3-pyridinyl)oxy]phenyl}-1-[5-(trifluoromethyl)-3-pyridinyl]-2,4-imidazolidinedione

¹H NMR (400 MHz, DMSO-d₆): δ 9.17, 8.77, 8.51, 8.00, 7.83-7.62, 7.43, 7.17, 7.00, 4.75;

LCMS: m/z 430 [M+H]+;

HPLC retention time: 1.036 min (method 5).

### Example 3(69): 3-[3-bromo-4-(3-pyridinyloxy)phenyl]-1-[5-(trifluoromethyl)-3-pyridinyl]-2,4-imidazolidinedione

¹H NMR (400 MHz, DMSO-d₆): δ 9.20, 8.80, 8.53, 8.45-8.41, 7.88, 7.52-7.46, 7.28, 4.78;

LCMS: m/z 493 [M+H]+;

HPLC retention time: 1.204 min (method 5).

### Example 3(70): 3-[4-(3-pyridinyloxy)-3-vinylphenyl]-1-[5-(trifluoromethyl)-3-pyridinyl]-2,4-imidazolidinedione

¹H NMR (400 MHz, DMSO-d₆): δ 9.20, 8.79, 8.54, 8.41-8.39, 7.80, 7.47-7.38, 7.13, 6.98-6.91, 5.91-5.86, 5.45-5.42, 4.79;

LCMS: m/z 441 [M+H]+;

HPLC retention time: 1.164 min (method 5).

### Example 3(71): 3-[2-ethyl-4-(7H-pyrrolo[2,3-d]pyrimidin-4-yloxy)phenyl]-1-[5-(trifluoromethyl)-3-pyridinyl]-2,4-imidazolidinedione

¹H NMR (400 MHz, DMSO-d₆): δ 12.26, 9.19, 8.79, 8.53, 8.35, 7.51, 7.41, 7.34, 7.27-7.25, 6.55, 4.94, 4.78, 2.57-2.52, 1.11;

LCMS: m/z 483 [M+H]+;

HPLC retention time: 1.437 min (method 5).

### Example 3(72): 3-{4-[(2-amino-4-pyrimidinyl)oxy]-2-ethylphenyl}-1-[5-(trifluoromethyl)-3-pyridinyl]-2,4-imidazolidinedione

¹H NMR (400 MHz, DMSO-d₆): δ 9.17, 8.79, 8.50, 8.15, 7.38, 7.24, 7.18-7.15, 6.73, 6.20, 4.92, 4.77, 2.56, 1.11;

LCMS: m/z 459 [M+H]+;

HPLC retention time: 1.111 min (method 5).

### Example 3(73): 3-[2-ethyl-4-(pyrazolo[1,5-a]pyrimidin-7-yloxy)phenyl]-1-[5-(trifluoromethyl)-3-pyridinyl]-2,4-imidazolidinedione

¹H NMR (400 MHz, DMSO-d₆): δ 9.17, 9.10, 8.79, 8.53, 8.08, 7.44, 7.34, 7.29-7.26, 6.86, 6.46, 4.94, 4.77, 2.59-2.53, 1.13;

LCMS: m/z 483 [M+H]+;

HPLC retention time: 2.826 min (method 16).

### Example 3(74): 3-[2-ethyl-4-(pyrazolo[1,5-a]pyrimidin-5-yloxy)phenyl]-1-[5-(trifluoromethyl)-3-pyridinyl]-2,4-imidazolidinedione

¹H NMR (400 MHz, DMSO-d₆): δ 9.18, 9.10, 8.79, 8.53, 8.08, 7.44, 7.34, 7.29-7.27, 6.86, 6.46, 4.94, 4.77, 2.59-2.53, 1.12;

LCMS: m/z 483 [M+H]+;

HPLC retention time: 2.821 min (method 16).

### Example 3(75): 3-[2-isopropyl-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.84, 9.18, 8.79, 8.54, 8.17, 7.42-7.33, 7.13, 6.58, 6.22, 4.94, 4.77, 3.07-2.93, 1.13;

LCMS: m/z 496 [M+H]+;

HPLC retention time: 1.268 min (method 5).

### Example 3(76): 2-{2,5-dioxo-3-[5-(trifluoromethyl)-3-pyridinyl]-1-imidazolidinyl}-5-(1H-pyrrolo[2,3-b]pyridin-4-yloxy)benzonitrile

¹H NMR (400 MHz, DMSO-d₆): δ 11.94, 9.19, 8.82, 8.52, 8.22, 7.98, 7.73, 7.67, 7.46, 6.73, 6.21, 5.00-4.94;

LCMS: m/z 479 [M+H]+;

HPLC retention time: 2.307 min (method 28).

### Example 3(77): 3-{4-[(2-amino-4-pyrimidinyl)oxy]-3-isopropylphenyl}-1-[3-(trifluoromethyl)phenyl]-2,4-imidazolidinedione

¹H NMR (400 MHz, DMSO-d₆): δ 8.17-8.13, 7.90, 7.68, 7.53, 7.43, 7.31, 7.24, 6.71, 6.21, 4.70, 3.05-2.98, 1.16;

LCMS: m/z 472 [M+H]+;

HPLC retention time: 1.343 min (method 5).

### Example 3(78): 3-{4-[(2-amino-4-pyrimidinyl)oxy]-3-isopropylphenyl}-1-{4-[(4-methyl-1-piperazinyl)methyl]-3-(trifluoromethyl)phenyl}-2,4-imidazolidinedione

¹H NMR (400 MHz, DMSO-d₆): δ 8.16-8.13, 7.83-7.76, 7.43, 7.30-7.21, 6.70, 6.22, 4.68, 3.58, 3.02, 2.52-2.32, 2.19, 1.16;

LCMS: m/z 584 [M+H]+;

HPLC retention time: 2.169 min (method 52).

### Example 3(79): 3-{4-[(2-amino-4-pyrimidinyl)oxy]-3-isopropylphenyl}-1-[3-(trifluoromethoxy)phenyl]-2,4-imidazolidinedione

¹H NMR (400 MHz, DMSO-d₆): δ 8.13, 7.88, 7.62-7.54, 7.42, 7.29, 7.23, 7.20-7.13, 6.72, 6.22, 4.64, 3.04-2.97, 1.16;

LCMS: m/z 488 [M+H]+;

HPLC retention time: 2.773 min (method 53).

### Example 3(80): 3-{4-[(2-amino-4-pyrimidinyl)oxy]-3-isopropylphenyl}-1-[3-(cyclopentyloxy)phenyl]-2,4-imidazolidinedione

¹H NMR (300 MHz, DMSO-d₆): δ 8.14, 7.42, 7.32-7.23, 7.21, 6.73-6.70, 6.22, 4.84-4.80, 4.60, 3.05-2.96, 1.93, 1.72-1.61, 1.15;

LCMS: m/z 488 [M+H]+;

HPLC retention time: 1.603 min (method 43).

### Example 3(81): 1-[3-(2-methyl-2-propanyl)-1-phenyl-1H-pyrazol-5-yl]-3-[4-(1H-pyrrolo[2,3-b]pyridin-4-yloxy)phenyl]-2,4-imidazolidinedione

¹H NMR (400 MHz, DMSO-d₆): δ 11.78, 8.12, 7.59-7.57, 7.51-7.47, 7.39-7.33, 7.28-7.25, 6.55-6.51, 6.19, 4.56, 1.31;

LCMS: m/z 507 [M+H]+;

HPLC retention time: 2.027 min (method 54).

### Example 3(82): 3-[5-(7H-pyrrolo[2,3-d]pyrimidin-4-yloxy)bicyclo[2.2.1]hept-2-yl]-1-[5-(trifluoromethyl)-3-pyridinyl]-2,4-imidazolidinedione

¹H NMR (300 MHz, DMSO-d₆): δ 12.00, 9.08, 8.74, 8.49, 8.36, 7.34, 6.45, 5.17, 4.60-4.48, 3.98-3.93, 2.74-2.50, 2.47-2.40, 2.27-2.22, 2.17-1.99, 1.84-1.56;

LCMS: m/z 473 [M+H]+;

HPLC retention time: 2.777 min (method 16-2).

### Example 3(83): 3-[5-(7H-pyrrolo[2,3-d]pyrimidin-4-yloxy)bicyclo[2.2.1]hept-2-yl]-1-[5-(trifluoromethyl)-3-pyridinyl]-2,4-imidazolidinedione

¹H NMR (400 MHz, DMSO-d₆): δ 12.00, 9.10, 8.75, 8.49, 8.36, 7.33, 6.45, 5.45, 4.60, 4.26-4.22, 2.74-2.50, 2.47-2.40, 2.27-2.22, 2.17-1.99, 1.84-1.56, 1.19-1.04, LCMS: m/z 495 [M+Na]+;

HPLC retention time: 1.367 min (method 5).

### Example 3(84): 3-{4-[(2-amino-4-pyrimidinyl)oxy]-2-isopropenylphenyl}-1-[4-fluoro-3-(trifluoromethyl)phenyl]-2,4-imidazolidinedione

¹H NMR (400 MHz, DMSO-d₆): δ 8.17-8.10, 7.95, 7.64, 7.46-7.43, 7.28, 6.75, 6.24, 5.14, 4.85-4.80, 4.70, 2.01;

LCMS: m/z 488 [M+H]+;

HPLC retention time: 1.616 min (method 55).

### Example 3(85): 3-{4-[(2-amino-4-pyrimidinyl)oxy]-3-ethylphenyl}-1-[4-fluoro-3-(trifluoromethoxy)phenyl]-2,4-imidazolidinedione

¹H NMR (300 MHz, DMSO-d₆): δ 8.14, 8.06-8.04, 7.68-7.59, 7.38, 7.32-7.23, 6.71, 6.22, 4.65, 2.56-2.49, 1.13;

LCMS: m/z 492 [M+H]+;

HPLC retention time: 1.548 min (method 1).

### Example 3(86): 3-{4-[(2-amino-4-pyrimidinyl)oxy]-3-ethylphenyl}-1-[3-(difluoromethyl)phenyl]-2,4-imidazolidinedione

¹H NMR (400 MHz, DMSO-d₆): δ 8.14, 8.01, 7.78-7.76, 7.60, 7.39-7.35, 7.32, 7.29-6.93, 6.70, 6.22, 4.67, 2.56-2.49, 1.13;

LCMS: m/z 440 [M+H]+;

HPLC retention time: 2.400 min (method 16-2).

### Example 3(87): 3-{4-[(2-amino-4-pyrimidinyl)oxy]-3-ethylphenyl}-1-[2-fluoro-5-(trifluoromethyl)phenyl]-2,4-imidazolidinedione

¹H NMR (400 MHz, DMSO-d₆): δ 8.17-8.13, 7.80, 7.66, 7.40-7.24, 6.71, 6.22, 4.68, 2.55, 1.13;

LCMS: m/z 476 [M+H]+;

HPLC retention time: 1.246 min (method 5).

### Example 3(88): formic acid—3-{4-[(2-amino-4-pyrimidinyl)oxy]-3-ethylphenyl}-1-[3-fluoro-5-(trifluoromethyl)phenyl]-2,4-imidazolidinedione (1:1)

¹H NMR (400 MHz, DMSO-d₆): δ 8.14-8.13, 8.03, 7.83, 7.49, 7.38, 7.31, 7.25, 6.70, 6.21, 4.69, 2.54, 1.17;

LCMS: m/z 476 [M+H]+;

HPLC retention time: 1.322 min (method 5).

### Example 3(89): 3-{4-[(2-amino-4-pyrimidinyl)oxy]-3-ethylphenyl}-1-[2-ethoxy-5-(trifluoromethyl)phenyl]-2,4-imidazolidinedione

¹H NMR (400 MHz, DMSO-d₆): δ 8.14, 7.90, 7.70, 7.40-7.31, 7.25, 6.71, 6.22, 4.54, 4.24, 2.55, 1.41, 1.14;

LCMS: m/z 502 [M+H]+;

HPLC retention time: 1.315 min (method 5).

### Example 3(90): 3-{4-[(2-amino-4-pyrimidinyl)oxy]-3-ethylphenyl}-1-[4-(4-morpholinyl)-3-(trifluoromethyl)phenyl]-2,4-imidazolidinedione

¹H NMR (400 MHz, DMSO-d₆): δ 8.18-8.11, 7.83, 7.69, 7.39-7.23, 6.71, 6.22, 4.69, 3.72-3.69, 2.86-2.84, 2.55, 1.41;

LCMS: m/z 543 [M+H]+;

HPLC retention time: 1.289 min (method 5).

### Example 3(91): 3-{4-[(2-amino-4-pyrimidinyl)oxy]-3-ethylphenyl}-1-[4-fluoro-3-(trifluoromethyl)phenyl]-2,4-imidazolidinedione

¹H NMR (300 MHz, DMSO-d₆): δ 8.19-8.13, 7.97, 7.64, 7.39, 7.33-7.23, 6.71, 6.22, 4.71, 2.55, 1.13;

LCMS: m/z 476 [M+H]+;

HPLC retention time: 1.293 min (method 5).

### Example 3(92): 3-{4-[(2-amino-4-pyrimidinyl)oxy]-2-methylphenyl}-1-[3-(difluoromethoxy)phenyl]-2,4-imidazolidinedione

¹H NMR (400 MHz, DMSO-d₆): δ 8.15, 7.63, 7.53-7.46, 7.37, 7.27-7.22, 7.15, 7.00, 6.75, 6.23, 4.78, 4.64, 2.19;

LCMS: m/z 442 [M+H]+;

HPLC retention time: 1.675 min (method 17).

### Example 3(93): 3-{4-[(2-amino-4-pyrimidinyl)oxy]-2-ethylphenyl}-1-[3-(trifluoromethyl)phenyl]-2,4-imidazolidinedione

¹H NMR (400 MHz, DMSO-d₆): 8.15, 7.88, 7.68, 7.52, 7.37, 7.23, 7.15, 6.76, 6.23, 4.88, 4.72, 2.57, 1.14;

LCMS: m/z 458 [M+H]+;

HPLC retention time: 1.278 min (method 5).

### Example 3(94): 3-[2-ethyl-4-(7H-pyrrolo[2,3-d]pyrimidin-4-yloxy)phenyl]-1-[3-(trifluoromethyl)phenyl]-2,4-imidazolidinedione

¹H NMR (400 MHz, DMSO-d₆): δ 12.26, 8.36, 8.18, 7.92, 7.70, 7.55-7.50, 7.43-7.25, 6.56, 4.89, 4.73, 2.58-2.52, 1.13;

LCMS: m/z 482 [M+H]+;

HPLC retention time: 1.597 min (method 5).

### Example 3(95): 3-{4-[(2-amino-4-pyrimidinyl)oxy]-3-ethyl-2-methylphenyl}-1-[5-(trifluoromethyl)-3-pyridinyl]-2,4-imidazolidinedione

¹H NMR (400 MHz, DMSO-d₆): δ 9.18, 8.79, 8.52, 8.15, 7.25, 7.13, 6.72, 6.22, 4.90, 4.78, 2.60, 2.17, 1.06;

LCMS: m/z 473 [M+H]+;

HPLC retention time: 2.269 min (method 16-2).

### Example 3(96): 3-[3-ethyl-2-methyl-4-(7H-pyrrolo[2,3-d]pyrimidin-4-yloxy)phenyl]-1-[5-(trifluoromethyl)-3-pyridinyl]-2,4-imidazolidinedione

¹H NMR (400 MHz, DMSO-d₆): δ 12.26, 9.20, 8.79, 8.55, 8.32, 7.51, 7.34-7.18, 6.56, 4.92, 4.80, 2.60, 2.20, 1.05;

LCMS: m/z 497 [M+H]+;

HPLC retention time: 1.665 min (method 1).

### Example 3(97): 3-[3-ethyl-2-methyl-4-(7H-pyrrolo[2,3-d]pyrimidin-4-yloxy)phenyl]-1-[3-(trifluoromethyl)phenyl]-2,4-imidazolidinedione

¹H NMR (400 MHz, DMSO-d₆): δ 12.27, 8.33, 8.20, 7.92-7.90, 7.70, 7.54-7.49, 7.33-7.17, 6.57, 4.87, 4.72, 2.60, 2.19, 1.05;

LCMS: m/z 496 [M+H]+;

HPLC retention time: 1.815 min (method 1).

### Example 3(98): 3-{4-[(2-amino-4-pyrimidinyl)oxy]-3-ethyl-2-methylphenyl}-1-[3-(trifluoromethyl)phenyl]-2,4-imidazolidinedione

¹H NMR (400 MHz, DMSO-d₆): δ 8.18-8.13, 7.89, 7.70, 7.53, 7.25, 7.10, 6.72, 6.23, 4.85, 4.71, 2.59, 2.17, 1.04;

LCMS: m/z 472 [M+H]+;

HPLC retention time: 1.518 min (method 1).

### Example 3(99): 3-{4-[(2-amino-4-pyrimidinyl)oxy]-2-methylphenyl}-1-[3-(difluoromethyl)phenyl]-2,4-imidazolidinedione

¹H NMR (400 MHz, DMSO-d₆): δ 8.15, 8.00, 7.78, 7.58, 7.38-7.36, 7.22-6.93, 6.73, 6.22, 4.80, 4.66, 2.20;

LCMS: m/z 426 [M+H]+;

HPLC retention time: 1.493 min (method 38).

### Example 3(100): 3-{4-[(2-amino-4-pyrimidinyl)oxy]-2-methylphenyl}-1-[4-fluoro-3-(trifluoromethyl)phenyl]-2,4-imidazolidinedione

¹H NMR (400 MHz, DMSO-d₆): δ 8.16, 7.96, 7.64, 7.38, 7.31-7.15, 6.81, 6.24, 4.84, 4.70, 2.19;

LCMS: m/z 462 [M+H]+;

HPLC retention time: 1.253 min (method 5).

### Example 3(101): 3-{4-[(2-amino-4-pyrimidinyl)oxy]-3-ethylphenyl}-1-[5-(1,1,1-trifluoro-2-methyl-2-propanyl)-1,2-oxazol-3-yl]-2,4-imidazolidinedione

¹H NMR (400 MHz, DMSO-d₆): δ 8.14, 7.36, 7.30-7.24, 7.03, 6.73, 6.22, 4.51, 2.54, 1.58, 1.11;

LCMS: m/z 491 [M+H]+;

HPLC retention time: 1.513 min (method 1).

### Example 3(102): 3-{4-[(2-amino-4-pyrimidinyl)oxy]-2-ethylphenyl}-1-[3-(difluoromethoxy)phenyl]-2,4-imidazolidinedione

¹H NMR (400 MHz, DMSO-d₆): δ 8.16, 7.62, 7.52-7.46, 7.38, 7.27-7.23, 7.18-7.15, 7.09-6.98, 6.75, 6.23, 4.80, 4.64, 2.55, 1.11;

LCMS: m/z 456 [M+H]+;

HPLC retention time: 1.214 min (method 5).

### Example 3(103): 3-{4-[(2-amino-4-pyrimidinyl)oxy]-2-ethylphenyl}-1-[3-(difluoromethyl)phenyl]-2,4-imidazolidinedione

¹H NMR (400 MHz, DMSO-d₆): δ 8.16, 8.00, 7.79-7.77, 7.60, 7.38-7.36, 7.23-7.22, 7.18-6.94, 6.75, 6.22, 4.85, 4.67, 2.55, 1.12;

LCMS: m/z 440 [M+H]+;

HPLC retention time: 0.994 min (method 36).

### Example 3(104): 3-{4-[(2-amino-4-pyrimidinyl)oxy]-2-ethylphenyl}-1-[4-fluoro-3-(trifluoromethoxy)phenyl]-2,4-imidazolidinedione

¹H NMR (400 MHz, DMSO-d₆): δ 8.16, 8.03-8.02, 7.68-7.60, 7.37, 7.23, 7.17, 6.80, 6.24, 4.82, 4.65, 2.53, 1.11;

LCMS: m/z 492 [M+H]+;

HPLC retention time: 1.526 min (method 1).

### Example 3(105): 3-(3-{4-[(2-amino-4-pyrimidinyl)oxy]-3-ethylphenyl}-2,4-dioxo-1-imidazolidinyl)benzonitrile

¹H NMR (300 MHz, DMSO-d₆): δ 8.15-8.08, 7.69-7.62, 7.39, 7.33-7.24, 6.72, 6.23, 4.67, 2.55, 1.12;

LCMS: m/z 415 [M+H]+;

HPLC retention time: 1.262 min (method 1).

### Example 3(106): 3-{3-[3-ethyl-4-(7H-pyrrolo[2,3-d]pyrimidin-4-yloxy)phenyl]-2,4-dioxo-1-imidazolidinyl}benzonitrile

¹H NMR (400 MHz, DMSO-d₆): δ 12.26, 8.31, 8.13, 7.68-7.62, 7.51, 7.42, 7.34, 6.57, 4.69, 2.57, 1.10;

LCMS: m/z 439 [M+H]+;

HPLC retention time: 1.600 min (method 1).

### Example 3(107): 5-{3-[2-ethyl-4-(7H-pyrrolo[2,3-d]pyrimidin-4-yloxy)phenyl]-2,4-dioxo-1-imidazolidinyl}nicotinonitrile

¹H NMR (400 MHz, DMSO-d₆): δ 12.26, 9.30, 8.83, 8.54, 8.36, 7.52-7.25, 6.56, 4.87, 4.73, 2.58, 1.12;

LCMS: m/z 440 [M+H]+;

HPLC retention time: 1.463 min (method 1).

### Example 3(108): 3-[4-(3-pyridinyloxy)phenyl]-1-[3-(trifluoromethyl)phenyl]-2,4-imidazolidinedione trifluoroacetate (1:1)

¹H NMR (400 MHz, DMSO-d₆): δ 8.54, 8.48, 8.17, 7.89, 7.68, 7.58, 7.56-7.46, 7.23, 4.69;

LCMS: m/z 414 [M+H]+;

HPLC retention time: 1.347 min (method 51).

### Example 3(109): 3-[4-(5-pyrimidinyloxy)phenyl]-1-[3-(trifluoromethyl)phenyl]-2,4-imidazolidinedione

¹H NMR (400 MHz, DMSO-d₆): δ 9.06, 8.73, 8.17, 7.89, 7.68, 7.53-7.47, 7.31-7.27, 4.69;

LCMS: m/z 415 [M+H]+;

HPLC retention time: 1.346 min (method 51).

### Example 3(110): 3-{4-[(2-amino-4-pyrimidinyl)oxy]-2-methylphenyl}-1-[4-fluoro-3-(trifluoromethoxy)phenyl]-2,4-imidazolidinedione

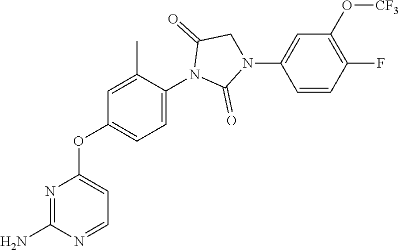 [SMILES: Cc1cc(Oc2ccnc(N)n2)ccc1N1C(=O)CN(c2ccc(F)c(OC(F)(F)F)c2)C1=O]

¹H NMR (400 MHz, DMSO-d₆): δ 8.15, 8.04-8.03, 7.68-7.60, 7.37, 7.22-7.13, 6.73, 6.22, 4.79, 4.65, 2.19;

LCMS: m/z 478 [M+H]+;

HPLC retention time: 1.453 min (method 1).

### Example 3(111): 3-{4-[(2-amino-4-pyrimidinyl)oxy]-2-methylphenyl}-1-[3-fluoro-5-(trifluoromethyl)phenyl]-2,4-imidazolidinedione

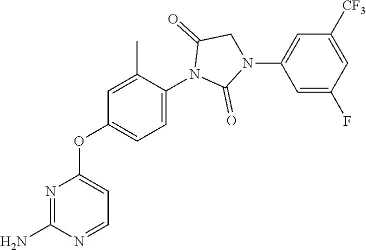 [SMILES: Cc1cc(Oc2ccnc(N)n2)ccc1N1C(=O)CN(c2cc(F)cc(C(F)(F)F)c2)C1=O]

¹H NMR (300 MHz, DMSO-d₆): δ 8.16, 8.03, 7.84, 7.52-7.49, 7.38, 7.24, 7.16, 6.76, 6.22, 4.83, 4.70, 2.19;

LCMS: m/z 462 [M+H]+;

HPLC retention time: 1.455 min (method 1).

### Example 3(112): 3-{4-[(2-amino-4-pyrimidinyl)oxy]-2-methylphenyl}-1-[5-(difluoromethoxy)-3-pyridinyl]-2,4-imidazolidinedione

¹H NMR (400 MHz, DMSO-d₆): δ 8.79, 8.32, 8.15, 8.08, 7.55-7.22, 7.19-7.13, 6.73, 6.21, 4.82, 4.70, 2.19;

LCMS: m/z 443 [M+H]+;

HPLC retention time: 1.330 min (method 55).

### Example 3(113): 3-(3-{4-[(2-amino-4-pyrimidinyl)oxy]-2-ethylphenyl}-2,4-dioxo-1-imidazolidinyl)benzonitrile

¹H NMR (400 MHz, DMSO-d₆): δ 8.15, 8.10, 7.68-7.62, 7.38, 7.23, 7.17, 6.74, 6.22, 4.82, 4.67, 2.54, 1.12;

LCMS: m/z 415 [M+H]+;

HPLC retention time: 2.409 min (method 16-1).

### Example 3(114): 3-{3-[2-ethyl-4-(7H-pyrrolo[2,3-d]pyrimidin-4-yloxy)phenyl]-2,4-dioxo-1-imidazolidinyl}benzonitrile

¹H NMR (300 MHz, DMSO-d₆): δ 12.27, 8.36, 8.14-8.11, 7.70-7.63, 7.52, 7.43-7.24, 6.57, 4.85, 4.70, 2.58, 1.13;

LCMS: m/z 439 [M+H]+;

HPLC retention time: 1.376 min (method 5).

### Example 3(115): 3-{4-[(6-methoxy-3-pyridinyl)oxy]phenyl}-1-[5-(trifluoromethyl)-3-pyridinyl]-2,4-imidazolidinedione

¹H NMR (300 MHz, DMSO-d₆): δ 9.18, 8.78, 8.53-8.52, 8.10, 7.61, 7.44-7.39, 7.12-7.07, 6.93, 4.76, 3.87;

LCMS: m/z 445 [M+H]+;

HPLC retention time: 1.573 min (method 5).

### Example 3(116): 3-{4-[(2-amino-4-pyrimidinyl)oxy]-2-(difluoromethoxy)phenyl}-1-[5-(trifluoromethyl)-3-pyridinyl]-2,4-imidazolidinedione

¹H NMR (400 MHz, DMSO-d₆): δ 9.18, 8.80, 8.50, 8.19, 7.59, 7.45-7.05, 6.79, 6.28, 5.02, 4.03;

LCMS: m/z 497 [M+H]+;

HPLC retention time: 1.483 min (method 38).

### Example 3(117): 3-{4-[(2-amino-4-pyrimidinyl)oxy]-2-(trifluoromethoxy)phenyl}-1-[5-(trifluoromethyl)-3-pyridinyl]-2,4-imidazolidinedione

¹H NMR (300 MHz, DMSO-d₆): δ 9.19, 8.81, 8.49, 8.22, 7.69, 7.55, 7.47, 6.91, 6.33-6.31, 5.05, 4.84;

LCMS: m/z 515 [M+H]+;

HPLC retention time: 1.475 min (method 55).

### Example 3(118): 3-[4-(pyrazolo[1,5-a]pyrimidin-6-yloxy)phenyl]-1-[5-(trifluoromethyl)-3-pyridinyl]-2,4-imidazolidinedione

¹H NMR (300 MHz, DMSO-d₆): δ 9.33, 9.17, 8.77, 8.63, 8.52, 8.25, 7.46-7.42, 7.29-7.25, 6.83, 4.75;

LCMS: m/z 455 [M+H]+;

HPLC retention time: 1.393 min (method 5).

### Example 3(119): 3-{4-[(2-amino-4-pyrimidinyl)oxy]-2-bromophenyl}-1-[5-(trifluoromethyl)-3-pyridinyl]-2,4-imidazolidinedione

¹H NMR (400 MHz, DMSO-d₆): δ 9.19, 8.80, 8.50, 8.18, 7.76, 7.63, 7.42, 6.80, 6.28, 5.03, 4.85;

LCMS: m/z 509 [M+H]+;

HPLC retention time: 1.297 min (method 1).

### Example 3(120): 3-{4-[(2-amino-4-pyrimidinyl)oxy]-2-(1-propyn-1-yl)phenyl}-1-[5-(trifluoromethyl)-3-pyridinyl]-2,4-imidazolidinedione

¹H NMR (400 MHz, DMSO-d₆): δ 9.21, 8.81, 8.50, 8.26, 7.84-7.39, 6.50, 5.03, 4.85, 2.01;

LCMS: m/z 469 [M+H]+;

HPLC retention time: 1.258 min (method 44).

### Example 3(121): 3-{4-[(2-amino-4-pyrimidinyl)oxy]-2-(3-methyl-1-butyn-1-yl)phenyl}-1-[5-(trifluoromethyl)-3-pyridinyl]-2,4-imidazolidinedione

¹H NMR (400 MHz, DMSO-d₆): δ 9.21, 8.80, 8.52, 8.17, 7.50, 7.37-7.33, 6.77, 6.26, 4.98, 4.89, 2.75, 1.07;

LCMS: m/z 497 [M+H]+;

HPLC retention time: 1.349 min (method 44).

### Example 3(122): 3-{5-[(2-amino-4-pyrimidinyl)oxy]-2-biphenylyl}-1-[5-(trifluoromethyl)-3-pyridinyl]-2,4-imidazolidinedione

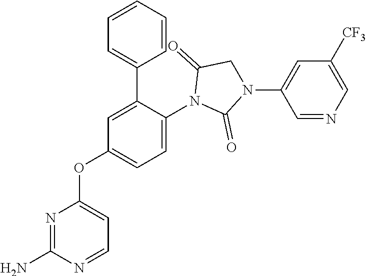 [SMILES: Nc1nccc(Oc2ccc(N3C(=O)CN(c4cncc(C(F)(F)F)c4)C3=O)c(-c3ccccc3)c2)n1]

¹H NMR (400 MHz, DMSO-d₆): δ 9.07, 8.76, 8.37, 8.18, 7.58, 7.44-7.29, 6.79, 6.28, 4.71;

LCMS: m/z 507 [M+H]+;

HPLC retention time: 1.403 min (method 1).

### Example 3(123): 3-{4-[(2-amino-4-pyrimidinyl)oxy]-2-methylphenyl}-1-[3-(trifluoromethyl)phenyl]-2,4-imidazolidinedione

¹H NMR (300 MHz, DMSO-d₆): δ 8.17-8.14, 7.90, 7.70, 7.54, 7.39, 7.24, 7.16, 6.77, 6.23, 4.85, 4.70, 2.20;

LCMS: m/z 444 [M+H]+;

HPLC retention time: 1.394 min (method 1).

### Example 3(124): 3-{4-[(2-amino-4-pyrimidinyl)oxy]-2-methylphenyl}-1-{4-[(4-methyl-1-piperazinyl)methyl]-3-(trifluoromethyl)phenyl}-2,4-imidazolidinedione

¹H NMR (400 MHz, DMSO-d₆): δ 8.15, 7.83-7.76, 7.37, 7.22, 7.15, 6.73, 4.83, 4.68, 3.59, 2.49-2.20, 2.09;

LCMS: m/z 556 [M+H]+;

HPLC retention time: 0.882 min (method 5).

### Example 3(125): 3-{4-[(2-amino-4-pyrimidinyl)oxy]-2-methylphenyl}-1-[4-(1-piperazinylmethyl)-3-(trifluoromethyl)phenyl]-2,4-imidazolidinedione

¹H NMR (400 MHz, DMSO-d₆): δ 8.15, 7.84-7.78, 7.37, 7.22, 7.15, 6.73, 6.21, 4.83, 4.68, 3.62, 2.89, 2.50-2.32, 2.15;

LCMS: m/z 542 [M+H]+;

HPLC retention time: 1.451 min (method 17).

### Example 3(126): 3-{4-[(2-amino-4-pyrimidinyl)oxy]-2-methylphenyl}-1-[4-(4-morpholinylmethyl)-3-(trifluoromethyl)phenyl]-2,4-imidazolidinedione

¹H NMR (400 MHz, DMSO-d₆): δ 8.15, 7.84-7.79, 7.37, 7.22, 7.15, 6.73, 6.21, 4.83, 4.68, 3.59, 2.89, 2.50-2.32, 2.18;

LCMS: m/z 543 [M+H]+;

HPLC retention time: 0.840 min (method 5).

### Example 3(127): 3-{4-[(2-amino-5-isopropyl-4-pyrimidinyl)oxy]-2-methylphenyl}-1-[3-(trifluoromethyl)phenyl]-2,4-imidazolidinedione trifluoroacetate (1:1)

¹H NMR (400 MHz, DMSO-d₆): δ 8.17, 8.12, 7.91-7.67, 7.54, 7.44, 7.32, 7.25, 4.85, 4.72, 3.11-3.04, 2.22, 1.29;

LCMS: m/z 486 [M+H]+;

HPLC retention time: 1.625 min (method 38).

### Example 3(128): 4-amino-6-(4-{2,5-dioxo-3-[3-(trifluoromethyl)phenyl]-1-imidazolidinyl}-3-methylphenoxy)-5-pyrimidinecarbonitrile

¹H NMR (400 MHz, DMSO-d₆): δ 8.22-7.89, 7.69, 7.53, 7.40, 7.30, 7.22, 4.85, 4.71, 2.20;

LCMS: m/z 469 [M+H]+;

HPLC retention time: 1.510 min (method 57-1).

### Example 3(129): 3-(4-{[6-amino-5-(trifluoromethyl)-4-pyrimidinyl]oxy}-2-methylphenyl-1-[3-(trifluoromethyl)phenyl]-2,4-imidazolidinedione

¹H NMR (400 MHz, DMSO-d₆): δ 8.18, 7.91, 7.70, 7.53, 7.38, 7.21, 7.14, 4.85, 4.71, 2.19;

LCMS: m/z 512 [M+H]+;

HPLC retention time: 1.911 min (method 44).

### Example 3(130): 3-{4-[(6-amino-5-isopropyl-4-pyrimidinyl)oxy]-2-methylphenyl}-1-[3-(trifluoromethyl)phenyl]-2,4-imidazolidinedione trifluoroacetate (1:1)

¹H NMR (400 MHz, DMSO-d₆): δ 8.16, 8.03, 7.91, 7.69, 7.53, 7.35, 7.21-6.99, 4.85, 4.71, 3.16, 2.19, 1.31;

LCMS: m/z 486 [M+H]+;

HPLC retention time: 1.415 min (method 5).

### Example 3(131): 3-{4-[(2-amino-4-pyrimidinyl)oxy]-2-ethylphenyl}-1-[3-fluoro-5-(trifluoromethyl)phenyl]-2,4-imidazolidinedione

¹H NMR (300 MHz, DMSO-d₆): δ 8.16, 8.02, 7.83, 7.51, 7.39, 7.24, 7.18, 6.76, 6.22, 4.86, 4.70, 2.55, 1.11;

LCMS: m/z 476 [M+H]+;

HPLC retention time: 1.528 min (method 1).

### Example 3(132): 3-{4-[(2-amino-4-pyrimidinyl)oxy]-2-ethylphenyl}-1-[4-fluoro-3-(trifluoromethyl)phenyl]-2,4-imidazolidinedione

¹H NMR (400 MHz, DMSO-d₆): δ 8.16, 7.96, 7.64, 7.37, 7.23-7.15, 6.74, 6.22, 4.87, 4.70, 2.54, 1.11;

LCMS: m/z 476 [M+H]+;

HPLC retention time: 1.293 min (method 5).

### Example 3(133): 3-{4-[(2-amino-4-pyrimidinyl)oxy]-2-ethylphenyl}-1-[5-(difluoromethoxy)-3-pyridinyl]-2,4-imidazolidinedione

¹H NMR (400 MHz, DMSO-d₆): δ 8.79, 8.33, 8.16, 8.08, 7.56-7.37, 7.24-7.16, 6.79, 6.24, 4.86, 4.70, 2.55, 1.12;

LCMS: m/z 457 [M+H]+;

HPLC retention time: 1.147 min (method 44).

### Example 3(134): 3-{4-[(2-amino-4-pyrimidinyl)oxy]-2-methylphenyl}-1-[5-(difluoromethyl)-3-pyridinyl]-2,4-imidazolidinedione

¹H NMR (400 MHz, DMSO-d₆): δ 9.03, 8.60, 8.40, 8.23, 7.53, 7.43, 7.30, 7.23-7.21, 6.46, 4.87, 4.75, 2.22;

LCMS: m/z 427 [M+H]+;

HPLC retention time: 1.898 min (method 63).

### Example 3(135): 3-{4-[(2-amino-4-pyrimidinyl)oxy]-2-ethylphenyl}-1-[5-(difluoromethyl)-3-pyridinyl]-2,4-imidazolidinedione

¹H NMR (300 MHz, DMSO-d₆): δ 9.02, 8.60, 8.40, 8.17, 7.41-7.37, 7.24-7.23, 7.19-7.16, 6.76, 6.23, 4.90, 4.74, 2.54, 1.12;

LCMS: m/z 463 [M+Na]+;

HPLC retention time: 1.216 min (method 38).

### Example 3(136): 3-{4-[(2-amino-4-pyrimidinyl)oxy]-2-isopropylphenyl}-1-[5-(trifluoromethyl)-3-pyridinyl]-2,4-imidazolidinedione

¹H NMR (400 MHz, DMSO-d₆): δ 9.17, 8.79, 8.50, 8.16, 7.37, 7.29, 7.16, 6.75, 6.21, 4.93, 4.76, 2.95, 1.13;

LCMS: m/z 473 [M+H]+;

HPLC retention time: 1.542 min (method 55).

### Example 3(137): 3-{4-[(6-amino-4-pyrimidinyl)oxy]-2-isopropylphenyl}-1-[5-(trifluoromethyl)-3-pyridinyl]-2,4-imidazolidinedione trifluoroacetate (1:1)

¹H NMR (400 MHz, DMSO-d₆): δ 9.18, 8.78, 8.50, 8.32, 7.65-7.53, 7.40, 7.33, 7.18, 5.98, 4.93, 4.78, 2.95, 1.14;

LCMS: m/z 473 [M+1H]+

HPLC retention time: 1.628 min (method 55).

### Example 3(138): 3-{4-[(2-amino-4-pyrimidinyl)oxy]-2-isopropylphenyl}-1-[5-(difluoromethyl)-3-pyridinyl]-2,4-imidazolidinedione trifluoroacetate (1:1)

¹H NMR (300 MHz, DMSO-d₆): δ 9.01, 8.60, 8.40, 8.20, 7.41-7.37, 7.32-7.31, 7.23-7.04, 6.33, 4.90, 4.73, 2.95, 1.14;

LCMS: m/z 477 [M+Na]+;

HPLC retention time: 1.272 min (method 38).

### Example 3(139): 3-{4-[(2-amino-4-pyrimidinyl)oxy]-2-isopropylphenyl}-1-[3-(difluoromethyl)phenyl]-2,4-imidazolidinedione

¹H NMR (300 MHz, DMSO-d₆): δ 8.16, 7.99, 7.76, 7.59, 7.37-7.34, 7.27-7.07, 6.88-6.74, 6.20, 4.86, 4.65, 2.93, 1.13;

LCMS: m/z 454 [M+H]+;

HPLC retention time: 1.506 min (method 55).

### Example 3(140): 3-{4-[(2-amino-4-pyrimidinyl)oxy]-2-isopropylphenyl}-1-[3-(difluoromethoxy)phenyl]-2,4-imidazolidinedione

¹H NMR (400 MHz, DMSO-d₆): δ 8.16, 7.62, 7.52-7.46, 7.36, 7.28, 7.16, 6.99, 6.76, 6.22, 4.82, 4.63, 2.93, 1.18-1.10;

LCMS: m/z 470 [M+H]+;

HPLC retention time: 2.458 min (method 63).

### Example 3(141): 3-{4-[(2-amino-4-pyrimidinyl)oxy]-2-isopropylphenyl}-1-[5-(difluoromethoxy)-3-pyridinyl]-2,4-imidazolidinedione

¹H NMR (400 MHz, DMSO-d₆): δ 8.79, 8.33, 8.16, 8.08, 7.37, 7.28, 7.19-7.15, 6.74, 6.22, 4.88, 4.69, 2.94, 1.16;

LCMS: m/z 471 [M+H]+;

HPLC retention time: 1.347 min (method 55).

### Example 3(142): 3-{4-[(2-amino-4-pyrimidinyl)oxy]-2-isopropylphenyl}-1-[4-fluoro-3-(trifluoromethyl)phenyl]-2,4-imidazolidinedione

¹H NMR (400 MHz, DMSO-d₆): δ 8.15, 7.95, 7.64, 7.35, 7.27, 7.17, 6.75, 6.22, 4.88, 4.69, 2.93, 1.14;

LCMS: m/z 490 [M+H]+;

HPLC retention time: 1.512 min (method 44).

### Example 3(143): 3-{4-[(2-amino-4-pyrimidinyl)oxy]-2-isopropylphenyl}-1-[3-fluoro-5-(trifluoromethyl)phenyl]-2,4-imidazolidinedione

¹H NMR (300 MHz, DMSO-d₆): δ 8.22, 8.02, 7.83, 7.51, 7.40, 7.34, 7.22, 6.41, 4.87, 4.69, 2.95, 1.14-1.12;

LCMS: m/z 490 [M+H]+;

HPLC retention time: 1.659 min (method 55).

### Example 3(144): 3-{4-[(2-amino-4-pyrimidinyl)oxy]-2-isopropylphenyl}-1-[4-fluoro-3-(trifluoromethoxy)phenyl]-2,4-imidazolidinedione

¹H NMR (400 MHz, DMSO-d₆): δ 8.16, 8.02-8.01, 7.67-7.60, 7.36, 7.27, 7.16, 6.74, 6.22, 4.83, 4.64, 2.93, 1.14;

LCMS: m/z 506 [M+H]+;

HPLC retention time: 1.477 min (method 2).

### Example 3(145): 3-{4-[(6-amino-4-pyrimidinyl)oxy]-2-ethylphenyl}-1-[4-fluoro-3-(trifluoromethyl)phenyl]-2,4-imidazolidinedione

¹H NMR (400 MHz, DMSO-d₆): δ 8.16-8.10, 7.96, 7.63, 7.36, 7.20, 7.12, 6.94, 5.84, 4.87, 4.69, 2.50, 1.10;

LCMS: m/z 476 [M+H]+;

HPLC retention time: 1.266 min (method 65).

### Example 3(146): 3-{4-[(2-amino-4-pyrimidinyl)oxy]-3-isopropylphenyl}-1-[3-(difluoromethoxy)phenyl]-2,4-imidazolidinedione

¹H NMR (300 MHz, DMSO-d₆): δ 8.13, 7.62, 7.53-7.50, 7.47-7.42, 7.30-6.97, 6.71, 6.22, 4.63, 3.01, 1.15;

LCMS: m/z 470 [M+H]+;

HPLC retention time: 1.327 min (method 44).

### Example 3(147): 3-(3-{4-[(2-amino-4-pyridinyl)oxy]phenyl}-2,4-dioxo-1-imidazolidinyl)benzonitrile

¹H NMR (400 MHz, DMSO-d₆): δ 8.17-8.10, 7.85, 7.68-7.62, 7.54-7.47, 7.30-7.26, 6.21-6.11, 6.03, 5.91, 4.67;

LCMS: m/z 386 [M+H]+;

HPLC retention time: 2.534 min (method 66).

### Example 3(148): 3-[2-methoxy-4-(4-pyridinyloxy)phenyl]-1-[3-(trifluoromethyl)phenyl]-2,4-imidazolidinedione

¹H NMR (400 MHz, DMSO-d₆): δ 8.59-8.58, 8.15, 7.89, 7.70, 7.53, 7.46, 7.14-7.13, 6.91, 4.92, 4.73, 3.79;

LCMS: m/z 444 [M+H]+;

HPLC retention time: 1.181 min (method 5).

### Example 3(149): 3-{3-isopropyl-4-[(2-{[2-(methylsulfonyl)ethyl]amino}-4-pyrimidinyl)oxy]phenyl}-1-[5-(trifluoromethyl)-3-pyridinyl]-2,4-imidazolidinedione

¹H NMR (300 MHz, DMSO-d₆): δ 9.18, 8.78, 8.52, 8.25, 7.45-7.43, 7.33-7.25, 6.32, 4.77, 3.58-3.45, 3.45-3.19, 3.04-2.72, 1.15;

LCMS: m/z 579 [M+H]+;

HPLC retention time: 1.620 min (method 67).

### Example 3(150):3-{4-[(6-amino-1H-pyrrolo[2,3-b]pyridin-4-yl)oxy]phenyl}-1-[3-(trifluoromethyl)phenyl]-2,4-imidazolidinedione

¹H NMR (300 MHz, DMSO-d₆): δ 4.45-4.78, 5.59-5.68, 5.68-5.74, 5.92-6.07, 6.77-6.90, 7.16-7.33, 7.39-7.55, 7.58-7.72, 7.78-7.93, 8.08-8.29, 10.79-10.99;

TLC Rf: 0.45 (AcOEt).

### Example 3(151): 3-{4-[(2-{[2-(methylsulfonyl)ethyl]amino}-4-pyrimidinyl)oxy]phenyl}-1-[3-(trifluoromethyl)phenyl]-2,4-imidazolidinedione

¹H NMR (400 MHz, DMSO-d₆): δ 8.26, 8.18, 7.90, 7.69, 7.54-7.51, 7.48-7.36, 6.31, 4.71, 3.59, 3.31-3.29, 2.95;

LCMS: m/z 536 [M+H]+;

HPLC retention time: 1.643 min (method 38).

### Example 3(152): 3-[3-fluoro-4-(imidazo[1,2-b]pyridazin-6-yloxy)phenyl]-1-(3-isopropoxyphenyl)-2,4-imidazolidinedione

¹H NMR (400 MHz, DMSO-d₆): δ 8.26, 8.17, 7.69-7.65, 7.59-7.56, 7.42-7.39, 7.34-7.29, 7.26-7.21, 6.75, 4.65-4.62, 1.28;

LCMS: m/z 462 [M+H]+;

HPLC retention time: 1.288 min (method 5).

### Example 3(153): 3-[4-(1H-pyrrolo[2,3-b]pyridin-4-yloxy)phenyl]-1-[3-(trifluoromethoxy)phenyl]-2,4-imidazolidinedione

¹H NMR (400 MHz, DMSO-d₆): δ 11.81, 8.16, 7.91, 7.65-7.55, 7.51, 7.41, 7.33, 7.18, 6.57, 6.22, 4.67;

MS: m/z 469 (M+H)+;

HPLC retention time: 3.20 min (method 76).

### Example 3(154): 1-[4-bromo-3-(trifluoromethyl)phenyl]-3-[4-(1H-pyrrolo[2,3-b]pyridin-4-yloxy)phenyl]-2,4-imidazolidinedione

¹H NMR (400 MHz, DMSO-d₆): δ 11.81, 8.30, 8.16, 7.97, 7.82, 7.51, 7.41, 7.33, 6.57, 6.21, 4.72;

MS: m/z 531, 533 (M+H)+;

HPLC retention time: 3.26 min (method 76).

### Example 3(155): 1-(3-cyclopropylphenyl)-3-[4-(1H-pyrrolo[2,3-b]pyridin-4-yloxy)phenyl]-2,4-imidazolidinedione

¹H NMR (400 MHz, DMSO-d₆): δ 11.82, 8.16, 7.53-7.47, 7.42-7.39, 7.34-7.27, 6.90, 6.56, 6.23, 4.64, 2.00-1.92, 1.02-0.96, 0.74-0.69;

MS: m/z 425 (M+H)+;

HPLC retention time: 3.14 min (method 76).

### Example 3(156): 1-{4-[(4-methyl-1-piperazinyl)methyl]-3-(trifluoromethyl)phenyl}-3-[4-(1H-pyrrolo[2,3-b]pyridin-4-yloxy)phenyl]-2,4-imidazolidinedione

¹H NMR (400 MHz, DMSO-d₆): δ 11.81, 8.20, 8.15, 7.84-7.78, 7.51, 7.40, 7.33, 6.58, 6.24-6.21, 4.70, 3.61, 2.41, 2.20;

MS: m/z 565 (M+H)+;

HPLC retention time: 2.56 min (method 76).

### Example 3(157): 1-{4-[(1-methyl-4-piperidinyl)oxy]-3-(trifluoromethyl)phenyl}-3-[4-(1H-pyrrolo[2,3-b]pyridin-4-yloxy)phenyl]-2,4-imidazolidinedione

¹H NMR (400 MHz, DMSO-d₆): δ 11.81, 8.16, 8.03, 7.79, 7.53-7.50, 7.43-7.39, 7.34-7.31, 6.56, 6.22, 4.67, 4.64, 2.28-2.23, 2.18, 1.96-1.89, 1.77-1.65, 2H obscured by water peak;

MS: m/z 566 (M+H)+;

HPLC retention time: 2.56 min (method 76).

### Example 3(158): 3-[3-(1-hydroxyethyl)-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.79, 9.19-9.17, 8.79-8.77, 8.56-8.54, 8.12, 7.74-7.73, 7.40-7.33, 7.17, 6.45, 6.23-6.21, 5.38, 5.01-4.94, 4.77, 1.32;

MS: m/z 498 (M+H)+;

HPLC retention time: 3.88 min (method 79).

### Example 3(159): 1-[4-(4-morpholinylmethyl)-3-(trifluoromethyl)phenyl]-3-[4-(1H-pyrrolo[2,3-b]pyridin-4-yloxy)phenyl]-2,4-imidazolidinedione

¹H NMR (400 MHz, DMSO-d₆): δ 11.81, 8.16, 8.03, 7.79, 7.53-7.50, 7.43-7.39, 7.34-7.31, 6.56, 6.22, 4.67, 4.64, 2.28-2.23, 2.18, 1.96-1.89, 1.77-1.65, 2H obscured by water peak;

MS: m/z 552 (M+H)+;

HPLC retention time: 2.53 min (method 76).

### Example 3(160): 3-[3-methyl-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.78, 8.22, 8.11, 7.91, 7.70, 7.54, 7.47, 7.40-7.33, 7.25, 6.37, 6.23, 4.73, 2.23;

MS: m/z 467 (M+H)+;

HPLC retention time: 3.17 min (method 76).

### Example 3(161): 3-[3-fluoro-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.89, 8.24, 8.18, 7.94, 7.76-7.71, 7.64, 7.60-7.54, 7.47-7.43, 6.53, 6.30-6.27, 4.77;

MS: m/z 471 (M+H)+;

HPLC retention time: 3.21 min (method 76).

### Example 3(162): 3-[3-methoxy-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.77, 8.27, 8.11, 7.96, 7.75, 7.59, 7.42-7.37, 7.16, 6.35-6.29, 4.79, 3.79;

MS: m/z 483 (M+H)+;

HPLC retention time: 3.06 min (method 76).

### Example 3(163): 3-[3-bromo-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.85, 8.21, 8.15, 7.94-7.88, 7.70, 7.56, 7.48-7.41, 6.45, 6.20, 4.73;

MS: m/z 531, 533 (M+H)+;

HPLC retention time: 3.30 min (method 76).

### Example 3(164): 5-{2,5-dioxo-3-[3-(trifluoromethyl)phenyl]-1-imidazolidinyl}-2-(1H-pyrrolo[2,3-b]pyridin-4-yloxy)benzonitrile

¹H NMR (400 MHz, DMSO-d₆): δ 11.99, 8.26, 8.20, 8.07, 7.91, 7.80, 7.71, 7.55, 7.49, 7.35, 6.81, 6.22, 4.75;

MS: m/z 478 (M+H)+;

HPLC retention time: 3.26 min (method 76).

### Example 3(165): 3-[3-ethyl-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.79, 8.22, 8.12, 7.93-7.90, 7.70, 7.54, 7.48, 7.40-7.33, 7.23, 6.40, 6.22, 4.73, 2.62, 1.16;

MS: m/z 481 (M+H)+;

HPLC retention time: 3.51 min (method 77).

### Example 3(166): 3-[3-isopropyl-4-(1H-pyrrolo[2,3-b]pyridin-4-yloxy)phenyl]-1-[3-(trifluoromethyl)phenyl]-2,4-imidazolidinedione trifluoroacetate (1:1)

¹H NMR (300 MHz, DMSO-d₆): δ 1.04-1.31, 3.06-3.23, 4.56-4.80, 6.12-6.24, 6.32-6.44, 7.13-7.25, 7.25-7.43, 7.43-7.61, 7.61-7.75, 7.80-7.95, 8.04-8.14, 8.15-8.28, 11.71-11.83;

MS: m/z 495 (M+H)+;

HPLC retention time: 0.96 min (method 83).

### Example 3(167): 3-[3-chloro-4-(1H-pyrrolo[2,3-b]pyridin-4-yloxy)phenyl]-1-[3-(trifluoromethyl)phenyl]-2,4-imidazolidinedione trifluoroacetate (1:1)

¹H NMR (400 MHz, DMSO-d₆) δ 4.58-4.89, 6.14-6.34, 6.41-6.53, 7.40-7.47, 7.47-7.60, 7.64-7.75, 7.75-7.84, 7.85-7.99, 8.10-8.25, 11.73-12.19;

MS: m/z 487 (M+H)+;

HPLC retention time: 0.90 min (method 83).

### Example 3(168): 3-[4-(1H-pyrrolo[2,3-b]pyridin-4-yloxy)phenyl]-1-[5-(trifluoromethyl)-3-pyridinyl]-2,4-imidazolidinedione trifluoroacetate (1:1)

¹H NMR (400 MHz, DMSO-d₆) δ 4.73-4.84, 6.16-6.34, 6.52-6.68, 7.31-7.41, 7.41-7.49, 7.49-7.63, 8.03-8.32, 8.46-8.63, 8.64-8.92, 9.02-9.36, 11.81-12.21;

MS: m/z 454 (M+H)+;

HPLC retention time: 0.76 min (method 83).

### Example 3(169): 3-[4-(1H-pyrrolo[2,3-b]pyridin-4-yloxy)phenyl]-1-[2-(trifluoromethyl)-1,3-thiazol-5-yl]-2,4-imidazolidinedione trifluoroacetate (1:1)

¹H NMR (400 MHz, DMSO-d₆) δ 4.72, 6.17-6.31, 6.55-6.71, 7.30-7.40, 7.40-7.47, 7.47-7.57, 7.90, 8.18, 11.63-12.21;

MS: m/z 460 (M+H)+;

HPLC retention time: 0.82 min (method 83).

### Example 3(170): 3-{4-[(3-fluoro-1H-pyrrolo[2,3-b]pyridin-4-yl)oxy]phenyl}-1-[3-(trifluoromethyl)phenyl]-2,4-imidazolidinedione

¹H NMR (400 MHz, DMSO-d₆): δ 11.68, 8.26-8.21, 7.97-7.93, 7.77-7.71, 7.60-7.56, 7.47-7.44, 7.42-7.38, 6.55, 4.77;

MS: m/z 471 (M+H)+;

HPLC retention time: 3.36 min (method 77).

### Example 3(171): 3-[3-(difluoromethyl)-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.93, 8.27-8.23, 7.98-7.94, 7.90-7.88, 7.78-7.72, 7.70-7.66, 7.59, 7.50-7.46, 7.38, 7.32, 6.71, 6.24-6.21, 4.76;

MS: m/z 503 (M+H)+;

HPLC retention time: 3.38 min (method 77).

### Example 3(172): 3-[3-(difluoromethyl)-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.93, 9.26-9.24, 8.86-8.84, 8.61-8.59, 8.25, 7.91-7.88, 7.71-7.67, 7.50-7.48, 7.39, 7.33, 6.72, 6.23, 4.85-4.83;

MS: m/z 504 (M+H)+;

HPLC retention time: 3.20 min (method 77).

### Example 3(173): 3-[3-(difluoromethyl)-4-(1H-pyrrolo[2,3-b]pyridin-4-yloxy)phenyl]-1-[3-(trifluoromethoxy)phenyl]-2,4-imidazolidinedione

¹H NMR (400 MHz, DMSO-d₆): δ 11.93, 8.25, 7.96, 7.88, 7.69-7.60, 7.50-7.47, 7.38, 7.31, 7.25-7.22, 6.71, 6.24-6.22, 4.71;

MS: m/z 519 (M+H)+;

HPLC retention time: 3.37 min (method 76).

### Example 3(174): 3-{4-[(3-fluoro-1H-pyrrolo[2,3-b]pyridin-4-yl)oxy]phenyl}-1-[5-(trifluoromethyl)-3-pyridinyl]-2,4-imidazolidinedione

¹H NMR (400 MHz, DMSO-d₆): δ 11.70, 9.25-9.23, 8.85-8.83, 8.60-8.59, 8.23, 7.60-7.56, 7.47-7.44, 7.42-7.39, 6.56, 4.83;

MS: m/z 472 (M+H)+;

HPLC retention time: 3.18 min (method 76).

### Example 3(175): 3-[3-cyclopropyl-4-(1H-pyrrolo[2,3-b]pyridin-4-yloxy)phenyl]-1-[3-(trifluoromethyl)phenyl]-2,4-imidazolidinedione trifluoroacetate (1:1)

¹H NMR (400 MHz, DMSO-d₆) δ 0.53-0.73, 0.73-1.01, 1.92-2.08, 4.57-4.82, 6.23-6.37, 6.38-6.51, 7.09-7.20, 7.22-7.38, 7.39-7.47, 7.47-7.59, 7.63-7.75, 7.83-7.96, 8.09-8.26, 11.58-12.22;

MS: m/z 493 (M+H)+.

HPLC retention time: 0.92 min (method 76).

### Example 3(176): 3-[3-ethyl-4-(1H-pyrrolo[2,3-b]pyridin-4-yloxy)phenyl]-1-[5-(trifluoromethyl)-3-pyridinyl]-2,4-imidazolidinedione

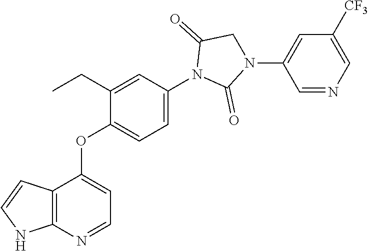 [SMILES: CCc1cc(N2C(=O)CN(c3cncc(C(F)(F)F)c3)C2=O)ccc1Oc1ccnc2[nH]ccc12]

1H NMR (400 MHz, DMSO-d₆): δ 11.76, 9.21-9.17, 8.80-8.77, 8.57-8.53, 8.13-8.09, 7.49-7.46, 7.40-7.32, 7.24-7.21, 6.40-6.38, 6.22-6.19, 4.78, 2.65-2.58, 1.19-1.12;

MS: m/z 482 (M+H)+;

HPLC retention time: 3.28 min (method 77).

### Example 3(177): 3-[3-(fluoromethyl)-4-(1H-pyrrolo[2,3-b]pyridin-4-yloxy)phenyl]-1-[3-(trifluoromethyl)phenyl]-2,4-imidazolidinedione

¹H NMR (400 MHz, DMSO-d₆): δ 11.88, 8.27-8.25, 8.21, 7.97-7.93, 7.77-7.72, 7.61-7.55, 7.47-7.45, 7.32-7.28, 6.60, 6.27-6.24, 5.60, 4.77;

MS: m/z 485 (M+H)+;

HPLC retention time: 3.34 min (method 77).

### Example 3(178): 3-[3-(fluoromethyl)-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.82, 9.20-9.16, 8.80-8.77, 8.56-8.52, 8.15, 7.71-7.68, 7.54-7.49, 7.42-7.38, 7.24, 6.54, 6.21-6.17, 5.55, 4.78;

MS: m/z 486 (M+H)+;

HPLC retention time: 3.00 min (method 76).

### Example 3(179): 3-[3-Cyclopropyl-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.79, 9.20, 8.80, 8.57-8.55, 8.12, 7.40-7.38, 7.33-7.24, 7.14, 6.40, 6.27-6.24, 4.79, 2.05-1.97, 0.93-0.87, 0.70-0.64;

MS: m/z 494 (M+H)+;

HPLC retention time: 3.02 min (method 76).

### Example 3(180): 3-[3-isopropyl-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.79, 9.21-9.19, 8.80-8.80, 8.58-8.56, 8.13, 7.53, 7.40-7.38, 7.36-7.33, 7.23, 6.42, 6.22-6.20, 4.80, 3.25-3.16, 1.20;

MS: m/z 496 (M+H)+;

HPLC retention time: 3.12 min (method 76).

### Example 3(181): 3-{4-[(3-fluoro-1H-pyrrolo[2,3-b]pyridin-4-yl)oxy]-3-isopropylphenyl}-1-[3-(trifluoromethyl)phenyl]-2,4-imidazolidinedione

¹H NMR (400 MHz, DMSO-d₆): δ 11.61, 8.21-8.19, 8.12, 7.92-7.88, 7.69, 7.54-7.51, 7.40-7.38, 7.37-7.33, 7.27-7.24, 6.30, 4.71, 3.22-3.11, 1.20;

MS: m/z 513 (M+H)+;

HPLC retention time: 3.54 min (method 77).

### Example 3(182): 3-{4-[(3-fluoro-1H-pyrrolo[2,3-b]pyridin-4-yl)oxy]-3-isopropylphenyl}-1-[5-(trifluoromethyl)-3-pyridinyl]-2,4-imidazolidinedione

¹H NMR (400 MHz, DMSO-d₆): δ 11.61, 9.19, 8.79, 8.56-8.54, 8.12, 7.52, 7.40-7.34, 7.26, 6.31, 4.78, 3.22-3.12, 1.20;

MS: m/z 514 (M+H)+;

HPLC retention time: 3.39 min (method 76).

### Example 3(183): 3-[trans-4-(1H-pyrrolo[2,3-b]pyridin-4-yloxy)cyclohexyl]-1-[5-(trifluoromethyl)-3-pyridinyl]-2,4-imidazolidinedione trifluoroacetate (1:1)

¹H NMR (300 MHz, DMSO-d₆): δ 1.44-1.96, 2.11-2.45, 3.88-3.90, 3.92-4.15, 4.36-4.70, 6.15-6.47, 6.57-6.85, 7.07-7.44, 7.87-8.17, 8.33-8.57, 8.57-8.84, 8.93-9.21, 11.34-11.64;

MS: m/z 460 (M+H)+;

HPLC retention time: 0.80 min (method 83).

### Example 3(184): 3-[3-isopropyl-4-(1H-pyrazolo[3,4-b]pyridin-4-yloxy)phenyl]-1-[5-(trifluoromethyl)-3-pyridinyl]-2,4-imidazolidinedione

¹H NMR (400 MHz, DMSO-d₆): δ 13.80, 9.27-9.24, 8.85, 8.62, 8.44, 7.71, 7.65-7.62, 7.47-7.42, 6.53, 4.85, 3.21-3.13, 1.22;

MS: m/z 497 (M+H)+;

HPLC retention time: 3.28 min (method 76).

### Example 3(185): 3-[3-isopropyl-4-(1H-pyrazolo[3,4-b]pyridin-4-yloxy)phenyl]-1-[3-(trifluoromethyl)phenyl]-2,4-imidazolidinedione

¹H NMR (400 MHz, DMSO-d₆): δ 13.75, 8.39, 8.21, 7.91, 7.72-7.67, 7.65, 7.59-7.57, 7.54-7.52, 7.43-7.35, 6.47, 4.73, 3.15-3.07, 1.17-1.14;

MS: m/z 496 (M+H)+;

HPLC retention time: 3.46 min (method 77).

### Example 3(186): 3-{4-[(3-fluoro-1H-pyrrolo[2,3-b]pyridin-4-yl)oxy]phenyl}-1-[3-(trifluoromethoxy)phenyl]-2,4-imidazolidinedione

¹H NMR (400 MHz, DMSO-d₆): δ 11.62, 8.16, 7.91-7.88, 7.63-7.55, 7.54-7.48, 7.40, 7.36-7.31, 7.19-7.15, 6.49, 4.66;

MS: m/z 487 (M+H)+;

HPLC retention time: 3.42 min (method 77).

### Example 3(187): 3-[3-isopropyl-4-(1H-pyrazolo[3,4-b]pyridin-4-yloxy)phenyl]-1-[3-(trifluoromethoxy)phenyl]-2,4-imidazolidinedione

¹H NMR (400 MHz, DMSO-d₆): δ 13.76-13.72, 8.38, 7.91, 7.67-7.54, 7.40-7.34, 7.20-7.15, 6.47, 4.67, 3.18-3.07, 1.17-1.12;

MS: m/z 512 (M+H)+;

HPLC retention time: 3.51 min (method 77).

### Example 3(188): 3-[3-isopropyl-4-(1H-pyrrolo[2,3-b]pyridin-4-yloxy)phenyl]-1-[3-(trifluoromethoxy)phenyl]-2,4-imidazolidinedione

¹H NMR (400 MHz, DMSO-d₆): δ 11.77, 8.12-8.10, 7.90-7.89, 7.63-7.55, 7.51-7.49, 7.39-7.36, 7.34-7.30, 7.22-7.16, 6.41-6.38, 6.21-6.19, 4.66, 3.20-3.14, 1.18;

MS: m/z 511 (M+H)+;

HPLC retention time: 3.38 min (method 76).

### Example 3(189): 1-(5-cyclopropyl-3-pyridinyl)-3-[3-isopropyl-4-(1H-pyrrolo[2,3-b]pyridin-4-yloxy)phenyl]-2,4-imidazolidinedione

¹H NMR (400 MHz, DMSO-d₆): δ 11.77-11.76, 8.75-8.72, 8.23-8.20, 8.11, 7.71, 7.52-7.49, 7.39-7.36, 7.34-7.29, 7.22-7.18, 6.39, 6.22-6.18, 4.68, 3.20-3.14, 2.05-1.97, 1.18, 1.08-1.01, 0.81-0.76;

MS: m/z 468 (M+H)+;

HPLC retention time: 3.28 min (method 77).

### Example 3(190): 3-[2-ethyl-4-(1H-pyrrolo[2,3-b]pyridin-4-yloxy)phenyl]-1-[3-(trifluoromethyl)phenyl]-2,4-imidazolidinedione

¹H NMR (400 MHz, CDCl₃): δ 9.36, 8.22, 7.97, 7.86-7.82, 7.57, 7.49-7.45, 7.26-7.22, 7.21, 7.10, 6.64, 6.46-6.43, 4.64-4.52, 2.57, 1.21;

MS: m/z 481 (M+H)+;

HPLC retention time: 3.47 min (method 77).

### Example 3(191): 3-[2-methyl-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, DMSO-d₆): δ 11.81, 9.19, 8.80, 8.55, 8.17, 7.43-7.40, 7.24, 7.15, 6.58, 6.25-6.23, 4.91, 4.79, 2.22;

MS: m/z 468 (M+H)+;

HPLC retention time: 2.96 min (method 76).

### Example 3(192): 3-[2-ethyl-4-(1H-pyrrolo[2,3-b]pyridin-4-yloxy)phenyl]-1-[5-(trifluoromethyl)-3-pyridinyl]-2,4-imidazolidinedione

¹H NMR (400 MHz, CDCl₃): δ 9.06, 8.96, 8.74-8.72, 8.54-8.53, 8.22, 7.25-7.21, 7.11, 6.64, 6.45-6.43, 4.69-4.58, 2.56, 1.21;

MS: m/z 482 (M+H)+;

HPLC retention time: 3.03 min (method 76).

### Example 3(193): 3-{4-[(2-amino-4-pyridinyl)oxy]phenyl}-1-[3-(trifluoromethyl)phenyl]-2,4-imidazolidinedione

¹H NMR (300 MHz, CDCl₃): δ 4.39-4.50, 4.54, 5.95-6.07, 6.29-6.42, 7.14-7.29, 7.40-7.52, 7.52-7.66, 7.74-7.88, 7.90-8.07; TLC Rf: 0.20 (CHCl₃/MeOH=10/1).

### Example 3(194): 3-[4-(9H-purin-6-yloxy)phenyl]-1-[3-(trifluoromethyl)phenyl]-2,4-imidazolidinedione hydrochloride (1:1)

¹H NMR (300 MHz, DMSO-d₆): δ 4.65-4.80, 7.49, 7.61-7.76, 7.82-7.97, 8.12-8.24, 8.42-8.51, 8.53-8.63; TLC Rf: 0.06 (CHCl₃/MeOH=10/1).

### Example 3(195): 3-(4-{[6-(benzylamino)-4-pyrimidinyl]oxy}-3-ethylphenyl)-1-[5-(trifluoromethyl)-3-pyridinyl]-2,4-imidazolidinedione

¹H NMR (400 MHz, CD₃OD): δ 9.15, 8.68, 8.59, 8.32, 7.51, 7.43, 7.41-7.23, 5.78, 4.89-4.36, 2.65, 1.18;

LCMS: m/z 549 [M+H]+;

HPLC retention time: 1.811 min (method 1).

### Example 3(196): 3-{4-[(6-amino-4-pyrimidinyl)oxy]-3-isopropylphenyl}-1-[5-(trifluoromethyl)-3-pyridinyl]-2,4-imidazolidinedione trifluoroacetate (1:1)

¹H NMR (400 MHz, DMSO-d₆): δ 9.18, 8.78, 8.53, 8.12, 7.45, 7.31, 7.21, 7.06, 5.82, 4.77, 3.03, 1.16;

LCMS: m/z 495 [M+Na]+;

HPLC retention time: 1.210 min (method 5).

### Example 3(197): 3-{4-[(6-amino-4-pyrimidinyl)oxy]-3-isopropylphenyl}-1-[3-(trifluoromethyl)phenyl]-2,4-imidazolidinedione trifluoroacetate (1:1)

¹H NMR (400 MHz, DMSO-d₆): δ 8.18-8.17, 7.90, 7.68, 7.53-7.51, 7.46, 7.33-7.21, 5.85, 4.70, 3.02, 1.15;

LCMS: m/z 494 [M+Na]+;

HPLC retention time: 1.365 min (method 5).

### Example 3(198): 3-{4-[(6-amino-4-pyrimidinyl)oxy]-2-ethylphenyl}-1-[5-(trifluoromethyl)-3-pyridinyl]-2,4-imidazolidinedione

¹H NMR (400 MHz, DMSO-d₆): δ 9.19, 8.79, 8.52, 8.11, 7.37, 7.21-7.12, 6.93, 5.85, 4.92, 4.77, 2.51, 1.11;

LCMS: m/z 481 [M+Na]+;

HPLC retention time: 1.148 min (method 5).

### Example 3(199): 3-{4-[(6-amino-4-pyrimidinyl)oxy]-2-ethylphenyl}-1-[3-(trifluoromethyl)phenyl]-2,4-imidazolidinedione

¹H NMR (400 MHz, DMSO-d₆): δ 8.17-8.10, 7.90, 7.69-7.67, 7.53, 7.37, 7.20-7.11, 6.93, 5.84, 4.88, 4.71, 2.54, 1.11;

LCMS: m/z 480 [M+Na]+;

HPLC retention time: 1.308 min (method 5).

### Example 3(200): 3-{4-[(6-amino-4-pyrimidinyl)oxy]-3-ethyl-2-methylphenyl}-1-[5-(trifluoromethyl)-3-pyridinyl]-2,4-imidazolidinedione

¹H NMR (300 MHz, DMSO-d₆): δ 9.19, 8.79, 8.53, 8.09, 7.25, 7.07, 6.93, 5.80, 4.90, 4.77, 2.59, 2.17, 1.03;

LCMS: m/z 473 [M+H]+;

HPLC retention time: 1.470 min (method 1).

### Example 3(201): 3-{4-[(6-amino-4-pyrimidinyl)oxy]-3-ethyl-2-methylphenyl}-1-[3-(trifluoromethyl)phenyl]-2,4-imidazolidinedione

¹H NMR (300 MHz, DMSO-d₆): δ 8.19, 8.09, 7.90, 7.70, 7.58-7.49, 7.24, 7.06, 6.92, 5.80, 4.86, 4.71, 2.58, 2.17, 1.24;

LCMS: m/z 494 [M+Na]+;

HPLC retention time: 1.344 min (method 5).

### Example 3(202): 3-{4-[(6-amino-4-pyrimidinyl)oxy]bicyclo[2.2.1]hept-1-yl}-1-[3-(trifluoromethyl)phenyl]-2,4-imidazolidinedione

¹H NMR (400 MHz, DMSO-d₆): δ 8.13, 8.08, 7.78, 7.64, 7.48, 6.60, 5.69, 4.47, 2.68, 2.41, 2.20, 1.98;

LCMS: m/z 448 [M+H]+;

HPLC retention time: 2.574 min (method 16-2).

### Example 3(203): 3-{4-[(6-amino-4-pyrimidinyl)oxy]-2-methylphenyl}-1-[3-(trifluoromethyl)phenyl]-2,4-imidazolidinedione trifluoroacetate (1:1)

¹H NMR (400 MHz, DMSO-d₆): δ 8.18, 7.90, 7.69, 7.53, 7.38, 7.22-7.19, 7.15-7.12, 5.87, 4.84, 4.70, 2.19;

LCMS: m/z 485 [M+MeCN+H]+;

HPLC retention time: 1.668 min (method 10).

### Example 3(204): 3-{4-[(2-amino-4-pyrimidinyl)oxy]-2-isopropylphenyl}-1-[3-(trifluoromethyl)phenyl]-2,4-imidazolidinedione

¹H NMR (400 MHz, DMSO-d₆): δ 8.16-8.14, 7.90-7.88, 7.69, 7.53, 7.36, 7.27, 7.16, 6.73, 6.21, 4.88, 4.69, 2.93, 1.13;

LCMS: m/z 513 [M+MeCN+H]+;

HPLC retention time: 1.558 min (method 10).

### Example 3(205): 3-{4-[(6-amino-4-pyrimidinyl)oxy]-2-isopropylphenyl}-1-[3-(trifluoromethyl)phenyl]-2,4-imidazolidinedione

¹H NMR (400 MHz, DMSO-d₆): δ 8.16-8.08, 7.91, 7.69, 7.58-7.49, 7.36, 7.26, 7.12, 6.94, 5.87, 4.89, 4.70, 2.97-2.90, 1.14;

LCMS: m/z 472 [M+H]+;

HPLC retention time: 1.481 min (method 2).

### Example 3(206): 3-{5-[(6-amino-4-pyrimidinyl)oxy]bicyclo[2.2.1]hept-2-yl}-1-[5-(trifluoromethyl)-3-pyridinyl]-2,4-imidazolidinedione

¹H NMR (400 MHz, DMSO-d₆): δ 9.08, 8.74, 8.47, 8.07, 6.59, 5.65, 4.86, 4.53, 3.94, 3.51-3.40, 2.60, 2.56-2.50, 2.17, 2.08, 1.83-1.80, 1.78, 1.68, 1.62;

LCMS: m/z 449 [M+H]+;

HPLC retention time: 1.282 min (method 1).

### Example 3(207): 3-{5-[(6-amino-4-pyrimidinyl)oxy]bicyclo[2.2.1]hept-2-yl}-1-[5-(trifluoromethyl)-3-pyridinyl]-2,4-imidazolidinedione

¹H NMR (400 MHz, DMSO-d₆): δ 9.07, 8.74, 8.48, 8.08, 6.58, 5.65, 4.78-4.76, 4.55, 3.89, 2.47, 2.37, 2.11-2.08, 1.85, 1.66, 1.56, 1.45;

LCMS: m/z 449 [M+H]+;

HPLC retention time: 1.239 min (method 1).

### Example 3(208): 3-{5-[(6-amino-4-pyrimidinyl)oxy]bicyclo[2.2.1]hept-2-yl}-1-[5-(trifluoromethyl)-3-pyridinyl]-2,4-imidazolidinedione

¹H NMR (400 MHz, DMSO-d₆): δ 9.07, 8.74, 8.48, 8.08, 6.58, 5.65, 4.78-4.76, 4.53, 3.89, 2.47, 2.37, 2.11-2.08, 1.85, 1.71-1.61, 1.56, 1.50-1.41;

LCMS: m/z 449 [M+H]+;

HPLC retention time: 1.243 min (method 1).

### Example 3(209): 3-{5-[(6-amino-4-pyrimidinyl)oxy]bicyclo[2.2.1]hept-2-yl}-1-[3-(trifluoromethyl)phenyl]-2,4-imidazolidinedione

¹H NMR (400 MHz, DMSO-d₆): δ 8.13, 8.08, 7.79, 7.64, 7.49, 6.59, 5.65, 4.86, 4.47, 3.93, 3.51, 3.42, 2.59-2.50, 2.42, 2.18, 2.10-2.07, 1.83-1.78, 1.64, 1.58, 1.45, 1.24;

LCMS: m/z 448 [M+H]+;

HPLC retention time: 1.483 min (method 1).

### Example 3(210): 3-{5-[(6-amino-4-pyrimidinyl)oxy]bicyclo[2.2.1]hept-2-yl}-1-[3-(trifluoromethyl)phenyl]-2,4-imidazolidinedione

¹H NMR (400 MHz, DMSO-d₆): δ 8.13, 8.08, 7.79, 7.64, 7.49, 6.59, 5.65, 4.86, 4.47, 3.93, 3.51, 3.42, 2.59-2.50, 2.42, 2.18, 2.10-2.07, 1.83-1.78, 1.64, 1.58, 1.45, 1.24;

LCMS: m/z 448 [M+H]+;

HPLC retention time: 1.479 min (method 1).

### Example 3(211): 3-{5-[(6-amino-4-pyrimidinyl)oxy]bicyclo[2.2.1]hept-2-yl}-1-[3-(trifluoromethyl)phenyl]-2,4-imidazolidinedione

¹H NMR (400 MHz, DMSO-d₆): δ 8.12-8.08, 7.80, 7.64, 7.49, 6.58, 5.66, 4.77-4.76, 4.52, 3.89, 2.46, 2.37, 2.12-2.08, 1.85, 1.67-1.62, 1.56, 1.46-1.43;

LCMS: m/z 448 [M+H]+;

HPLC retention time: 1.449 min (method 1).

### Example 3(212): 3-{4-[(6-amino-4-pyrimidinyl)oxy]-2-methylphenyl}-1-[3-(difluoromethoxy)phenyl]-2,4-imidazolidinedione

¹H NMR (300 MHz, DMSO-d₆): δ 8.12, 7.63, 7.52-7.38, 7.35, 7.19, 7.10, 7.02-6.97, 5.83, 4.77, 4.63, 2.18;

LCMS: m/z 483 [M+MeCN+H]+;

HPLC retention time: 1.571 min (method 10).

### Example 3(213): 3-{4-[(6-amino-4-pyrimidinyl)oxy]-2-ethylphenyl}-1-[3-(difluoromethoxy)phenyl]-2,4-imidazolidinedione

¹H NMR (300 MHz, DMSO-d₆): δ 8.10, 7.62, 7.51-7.37, 7.34, 7.27, 7.18, 7.10, 7.02-6.93, 5.83, 4.80, 4.63, 2.50, 1.10;

LCMS: m/z 497 [M+MeCN+H]+;

HPLC retention time: 1.637 min (method 10).

### Example 3(214): 3-{4-[(6-amino-4-pyrimidinyl)oxy]-2-methylphenyl}-1-[3-(difluoromethyl)phenyl]-2,4-imidazolidinedione

¹H NMR (400 MHz, DMSO-d₆): δ 8.11, 8.00, 7.78, 7.58, 7.37, 7.19, 7.13-7.07, 6.97-6.93, 5.83, 4.81, 4.67, 2.19;

LCMS: m/z 426 [M+H]+;

HPLC retention time: 1.082 min (method 65).

### Example 3(215): 3-{4-[(6-amino-4-pyrimidinyl)oxy]-2-ethylphenyl}-1-[3-(difluoromethyl)phenyl]-2,4-imidazolidinedione

¹H NMR (400 MHz, DMSO-d₆): δ 8.11, 8.00, 7.78, 7.58, 7.37, 7.20, 7.13, 7.11-7.07, 6.97-6.93, 5.84, 4.84, 4.67, 2.50, 1.10;

LCMS: m/z 440 [M+H]+;

HPLC retention time: 1.149 min (method 65).

### Example 3(216): 3-{4-[(6-amino-4-pyrimidinyl)oxy]-2-methylphenyl}-1-[4-fluoro-3-(trifluoromethoxy)phenyl]-2,4-imidazolidinedione

¹H NMR (400 MHz, DMSO-d₆): δ 8.10, 8.04-8.02, 7.68-7.59, 7.35, 7.18, 7.11, 6.93, 5.82, 4.78, 4.64, 2.18;

LCMS: m/z 478 [M+H]+;

HPLC retention time: 1.738 min (method 10).

### Example 3(217): 3-{4-[(6-amino-4-pyrimidinyl)oxy]-2-ethylphenyl}-1-[4-fluoro-3-(trifluoromethoxy)phenyl]-2,4-imidazolidinedione

¹H NMR (400 MHz, DMSO-d₆): δ 8.12, 8.03-8.01, 7.68-7.59, 7.35, 7.20, 7.12, 6.98, 5.84, 4.81, 4.64, 2.50, 1.10;

LCMS: m/z 533 [M+MeCN+H]+;

HPLC retention time: 1.795 min (method 10).

### Example 3(218): 3-{4-[(6-amino-4-pyrimidinyl)oxy]-2-methylphenyl}-1-[3-fluoro-5-(trifluoromethyl)phenyl]-2,4-imidazolidinedione

¹H NMR (400 MHz, DMSO-d₆): δ 8.10, 8.02, 7.83, 7.49-7.47, 7.37, 7.19, 7.12, 6.94, 5.83, 4.82, 4.70, 2.18;

LCMS: m/z 503 [M+MeCN+H]+;

HPLC retention time: 1.736 min (method 10).

### Example 3(219): 3-{4-[(6-amino-4-pyrimidinyl)oxy]-2-ethylphenyl}-1-[3-fluoro-5-(trifluoromethyl)phenyl]-2,4-imidazolidinedione

¹H NMR (400 MHz, DMSO-d₆): δ 8.17-8.12, 7.96, 7.64, 7.37, 7.21, 7.13, 6.96, 5.85, 4.87, 4.70, 2.50, 1.11;

LCMS: m/z 476 [M+H]+;

HPLC retention time: 1.475 min (method 46).

### Example 3(220): 3-{4-[(6-amino-4-pyrimidinyl)oxy]-2-methylphenyl}-1-[4-fluoro-3-(trifluoromethyl)phenyl]-2,4-imidazolidinedione

¹H NMR (400 MHz, DMSO-d₆): δ 8.17-8.13, 7.96, 7.63, 7.36, 7.20, 7.12, 7.01, 5.84, 4.83, 4.70, 2.18;

LCMS: m/z 462 [M+H]+;

HPLC retention time: 1.584 min (method 55).

### Example 3(221): 3-{4-[(6-amino-4-pyrimidinyl)oxy]-3-isopropylphenyl}-1-(3-isopropoxyphenyl)-2,4-imidazolidinedione

¹H NMR (300 MHz, DMSO-d₆): δ 8.07, 7.42, 7.33-7.22, 7.19-7.16, 6.89, 6.74, 5.80, 4.64-4.61, 3.04-3.00, 1.27, 1.16;

LCMS: m/z 462 [M+H]+;

HPLC retention time: 1.622 min (method 38).

### Example 3(222): 3-{4-[(6-amino-4-pyrimidinyl)oxy]-3-isopropylphenyl}-1-[3-(difluoromethyl)phenyl]-2,4-imidazolidinedione trifluoroacetate (1:1)

¹H NMR (400 MHz, DMSO-d₆): δ 8.19, 8.02, 7.79-7.77, 7.58, 7.46, 7.38-7.19, 7.17-7.08, 5.86, 4.67, 3.03, 1.15;

LCMS: m/z 454 [M+H]+;

HPLC retention time: 1.285 min (method 5).

### Example 3(223): 3-(trans-4-{[6-(methylsulfonyl)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.10, 8.99, 8.75, 8.63, 8.49, 8.41, 8.15, 5.45-5.28, 4.59, 4.13-3.97, 3.35, 2.36-2.32, 1.89-1.77;

LCMS: m/z 550 [M+H]+;

HPLC retention time: 1.355 min (method 84).

### Example 3(224): 3-{4-[(6-methoxypyrido[3,4-d]pyrimidin-4-yl)oxy]phenyl}-1-[5-(trifluoromethyl)-3-pyridinyl]-2,4-imidazolidinedione

¹H NMR (400 MHz, DMSO-d₆) δ 9.21-9.20, 8.80-8.55, 7.60-7.53, 4.81, 4.06;

LCMS: m/z 497 [M+H]+;

HPLC retention time: 1.687 min (method 67).

### Example 3(225): 3-{trans-4-[(6-methoxypyrido[3,4-d]pyrimidin-4-yl)oxy]cyclohexyl}-1-[3-(trifluoromethyl)phenyl]-2,4-imidazolidinedione

¹H NMR (400 MHz, DMSO-d₆) δ ppm: 9.09, 8.76, 8.15, 7.83-7.80, 7.68-7.64, 7.51-7.49, 7.22, 5.34-5.28, 4.53, 4.09-4.03, 4.01, 2.42-2.39, 1.91-1.83, 1.79-1.69;

LCMS: m/z 502 [M+H]+;

HPLC retention time: 1.702 min (method 84).

### Example 3(226): 3-[trans-4-([1,3]dioxolo[4,5-g]quinazolin-8-yloxy)cyclohexyl]-1-[5-(trifluoromethyl)-3-pyridinyl]-2,4-imidazolidinedione

¹H NMR (400 MHz, DMSO-d₆) δ 9.10, 8.75, 8.62, 8.52-8.47, 7.33, 6.26, 5.29-5.21, 4.59, 4.06, 2.31-2.27, 1.89-1.83, 1.70-1.67;

LCMS: m/z 516 [M+H]+;

HPLC retention time: 1.224 min (method 84).

### Example 3(227): 3-[4-([1,3]dioxolo[4,5-g]quinazolin-8-yloxy)phenyl]-1-[5-(trifluoromethyl)-3-pyridinyl]-2,4-imidazolidinedione

¹H NMR (400 MHz, DMSO-d₆) δ 9.21, 8.80, 8.57, 7.68, 7.60-7.42 (s, 5H), 6.33, 4.80;

LCMS: m/z 510 [M+H]+;

HPLC retention time: 1.301 min (method 84).

### Example 3(228): 3-(cis-4-{[7-(difluoromethoxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.11, 8.82, 8.75, 8.49, 8.21-8.19, 7.77-7.40, 5.42-5.30, 4.59, 4.12-4.03, 2.42-2.30, 1.91-1.85, 1.78-1.68;

LCMS: m/z 538 [M+H]+;

HPLC retention time: 1.891 min (method 67).

### Example 3(229): 3-(4-{[7-(difluoromethoxy)-4-quinazolinyl]oxy}phenyl)-1-[5-(trifluoromethyl)-3-pyridinyl]-2,4-imidazolidinedione

¹H NMR (400 MHz, DMSO-d₆) δ 9.20, 8.79-8.78, 8.54, 8.49, 7.83-7.63, 7.61-7.46, 4.80;

LCMS: m/z 532 [M+H]+;

HPLC retention time: 2.631 min (method 16-3).

### Example 3(230): 3-(trans-4-{[7-(trifluoromethoxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.11, 8.88, 8.75, 8.49, 8.29, 7.85, 7.70, 5.39-5.29, 4.59, 4.13-4.03, 2.42-2.29, 1.90-1.86, 1.74-1.70;

LCMS: m/z 556 [M+H]+;

HPLC retention time: 1.739 min (method 84).

### Example 3(231): 3-(4-{[7-(trifluoromethoxy)-4-quinazolinyl]oxy}phenyl)-1-[5-(trifluoromethyl)-3-pyridinyl]-2,4-imidazolidinedione

¹H NMR (400 MHz, DMSO-d₆) δ 9.20, 8.85-8.83, 8.60, 7.98, 7.82, 7.63-7.51, 4.81;

LCMS: m/z 550 [M+H]+;

HPLC retention time: 1.674 min (method 84).

### Example 3(232): 3-{trans-4-[(7-methoxypyrido[3,2-d]pyrimidin-4-yl)oxy]cyclohexyl}-1-[5-(trifluoromethyl)-3-pyridinyl]-2,4-imidazolidinedione

¹H NMR (400 MHz, DMSO-d₆) δ 9.11, 8.79-8.68, 8.49, 7.69, 5.36-5.28, 4.59, 4.11-4.01, 2.40-2.30, 1.87, 1.72;

LCMS: m/z 503 [M+H]+;

HPLC retention time: 1.526 min (method 88).

### Example 3(233): 1-[5-(trifluoromethyl)-3-pyridinyl]-3-(trans-4-{[7-(trifluoromethyl)-4-quinazolinyl]oxy}cyclohexyl)-2,4-imidazolidinedione

¹H NMR (300 MHz, DMSO-d₆) δ 9.11, 8.96, 8.77-8.73, 8.49, 8.36, 8.29, 7.97, 5.39-5.32, 4.59, 4.12-4.04, 2.41-2.27, 1.89-1.82, 1.77-1.68;

LCMS: m/z 540 [M+H]+;

HPLC retention time: 1.795 min (method 84).

### Example 3(234): 3-[4-(pyrazolo[1,5-a]pyrimidin-7-yloxy)phenyl]-1-[5-(trifluoromethyl)-3-pyridinyl]-2,4-imidazolidinedione

¹H NMR (400 MHz, DMSO-d₆) δ 9.21, 8.81-8.80, 8.55, 8.45, 8.31, 7.64, 6.79, 6.24, 4.81;

LCMS: m/z 455 [M+H]+;

HPLC retention time: 1.195 min (method 84).

### Example 3(235): 3-[2,4-dioxo-3-(4-{[7-(trifluoromethoxy)-4-quinazolinyl]oxy}phenyl)-1-imidazolidinyl]-5-(trifluoromethyl)benzonitrile

¹H NMR (400 MHz, DMSO-d₆) δ 8.84, 8.62-8.53, 8.35, 8.16, 7.99, 7.83, 7.63-7.53, 4.77;

LCMS: m/z 573 [M+H]+;

HPLC retention time: 2.040 min (method 93).

### Example 3(236): 3-{trans-4-[(7-ethoxy-4-quinazolinyl)oxy]cyclohexyl}-1-[5-(trifluoromethyl)-3-pyridinyl]-2,4-imidazolidinedione

¹H NMR (400 MHz, DMSO-d₆) δ9.11, 8.65-8.88, 8.49, 7.89-8.11, 7.15-7.31, 5.31-5.28, 4.59, 4.22, 4.09-4.05, 2.22-2.42, 1.86-1.80, 1.72-1.68, 1.41;

LCMS: m/z 485 [M+H]+;

HPLC retention time: 1.420 min (method 84).

### Example 3(237): 3-{trans-4-[(7-isopropoxy-4-quinazolinyl)oxy]cyclohexyl}-1-[5-(trifluoromethyl)-3-pyridinyl]-2,4-imidazolidinedione

¹H NMR (400 MHz, DMSO-d₆) δ 9.10, 8.75, 8.70, 8.49, 8.00, 7.27, 7.28-7.22, 5.31-5.21, 4.89-4.81, 4.59, 4.08-4.00, 2.35-2.25, 1.89-1.80, 1.71-1.61, 1.34;

LCMS: m/z 530 [M+H]+;

HPLC retention time: 1.673 min (method 84-1).

### Example 3(238): 3-(trans-4-{[7-(2-methoxyethoxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ ppm: 9.11, 8.75-8.72, 8.49, 8.03, 7.31-7.27, 5.33-5.28, 4.59, 4.30-4.28, 4.12-4.02, 3.75-3.73, 3.35-3.30, 2.41-2.28, 1.91-1.82, 1.73-1.60;

LCMS: m/z 546 [M+H]+;

HPLC retention time: 1.593 min (method 88).

### Example 3(239): 4-[(trans-4-{2,5-dioxo-3-[5-(trifluoromethyl)-3-pyridinyl]-1-imidazolidinyl}cyclohexyl)oxy]-6-quinazolinecarbonitrile

¹H NMR (300 MHz, DMSO-d₆) δ 9.11, 8.96, 8.75, 8.67, 8.49, 8.28-8.24, 8.06, 5.40-5.29, 4.59, 4.10-3.98, 2.40-2.30, 1.95-1.66;

LCMS: m/z 490 [M+H]+;

HPLC retention time: 1.682 min (method 88).

### Example 3(240): 4-[(trans-4-{2,5-dioxo-3-[5-(trifluoromethyl)-3-pyridinyl]-1-imidazolidinyl}cyclohexyl)oxy]-7-quinazolinecarbonitrile

¹H NMR (400 MHz, DMSO-d₆) δ 9.12, 8.96, 8.77, 8.54-8.50, 8.30, 8.03, 5.38-5.33, 4.61, 4.12-4.06, 2.41-2.34, 1.90-1.80, 1.77-1.71;

LCMS: m/z 497 [M+H]+;

HPLC retention time: 1.450 min (method 84).

### Example 3(241): 3-{trans-4-[(6-methyl-4-quinazolinyl)oxy]cyclohexyl}-1-[5-(trifluoromethyl)-3-pyridinyl]-2,4-imidazolidinedione

¹H NMR (300 MHz, DMSO-d₆) δ 9.10, 8.73, 8.49, 7.91, 7.84-7.76, 5.35-5.28, 4.59, 4.11-4.07, 2.52, 2.36-2.29, 1.88-1.73, 1.78-1.65;

LCMS: m/z 486 [M+H]+;

HPLC retention time: 1.357 min (method 84).

### Example 3(242): 3-{trans-4-[(6-methoxy-4-quinazolinyl)oxy]cyclohexyl}-1-[5-(trifluoromethyl)-3-pyridinyl]-2,4-imidazolidinedione

¹H NMR (300 MHz, DMSO-d₆) δ 9.10, 8.75, 8.70, 8.69, 7.85, 7.58, 7.38, 5.35-5.28, 4.59, 4.11-4.03, 3.92, 2.37-2.33, 1.88-1.67;

LCMS: m/z 502 [M+H]+;

HPLC retention time: 1.798 min (method 67).

### Example 3(243): 3-{trans-4-[(6-fluoro-4-quinazolinyl)oxy]cyclohexyl}-1-[5-(trifluoromethyl)-3-pyridinyl]-2,4-imidazolidinedione

¹H NMR (400 MHz, DMSO-d₆) δ 9.11, 8.82, 8.75, 8.50, 8.04-8.00, 7.91-7.80, 5.38-5.28, 4.59, 4.10-4.04, 2.39-2.30, 1.88-1.86, 1.78-1.71;

LCMS: m/z 490 [M+H]+;

HPLC retention time: 1.733 min (method 88).

### Example 3(244): 3-{trans-4-[(7-fluoro-4-quinazolinyl)oxy]cyclohexyl}-1-[5-(trifluoromethyl)-3-pyridinyl]-2,4-imidazolidinedione

¹H NMR (400 MHz, DMSO-d₆) δ 9.10, 8.82, 8.76-8.70, 8.49, 8.22, 7.70, 7.62-7.57, 5.34-5.31, 4.59, 4.08-4.04, 2.39-2.30, 1.88-1.85, 1.74-1.70;

LCMS: m/z 490 [M+H]+;

HPLC retention time: 1.723 min (method 88).

### Example 3(245): 1-[5-(trifluoromethyl)-3-pyridinyl]-3-(trans-4-{[6-(trifluoromethyl)-4-quinazolinyl]oxy}cyclohexyl)-2,4-imidazolidinedione

¹H NMR (300 MHz, DMSO-d₆) δ 9.10, 8.96, 8.74, 8.49, 8.43-8.40, 8.25, 8.12, 5.40-5.32, 4.59, 4.11-4.03, 2.41-2.33, 1.89-1.72;

LCMS: m/z 540 [M+H]+;

HPLC retention time: 1.830 min (method 88).

### Reference Example 10: N-(2-chloroethyl)-4-(quinolin-4-yloxy)aniline

To a solution of 4-(quinolin-4-yloxy)aniline (CAS No. 863503-93-9, 200 mg) in MeOH (10 mL) and acetic acid (0.2 mL), was added chloroacetaldehyde (CAS No. 107-20-0, 0.16 mL), BH₃.Me₃N (123 mg), and the mixture stirred at room temperature for 4 h. The mixture was diluted with water, extracted with ethyl acetate and the combined organic extracts were dried over sodium sulphate. The solvent was removed under reduced pressure to give the title compound (100 mg) having the following physical property values.

ESI MS m/z 299 [M+H]+.

### Reference Example 11: 1-(2-chloroethyl)-3-(3-nitro-5-(trifluoromethyl)phenyl)-1-(4-(quinolin-4-yloxy)phenyl)urea

To a solution of the compound prepared in Reference Example 10 (300 mg) in DCM (10 mL) and pyridine (0.25 mL), was added 1-isocyanato-3-nitro-5-trifluoromethylbenzene (CAS No. 16588-71-9, 403 mg) and the mixture was stirred at room temperature for 16 h. The mixture was diluted with sodium bicarbonate solution, extracted with ethyl acetate and the combined organic extracts were dried over sodium sulphate. The solvent was removed under reduced pressure to give the title compound (350 mg) having the following physical property values.

ESI LC-MS m/z 531 [M+H]+.

### Reference Example 12: 1-(3-nitro-5-(trifluoromethyl)phenyl)-3-(4-(quinolin-4-yloxy)phenyl)imidazolidin-2-one

To a solution of the compound prepared in Reference Example 11 (350 mg) in THF (10 mL) was added sodium hydride (31 mg) and the mixture was stirred at room temperature for 2 h. The mixture was diluted with ice cooled water and extracted with ethyl acetate. The combined organic extracts were dried over sodium sulphate and the solvent was removed under reduced pressure to give the title compound (300 mg) having the following physical property values.

¹H NMR (300 MHz, CDCl₃): δ 8.68, 8.57, 8.46, 8.37, 8.19, 8.10, 7.80-7.57, 7.27-7.24, 6.57, 4.14.

### Example 4: 1-(3-amino-5-(trifluoromethyl)phenyl)-3-(4-(quinolin-4-yloxy)phenyl)imidazolidin-2-one

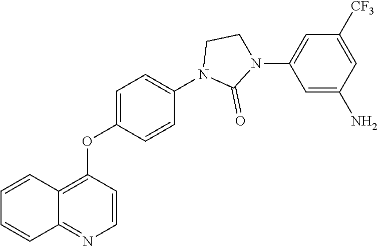 [SMILES: Nc1cc(N2CCN(c3ccc(Oc4ccnc5ccccc45)cc3)C2=O)cc(C(F)(F)F)c1]

Iron powder (45 mg) was added to a solution of the compound prepared in Reference Example 12 (200 mg) in ethanol (15 mL), followed by ammonium chloride (85 mg). The mixture was heated at 70° C. for 30 min. The reaction mixture was filtered and concentrated. The crude residue was treated with water and extracted with EtOAc. The organic layer was dried and concentrated to give the title compound (55 mg) having the following physical property values.

¹H NMR (400 MHz, CDCl₃): δ 8.66, 8.37, 8.09, 7.78-7.54, 7.21, 6.93, 6.65, 6.55, 4.81-4.72, 4.02-3.99;

ESI MS, m/z 465 [M+H]+;

HPLC retention time: 18.64 min (method 76).

### Example 4(1)˜4(12)

A procedure for a purpose similar to that for Reference Example 10→Reference Example 11→Reference Example 12 was carried out by using a corresponding amine compound instead of 4-(quinolin-4-yloxy)aniline, and a corresponding isocyanate compound instead of 1-isocyanato-3-nitro-5-trifluoromethylbenzene to give the following compounds of Examples.

### Example 4(1): 1-{4-[(2-amino-4-pyrimidinyl)oxy]-3-ethylphenyl}-3-[3-(trifluoromethyl)phenyl]-2-imidazolidinone

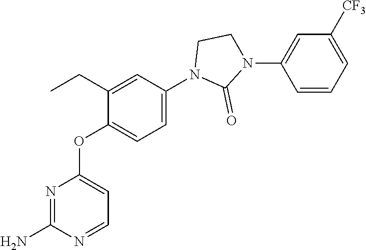 [SMILES: CCc1cc(N2CCN(c3cccc(C(F)(F)F)c3)C2=O)ccc1Oc1ccnc(N)n1]

¹H NMR (400 MHz, DMSO-d₆): δ 8.18, 8.09, 7.76, 7.61-7.60, 7.48, 7.41, 7.10, 6.60, 6.08, 4.03, 2.49, 1.11;

LCMS: m/z 444 [M+H]+;

HPLC retention time: 2.649 min (method 28).

### Example 4(2): 1-[3-ethyl-4-(7H-pyrrolo[2,3-d]pyrimidin-4-yloxy)phenyl]-3-[3-(trifluoromethyl)phenyl]-2-imidazolidinone

¹H NMR (400 MHz, DMSO-d₆): δ 12.20, 8.27, 8.20, 7.80, 7.66-7.60, 7.53, 7.45-7.40, 7.19, 6.44, 4.06, 2.48, 1.09;

LCMS: m/z 468 [M+H]+;

HPLC retention time: 3.155 min (method 29).

### Example 4(3): 1-[4-(1H-pyrrolo[2,3-b]pyridin-5-yloxy)phenyl]-3-[3-(trifluoromethyl)phenyl]-2-imidazolidinone

¹H NMR (400 MHz, DMSO-d₆): δ 11.71, 8.16, 8.07, 7.76, 7.64-7.58, 7.52-7.51, 7.40-7.38, 7.03-6.99, 6.42, 4.05-3.95;

LCMS: m/z 439 [M+H]+;

HPLC retention time: 1.915 min (method 1).

### Example 4(4): 5-(2-ethyl-4-{2-oxo-3-[3-(trifluoromethyl)phenyl]-1-imidazolidinyl}phenoxy)-3,4-dihydro-1,8-naphthyridin-2(1H)-one

¹H NMR (400 MHz, DMSO-d₆): δ 10.48, 8.19, 7.94, 7.78, 7.69, 7.62, 7.55, 7.42-7.40, 7.10, 6.13, 4.04, 2.98, 2.56, 1.13;

LCMS: m/z 497 [M+H]+;

HPLC retention time: 1.906 min (method 1).

### Example 4(5): 1-[3-ethyl-4-(pyrazolo[1,5-a]pyrimidin-7-yloxy)phenyl]-3-[3-(trifluoromethyl)phenyl]-2-imidazolidinone

¹H NMR (400 MHz, CD₃OD): δ 8.81, 8.15, 8.00, 7.81-7.78, 7.67, 7.60-7.51, 7.40-7.38, 7.17, 6.76, 6.27, 4.11, 2.60, 1.21;

LCMS: m/z 490 [M+Na]+;

HPLC retention time: 2.006 min (method 1).

### Example 4(6): 1-[3-ethyl-4-(1H-pyrrolo[2,3-b]pyridin-4-yloxy)phenyl]-3-[3-(trifluoromethyl)phenyl]-2-imidazolidinone

¹H NMR (400 MHz, DMSO-d₆): δ 11.72, 8.20, 8.05, 7.78, 7.71, 7.62, 7.54, 7.42-7.40, 7.34-7.33, 7.14, 6.28, 6.20, 4.05, 2.59-2.51, 1.12;

LCMS: m/z 508 [M+MeCN+H]+;

HPLC retention time: 1.453 min (method 36).

### Example 4(7): 1-[3-methyl-4-(1H-pyrrolo[2,3-b]pyridin-4-yloxy)phenyl]-3-[3-(trifluoromethyl)phenyl]-2-imidazolidinone

¹H NMR (400 MHz, DMSO-d₆): δ 11.71, 8.20, 8.04, 7.78, 7.67-7.55, 7.41, 7.34, 7.16, 6.25-6.19, 4.05, 2.15;

LCMS: m/z 453 [M+H]+;

HPLC retention time: 1.555 min (method 9)

### Example 4(8): 1-[2-ethyl-4-(7H-pyrrolo[2,3-d]pyrimidin-4-yloxy)phenyl]-3-[5-(trifluoromethyl)-3-pyridinyl]-2-imidazolidinone

¹H NMR (400 MHz, DMSO-d₆): δ 12.23, 9.04, 8.63, 8.52, 8.34, 7.49-7.43, 7.24-7.17, 6.53, 4.17, 3.96, 2.64, 1.17;

LCMS: m/z 469 [M+H]+;

HPLC retention time: 1.439 min (method 5).

### Example 4(9): 1-{4-[(6-amino-4-pyrimidinyl)oxy]phenyl}-3-[3-(trifluoromethyl)phenyl]-2-imidazolidinone

¹H NMR (400 MHz, DMSO-d₆): δ 8.17, 8.07, 7.79, 7.70-7.66, 7.62, 7.41, 7.19-7.15, 6.82, 5.71, 4.08-3.99;

LCMS: m/z 416 [M+H]+;

HPLC retention time: 1.604 min (method 1).

### Example 4(10): 1-{4-[(6-amino-4-pyrimidinyl)oxy]-3-ethylphenyl}-3-[3-(trifluoromethyl)phenyl]-2-imidazolidinone

¹H NMR (400 MHz, DMSO-d₆): δ 8.18, 8.07, 7.79, 7.63-7.59, 7.50, 7.41-7.39, 7.08, 6.87, 5.68, 4.08-3.99, 2.47, 1.13;

LCMS: m/z 466 [M+Na]+;

HPLC retention time: 1.394 min (method 5).

### Example 4(11): 1-[4-(1H-pyrrolo[2,3-b]pyridin-4-yloxy)phenyl]-3-[3-(trifluoromethyl)phenyl]-2-imidazolidinone

¹H NMR (300 MHz, DMSO-d₆): δ 4.04, 6.13-6.26, 6.34-6.45, 7.15-7.27, 7.30-7.35, 7.36-7.44, 7.54-7.65, 7.66-7.81, 8.02-8.10, 8.13-8.19, 11.59-11.71; TLC Rf: 0.10 (AcOEt/n-hexane=1/1).

### Example 4(12): 1-{4-[(6,7-dimethoxy-4-quinolinyl)oxy]phenyl}-3-[5-(trifluoromethyl)-3-pyridinyl]-2-imidazolidinone trifluoroacetate (1:1)

¹H NMR (400 MHz, DMSO-d₆): δ 8.78, 8.21, 7.89-7.88, 7.87-7.79, 7.73, 7.64, 7.54, 7.44, 6.80, 4.09, 4.04;

LCMS: m/z 511 [M+H]+;

HPLC retention time: 2.027 min (method 3).

### Example 5: (4S)-3-[3-ethyl-4-(7H-pyrrolo[2,3-d]pyrimidin-4-yloxy)phenyl]-4-hydroxy-1-[5-(trifluoromethyl)-3-pyridyl]imidazolidin-2-one

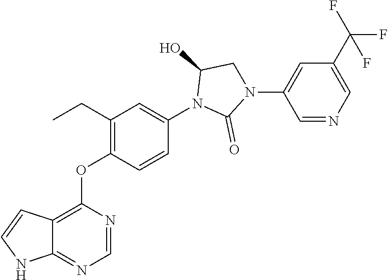 [SMILES: CCc1cc(N2C(=O)N(c3cncc(C(F)(F)F)c3)C[C@@H]2O)ccc1Oc1ncnc2[nH]ccc12]

To a stirred solution of the compound prepared in Example 3(4) (100 mg) in ethanol (8 mL) were added NaBH₄ (35 mg) in portions at room temperature under nitrogen atmosphere. The resulting mixture was stirred for 2 h at room temperature under nitrogen atmosphere. The reaction was quenched by the addition of water (a drop) at room temperature. The resulting mixture was concentrated under vacuum. The crude product was purified by Prep-HPLC with the following conditions (Column: Xselect CSH OBD Column (trademark) 30*150 mm 5 um; Mobile Phase A: Water (0.1% FA), Mobile Phase B: MeCN; Flow rate: 60 mL/min; Gradient: 40% B to 42% B in 7 min; 254/220 nm; Rt: 6.83 min) to afford racemate compound. The racemate (18 mg) was splitted by Chiral-HPLC to afford the title compound (6 mg) having the following physical property values.

¹H NMR (400 MHz, DMSO-d₆): δ 12.23, 9.29, 8.67, 8.59, 8.29, 7.65, 7.54, 7.48, 7.22, 6.90, 6.49, 5.87, 4.31, 3.87, 2.47, 1.10;

LCMS: m/z 485 [M+H]+;

HPLC retention time: 1.560 min (method 1);

Chiral HPLC retention time: 1.468 min (column: CHIRALPAK IF-3 (trademark), 0.46×5 cm: 3 um, Mobile phase: Hexane/AcOEt=50/50, Flow: 1.0 mL/min, Temp: 25 degree).

### Example 5(1)˜5(46)

A procedure for a purpose similar to that for Example 5 was carried out by using a corresponding hydantoin compound instead of the compound prepared in Example 3(4) to give the following compounds of Examples.

### Example 5(1): 5-[2-ethyl-4-[(5R)-5-hydroxy-2-oxo-3-[5-(trifluoromethyl)-3-pyridyl]imidazolidin-1-yl]phenoxy]-3,4-dihydro-1H-1,8-naphthyridin-2-one

¹H NMR (400 MHz, DMSO-d₆): δ 10.50, 9.08, 8.67, 8.58, 7.97, 7.69, 7.58, 7.12, 6.90, 6.17, 5.85, 4.32, 3.88, 2.98, 2.59-2.49, 1.24;

LCMS: m/z 514 [M+H]+;

HPLC retention time: 2.413 min (method 16-2); Chiral HPLC retention time: 3.727 min (column: CHIRALPAK IG-3 (trademark), 0.46×5 cm: 3 um, Mobile phase: MTBE/IPA=80/20, Flow: 1.0 mL/min, Temp: 25 degree).

### Example 5(2): 3-[3-ethyl-4-(3H-imidazo[4,5-b]pyridin-7-yloxy)phenyl]-4-hydroxy-1-[5-(trifluoromethyl)-3-pyridinyl]-2-imidazolidinone

¹H NMR (400 MHz, DMSO-d₆): δ 9.09, 8.67-8.58, 8.34, 8.17, 7.72, 7.58, 7.53, 7.18, 6.91, 6.37, 5.87, 4.31, 3.88, 2.59-2.51, 1.14;

LCMS: m/z 485 [M+H]+;

HPLC retention time: 1.306 min (method 10).

### Example 5(3): 3-[3-ethyl-4-(pyrazolo[1,5-a]pyrimidin-7-yloxy)phenyl]-4-hydroxy-1-[5-(trifluoromethyl)-3-pyridinyl]-2-imidazolidinone

¹H NMR (400 MHz, DMSO-d₆): δ 9.08, 8.67-8.58, 8.06, 7.65, 7.55, 7.24, 6.92, 6.86, 6.38, 5.89-5.85, 4.31, 3.88, 2.53-2.50, 1.14;

LCMS: m/z 485 [M+H]+;

HPLC retention time: 1.547 min (method 9).

### Example 5(4): 3-{4-[(2-amino-4-pyrimidinyl)oxy]-3-ethylphenyl}-4-hydroxy-1-[5-(trifluoromethyl)-3-pyridinyl]-2-imidazolidinone

¹H NMR (400 MHz, DMSO-d₆): δ 9.07, 8.66, 8.56, 8.11, 7.60, 7.49, 7.13, 6.87, 6.63, 6.13, 5.82, 4.39, 3.86, 2.54-2.50, 1.15;

LCMS: m/z 502 [M+MeCN+H]+;

HPLC retention time: 0.878 min (method 36).

### Example 5(5): 3-[2-ethyl-4-(7H-pyrrolo[2,3-d]pyrimidin-4-yloxy)phenyl]-4-hydroxy-1-[5-(trifluoromethyl)-3-pyridinyl]-2-imidazolidinone

¹H NMR (400 MHz, DMSO-d₆): δ 12.22, 9.06, 8.63, 8.54, 8.33, 7.49, 7.40, 7.24, 7.17, 6.82, 6.54, 5.51, 4.38, 3.90, 2.65, 1.16;

LCMS: m/z 485 [M+H]+;

HPLC retention time: 1.302 min (method 5).

### Example 5(6): (4R)-3-[3-ethyl-4-(7H-pyrrolo[2,3-d]pyrimidin-4-yloxy)phenyl]-4-hydroxy-1-[5-(trifluoromethyl)-3-pyridyl]imidazolidin-2-one

¹H NMR (400 MHz, DMSO-d₆): δ 12.23, 9.08, 8.67, 8.58, 8.29, 7.65, 7.54-7.49, 7.22, 6.92, 6.49, 5.87, 4.31, 3.87, 2.51, 1.10;

LCMS: m/z 485 [M+H]+;

HPLC retention time: 1.564 min (method 1); Chiral HPLC retention time: 2.441 min (column: CHIRALPAK IF-3 (trademark), 0.46×5 cm: 3 um, Mobile phase: Hexane/AcOEt=50/50, Flow: 1.0 mL/min, Temp: 25 degree).

### Example 5(7): 3-{4-[(2-amino-4-pyrimidinyl)oxy]-2-ethylphenyl}-4-hydroxy-1-[5-(trifluoromethyl)-3-pyridinyl]-2-imidazolidinone

¹H NMR (400 MHz, DMSO-d₆): δ 9.06, 8.63, 8.52, 8.15-8.12, 7.37, 7.15, 7.09, 6.76-6.69, 6.17, 5.48, 4.39-4.35, 3.89-3.86, 2.67, 1.16;

LCMS: m/z 461 [M+H]+;

HPLC retention time: 1.044 min (method 5).

### Example 5(8): 3-{4-[(2-amino-4-pyrimidinyl)oxy]-2-ethylphenyl}-4-hydroxy-1-[5-(trifluoromethyl)-3-pyridinyl]-2-imidazolidinone

¹H NMR (400 MHz, DMSO-d₆): δ 9.06, 8.64, 8.52, 8.14, 7.37, 7.16-7.08, 6.77-6.73, 6.18, 5.48, 4.40-4.35, 3.90-3.87, 2.67, 1.17;

LCMS: m/z 461 [M+H]+;

HPLC retention time: 1.457 min (method 55).

### Example 5(9): 3-{4-[(2-amino-4-pyrimidinyl)oxy]-2-ethylphenyl}-4-hydroxy-1-[5-(trifluoromethyl)-3-pyridinyl]-2-imidazolidinone

¹H NMR (400 MHz, DMSO-d₆): δ 9.06, 8.64, 8.52, 8.14, 7.37, 7.16-7.08, 6.77-6.70, 6.18, 5.48, 4.40-4.35, 3.90-3.87, 2.67, 1.14;

LCMS: m/z 461 [M+H]+;

HPLC retention time: 1.465 min (method 55).

### Example 5(10): 3-{4-[(2-amino-4-pyrimidinyl)oxy]-2-ethylphenyl}-4-hydroxy-1-[3-(trifluoromethyl)phenyl]-2-imidazolidinone

¹H NMR (400 MHz, DMSO-d₆): δ 8.17, 8.13, 7.76, 7.59, 7.38-7.35, 7.14, 7.08, 6.69-6.66, 6.17, 5.43, 4.33, 3.78, 2.65, 1.15;

LCMS: m/z 460 [M+H]+;

HPLC retention time: 1.216 min (method 5).

### Example 5(11): 3-[2-ethyl-4-(7H-pyrrolo[2,3-d]pyrimidin-4-yloxy)phenyl]-4-hydroxy-1-[3-(trifluoromethyl)phenyl]-2-imidazolidinone

¹H NMR (400 MHz, DMSO-d₆): δ 12.24, 8.34, 8.20, 7.78, 7.61, 7.50, 7.41-7.37, 7.24-7.16, 6.76, 6.55, 5.50-5.47, 4.35, 3.80, 2.65, 1.17;

LCMS: m/z 484 [M+H]+;

HPLC retention time: 1.491 min (method 5).

### Example 5(12): 3-{4-[(2-amino-4-pyrimidinyl)oxy]-3-isopropylphenyl}-4-hydroxy-1-{4-[(4-methyl-1-piperazinyl methyl]-3-(trifluoromethyl)phenyl}-2-imidazolidinone

¹H NMR (400 MHz, DMSO-d₆): δ 8.17-8.16, 8.10, 7.72, 7.66, 7.46, 7.08, 6.77, 6.63, 6.12, 5.76, 4.24, 3.72, 3.56, 2.96, 2.38-2.32, 2.17, 1.15;

LCMS: m/z 586 [M+H]+;

HPLC retention time: 1.019 min (method 59).

### Example 5(13): 3-{4-[(2-amino-4-pyrimidinyl)oxy]-3-ethylphenyl}-1-[4-fluoro-3-(trifluoromethoxy)phenyl]-4-hydroxy-2-imidazolidinone

¹H NMR (300 MHz, DMSO-d₆): δ 8.13-8.06, 7.85-7.34, 7.11, 6.80, 6.65, 6.15, 5.76, 4.21, 3.71, 2.47, 1.12;

LCMS: m/z 535 [M+MeCN+H]+;

HPLC retention time: 1.630 min (method 2).

### Example 5(14): formic acid—3-{4-[(2-amino-4-pyrimidinyl)oxy]-3-ethylphenyl}-1-[3-fluoro-5-(trifluoromethyl)phenyl]-4-hydroxy-2-imidazolidinone (1:1)

¹H NMR (400 MHz, DMSO-d₆): δ 8.12, 8.02, 7.77, 7.60, 7.50, 7.34, 7.13, 6.84, 6.72, 6.15, 5.79, 4.24, 3.79, 2.50, 1.11;

LCMS: m/z 519 [M+MeCN+H]+;

HPLC retention time: 1.640 min (method 2).

### Example 5(15): 3-{4-[(2-amino-4-pyrimidinyl)oxy]-3-ethyl-2-methylphenyl}-4-hydroxy-1-[5-(trifluoromethyl)-3-pyridinyl]-2-imidazolidinone

¹H NMR (400 MHz, DMSO-d₆): δ 9.06, 8.64, 8.54, 8.13, 7.25, 7.03, 6.74-6.69, 6.17, 5.51, 4.38, 3.89, 2.57, 2.23, 1.05;

LCMS: m/z 475 [M+H]+;

HPLC retention time: 1.241 min (method 1).

### Example 5(16): 3-{4-[(2-amino-4-pyrimidinyl)oxy]-3-ethylphenyl}-1-[4-fluoro-3-(trifluoromethyl)phenyl]-4-hydroxy-2-imidazolidinone

¹H NMR (300 MHz, DMSO-d₆): δ 8.20, 8.11, 7.85, 7.59-7.46, 7.11, 6.89-6.82, 6.72, 6.13, 5.78, 4.25, 3.75, 2.47, 1.12;

LCMS: m/z 478 [M+H]+;

HPLC retention time: 1.417 min (method 1).

### Example 5(17): 3-{4-[(2-amino-4-pyrimidinyl)oxy]-3-ethylphenyl}-4-hydroxy-1-[4-(4-morpholinyl)-3-(trifluoromethyl)phenyl]-2-imidazolidinone

¹H NMR (400 MHz, DMSO-d₆): δ 8.15-8.10, 7.73, 7.62-7.59, 7.50, 7.10, 6.78-6.75, 6.16-6.14, 5.76, 4.23, 3.72-3.69, 2.89-2.82, 2.46, 1.13;

LCMS: m/z 567 [M+Na]+;

HPLC retention time: 1.239 min (method 5).

### Example 5(18): (4S)-3-[4-(2-aminopyrimidin-4-yl)oxy-2-methyl-phenyl]-4-hydroxy-1-[5-(trifluoromethyl)-3-pyridyl]imidazolidin-2-one

¹H NMR (300 MHz, DMSO-d₆): δ 9.07, 8.64, 8.53, 8.14, 7.38, 7.16, 7.09, 6.77-6.72, 6.18, 5.53, 4.37, 3.90-3.86, 2.25;

LCMS: m/z 447 [M+H]+;

HPLC retention time: 1.099 min (method 1);

Chiral HPLC retention time: 1.879 min (column: CHIRAL Cellulose-SB4 (trademark), 4.6×100 cm: 3 um, Mobile phase: Hexane/AcOEt=50/50, Flow: 1.0 mL/min, Temp: 25 degree).

### Example 5(19): (4R)-3-[4-(2-aminopyrimidin-4-yl)oxy-2-methyl-phenyl]-4-hydroxy-1-[5-(trifluoromethyl)-3-pyridyl]imidazolidin-2-one

¹H NMR (300 MHz, DMSO-d₆): δ 9.07, 8.64, 8.53, 8.14, 7.38, 7.16, 7.09, 6.78-6.72, 6.18, 5.53, 4.37, 3.90-3.86, 2.27;

LCMS: m/z 447 [M+H]+;

HPLC retention time: 1.083 min (method 1);

Chiral HPLC retention time: 3.023 min (column: CHIRAL Cellulose-SB4 (trademark), 4.6×100 cm: 3 um, Mobile phase: Hexane/AcOEt=50/50, Flow: 1.0 mL/min, Temp: 25 degree).

### Example 5(20): (4R)-3-[4-(2-aminopyrimidin-4-yl)oxy-2-methyl-phenyl]-4-hydroxy-1-[3-(trifluoromethyl)phenyl]imidazolidin-2-one

¹H NMR (400 MHz, DMSO-d₆): δ 8.18, 8.13, 7.78, 7.61, 7.38, 7.15, 7.08, 6.71-6.67, 6.17, 5.48, 4.32, 3.78, 2.25;

LCMS: m/z 446 [M+H]+;

HPLC retention time: 1.145 min (method 5);

Chiral HPLC retention time: 1.19 min (column: CHIRALPAK IF-3, 0.46×5 cm: 3 um, Mobile phase: Hexane/AcOEt=50/50, Flow: 1.0 mL/min, Temp: 25 degree).

### Example 5(21): (4S)-3-[4-(2-aminopyrimidin-4-yl)oxy-2-methyl-phenyl]-4-hydroxy-1-[3-(trifluoromethyl)phenyl]imidazolidin-2-one

¹H NMR (400 MHz, DMSO-d₆): δ 8.18, 8.13, 7.78, 7.61, 7.38, 7.15, 7.08, 6.71-6.67, 6.17, 5.48, 4.32, 3.78, 2.25;

LCMS: m/z 468 [M+Na]+;

HPLC retention time: 1.152 min (method 5);

Chiral HPLC retention time: 1.55 min (column: CHIRALPAK IF-3 (trademark), 0.46×5 cm: 3 um, Mobile phase: Hexane/AcOEt=50/50, Flow: 1.0 mL/min, Temp: 25 degree).

### Example 5(22): (4S)-3-[4-[(6,7-dimethoxy-4-quinolyl)oxy]phenyl]-4-hydroxy-1-[5-(trifluoromethyl)-3-pyridyl]imidazolidin-2-one

¹H NMR (400 MHz, DMSO-d₆): δ 9.10, 8.68, 8.58, 8.52, 7.82-7.79, 7.53, 7.41, 7.35-7.33, 6.94, 6.51, 5.89-5.85, 4.34, 3.96, 3.90-3.87;

LCMS: m/z 527 [M+H]+;

HPLC retention time: 2.627 min (method 16-1);

Chiral HPLC retention time: 1.31 min (column: CHIRALPAK ID-3 (trademark), 0.46×5 cm: 3 um, Mobile phase: MTBE (0.1% DEA):EtOH=70/30, Flow: 1.0 mL/min, Temp: 25 degree).

### Example 5(23): 3-{4-[(6,7-dimethoxy-4-quinolinyl)oxy]phenyl}-4-hydroxy-1-[3-(trifluoromethyl)phenyl]-2-imidazolidinone

¹H NMR (300 MHz, DMSO-d₆): δ 8.52, 8.23, 7.84-7.78, 7.65, 7.54, 7.43, 7.35-7.30, 6.88, 6.52, 5.82, 4.28, 3.96, 3.79;

LCMS: m/z 526 [M+H]+;

HPLC retention time: 1.408 min (method 9).

### Example 5(24): 5-[2-ethyl-4-[(5S)-5-hydroxy-2-oxo-3-[5-(trifluoromethyl)-3-pyridyl]imidazolidin-1-yl]phenoxy]-3,4-dihydro-1H-1,8-naphthyridin-2-one

¹H NMR (400 MHz, DMSO-d₆): δ 10.50, 9.08, 8.67-8.57, 7.97, 7.69, 7.57, 7.11, 6.90, 6.17, 5.85, 4.30, 3.87, 2.99, 2.59-2.51, 1.14;

LCMS: m/z 514 [M+H]+;

HPLC retention time: 2.405 min (method 16-2);

Chiral HPLC retention time: 1.901 min (column: CHIRALPAK IG-3 (trademark), 4.6×5 cm: 3 um, Mobile phase: MTBE:IPA=80/20, Flow: 1.0 mL/min, Temp: 25 degree).

### Example 5(25)

### (4S)-3-[4-[(6,7-dimethoxy-4-quinolyl)oxy]phenyl]-4-hydroxy-1-[3-(trifluoromethyl)phenyl]imidazolidin-2-one

¹H NMR (400 MHz, DMSO-d₆): δ 8.51, 8.23, 7.81, 7.63, 7.53, 7.43, 7.35-7.31, 6.87, 6.49, 5.83, 4.29, 3.95, 3.78;

LCMS: m/z 526 [M+H]+;

HPLC retention time: 1.402 min (method 9);

Chiral HPLC retention time: 1.430 min (column: CHIRALPAK IG-3 (trademark), 4.6×5 cm: 3 um, Mobile phase: MTBE:IPA=80/20, Flow: 1.0 mL/min, Temp: 25 degree).

### Example 5(26): (4R)-3-[4-[(6,7-dimethoxy-4-quinolyl)oxy]phenyl]-4-hydroxy-1-[3-(trifluoromethyl)phenyl]imidazolidin-2-one

¹H NMR (400 MHz, DMSO-d₆): δ 8.51, 8.23, 7.81, 7.63, 7.53, 7.43-7.41, 7.35-7.31, 6.87, 6.50, 5.83, 4.29, 3.95, 3.78;

LCMS: m/z 526 [M+H]+;

HPLC retention time: 1.405 min (method 9);

Chiral HPLC retention time: 2.289 min (column: CHIRALPAK IG-3 (trademark), 4.6×5 cm: 3 um, Mobile phase: MTBE:IPA=80/20, Flow: 1.0 mL/min, Temp: 25 degree).

### Example 5(27): 3-[3-ethyl-4-(imidazo[1,2-b]pyridazin-8-yloxy)phenyl]-4-hydroxy-1-[5-(trifluoromethyl)-3-pyridinyl]-2-imidazolidinone

¹H NMR (400 MHz, DMSO-d₆): δ 9.09, 8.68, 8.58, 8.33-8.29, 7.78-7.76, 7.65, 7.32, 6.99, 6.16, 5.90, 4.31, 3.88, 2.58-2.50, 1.15;

LCMS: m/z 485 [M+H]+;

HPLC retention time: 1.222 min (method 9).

### Example 5(28): (4S)-4-hydroxy-3-[4-(7-methoxyquinazolin-4-yl)oxycyclohexyl]-1-[5-(trifluoromethyl)-3-pyridyl]imidazolidin-2-one

¹H NMR (400 MHz, DMSO-d₆): δ 8.97, 8.71, 8.58, 8.52, 8.04, 7.30-7.25, 6.45, 5.42, 5.28, 4.09, 3.94, 3.71-3.65, 2.32-2.22, 2.08-1.85, 1.71-1.60;

LCMS: m/z 504 [M+H]+;

HPLC retention time: 1.214 min (method 5);

Chiral HPLC retention time: 1.711 min (column: CHIRALPAK IE-3 (trademark), 0.46×5 cm: 3 um, Mobile phase: (Hexane:DCM=3:1)(0.1% DEA):EtOH=50/50, Flow: 1.0 mL/min, Temp: 25 degree).

### Example 5(29): (4R)-4-hydroxy-3-[4-(7-methoxyquinazolin-4-yl)oxycyclohexyl]-1-[5-(trifluoromethyl)-3-pyridyl]imidazolidin-2-one

¹H NMR (400 MHz, DMSO-d₆): δ 8.97, 8.71, 8.58, 8.52, 8.04, 7.30-7.25, 6.45, 5.42, 5.28, 4.09, 3.94, 3.71-3.65, 2.32-2.22, 2.08-1.85, 1.71-1.60;

LCMS: m/z 504 [M+H]+;

HPLC retention time: 1.214 min (method 5);

Chiral HPLC retention time: 2.697 min (column: CHIRALPAK IE-3 (trademark), 0.46×5 cm: 3 um, Mobile phase: (Hexane:DCM=3:1)(0.1% DEA):EtOH=50/50, Flow: 1.0 mL/min, Temp: 25 degree).

### Example 5(30): (4R)-3-[4-[(6,7-dimethoxy-4-quinolyl)oxy]phenyl]-4-hydroxy-1-[5-(trifluoromethyl)-3-pyridyl]imidazolidin-2-one

¹H NMR (400 MHz, DMSO-d₆): δ 9.10, 8.68, 8.58, 8.52, 7.82-7.79, 7.53, 7.41, 7.35-7.33, 6.94, 6.51, 5.89-5.85, 4.34, 3.96, 3.90-3.87;

LCMS: m/z 527 [M+H]+;

HPLC retention time: 2.627 min (method 16-1);

Chiral HPLC retention time: 2.20 min (column: CHIRALPAK ID-3 (trademark), 0.46×5 cm: 3 um, Mobile phase: MTBE (0.1% DEA):EtOH=70/30, Flow: 1.0 mL/min, Temp: 25 degree).

### Example 5(31): 5-(2-ethyl-4-{5-hydroxy-2-oxo-3-[3-(trifluoromethoxy)phenyl]-1-imidazolidinyl}phenoxy)-3,4-dihydro-1,8-naphthyridin-2(1H)-one

¹H NMR (400 MHz, DMSO-d₆): δ 10.50, 7.99-7.89, 7.68, 7.65-7.42, 7.21-6.98, 6.86, 6.16, 5.78, 4.22, 3.72, 2.99, 2.57-2.49, 1.14;

LCMS: m/z 529 [M+H]+;

HPLC retention time: 1.736 min (method 1).

### Example 5(32): 3-[4-[(6,7-dimethoxy-4-quinolyl)oxy]-3-ethyl-2-methyl-phenyl]-4-hydroxy-1-[5-(trifluoromethyl)-3-pyridyl]imidazolidin-2-one

¹H NMR (400 MHz, DMSO-d₆): δ 9.07, 8.65-8.48, 7.56, 7.43-7.34, 7.31-6.98, 6.78, 6.41, 5.71-5.46, 4.40, 3.99-3.87, 2.62, 2.28, 1.08;

LCMS: m/z 569 [M+H]+;

HPLC retention time: 1.207 min (method 5).

### Example 5(33): formic acid—3-{4-[(6,7-dimethoxy-4-quinolinyl)oxy]-3-ethyl-2-fluorophenyl}-4-hydroxy-1-[5-(trifluoromethyl)-3-pyridinyl]-2-imidazolidinone (1:1)

¹H NMR (400 MHz, DMSO-d₆): δ 9.08, 8.67, 8.57-8.53, 7.54-7.44, 7.15, 6.92, 6.51, 5.67, 4.39, 3.96-3.93, 3.93-3.91, 2.67, 1.15;

LCMS: m/z 573 [M+H]+;

HPLC retention time: 1.726 min (method 22).

### Example 5(34): 3-{2-ethyl-4-[(7-methoxy-4-quinazolinyl)oxy]phenyl}-4-hydroxy-1-[5-(trifluoromethyl)-3-pyridinyl]-2-imidazolidinone

¹H NMR (400 MHz, DMSO-d₆): δ 9.07, 8.64, 8.63, 8.54, 8.28, 7.40-7.32, 7.26, 7.24, 6.83, 5.54, 4.41-4.36, 3.98, 3.91-3.88, 2.65, 1.16;

LCMS: m/z 526 [M+H]+;

HPLC retention time: 1.434 min (method 5).

### Example 5(35): 3-{2-ethyl-4-[(7-methoxy-4-quinazolinyl)oxy]phenyl}-4-hydroxy-1-[3-(trifluoromethyl)phenyl]-2-imidazolidinone

¹H NMR (400 MHz, DMSO-d₆): δ 8.68, 8.30-8.20, 7.79, 7.61, 7.44-7.23, 6.77, 5.50, 4.35, 3.99-3.78, 2.66, 1.17;

LCMS: m/z 525 [M+H]+;

HPLC retention time: 1.598 min (method 5).

### Example 5(36): 3-{3-ethyl-4-[(7-methoxy-4-quinazolinyl)oxy]-2-methylphenyl}-4-hydroxy-1-[5-(trifluoromethyl)-3-pyridinyl]-2-imidazolidinone

¹H NMR (400 MHz, DMSO-d₆): δ 9.07, 8.69-8.56, 8.33, 7.42-7.28, 7.21, 6.82, 5.58, 4.39-4.30, 3.99-3.90, 2.57, 2.26, 1.06;

LCMS: m/z 540 [M+H]+;

HPLC retention time: 1.810 min (method 17).

### Example 5(37): 3-{3-ethyl-4-[(7-methoxy-4-quinazolinyl)oxy]-2-methylphenyl}-4-hydroxy-1-[3-(trifluoromethyl)phenyl]-2-imidazolidinone

¹H NMR (400 MHz, DMSO-d₆): δ 8.66, 8.35, 8.22, 7.79, 7.61, 7.43-7.29, 7.20, 6.74, 5.52, 4.36, 3.99, 3.80, 2.57, 2.26, 1.02;

LCMS: m/z 539 [M+H]+;

HPLC retention time: 1.626 min (method 5).

### Example 5(38): 4-hydroxy-3-{4-[(7-methoxy-4-quinazolinyl)oxy]bicyclo[2.2.1]hept-1-yl}-1-[5-(trifluoromethyl)-3-pyridinyl]-2-imidazolidinone

¹H NMR (400 MHz, DMSO-d₆): δ 9.07, 8.73-8.70, 8.49, 8.06, 7.28-7.25, 4.56, 3.94, 2.88, 2.42-2.32, 2.14-2.04;

LCMS: m/z 516 [M+H]+;

HPLC retention time: 1.731 min (method 1).

### Example 5(39): (4S)-4-hydroxy-3-[4-(1H-pyrrolo[2,3-b]pyridin-5-yloxy)phenyl]-1-[3-(trifluoromethyl)phenyl]imidazolidin-2-one

¹H NMR (400 MHz, DMSO-d₆): δ 11.73, 8.19, 8.09, 7.77-7.72, 7.68, 7.66-7.53, 7.39, 7.07-6.98, 6.74, 6.44-6.42, 5.70, 4.25-4.21, 3.73-3.70;

LCMS: m/z 455 [M+H]+;

HPLC retention time: 1.553 min (method 9);

Chiral HPLC retention time: 1.286 min (column: CHIRALPAK IG-3 (trademark), 0.46×5 cm: 3 um, Mobile phase: (Hexane:DCM=3:1):EtOH=70:30, Flow: 1.0 mL/min, Temp: 25 degree).

### Example 5(40): 4-hydroxy-3-[4-(1H-pyrrolo[2,3-b]pyridin-5-yloxy)phenyl]-1-[5-(trifluoromethyl)-3-pyridinyl]-2-imidazolidinone

¹H NMR (400 MHz, DMSO-d₆): δ 11.74, 9.05, 8.64-8.54, 8.08, 7.68, 7.58-7.52, 7.02-6.99, 6.82, 6.44, 5.75, 4.27, 3.83;

LCMS: m/z 456 [M+H]+;

HPLC retention time: 2.699 min (method 50).

### Example 5(41): 3-[3-ethyl-4-(1H-pyrrolo[2,3-b]pyridin-5-yloxy)phenyl]-4-hydroxy-1-[3-(trifluoromethyl)phenyl]-2-imidazolidinone

¹H NMR (400 MHz, DMSO-d₆): δ 11.67, 8.21, 8.05, 7.76, 7.62-7.58, 7.51-7.40, 7.39, 6.77, 6.40, 5.73, 4.24, 3.72, 2.69, 1.22;

LCMS: m/z 483 [M+H]+;

HPLC retention time: 1.838 min (method 1).

### Example 5(42): (4R)-4-hydroxy-3-[4-(1H-pyrrolo[2,3-b]pyridin-5-yloxy)phenyl]-1-[3-(trifluoromethyl)phenyl]imidazolidin-2-one

¹H NMR (400 MHz, DMSO-d₆): δ 11.73, 8.19, 8.09, 7.77-7.72, 7.68, 7.66-7.53, 7.39, 7.07-6.98, 6.74, 6.44-6.42, 5.70, 4.25-4.21, 3.73-3.70;

LCMS: m/z 455 [M+H]+;

HPLC retention time: 1.553 min (method 9);

Chiral HPLC retention time: 1.533 min (column: CHIRALPAK IG-3 (trademark), 0.46×5 cm: 3 um, Mobile phase: (Hexane:DCM=3:1):EtOH=70:30, Flow: 1.0 mL/min, Temp: 25 degree).

### Example 5(43): 4-hydroxy-3-[4-(1H-pyrrolo[2,3-b]pyridin-4-yloxy)phenyl]-1-[3-(trifluoromethyl)phenyl]-2-imidazolidinone

¹H NMR (300 MHz, CDCl₃): δ 3.38, 3.77-3.92, 4.20-4.32, 5.67-5.77, 6.37-6.44, 6.46-6.56, 7.22, 7.33-7.41, 7.44-7.56, 7.68, 7.76-7.84, 7.88-7.97, 8.00-8.10;

LCMS: m/z 455 [M+H]+;

HPLC retention time: 0.86 min (method 83).

### Example 5(44): 8-[2-ethyl-4-[(5S)-5-hydroxy-2-oxo-3-[5-(trifluoromethyl)-3-pyridyl]imidazolidin-1-yl]phenoxy]-4H-pyrido[2,3-b]pyrazin-3-one

¹H NMR (400 MHz, DMSO-d₆): δ 12.91, 9.08, 8.67, 8.57, 8.34, 8.21, 7.74, 7.61, 7.21, 6.91, 6.43, 5.87, 4.30, 3.87, 2.54, 1.14;

LCMS: m/z 513 [M+H]+;

HPLC retention time: 1.422 min (method 1);

Chiral HPLC retention time: 2.112 min (column: CHIRALPAK ID-3 (trademark), 0.46×5 cm: 3 um, Mobile phase: MTBE:MeOH=90:10, Flow: 1.0 mL/min, Temp: 25 degree).

### Example 5(45): 8-[2-ethyl-4-[(5R)-5-hydroxy-2-oxo-3-[5-(trifluoromethyl)-3-pyridyl]imidazolidin-1-yl]phenoxy]-4H-pyrido[2,3-b]pyrazin-3-one

¹H NMR (400 MHz, DMSO-d₆): δ 12.91, 9.08, 8.67, 8.57, 8.34, 8.21, 7.74, 7.61, 7.21, 6.91, 6.43, 5.88, 4.31, 3.87, 2.59-2.50, 1.14;

LCMS: m/z 513 [M+H]+;

HPLC retention time: 1.412 min (method 1);

Chiral HPLC retention time: 3.359 min (column: CHIRALPAK ID-3 (trademark), 0.46×5 cm: 3 um, Mobile phase: MTBE:MeOH=90:10, Flow: 1.0 mL/min, Temp: 25 degree).

### Example 5(46): 8-(2-ethyl-4-{5-hydroxy-2-oxo-3-[5-(trifluoromethyl)-3-pyridinyl]-1-imidazolidinyl}phenoxy)pyrido[2,3-b]pyrazin-3(4H)-one

¹H NMR (400 MHz, DMSO-d₆): δ 12.92, 9.08, 8.66, 8.57, 8.34, 8.21, 7.74, 7.61, 7.21, 6.92, 6.42, 5.88, 4.30, 3.87, 2.55, 1.14;

LCMS: m/z 513 [M+H]+;

HPLC retention time: 2.200 min (method 28).

### Reference Example 13: 4-(4-nitrobenzyl)pyridine

To a solution of t-BuOK (2.05 g) in DMSO (7 mL) the solution of 1,4-dinitrobenzene (CAS No. 100-25-4, 0.32 mL) in DMSO (7 mL) and 4-(chloromethyl)pyridine hydrochloride (CAS No. 1822-51-1, 0.32 mL) in DMSO (6 mL) were added at dropwise under nitrogen atmosphere. The reaction mixture was stirred for 1 h at room temperature. The reaction mixture was quenched with saturated aqueous NH₄Cl. The resulting solution was extracted with ethyl acetate and the organic layers were combined, dried over anhydrous sodium sulfate, filtered and concentrated under reduced pressure to afford crude product. The crude product was purified by column chromatography, eluted with ethyl acetate/petroleum ether to afford the title compound (300 mg) having the following physical property values.

¹H-NMR (DMSO-d₆): δ 8.50, 8.22, 7.56, 7.25, 4.15.

### Example 6: 1-(3-isopropoxyphenyl)-3-[4-(4-pyridinylmethyl)phenyl]-2,4-imidazolidinedione

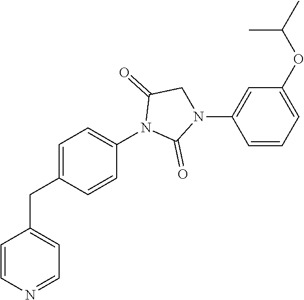 [SMILES: CC(C)Oc1cccc(N2CC(=O)N(c3ccc(Cc4ccncc4)cc3)C2=O)c1]

A procedure for a purpose similar to that for Reference Example 7→Example 1 was carried out by using a compound produced in Reference Example 13 instead of a compound produced in Reference Example 6 and a corresponding chlorocarbonyl compound instead of the compound prepared in Reference Example 5, to give the title compound having the following physical property values; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

¹H NMR (400 MHz, DMSO-d₆): δ 8.49-8.48, 7.45-7.40, 7.35-7.32, 7.31-7.25, 7.20-7.17, 6.74-6.71, 4.63-4.60, 4.03, 1.28-1.26;

LCMS: m/z 402 [M+H]+;

HPLC retention time: 2.743 min (method 16-2).

### Example 6(1)

A procedure for a purpose similar to that for Reference Example 13→Reference Example 7→Example 1 was carried out by using 1,4-dinitrobenzene, a corresponding halogen compound instead of 4-(chloromethyl)pyridine hydrochloride, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 6(1): 3-{4-[(6,7-dimethoxy-4-quinolinyl)methyl]phenyl}-1-[3-(trifluoromethyl)phenyl]-2,4-imidazolidinedione

¹H NMR (400 MHz, DMSO-d₆): δ 8.62, 8.16, 7.86, 7.67, 7.52, 7.45, 7.41-7.22, 7.20, 4.68, 4.50, 3.92, 3.89;

LCMS: m/z 522 [M+H]+;

HPLC retention time: 1.861 min (method 3).

### Reference Example 14: 5-(2-bromo-4-nitroanilino)-3,4-dihydro-1H-1,8-naphthyridin-2-one

To a mixture of 2-bromo-4-nitroaniline (CAS No. 13296-94-1, 867 mg) and Cs₂CO₃ (1950 mg) in DMSO (10 mL) was added 5-fluoro-3,4-dihydro-1H-1,8-naphthyridin-2-one (CAS No. 1237535-78-2, 332 mg) at room temperature. The reaction was heated at 100° C. and stirred overnight. Cooled down to room temperature, the reaction was quenched by water. After filtration, the precipitate was washed with water, EtOH and Et₂O, then evacuated under vacuum to afford the title compound (637 mg).

### Example 7: 3-{3-bromo-4-[(7-oxo-5,6,7,8-tetrahydro-1,8-naphthyridin-4-yl)amino]phenyl}-1-[5-(trifluoromethyl)-3-pyridinyl]-2,4-imidazolidinedione

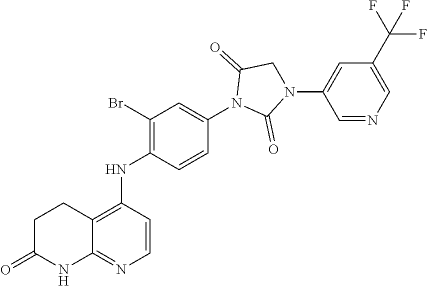 [SMILES: O=C1CCc2c(Nc3ccc(N4C(=O)CN(c5cncc(C(F)(F)F)c5)C4=O)cc3Br)ccnc2N1]

A procedure for a purpose similar to that for Reference Example 7→Example 1 was carried out by using a compound produced in Reference Example 14 instead of the compound produced in Reference Example 6 and the compound prepared in Reference Example 5 to give the title compound having the following physical property values.

¹H NMR (400 MHz, DMSO-d₆): δ 10.52, 9.19, 8.79, 8.53, 7.87-7.83, 7.55, 7.47, 6.19, 4.78, 2.87, 2.60;

LCMS: m/z 561 [M+H]+;

HPLC retention time: 1.213 min (method 43).

### Example 7(1)˜7(7)

A procedure for a purpose similar to that for Reference Example 14→Reference Example 7→Example 1 was carried out by using a corresponding halogen compound instead of 5-fluoro-3,4-dihydro-1H-1,8-naphthyridin-2-one, a corresponding aniline compound instead of 2-bromo-4-nitroaniline, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 7(1): 3-{4-[(6,7-dimethoxy-4-quinolinyl)(methyl)amino]phenyl}-1-[5-(trifluoromethyl)-3-pyridinyl]-2,4-imidazolidinedione

¹H NMR (400 MHz, DMSO-d₆): δ 9.17, 8.78, 8.70, 8.51, 7.38-7.34, 7.29, 7.13, 6.77, 4.75, 3.93, 3.56, 3.51;

LCMS: m/z 538 [M+H]+;

HPLC retention time: 2.440 min (method 42).

### Example 7(2): 3-{4-[(6,7-dimethoxy-4-quinolinyl)amino]phenyl}-1-[5-(trifluoromethyl)-3-pyridinyl]-2,4-imidazolidinedione

¹H NMR (400 MHz, DMSO-d₆): δ 9.19, 8.88, 8.79, 8.54, 8.35, 7.73-7.66, 7.48-7.42, 7.28, 6.99, 4.77, 3.95, 3.92;

LCMS: m/z 524 [M+H]+;

HPLC retention time: 1.661 min (method 3).

### Example 7(3): 3-[4-(1H-pyrrolo[2,3-b]pyridin-4-ylamino)phenyl]-1-[3-(trifluoromethyl)phenyl]-2,4-imidazolidinedione

¹H NMR (300 MHz, DMSO-d₆): δ 4.41-4.91, 6.50-6.67, 6.70-6.86, 7.14-7.29, 7.37, 7.45-7.58, 7.58-7.74, 7.81-7.98, 8.09-8.27, 8.66-8.89, 11.11-11.61;

LCMS: m/z 452 [M+H]+;

TLC Rf: 0.32 (AcOEt/MeOH, 19/1).

### Example 7(4): 3-{3-ethyl-4-[(5-fluoro-7-methoxy-4-quinazolinyl)amino]phenyl}-1-[5-(trifluoromethyl)-3-pyridinyl]-2,4-imidazolidinedione

¹H NMR (400 MHz, DMSO-d₆): δ 9.19, 9.00, 8.79, 8.55, 8.39, 7.64, 7.39-7.31, 7.16-7.06, 4.79-4.76, 3.94, 2.64, 1.15;

LCMS: m/z 541 [M+H]+;

HPLC retention time: 1.200 min (method 5).

### Example 7(5): 3-{3-bromo-4-[(5-fluoro-7-methoxy-4-quinazolinyl)amino]phenyl}-1-[5-(trifluoromethyl)-3-pyridinyl]-2,4-imidazolidinedione

¹H NMR (400 MHz, DMSO-d₆): δ 9.20-9.17, 8.80, 8.54-8.52, 8.16, 7.85, 7.55, 7.22-7.12, 4.80, 3.95;

LCMS: m/z 591 [M+H]+;

HPLC retention time: 1.265 min (method 5).

### Example 7(6): 3-{4-[(5-fluoro-7-methoxy-4-quinazolinyl)amino]-3-vinylphenyl}-1-[5-(trifluoromethyl)-3-pyridinyl]-2,4-imidazolidinedione

¹H NMR (400 MHz, DMSO-d₆): δ 9.21-9.11, 8.80, 8.55, 8.37, 7.76, 7.61, 7.41, 7.16-7.07, 6.82, 5.78, 5.34, 4.81, 3.94;

LCMS: m/z 539 [M+H]+;

HPLC retention time: 1.192 min (method 5).

### Example 7(7): 3-{4-[(2-amino-5-pyrimidinyl)amino]phenyl}-1-[5-(trifluoromethyl)-3-pyridinyl]-2,4-imidazolidinedione

¹H NMR (400 MHz, DMSO-d₆) δ 9.15, 8.76, 8.50, 8.16, 7.79, 7.14, 6.79, 6.48, 4.71;

LCMS: m/z 430 [M+H]+;

HPLC retention time: 1.240 min (method 67).

### Reference Example 15: 1-(2-bromo-4-nitrophenyl)-3,4-dihydro-2H-1,6-naphthyridine

To a stirred mixture of 1,2,3,4-tetrahydro-1,6-naphthyridine (CAS No. 13623-84-2, 500 mg) and K₂CO₃ (1028 mg) in DMF (30 mL) were added 2-bromo-1-fluoro-4-nitrobenzene (CAS No. 701-45-1, 820 mg) in portions at room temperature under nitrogen atmosphere. The resulting mixture was stirred for 2 h at 80° C. The reaction was then quenched by water, extracted with EtOAc. The organic extracts were washed with water and brine, dried over anhydrous sodium sulfate. After filtration, the filtrate was concentrated under reduced pressure. The residue was purified by Prep-TLC (DCM/MeOH=10:1) to afford the title compound (720 mg).

### Example 8: 3-[3-bromo-4-(3,4-dihydro-1,6-naphthyridin-1(2H)-yl)phenyl]-1-[5-(trifluoromethyl)-3-pyridinyl]-2,4-imidazolidinedione

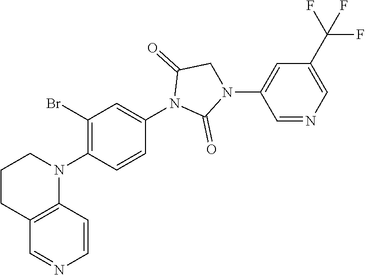 [SMILES: O=C1CN(c2cncc(C(F)(F)F)c2)C(=O)N1c1ccc(N2CCCc3cnccc32)c(Br)c1]

A procedure for a purpose similar to that for Reference Example 7→Example 1 was carried out by using a compound produced in Reference Example 15 instead of a compound produced in Reference Example 6 and the compound prepared in Reference Example 5 to give the title compound having the following physical property values.

¹H NMR (400 MHz, DMSO-d₆): δ 9.19, 8.79, 8.52, 8.26, 8.07, 7.80, 7.74, 6.14, 4.80, 3.82-3.78, 3.68-3.45, 2.92, 2.19-2.16, 2.07-2.04;

LCMS: m/z 533 [M+H]+;

HPLC retention time: 2.329 min (method 73).

### Example 8(1)˜8(5)

A procedure for a purpose similar to that for Reference Example 15→Reference Example 7→Example 1 was carried out by using a corresponding amine compound instead of 1,2,3,4-tetrahydro-1,6-naphthyridine, a corresponding halogen compound instead of 2-bromo-1-fluoro-4-nitrobenzene, and the compound prepared in Reference Example 5, to give the following compounds of Examples.

### Example 8(1): 3-[4-(3,4-dihydro-1,6-naphthyridin-1(2H)-yl)-3-vinylphenyl]-1-[5-(trifluoromethyl)-3-pyridinyl]-2,4-imidazolidinedione

¹H NMR (300 MHz, DMSO-d₆): δ 9.17, 8.77, 8.53, 7.98, 7.85-7.81, 7.51-7.44, 6.67-6.60, 5.87-5.76, 5.38, 4.77, 3.65-3.62, 2.82-2.79, 2.07-2.02;

LCMS: m/z 480 [M+H]+;

HPLC retention time: 1.141 min (method 5).

### Example 8(2): formic acid—3-[4-(3,4-dihydro-1,6-naphthyridin-1(2H)-yl)-3-ethylphenyl]-1-[5-(trifluoromethyl)-3-pyridinyl]-2,4-imidazolidinedione (1:1)

¹H NMR (400 MHz, DMSO-d₆): δ 9.19, 8.79, 8.55, 8.19, 8.01, 7.85, 7.51, 7.44-7.40, 5.77, 4.79, 3.65, 3.49, 2.84-2.78, 2.62-2.51, 2.11-2.01, 1.14;

LCMS: m/z 482 [M+H]+;

HPLC retention time: 1.204 min (method 1).

### Example 8(3): 3-[4-(2,3-dihydro-1H-pyrrolo[3,2-c]pyridin-1-yl)-3-ethylphenyl]-1-[5-(trifluoromethyl)-3-pyridinyl]-2,4-imidazolidinedione

¹H NMR (400 MHz, DMSO-d₆): δ 9.19, 8.79, 8.54, 8.16, 8.01, 7.47-7.35, 6.05, 4.78, 3.95, 3.22, 2.64, 1.17;

LCMS: m/z 468 [M+H]+;

HPLC retention time: 1.128 min (method 5).

### Example 8(4): 3-[4-(2,3-dihydro-1H-pyrrolo[2,3-c]pyridin-1-yl)-3-ethylphenyl]-1-[5-(trifluoromethyl)-3-pyridinyl]-2,4-imidazolidinedione trifluoroacetate (1:1)

¹H NMR (400 MHz, DMSO-d₆): δ 9.19, 8.79, 8.53, 8.12, 7.74, 7.58 (z, 1H), 7.52-7.50, 7.42-7.39, 4.79, 4.05, 3.48, 2.64, 1.19;

LCMS: m/z 468 [M+H]+;

HPLC retention time: 1.406 min (method 57).

### Example 8(5): 3-[3-bromo-4-(2,3-dihydro-1H-pyrrolo[3,2-c]pyridin-1-yl)phenyl]-1-[5-(trifluoromethyl)-3-pyridinyl]-2,4-imidazolidinedione

¹H NMR (400 MHz, DMSO-d₆): δ 9.20, 8.81, 8.53, 8.22, 8.14, 7.92, 7.73, 7.63, 6.29, 4.80, 4.10, 3.26;

LCMS: m/z 518 [M+H]+;

HPLC retention time: 1.123 min (method 5).

### Reference Example 16: tert-butyl 6-[2,5-dioxo-3-[5-(trifluoromethyl)-3-pyridyl]imidazolidin-1-yl]-3,4-dihydro-2H-quinoline-1-carboxylate

To a stirred solution of tert-butyl 6-amino-3,4-dihydro-2H-quinoline-1-carboxylate (CAS No. 1152923-36-8, 200 mg) in THF (10 mL) was added DIEA (520 mg) and the compound prepared in Reference Example 5 (239 mg) at room temperature under N₂ atmosphere. The resulting mixture was stirred for 4 hours at 80° C. under N₂ atmosphere. The mixture was allowed to cool down to 25° C. The mixture was concentrated under reduced pressure. The residue was purified by silica gel column chromatography, eluted with (ethyl acetate:Petroleum ether=1:4) to afford the title compound (360 mg).

### Reference Example 17: 3-(1,2,3,4-tetrahydroquinolin-6-yl)-1-[5-(trifluoromethyl)-3-pyridyl]imidazolidine-2,4-dione

To a stirred solution of the compound prepared in Reference Example 16 (360 mg) in DCM (36 mL) was added NH₃ in MeOH (7N, 5 mL) and TFA (5.5 g) at room temperature under N₂ atmosphere. The resulting mixture was stirred for 4 hours at 80° C. under N₂ atmosphere. The mixture was allowed to cool down to 25° C. The mixture was concentrated under reduced pressure. The residue was purified by silica gel column chromatography, eluted with (ethyl acetate:Petroleum ether=1:4) to afford the title compound (298 mg).

### Example 9: 3-(6′,7′-dimethoxy-3,4-dihydro-2H-1,4′-biquinolin-6-yl)-1-[5-(trifluoromethyl)-3-pyridinyl]-2,4-imidazolidinedione trifluoroacetate (1:1)

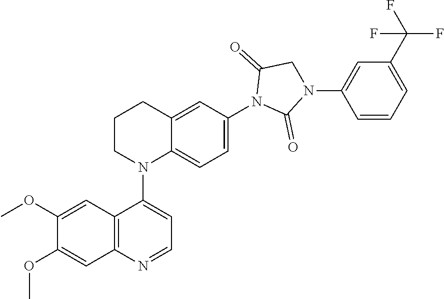 [SMILES: COc1cc2nccc(N3CCCc4cc(N5C(=O)CN(c6cccc(C(F)(F)F)c6)C5=O)ccc43)c2cc1OC]

To a stirred solution of the compound prepared in Reference Example 17 (100 mg) and 4-chloro-6,7-dimethoxyquinoline (CAS No. 35654-56-9, 89 mg) and Cs₂CO₃ (260 mg) in toluene (2 mL) were added Xphos (51 mg) and Pd(OAc)₂ (12 mg) at room temperature under N₂ atmosphere. The resulting mixture was stirred overnight at 105° C. under N₂ atmosphere. The mixture was allowed to cool down to 25° C. The mixture was concentrated under reduced pressure. The crude product was purified by Prep-HPLC to afford the title compound (20 mg) having the following physical property values.

¹H NMR (400 MHz, DMSO-d₆): δ 9.17, 8.78, 8.51, 7.52, 7.51-7.41, 7.36, 7.09, 6.93, 6.87, 4.75, 4.03-3.99, 3.62, 2.99, 2.11-2.07;

LCMS: m/z 564 [M+H]+;

HPLC retention time: 1.382 min (method 43).

### Example 9(1)

A procedure for a purpose similar to that for Reference Example 16→Reference Example 17→Example 9 was carried out by using a corresponding amine compound instead of tert-butyl 6-amino-3,4-dihydro-2H-quinoline-1-carboxylate, the compound prepared in Reference Example 5, and 4-chloro-6,7-dimethoxyquinoline, to give the following compounds of Examples.

### Example 9(1): 3-[1-(6,7-dimethoxy-4-quinolinyl)-2,3-dihydro-1H-indol-5-yl]-1-[5-(trifluoromethyl)-3-pyridinyl]-2,4-imidazolidinedione

¹H NMR (300 MHz, DMSO-d₆): δ 9.16, 8.76, 8.63, 8.52, 7.42, 7.29, 7.11, 7.02, 6.43, 4.73, 4.13, 3.99, 3.76, 3.29-3.24;

LCMS: m/z 550 [M+H]+;

HPLC retention time: 2.490 min (method 42).

### Reference Example 18: 7-methoxyquinoline-4-carbonitrile

To a solution of 4-chloro-7-methoxyquinoline (CAS No. 68500-37-8, 600 mg) in DMF (10 mL) was added Zn(CN)₂ (379 mg), 1,1′-Bis(diphenylphosphino)ferrocene (CAS No. 12150-46-8, 387 mg), Pd₂(dba)₃ (341 mg) and Zn (15.8 mg) under nitrogen atmosphere. The resulting mixture was stirred at 80° C. overnight. Then the reaction cooled to room temperature, diluted with ethyl acetate, washed with brine, dried over anhydrous Na₂SO₄ and evaporated to give the crude. The residue was purified by silica gel column chromatography, eluted with Petroleum ether/ethyl acetate=3/1 to afford the title compound (170 mg) having the following physical property values.

LCMS condition: Column: Ascentis Express C18 50 (trademark); Length: 50 mm, Internal Diameter: 3.0 mm; Mobile phase A: Water+0.05% TFA; Mobile phase B: MeCN+0.05% TFA; Time: 1.10 min; Rt: 0.846 min.

ESI-MS m/z: 185.1 [M+H]+.

### Reference Example 19: 7-methoxyquinoline-4-carbaldehyde

To a solution of the compound prepared in Reference Example 18 (90 mg) in toluene (5 mL) was add DIBAL-H (0.49 mL, 1M in toluene) at 0° C. Then warmed to room temperature and stirred overnight. The resulting mixture was concentrated under vacuum to afford the title compound (54 mg) having the following physical property values.

LCMS condition: Column: X select CSH C18 (trademark); Length: 50 mm, Internal Diameter: 3.0 mm; Mobile phase A: Water+0.1% FA; Mobile phase B: MeCN+0.1% FA; Time: 1.10 min; Rt: 1.030 min; ESI-MS m/z: 188.1 [M+H]+.

### Reference Example 20: 3-(piperidin-4-yl)-1-[5-(trifluoromethyl)pyridin-3-yl]imidazolidine-2,4-dione

A procedure for a purpose similar to that for Reference Example 16→Reference Example 17 was carried out by using 4-amino-1-tert-butoxycarbonylpiperidine (CAS No. 87120-72-7) instead of tert-butyl 6-amino-3,4-dihydro-2H-quinoline-1-carboxylate, and the compound prepared in Reference Example 5, to give the title compound.

### Example 10: 3-{1-[(7-methoxy-4-quinolinyl)methyl]-4-piperidinyl}-1-[5-(trifluoromethyl)-3-pyridinyl]-2,4-imidazolidinedione

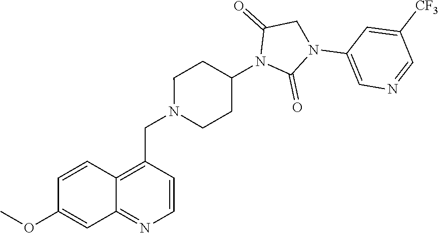 [SMILES: COc1ccc2c(CN3CCC(N4C(=O)CN(c5cncc(C(F)(F)F)c5)C4=O)CC3)ccnc2c1]

To a solution of the compound prepared in Reference Example 19 (54 mg) in THF (3 mL) was added the compound prepared in Reference Example 20 (95 mg). After stirring for 15 min, the reaction mixture was added NaBH(AcO)₃ (123 mg). The reaction was stirred at room temperature overnight. The resulting mixture was extracted with DCM, washed with water, brine and dried over anhydrous Na₂SO₄. After filtration, the filtrate was concentrated under reduced pressure. The residue was purified by Prep-TLC (DCM/MeOH=10:1) to afford the title compound (49 mg) having the following physical property values.

¹H NMR (400 MHz, DMSO-d₆): δ 9.00, 8.71-8.66, 8.39, 8.15, 7.34-7.22, 4.48, 3.88-3.85, 3.09-2.94, 2.29-2.11, 1.61-1.58;

LCMS: m/z 500 [M+H]+;

HPLC retention time: 1.807 min (method 3).

### Example 10(1)˜ 10(2)

A procedure for a purpose similar to that for Example 10 was carried out by using a corresponding carbonyl compound instead of the compound prepared in Reference Example 19 and the compound prepared in Reference Example 20 to give the following compounds of Examples.

### Example 10(1): 3-{1-[1-(1H-pyrrolo[2,3-b]pyridin-4-yl)ethyl]-4-piperidinyl}-1-[5-(trifluoromethyl)-3-pyridinyl]-2,4-imidazolidinedione

¹H NMR (400 MHz, CD₃OD): δ 9.06, 8.64, 8.53, 8.38, 8.28, 7.49, 7.27, 6.81, 4.50, 4.20-4.14, 3.68, 3.25, 2.82-2.59, 1.96-1.88, 1.81, 1.71;

LCMS: m/z 473 [M+H]+;

HPLC retention time: 1.013 min (method 19).

### Example 10(2): 3-{1[1-(6,7-dimethoxy-4-quinolinyl)ethyl]-4-piperidinyl}-1-[5-(trifluoromethyl)-3-pyridinyl]-2,4-imidazolidinedione

¹H NMR (400 MHz, CD₃OD): δ 9.07, 8.65, 8.59, 8.53, 8.19, 7.45, 7.38, 4.48, 4.20-4.15, 4.12-4.03, 3.34-3.29, 2.95, 2.61-2.49, 2.28-2.22, 1.82-1.76, 1.69-1.63, 1.54-1.52;

LCMS: m/z 544 [M+H]+;

HPLC retention time: 0.993 min (method 5).

### Reference Example 21: 4-[(6,7-dimethoxyquinolin-4-yl)thio]aniline

To a solution of 4-chloro-6,7-dimethoxyquinoline (3.0 g) in DMF (30 mL) was added 4-aminothiphenol (CAS No. 1193-02-8, 2.5 g) and the mixture was stirred at room temperature for 18 h. The mixture was concentrated under reduced pressure and the residue was dissolved in ethyl acetate and washed with water and brine dried (MgSO₄) and concentrated under reduced pressure. The residue was purified by column chromatography on silica gel (elution with 0-5% methanol in ethyl acetate) to give the title compound (2.6 g) having the following physical property values.

¹H NMR (400 MHz, DMSO): δ 8.38, 7.37, 7.30-7.25, 6.73-6.70, 6.54, 5.70, 3.95, 3.94.

### Example 11: 3-{4-[(6,7-dimethoxy-4-quinolinyl)thio]phenyl}-1-[3-(trifluoromethyl)phenyl]-2,4-imidazolidinedione

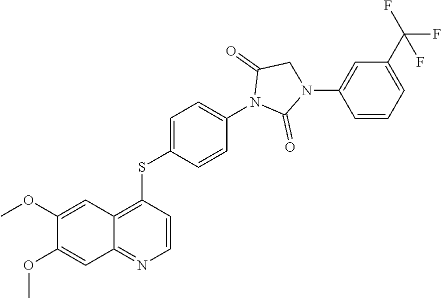 [SMILES: COc1cc2nccc(Sc3ccc(N4C(=O)CN(c5cccc(C(F)(F)F)c5)C4=O)cc3)c2cc1OC]

Into a solution of methyl (3-(trifluoromethyl)phenyl)glycinate (CAS No. 126689-78-9, 0.06 g) and DIEA (0.13 mL) in toluene (2.0 mL) at 0° C., was added trichloromethyl chloroformate (0.037 mL) in toluene (2.0 ml) dropwise. The resulting solution was stirred at 70° C. under nitrogen for 1 h. The resulting mixture was cooled to room temperature and concentrated under reduced pressure. The residue was suspended in THF (2.0 mL) and added to a solution of the compound prepared in Reference Example 21 (0.08 g) and triethylamine (0.11 mL) in THF (2.0 mL) and the resulting mixture was heated at reflux for 16 h. The mixture was cooled to room temperature and diluted with ethyl acetate then washed with saturated aqueous ammonium chloride solution and brine, dried (MgSO4), filtered and concentrated under reduced pressure. The residue was purified by preparative HPLC to give the title compound having the following physical property values.

¹H NMR (400 MHz, DMSO-d₆): δ 8.56, 8.20, 7.94-7.89, 7.74-7.67, 7.61-7.53, 7.44, 7.37, 6.97, 4.73, 3.97, 3.91;

MS: m/z 540 (M+H)+;

HPLC retention time: 3.08 min (method 76).

### Example 11(1)˜11(5)

A procedure for a purpose similar to that for Reference Example 20→Example 1 was carried out by using a corresponding halide compound instead of 4-chloro-6,7-dimethoxyquinoline, a corresponding amine compound instead of 4-aminothiphenol, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 11(1): 3-(1-{[6-(benzylamino)-4-pyrimidinyl]sulfonyl}-4-piperidinyl)-1-[3-(trifluoromethyl)phenyl]-2,4-imidazolidinedione

¹H NMR (400 MHz, DMSO-d₆): δ 8.57-8.55, 8.10, 7.80, 7.64, 7.48, 7.35-7.29, 7.28-7.27, 7.04, 4.63, 4.49, 4.10-3.98, 3.83-3.80, 2.91, 2.33-2.27, 1.74-1.71;

LCMS: m/z 575 [M+H]+;

HPLC retention time: 2.037 min (method 38).

### Example 11(2): 3-{4-[(6,7-dimethoxy-4-quinolinyl)sulfinyl]phenyl}-1-[5-(trifluoromethyl)-3-pyridinyl]-2,4-imidazolidinedione

¹H NMR (300 MHz, DMSO-d₆): δ 9.14, 8.95, 8.77, 8.48, 8.03-7.97, 7.62, 7.49, 7.39, 4.74, 3.93;

LCMS: m/z 557 [M+H]+;

HPLC retention time: 1.177 min (method 51).

### Example 11(3): 3-[4-(1H-pyrrolo[2,3-b]pyridin-4-ylthio)phenyl]-1-[3-(trifluoromethyl)phenyl]-2,4-imidazolidinedione trifluoroacetate (1:1)

¹H NMR (300 MHz, DMSO-d₆): δ 8.16, 8.03, 7.84, 7.52-7.49, 7.38, 7.24, 7.16, 6.76, 6.22, 4.83, 4.70, 2.19;

LCMS: m/z 462 [M+H]+;

HPLC retention time: 1.455 min (method 1).

### Example 11(4): 3-[4-(1H-pyrrolo[2,3-b]pyridin-4-ylsulfonyl)phenyl]-1-[3-(trifluoromethyl)phenyl]-2,4-imidazolidinedione

¹H NMR (400 MHz, DMSO-d₆): δ 9.19, 8.80, 8.50, 8.18, 7.76, 7.63, 7.42, 6.80, 6.28, 5.03, 4.85;

LCMS: m/z 509 [M+H]+;

HPLC retention time: 1.297 min (method 1).

### Example 11(5): 3-{4-[(6,7-dimethoxy-4-quinolinyl)thio]phenyl}-1-[5-(trifluoromethyl)-3-pyridinyl]-2,4-imidazolidinedione

¹H NMR (400 MHz, DMSO-d₆): δ 9.20, 8.82-8.80, 8.57-8.54, 7.74-7.69, 7.61-7.57, 7.45, 7.37, 6.99, 4.81-4.79, 3.97, 3.91;

MS: m/z 541 (M+H)+;

HPLC retention time: 2.92 min (method 76).

### Reference Example 22: 4-(4-bromo-2-ethyl-phenoxy)pyrimidin-2-amine

To a stirred solution of 4-bromo-2-ethylphenol (CAS No. 18980-21-7, 500 mg) in DMF (50 mL) was added NaH (200 mg) and 2-amino-4-chloropyrimidine (483 mg) in portions at 0oC under N₂ atmosphere. The resulting mixture was stirred for 4 h at 110° C. under N₂ atmosphere. The mixture was allowed to cool down to 25° C. The resulting mixture was diluted with ethyl acetate and water. The resulting mixture was extracted with ethyl acetate. The combined organic layers were washed with water then washed with saturated brine solution, dried over anhydrous Na₂SO₄. After filtration, the filtrate was concentrated under reduced pressure. The residue was purified by silica gel column chromatography, eluted with (DCM:MeOH=25:1) to afford the title compound (585 mg).

### Reference Example 23: 3-[4-(2-aminopyrimidin-4-yl)oxy-3-ethyl-phenyl]-5,5-dimethyl-imidazolidine-2,4-dione

To a stirred solution of the compound prepared in Reference Example 22 (140 mg) in DMF (5 mL) was added Cu₂O (68 mg) and 5,5-dimethylimidazolidine-2,4-dione (CAS No. 77-71-4, 92 mg) at room temperature under N₂ atmosphere. The resulting mixture was stirred for 14 h at 150° C. under N₂ atmosphere. The mixture was allowed to cool down to 25° C. The resulting mixture was diluted with DCM and water. The resulting mixture was extracted with DCM. The combined organic layers were washed with water, dried over anhydrous Na₂SO₄. After filtration, the filtrate was concentrated under reduced pressure. The residue was purified by silica gel column chromatography, eluted with (acetone:toluene=1:2) to afford the title compound (28 mg) having the following physical property values.

¹H NMR (400 MHz, CDCl₃): δ 8.11, 7.39, 7.32, 7.15, 6.25, 5.50.

### Example 12: 3-{4-[(2-amino-4-pyrimidinyl)oxy]-3-ethylphenyl}-5,5-dimethyl-1-[5-(trifluoromethyl)-3-pyridinyl]-2,4-imidazolidinedione

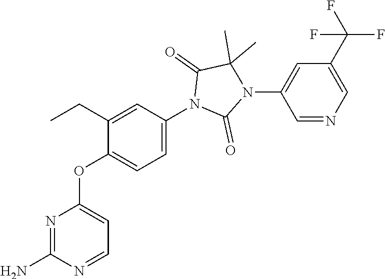 [SMILES: CCc1cc(N2C(=O)N(c3cncc(C(F)(F)F)c3)C(C)(C)C2=O)ccc1Oc1ccnc(N)n1]

To a stirred solution of the compound prepared in Reference Example 23 (11 mg) and 3-iodo-5-(trifluoromethyl)pyridine (CAS No. 1214333-22-8, 12 mg) and K₂CO₃ (8.9 mg) and trans-N,N′-dimethylcyclohexane-1,2-diamine (CAS No. 67579-81-1, 1.8 mg) in toluene (1 mL) was added CuI (1.2 mg) at room temperature under N₂ atmosphere. The resulting mixture was stirred overnight at 110° C. under N₂ atmosphere. The mixture was allowed to cool down to 25° C. The resulting mixture was filtered, the filter cake was washed with DCM and MeOH. After filtration, the filtrate was concentrated under reduced pressure. The residue was purified by silica gel column chromatography, and by Prep-TLC (DCM:MeOH=30:1) to afford the title compound (1.2 mg) having the following physical property values.

¹H NMR (400 MHz, CD₃OD): δ 8.87, 8.60, 8.47-8.43, 8.38, 7.42, 7.35, 7.23, 6.61, 2.64, 1.53, 1.21;

LCMS: m/z 487 [M+H]+;

HPLC retention time: 2.097 min (method 61).

### Example 12(1)˜ 12(4)

A procedure for a purpose similar to that for Reference Example 22→Reference Example 23→Example 12 was carried out by using a corresponding alcohol compound instead of 4-bromo-2-ethylphenol, a corresponding halide compound instead of 2-amino-4-chloropyrimidine, a corresponding cyclic compound instead of 5,5-dimethylimidazolidine-2,4-dione, and a corresponding halide compound instead of 3-iodo-5-(trifluoromethyl)pyridine, to give the following compounds of Examples.

### Example 12(1): 3-{4-[(6,7-dimethoxy-4-quinolinyl)oxy]phenyl}-4-methoxy-1-[3-(trifluoromethyl)phenyl]-2-imidazolidinone trifluoroacetate (1:1)

¹H NMR (400 MHz, DMSO-d₆): δ 8.60, 8.19, 7.87-7.78, 7.66-7.59, 7.46-7.37, 6.63, 5.81, 4.20, 4.08, 3.99, 3.98, 3.31;

LCMS: m/z 540 [M+H]+;

HPLC retention time: 1.401 min (method 9).

### Example 12(2): 1-{4-[(6,7-dimethoxy-4-quinolinyl)oxy]phenyl}-3-[3-(trifluoromethoxy)phenyl]-2,4-imidazolidinedione

¹H NMR (400 MHz, DMSO-d₆): δ 8.65, 7.89-7.87, 7.71-7.64, 7.55-7.44, 6.63, 4.70, 4.00, 3.99;

LCMS: m/z 540 [M+H]+;

HPLC retention time: 1.442 min (method 9).

### Example 12(3): 3-(3-cyclopropylphenyl)-1-{4-[(6,7-dimethoxy-4-quinolinyl)oxy]phenyl}-2,4-imidazolidinedione

¹H NMR (400 MHz, DMSO-d₆): δ 8.71, 7.90-7.88, 7.68, 7.52-7.48, 7.45-7.38, 7.20-7.14, 6.70, 4.69, 4.02, 2.00, 1.03-0.99, 0.72-0.68;

LCMS: m/z 496 [M+H]+;

HPLC retention time: 1.406 min (method 9).

### Example 12(4): 3-{4-[(6-amino-3-pyridinyl)oxy]phenyl}-5-methyl-1-[5-(trifluoromethyl)-3-pyridinyl]-2,4-imidazolidinedione

¹H NMR (400 MHz, DMSO-d₆): δ 9.13, 8.82, 8.47, 7.85, 7.44-7.37, 7.07-7.03, 6.63, 6.29, 5.25, 1.47;

LCMS: m/z 444 [M+H]+;

HPLC retention time: 1.237 min (method 43).

### Reference Example 24: tert-butyl N—[5-(2,2,2-trifluoro-1-hydroxy-ethyl)-3-pyridyl]carbamate

To a stirred solution of tert-butyl N-(5-formyl-3-pyridyl)carbamate (CAS No. 337904-94-6, 850 mg) in DMF (30 mL) was added K₂CO₃ (1057 mg) and trimethyl(trifluoromethyl)silane (CAS No. 81290-20-2, 1088 mg) at 0° C. under N₂ atmosphere. The resulting mixture was stirred two days at room temperature under N₂ atmosphere. The resulting mixture was diluted with ethyl acetate and water. The resulting mixture was extracted with ethyl acetate. The combined organic layers were washed with water then washed with saturated brine solution, dried over anhydrous Na₂SO₄. After filtration, the filtrate was concentrated under reduced pressure. The crude product was used in the next step directly without further purification.

### Reference Example 25: tert-butyl N—[5-(2,2,2-trifluoroacetyl)-3-pyridyl]carbamate

To a stirred solution of the compound prepared in Reference Example 24 (785 mg) in ethyl acetate (10 mL) was added 2-iodoxybenzoic acid (CAS No. 61717-82-6, 1.5 g) at room temperature. The resulting mixture was stirred overnight at 78° C. The mixture was allowed to cool down to 25° C. The resulting mixture was filtered, and the filter cake was washed with ethyl acetate. After filtration, the filtrate was concentrated under reduced pressure. The crude product was purified by Prep-HPLC to afford the title compound (540 mg).

### Reference Example 26: tert-butyl N—[5-[2-(trifluoromethyl)oxetan-2-yl]-3-pyridyl]carbamate

To a stirred solution of 1 M t-BuOK in THF (2.7 mL) in DMSO (3 mL) was added trimethylsulfoxonium iodide (CAS No. 1774-47-6, 606 mg), and the reaction was stirred at room temperature for 10 min. A solution of the compound prepared in Reference Example 25 (200 mg) in DMSO (2 mL) was added dropwise to the reaction, and the resulting solution was stirred at room temperature overnight (15 h). The crude reaction was partitioned between diethyl ether and brine. The organic layer was separated, washed once more with brine, dried over anhydrous sodium sulfate, and concentrated under reduced pressure. The residue was applied onto a silica gel column with ethyl acetate/petroleum ether (1:1) to give the title compound (70 mg).

### Reference Example 27: methyl 2-[tert-butoxycarbonyl-[5-[2-(trifluoromethyl)oxetan-2-yl]-3-pyridyl]amino]acetate

To a stirred solution of the compound prepared in Reference Example 26 (70 mg) in MeCN (15 mL) were added Cs₂CO₃ (143 mg) and methyl 2-bromoacetate (50 mg) at room temperature. The resulting mixture was stirred for 1 h at 45° C. under N₂ atmosphere. The resulting solution was extracted with ethyl acetate. The organic layers were combined, washed with sodium carbonate (aq.) and brine, dried and concentrated under vacuum. The residue was applied on a silica gel column and eluted with ethyl acetate/petroleum ether (1/1) to give the title compound (60 mg).

### Reference Example 28: methyl 2-[[5-[2-(trifluoromethyl)oxetan-2-yl]-3-pyridyl]amino]acetate

To a stirred solution of the compound prepared in Reference Example 27 (75 mg) in DCM (15 mL) was added TFA (1 mL) at room temperature. The resulting mixture was stirred 0.5 h at room temperature. The solvent was removed under reduced pressure. The residue was diluted with water, then adjusted to pH 6˜7 with sodium bicarbonate. The resulting solution was extracted with ethyl acetate. The organic layers were combined, washed with sodium carbonate (aq.) and brine, dried and concentrated under vacuum. The product was used in the next step directly without further purification.

### Reference Example 29: methyl 2-[chlorocarbonyl-[5-[2-(trifluoromethyl)oxetan-2-yl]-3-pyridyl]amino]acetate

To a stirred solution of the compound prepared in Reference Example 28 (80 mg) in toluene (5 mL) were added DIEA (0.14 mL) and trichloromethyl carbonochloridate (0.05 mL) at room temperature under N₂ atmosphere. The resulting mixture was stirred 1 h at 75° C. The product was used in the next step directly without further purification.

### Example 13: 3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione

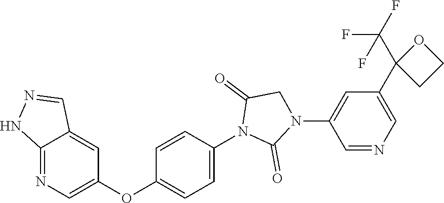 [SMILES: O=C1CN(c2cncc(C3(C(F)(F)F)CCO3)c2)C(=O)N1c1ccc(Oc2cnc3[nH]ncc3c2)cc1]

A procedure for a purpose similar to that for Reference Example 6→Reference Example 7→Example 1 was carried out by using 1H-pyrazolo[3,4-b]pyridin-5-ol (CAS No. 1256818-99-1) instead of 2-aminopyrimidin-5-ol, 1-fluoro-4-nitrobenzene, and the compound prepared in Reference Example 29 instead of the compound prepared in Reference Example 5 to afford the title compound having the following physical property values.

¹H NMR (300 Hz, DMSO-d₆): δ 13.77, 8.84, 8.47, 8.42, 8.32, 8.13, 8.02, 7.44-7.39, 7.16-7.11, 4.81-4.74, 4.71, 4.65-4.58, 3.34-3.25, 3.11-3.01;

LCMS: m/z 511 [M+H]+;

HPLC retention time: 1.254 min (method 5).

### Example 13(1)

A procedure for a purpose similar to that for Reference Example 24→Reference Example 25→Reference Example 26→Reference Example 27→Reference Example 28→Reference Example 29→Example 13 was carried out by using tert-butyl (3-formylphenyl)carbamate instead of tert-butyl N-(5-formyl-3-pyridyl)carbamate, 1H-pyrazolo[3,4-b]pyridin-5-ol, and 1-fluoro-4-nitrobenzene, to give the following compounds of Examples.

### Example 13(1): 3-[4-(1H-pyrazolo[3,4-b]pyridin-5-yloxy)phenyl]-1-{3-[2-(trifluoromethyl)-2-oxetanyl]phenyl}-2,4-imidazolidinedione

¹H NMR (400 MHz, DMSO-d₆): δ 13.78, 8.48, 8.13, 8.02, 7.92, 7.59-7.50, 7.42-7.39, 7.20-7.12, 4.77-4.72, 4.64, 4.59-4.54, 3.33-3.22, 3.00-2.93;

LCMS: m/z 510 [M+H]+;

HPLC retention time: 2.010 min (method 38).

### Example 14: 3-{4-[(7-oxido-1H-pyrrolo[2,3-b]pyridin-4-yl)oxy]phenyl}-1-[3-(trifluoromethyl)phenyl]-2,4-imidazolidinedione

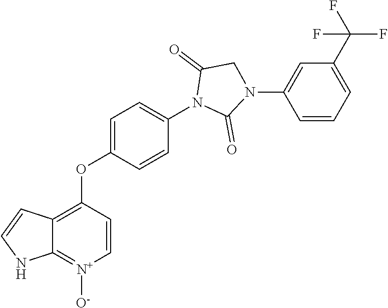 [SMILES: O=C1CN(c2cccc(C(F)(F)F)c2)C(=O)N1c1ccc(Oc2cc[n+]([O-])c3[nH]ccc23)cc1]

To a stirred mixture of the compound prepared in Example 1(32) (100 mg) in DCM (2 mL) was added mCPBA (CAS No. 937-14-4, 176 mg) at 0° C. The resulting mixture was stirred for 14 h at room temperature. The reaction was quenched with sat NaHCO₃ and the organic layer was extracted with DCM and the organic layer was washed with brine, dried over Na₂SO₄, and concentrated. The crude product was purified with column chromatography (CHCl₃:MeOH=10:1) to give the title compound (34 mg) having the following physical property values.

¹H NMR (300 MHz, CDCl₃): δ 4.54, 6.32-6.69, 7.26, 7.50 7.73-7.88, 7.88-7.99, 8.03-8.20;

TLC Rf: 0.45 (CHCl₃/MeOH=10/1).

### Reference Example 30: (6,7-dimethoxy-4-quinolinyl)(4-nitrophenyl)methanol

To an oven-dried three-neck round-bottom flask was added 4-bromo-6,7-dimethoxy-quinoline (CAS No. 666734-51-6, 2000 mg). The flask was then evacuated and charged with N₂ for three times, followed by addition of THF (25 mL) to dissolve the compound. n-BuLi (in hexane) (4.5 mL, 11 mmol) was added dropwise to the solution at −78° C. At this temperature, the mixture was stirred for 1 h, followed by addition of the solution of 4-nitrobenzaldehyde (CAS No. 555-16-8, 1353 mg) in THF (13 mL). Stirred for 1 h, the temperature was allowed to rise up to 0° C. for an additional hour of stirring. Quenched by saturated aqueous NaHCO₃, the mixture was extracted by ethyl acetate. The combined organic layer was then washed by brine and dried over anhydrous Na₂SO₄. After filtration, the filtrate was concentrated under vacuum. To the residue was added ethyl acetate and the resulting mixture was filtered. The filter cake was washed with ethyl acetate and collected. The combined filtrate was evaporated and the resulting crude product was further purified by silica gel column chromatography, eluted with Petroleum ether:ethyl acetate=1:4. Combined with the filter cake, the reaction finally afford the title compound (1353 mg).

### Reference Example 31: (4-aminophenyl)(6,7-dimethoxy-4-quinolinyl)methanone

To a mixture of the compound prepared in Reference Example 30 (340 mg) in DCM (10 mL) was added Dess-Martin Periodinane (CAS No. 87413-09-0, 1271 mg) at 0° C. The mixture was stirred at room temperature overnight. Quenched by saturated aqueous NaHCO₃, the mixture was extracted by DCM. The combined organic layer was washed by brine and dried over anhydrous Na₂SO₄. After filtration, the filtrate was concentrated under vacuum. The crude product was purified by prep-TLC (Petroleum ether:ethyl acetate=1:1) to afford the title compound (262 mg).

### Example 15: 3-{4-[(6,7-dimethoxy-4-quinolinyl)carbonyl]phenyl}-1-[5-(trifluoromethyl)-3-pyridinyl]-2,4-imidazolidinedione

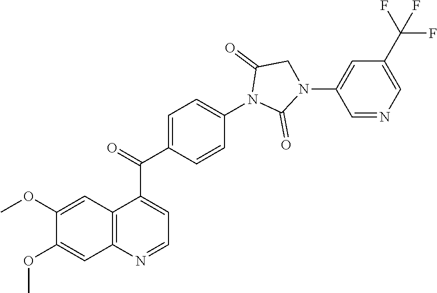 [SMILES: COc1cc2nccc(C(=O)c3ccc(N4C(=O)CN(c5cncc(C(F)(F)F)c5)C4=O)cc3)c2cc1OC]

A procedure for a purpose similar to that for Reference Example 7→Example 1 was carried out by using a compound produced in Reference Example 31 instead of a compound produced in Reference Example 6, and the compound prepared in Reference Example 5 to give the title compound having the following physical property values.

¹H NMR (400 MHz, DMSO-d₆): δ 9.19, 8.85, 8.80, 8.53, 7.98, 7.69, 7.52, 7.47, 7.14, 4.79, 3.97, 3.76;

LCMS: m/z 537 [M+H]+;

HPLC retention time: 1.711 min (method 3).

### Reference Example 32: tert-butyl N—[4-[(3-formyl-4-pyridyl)oxy]phenyl]carbamate

To a mixture of 4-chloropyridine-3-carbaldehyde (CAS No. 114077-82-6, 3.1 g) and tert-butyl N-(4-hydroxyphenyl)carbamate (CAS No. 54840-15-2, 6.0 g) in DMF (30 mL) was added K₂C03 (6.1 g) at room temperature. The mixture was stirred at 90 degree for 7 h. Ice-water was added to the reaction mixture and the mixture was extracted with AcOEt. The combined organic layer was washed by brine and dried over anhydrous Na₂SO₄. After filtration, the filtrate was concentrated under vacuum. The crude product was purified by column chromatography (Hexane:AcOEt=1:1) to afford the title compound (6.9 g).

### Reference Example 33: ethyl (Z)-2-azido-3-[4-[4-(tert-butoxycarbonylamino)phenoxy]-3-pyridyl]prop-2-enoate

To a 3 mL of EtOH solution of the compound prepared in Reference Example 32 (500 mg), ethyl 2-azidoacetate (CAS No. 637-81-0, 821 mg) was added slowly at 0 degree. Then, Sodium ethoxide (240 mg) was added slowly. The mixture was stirred at room temperature for 2 h, then stirred at 50 degree for 3 h. To the reaction mixture, AcOEt was added and the organic layer was washed with water, NaHCO3 aq, and brine, then dried over Na2SO4. After filtration, the filtrate was concentrated under vacuum. The crude product was purified by column chromatography (Hexane:AcOEt=8:2 to 2/8) to afford the title compound (186 mg).

### Reference Example 34: ethyl 4-[4-(tert-butoxycarbonylamino)phenoxy]-1H-pyrrolo[2,3-b]pyridine-2-carboxylate

The solution of the compound prepared in Reference Example 33 (180 mg) in m-xylene (3 mL) was stirred at 170 degree for 3 h. The reaction mixture was cooled to room temperature. The resulting precipitate was collected and washed with hexane/MTBE and dried under vacuum to afford the title compound (168 mg).

### Reference Example 35: 4-[4-({[(2-methyl-2-propanyl)oxy]carbonyl}amino)phenoxy]-1H-pyrrolo[2,3-b]pyridine-2-carboxylic acid

To a mixture of the compound prepared in Reference Example 34 (200 mg), MeOH (4 mL) and THF (2 mL) was added 1.5 mL of 5N NaOH at room temperature. The mixture was stirred at 60 degree for 1.5 h. To the reaction mixture, 1N HCl was added and the resulting precipitate was collected and dried under vacuum to afford the title compound (130 mg).

### Reference Example 36: 2-methyl-2-propanyl (4-{[2-(tetrahydro-2H-pyran-4-ylcarbamoyl)-1H-pyrrolo[2,3-b]pyridin-4-yl]oxy}phenyl)carbamate

To a mixture of the compound prepared in Reference Example 35 (50 mg), DIEA (0.02 mL), HATU (CAS No. 148893-10-1, 77 mg) in DMF (2 mL) was added 4-aminotetrahydropyran (1.5 eq) at room temperature. The mixture was stirred at room temperature for 15 h. The reaction was quenched with NH₄Claq and the aqueous layer was extracted with AcOEt, and the organic layer was washed with brine and dried over Na₂SO₄. After filtration, the filtrate was concentrated under vacuum. The crude product was purified by column chromatography (NH silica) to afford the title compound (44 mg).

### Reference Example 37: 4-(4-aminophenoxy)-N-(tetrahydro-2H-pyran-4-yl)-1H-pyrrolo[2,3-b]pvridine-2-carboxamide

To a DCM (0.5 mL) solution of the compound prepared in Reference Example 36 (36 mg), 0.5 mL of TFA was added at room temperature and the reaction mixture was stirred at room temperature for 1.5 h. The reaction mixture was concentrated to give the title compound (28 mg).

### Examples 16: 4-(4-{2,5-dioxo-3-[3-(trifluoromethyl)phenyl]-1-imidazolidinyl}phenoxy)-N-(tetrahydro-2H-pyran-4-yl)-1H-pyrrolo[2,3-b]pyridine-2-carboxamide

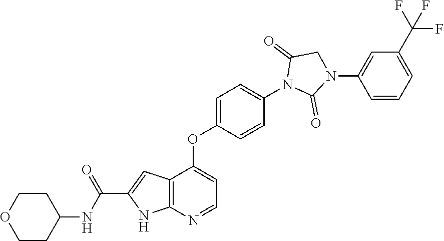 [SMILES: O=C(NC1CCOCC1)c1cc2c(Oc3ccc(N4C(=O)CN(c5cccc(C(F)(F)F)c5)C4=O)cc3)ccnc2[nH]1]

To a mixture of the compound prepared in Reference Example 37 (28 mg) and DIEA (2.5 eq) in THE (1.5 mL), methyl 2-(N-chlorocarbonylanilino)acetate (1.1 eq) was added slowly at room temperature and the reaction mixture was stirred at reflux for 16 h. The reaction mixture was cooled to room temperature and quenched with NH₄Claq and the aqueous layer was extracted with AcOEt, and the organic layer was washed with brine and dried over Na₂SO₄. After filtration, the filtrate was concentrated. The crude mixture was purified with column chromatography (amino silica, AcOEt/MeOH=1/0 to 9/1) to afford the title compound having the following physical property values.

LCMS: m/z 580 [M+H]+;

HPLC retention time: 0.91 min (method 83).

### Example 16(1)˜16(2)

A procedure for a purpose similar to that for Reference Example 36→Reference Example 37→Example 16 was carried out by using the compound prepared in Reference Example 35, a corresponding amine compound instead of 4-aminotetrahydropyran, methyl 2-(N-chlorocarbonylanilino)acetate, to give the following compounds of Examples.

### Example 16(1): N-[2-(dimethylamino)-2-oxoethyl]-4-(4-{2,5-dioxo-3-[3-(trifluoromethyl)phenyl]-1-imidazolidinyl}phenoxy)-1H-pyrrolo[2,3-b]pyridine-2-carboxamide

LCMS: m/z 580 [M+H]+;

HPLC retention time: 0.87 min (method 38).

### Example 16(2): 4-(4-{2,5-dioxo-3-[3-(trifluoromethyl)phenyl]-1-imidazolidinyl}phenoxy)-1H-pyrrolo[2,3-b]pyridine-2-carboxamide

¹H NMR (400 MHz, DMSO-d₆) δ ppm 3.85-4.22, 4.71, 6.41-6.71, 7.05-7.20, 7.37, 7.53, 7.59-7.75, 7.79-7.97, 8.04-8.33, 8.50-8.75, 11.97-12.65;

LCMS: m/z 496 [M+H]+;

HPLC retention time: 0.83 min (method 38).

### Reference Example 38: 7-(cyclopropoxy)quinazolin-4-ol

The mixture of quinazoline-4,7-diol (CAS No. 16064-25-8, 400 mg), bromocyclopropane (CAS No. 4333-56-6, 0.26 mL) and CS₂CO₃ (341 mg) in DMSO (5 mL) was stirred at 120° C. overnight. The resulting solution was diluted with water and extracted with ethyl acetate. The organic layers were combined, washed with brine, dried over anhydrous sodium sulfate and concentrated under vacuum. The crude product was purified by column chromatography to afford the title compound (43 mg).

### Reference Example 39: 4-chloro-7-(cyclopropoxy)quinazoline

To a solution of the compound prepared in Reference Example 38 (43 mg) in CHCl₃ (1.0 mL) was added POCl₃ (100 mg) and Et₃N (15 mg). The resulting solution was stirred for 8 h at 70° C. The mixture solution was vacuum suction filter to obtain the title compound (crude, 700 mg). The product was used in the next step directly without further purification.

### Example 17: 3-(trans-4-{[7-(cyclopropyloxy)-4-quinazolinyl]oxy}cyclohexyl)-1-[5-(trifluoromethyl)-3-pyridinyl]-2,4-imidazolidinedione

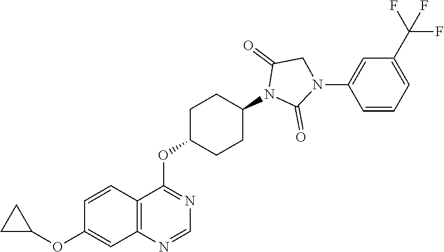 [SMILES: O=C1CN(c2cccc(C(F)(F)F)c2)C(=O)N1[C@H]1CC[C@H](Oc2ncnc3cc(OC4CC4)ccc23)CC1]

A procedure for a purpose similar to that for Reference Example 9→Example 3 was carried out by using a corresponding alcohol compound instead of 4-amino-2-ethylphenol, and the compound prepared in Reference Example 39 instead of 2-amino-4-chloropyrimidine, to give the title compound.

¹H NMR (400 MHz, DMSO-d₆) δ 9.11, 8.78-8.71, 8.50, 8.04, 7.53, 7.28, 5.30, 4.59, 4.08, 2.42-2.24, 1.86, 1.77-1.63, 0.90, 0.84-0.68;

LCMS: m/z 528 [M+H]+;

HPLC retention time: 1.402 min (method 84).

### Example 17(1)˜17(10)

A procedure for a purpose similar to that for Reference Example 38→Reference Example 39→Example 17 was carried out by using a corresponding halide compound instead of bromocyclopropane, to give the following compounds of Examples.

### Example 17(1): 3-(trans-4-{[7-(2-hydroxyethoxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ ppm: 9.10, 8.75-8.72, 8.49, 8.03, 7.29-7.28, 5.32-5.25, 4.96, 4.59, 4.19-4.17, 4.11-4.01, 3.80-3.78, 2.40-2.29, 1.90-1.80, 1.78-1.67;

LCMS: m/z 532 [M+H]+;

HPLC retention time: 1.427 min (method 88).

### Example 17(2): 3-(trans-4-{[7-(fluoromethoxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.10, 8.80-8.72, 8.49, 8.14, 7.52, 7.44, 6.15, 6.01, 5.35-5.29, 4.58, 4.09-4.05, 2.38-2.29, 1.87-1.85, 1.75-1.66;

LCMS: m/z 520 [M+H]+;

HPLC retention time: 1.381 min (method 84).

### Example 17(3): 3-(trans-4-{[7-(cyclobutyloxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.11, 8.73, 8.69, 8.49, 8.03, 7.25-7.20, 7.12, 5.32-5.25, 4.98-4.90, 4.59, 4.12-4.01, 2.58-2.55, 2.40-2.28, 2.19-2.05, 1.90-1.80, 1.75-1.68;

LCMS: m/z 542 [M+H]+;

HPLC retention time: 1.478 min (method 84).

### Example 17(4): 3-(trans-4-{[7-(tetrahydro-2H-pyran-4-yloxy)-4-quinazolinyl]oxy}cyclohexyl)-1-[5-(trifluoromethyl)-3-pyridinyl]-2,4-imidazolidinedione

¹H NMR (400 MHz, DMSO-d₆) δ 9.11, 8.78-8.70, 8.49, 8.03, 7.39, 7.29, 5.29-5.26, 4.88-4.83, 4.59, 4.12-4.00, 3.90-3.85, 3.58-3.52, 2.41-2.31, 2.06-2.03, 1.86-1.83, 1.77-1.59;

LCMS: m/z 572 [M+H]+;

HPLC retention time: 2.878 min (method 16-2).

### Example 17(5): 3-(trans-4-{[7-(3-oxetanyloxy)-4-quinazolinyl]oxy}cyclohexyl)-1-[5-(trifluoromethyl)-3-pyridinyl]-2,4-imidazolidinedione

¹H NMR (300 MHz, DMSO-d₆) δ 9.11-9.09, 8.75-8.73, 8.49, 8.07, 7.31-7.27, 7.00, 5.54-5.49, 5.35-5.22, 5.02, 4.63-4.59, 4.12-4.00, 2.40-2.25, 1.95-1.82, 1.75-1.63;

LCMS: m/z 544 [M+H]+;

HPLC retention time: 1.618 min (method 88).

### Example 17(6): formic acid—3-(trans-4-{[7-(3-azetidinyloxy)-4-quinazolinyl]oxy}cyclohexyl)-1-[5-(trifluoromethyl)-3-pyridinyl]-2,4-imidazolidinedione (1:1)

¹H NMR (400 MHz, DMSO-d₆) δ 9.10, 8.75, 8.49, 8.22, 8.06, 7.27, 7.09, 5.29-5.22, 4.58, 4.12-4.03, 3.74, 2.38-2.28, 1.85-1.82, 1.71-1.68;

LCMS: m/z 543 [M+H]+;

HPLC retention time: 1.022 min (method 84).

### Example 17(7): 3-(trans-4-{[7-(4-piperidinyloxy)-4-quinazolinyl]oxy}cyclohexyl)-1-[5-(trifluoromethyl)-3-pyridinyl]-2,4-imidazolidinedione trifluoroacetate (1:1)

¹H NMR (400 MHz, DMSO-d₆) δ 9.08, 8.74-8.72, 8.48, 8.06, 7.43, 7.31, 5.34-5.28, 4.99-4.90, 4.58, 4.11-4.00, 3.35-3.24, 3.18-3.11, 2.34-2.28, 2.23-2.15, 1.95-1.82, 1.78-1.62;

LCMS: m/z 571 [M+H]+;

HPLC retention time: 1.039 min (method 84).

### Example 17(8): 3-[trans-4-({7-[(1-methyl-4-piperidinyl)oxy]-4-quinazolinyl}oxy)cyclohexyl]-1-[5-(trifluoromethyl)-3-pyridinyl]-2,4-imidazolidinedione trifluoroacetate (1:1)

¹H NMR (300 MHz, DMSO-d₆) δ 9.65-9.42, 9.11, 8.76, 8.49, 8.12-8.05, 7.50-7.23, 5.38-5.25, 5.07-5.02, 4.89-4.80, 4.60, 4.12-4.00, 3.59-3.50, 3.41-3.34, 3.26-3.12, 2.85, 2.40-2.26, 2.12-1.95, 1.90-1.60;

LCMS: m/z 584 [M+H]+;

HPLC retention time: 1.801 min (method 91).

### Example 17(9): formic acid—3-(trans-4-{[7-(3-pyrrolidinyloxy)-4-quinazolinyl]oxy}cyclohexyl)-1-[5-(trifluoromethyl)-3-pyridinyl]-2,4-imidazolidinedione (1:1)

¹H NMR (400 MHz, DMSO-d₆) δ 9.10, 8.75-8.72, 8.48, 8.27, 8.03, 7.28-7.22, 5.32-5.25, 5.22-5.17, 4.59, 4.10-3.97, 3.08-2.97, 2.38-2.29, 2.18-2.14, 1.95-1.80, 1.72-1.62;

LCMS: m/z 557 [M+H]+;

HPLC retention time: 1.165 min (method 88).

### Example 17(10): 3-[trans-4-({7-[(1-methyl-3-pyrrolidinyl)oxy]-4-quinazolinyl}oxy)cyclohexyl]-1-[5-(trifluoromethyl-3-pyridinyl]-2,4-imidazolidinedione trifluoroacetate (1:1)

¹H NMR (400 MHz, DMSO-d₆) δ 10.10, 9.10, 8.76, 8.49, 8.09-8.07, 7.36, 7.30-7.27, 5.42-5.39, 5.33-5.28, 4.59, 4.11-4.00, 3.89-3.47, 2.89, 2.70-2.60, 2.40-2.28, 2.20-2.09, 1.90-1.80, 1.75-1.62;

LCMS: m/z 571 [M+H]+;

HPLC retention time: 1.209 min (method 88).

### Reference Example 40: 3-{trans-4-[(7-nitro-4-quinazolinyl)oxy]cyclohexyl}-1-[5-(trifluoromethyl)-3-pyridinyl]-2,4-imidazolidinedione

A procedure for a purpose similar to that for Reference Example 9→Example 3 was carried out by using trans-4-aminocyclohexanol (CAS No. 27489-62-9) instead of 4-amino-2-ethylphenol, and 4-chloro-7-nitroquinazoline (CAS No. 19815-17-9) instead of 2-amino-4-chloropyrimidine, to give the title compound.

LCMS: m/z 517 [M+H]+.

### Example 18: 3-{trans-4-[(7-amino-4-quinazolinyl)oxy]cyclohexyl}-1-[5-(trifluoromethyl)-3-pyridinyl]-2,4-imidazolidinedione

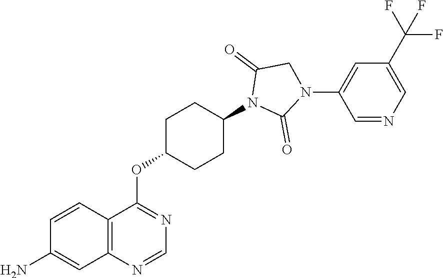 [SMILES: Nc1ccc2c(O[C@H]3CC[C@H](N4C(=O)CN(c5cncc(C(F)(F)F)c5)C4=O)CC3)ncnc2c1]

To a solution of the compound prepared in Reference Example 40 (60 mg) in water (1 mL) and ethanol (2 mL) was added Fe (52 mg) and NH₄Cl (49 mg). The resulting mixture was stirred at 85° C. for 2 h. The resulting suspension was filtered, the filter cake was washed with methanol. The filtrate was concentrated under reduced pressure. The crude product was purified by Prep-HPLC to afford the title compound (14.3 mg).

¹H NMR (300 MHz, DMSO-d₆) δ 9.11, 8.75, 8.50, 7.78, 6.95, 6.76, 6.27, 5.30-5.26, 4.59, 4.10-3.96, 2.38-2.18, 1.84-1.80, 1.78-1.59;

LCMS: m/z 487 [M+H]+;

HPLC retention time: 1.119 min (method 84).

### Example 19: N-{4-[(trans-4-{2,5-dioxo-3-[5-(trifluoromethyl)-3-pyridinyl]-1-imidazolidinyl}cyclohexyl)oxy]-7-quinazolinyl}acetamide

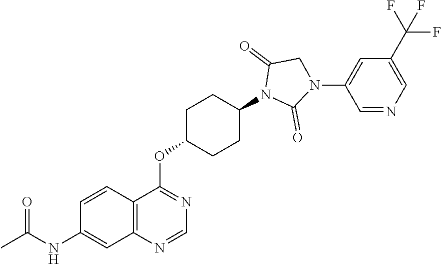 [SMILES: CC(=O)Nc1ccc2c(O[C@H]3CC[C@H](N4C(=O)CN(c5cncc(C(F)(F)F)c5)C4=O)CC3)ncnc2c1]

To a solution of the compound prepared in Example 18 (25 mg) in THF (1 mL) was added acetyl chloride (8 mg) and diethyl acetate (0.03 mL). The resulting mixture was stirred at room temperature for 1.5 h. The mixture was concentrated under vacuum. The crude product was purified by Prep-HPLC to afford the title compound (4.6 mg).

¹H NMR (300 MHz, DMSO-d₆) δ 10.49, 9.11, 8.74, 8.49, 8.30, 8.06, 7.78-7.68, 5.34-5.20, 4.59, 4.07-3.98, 2.35-2.28, 2.14, 1.95-1.85, 1.78-1.64;

LCMS: m/z 529 [M+H]+;

HPLC retention time: 1.145 min (method 84).

### Example 19(1): N—[5-(4-{2,5-dioxo-3-[5-(trifluoromethyl)-3-pyridinyl]-1-imidazolidinyl}phenoxy)-1H-pyrazolo[3,4-b]pyridin-3-yl]acetamide

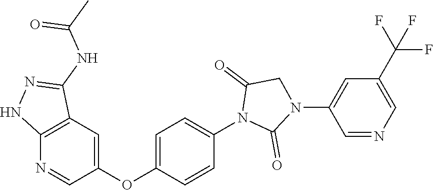 [SMILES: CC(=O)Nc1n[nH]c2ncc(Oc3ccc(N4C(=O)CN(c5cncc(C(F)(F)F)c5)C4=O)cc3)cc12]

A procedure for a purpose similar to that for Example 19 was carried out by using the compound prepared in Example 1(30) instead of the compound prepared in Example 18, to give the title compound.

¹H NMR (400 MHz, DMSO-d₆) δ ppm: 13.34, 10.70, 9.18, 8.77, 8.53-8.47, 8.12, 7.43, 7.15, 4.76, 2.09;

LCMS: m/z 512 [M+H]+;

HPLC retention time: 1.357 min (method 88).

### Example 20: N-{4-[(trans-4-{2,5-dioxo-3-[5-(trifluoromethyl)-3-pyridinyl]-1-imidazolidinyl}cyclohexyl)oxy]-7-quinazolinyl}methanesulfonamide

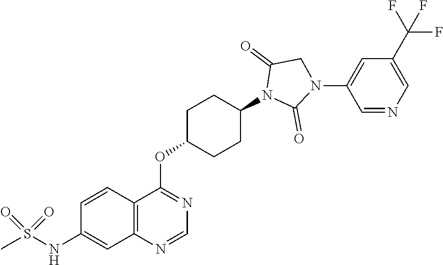 [SMILES: CS(=O)(=O)Nc1ccc2c(O[C@H]3CC[C@H](N4C(=O)CN(c5cncc(C(F)(F)F)c5)C4=O)CC3)ncnc2c1]

To a solution of the compound prepared in Example 18 (30 mg) in DCM (0.60 mL) was added pyridine (0.6 mL) and methanesulfonyl chloride (21 mg). The resulting mixture was stirred at room temperature overnight. The resulting mixture was concentrated under reduced pressure. The crude product was purified by Prep-HPLC to afford the title compound (11.1 mg).

¹H NMR (400 MHz, DMSO-d₆) δ 10.56, 9.10, 8.74, 8.49, 8.08, 7.62, 7.50-7.47, 5.34-5.25, 4.59, 4.10-4.02, 3.17, 2.35-2.29, 1.85, 1.75-1.65;

LCMS: m/z 565 [M+H]+;

HPLC retention time: 1.198 min (method 84).

### Example 21(1): 3-(trans-4-{[7-(methylamino)-4-quinazolinyl]oxy}cyclohexyl)-1-[5-(trifluoromethyl)-3-pyridinyl]-2,4-imidazolidinedione, and Example 21(2): 3-(trans-4-{[7-(dimethylamino)-4-quinazolinyl]oxy}cyclohexyl)-1-[5-(trifluoromethyl)-3-pyridinyl]-2,4-imidazolidinedione

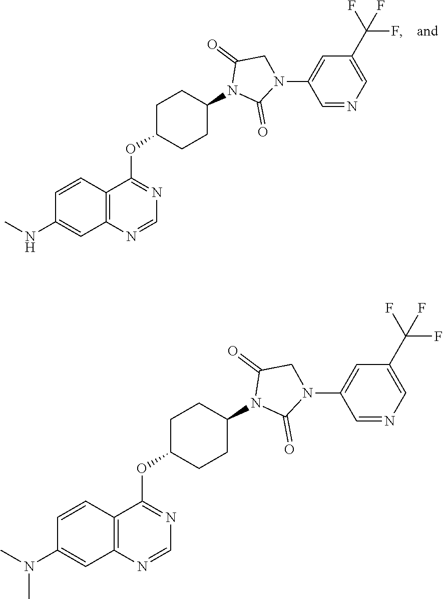 [SMILES: CNc1ccc2c(O[C@H]3CC[C@H](N4C(=O)CN(c5cncc(C(F)(F)F)c5)C4=O)CC3)ncnc2c1 ; CN(C)c1ccc2c(O[C@H]3CC[C@H](N4C(=O)CN(c5cncc(C(F)(F)F)c5)C4=O)CC3)ncnc2c1]

To a solution of the compound prepared in Example 18 (100 mg) in methanol (4 mL) was added AcOH (14 mg) and formaldehyde (37% in H₂O; 18.5 mg). After 0.5 h, NaBH₃CN (19.4 mg) was added at 0° C. The resulting mixture was stirred at room temperature overnight. The mixture was diluted with water, then adjusted to pH=7 with sodium bicarbonate. The resulting solution was extracted with ethyl acetate. The organic layers were combined, washed with brine, dried over anhydrous Na₂SO₄ and concentrated under vacuum. The residue was purified by Prep-HPLC to afford the title compounds (Example 21(1): 11.7 mg) and (Example 21(2): 7.9 mg), respectively.

### Example 21(1)

¹H NMR (300 MHz, DMSO-d₆) δ 9.10, 8.75, 8.52, 7.77, 6.98-6.94, 6.84-6.76, 6.58, 5.29-5.18, 4.58, 4.09-4.01, 2.79, 2.34-2.26, 1.90-1.74, 1.70-1.55;

LCMS: m/z 501 [M+H]+;

HPLC retention time: 2.753 min (method 16-2).

### Example 21(2)

¹H NMR (300 MHz, DMSO-d₆) δ 9.10, 8.75, 8.55, 8.50, 7.87, 7.22-7.18, 6.81, 5.29-5.18, 4.58, 4.09-3.95, 3.09, 2.34-2.24, 1.90-1.76, 1.71-1.56;

LCMS: m/z 515 [M+H]+;

HPLC retention time: 2.867 min (method 16-2).

### Reference Example 41: methyl 4-[4-(trans-tert-butoxycarbonylamino)cyclohexoxy]quinazoline-7-carboxylate

To a solution of methyl 4-hydroxyquinazoline-7-carboxylate (CAS No. 313535-84-1, 1.0 g), tert-butyl ((1s,4s)-4-hydroxycyclohexyl)carbamate (CAS No. 167081-25-6, 5.2 g) and triphenylphosphine (6.4 g) in DMF (10 mL) was added isopropyl azodicarboxylate (4.9 g) at 0° C. The mixture was stirred at 30° C. for 2 h. The resulting solution was diluted with water and extracted with ethyl acetate. The organic layers were combined, washed with brine, dried over anhydrous Na₂SO₄ and concentrated under vacuum. The crude product was purified by column chromatography to afford the title compound (693 mg).

LCMS: m/z 402 [M+H]+.

### Reference Example 42: 4-[trans-4-(tert-butoxycarbonylamino)cyclohexoxy]quinazoline-7-carboxylic acid

The mixture of the compound prepared in Reference Example 41 (700 mg) and NaOH (348 mg) in THF (3 mL) and water (1 mL) was stirred at 30° C. for 1 h. The solvent was removed under reduced pressure. The residue was diluted with water, then adjusted to pH 5˜6 with HCl (2 M, aq). The resulting solution was extracted with ethyl acetate. The organic layers were combined, washed with brine and concentrated under vacuum to afford the title compound (550 mg). The product was used in the next step directly without further purification.

LCMS: m/z 388 [M+H]+.

### Reference Example 43: 4-(4-aminocyclohexoxy)quinazoline-7-carboxylic acid

The mixture of the compound prepared in Reference Example 42 (550 mg) and trifluoroacetic acid (1.5 mL) in DCM (3 mL) was stirred at 0° C. for 1 h. The reaction was concentrated under vacuum and the crude product was used directly on next step.

LCMS: m/z 288 [M+H]+.

### Example 22: 4-[(trans-4-{2,5-dioxo-3-[5-(trifluoromethyl)-3-pyridinyl]-1-imidazolidinyl}cyclohexyl)oxy]-7-quinazolinecarboxylic acid trifluoroacetate (1:1)

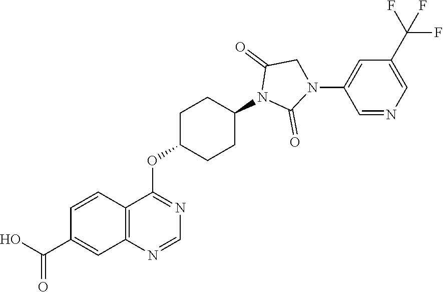 [SMILES: O=C(O)c1ccc2c(O[C@H]3CC[C@H](N4C(=O)CN(c5cncc(C(F)(F)F)c5)C4=O)CC3)ncnc2c1]

To a solution of the compound prepared in Reference Example 43 (180 mg) in THF (3 mL) was added ethyl acetate (3.1 mL) at 0° C. The mixture was stirred at 0° C. for 1 h. To the above solution the compound prepared in Reference Example 5 (279 mg) was added. The mixture was stirred at room temperature for 1 h. The reaction was concentrated under vacuum to and the crude product was purified by Prep-HPLC to afford the title compound (4 mg).

¹H NMR (300 MHz, DMSO-d₆) δ 9.12, 8.91, 8.77, 8.51, 8.42, 8.26, 8.16, 5.40-5.35, 4.61, 4.09-4.05, 2.41-2.35, 1.89-1.71;

LCMS: m/z 516 [M+H]+;

HPLC retention time: 1.568 min (method 88).

### Example 23: 4-[(trans-4-{2,5-dioxo-3-[5-(trifluoromethyl)-3-pyridinyl]-1-imidazolidinyl}cyclohexyl)oxy]-N-methyl-7-quinazolinecarboxamide

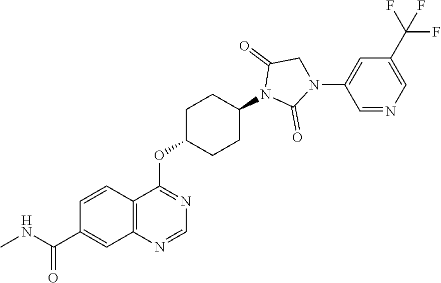 [SMILES: CNC(=O)c1ccc2c(O[C@H]3CC[C@H](N4C(=O)CN(c5cncc(C(F)(F)F)c5)C4=O)CC3)ncnc2c1]

The mixture of the compound prepared in Example 22 (20 mg), diethyl acetate (0.06 mL), PyBOP (61 mg) and methylamine hydrochloride (13.1 mg) in DMSO (1 mL) was stirred at room temperature for 1 h. The reaction was concentrated under reduced pressure. The crude product was purified by Prep-HPLC to afford the title compound (4.6 mg).

¹H NMR (400 MHz, DMSO-d₆) δ 9.11, 8.86, 8.75, 8.50, 8.37, 8.21, 8.08, 5.35-5.30, 4.60, 4.08-4.03, 2.86, 2.36-2.28, 1.94-1.84, 1.76-1.67;

LCMS: m/z 529 [M+H]+;

HPLC retention time: 2.510 min (method 87).

### Example 23(1): 4-[(trans-4-{2,5-dioxo-3-[5-(trifluoromethyl)-3-pyridinyl]-1-imidazolidinyl}cyclohexyl)oxy]-N,N-dimethyl-7-quinazolinecarboxamide

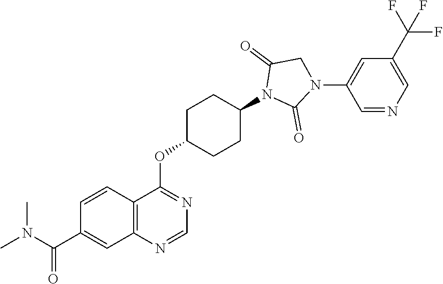 [SMILES: CN(C)C(=O)c1ccc2c(O[C@H]3CC[C@H](N4C(=O)CN(c5cncc(C(F)(F)F)c5)C4=O)CC3)ncnc2c1]

A procedure for a purpose similar to that for Example 23 was carried out by using dimethylamine hydrochloride instead of methylamine hydrochloride, to give the title compound.

¹H NMR (400 MHz, DMSO-d₆) δ 9.11, 8.86, 8.77-8.74, 8.50, 8.20, 7.88, 7.67, 5.37-5.33, 4.60, 4.09-4.07, 3.05, 2.92, 2.41-2.31, 1.88, 1.75-1.71;

LCMS: m/z 543 [M+H]+;

HPLC retention time: 2.567 min (method 87).

### Reference Example 44: 5-chloro-3-[(4-methoxyphenyl) methylsulfanyl]pyridine-2-carbaldehyde

To a stirred solution of 5-chloro-3-fluoro-pyridine-2-carbaldehyde (CAS No. 214055-11-5, 2.00 g) and PMB-SH (CAS No. 6258-60-2, 1.93 g) in THF (100 mL) were added t-BuOK (1.41 g) in portions at room temperature. The resulting mixture was stirred for 2 h at room temperature. The resulting mixture was quenched with water and extracted with EtOAc. The combined organic layers were dried over anhydrous Na₂SO₄. The residue was purified by column chromatography eluted with petroleum ether:ethyl acetate=3:1 to afford the title compound (3 g).

LCMS: m/z 294 [M+H]+.

### Reference Example 45: 6-chloroisothiazolo[4,5-b]pyridine

To a stirred solution of the compound prepared in Reference Example 44 (3 g) in DCE (20 mL) and were added SO₂Cl₂ (2.76 g). The resulting mixture was stirred for 30 min at room temperature. To the above mixture was added 7N NH₃ in MeOH (10.2 mL) in portions at room temperature. The resulting mixture was stirred for additional 2 h at room temperature. The resulting mixture was quenched with water and extracted with ethyl acetate. The combined organic layers over anhydrous Na₂SO₄. After filtration, the filtrate was concentrated under reduced pressure. The residue was purified by column chromatography eluted with petroleum ether:ethyl acetate=3:1 to afford the title compound (1 g).

### Reference Example 46: isothiazolo[4,5-b]pyridin-6-ol

To a solution of the compound prepared in Reference Example 45 (450 mg) in 1,4-dioxane (8 mL) was added 4,4,4′,4′,5,5,5′,5′-octamethyl-2,2′-bi(1,3,2-dioxaborolane) (CAS No. 73183-34-3, 2.01 g), Pd₂(dba)₃ (242 mg), PCy₃.HBF₄ (194 mg) and KOAc (775 mg), The reaction mixture was stirred for 16 h at 80° C. Then NaBO₃.4H₂O in Water was added to this mixture. The reaction mixture was stirred for 1 h at room temperature. The mixture was basified to pH=9˜11 with KOH. The reaction mixture was quenched with water. The resulting solution was extracted with ethyl acetate and the water layers were combined. Then the mixture was neutralized to pH=6-7 with 5 N HCl. The resulting solution was extracted with dichloromethane, washed with brine, dried over anhydrous sodium sulfate, filtered and concentrated under reduced pressure. The crude product was purified by prep-TLC to afford the title compound (150 mg).

LCMS: m/z 153 [M+H]+;

HPLC retention time: 0.438 min (method 67).

### Example 24: 3-[4-([1,2]thiazolo[4,5-b]pyridin-6-yloxy)phenyl]-1-[5-(trifluoromethyl)-3-pyridinyl]-2,4-imidazolidinedione

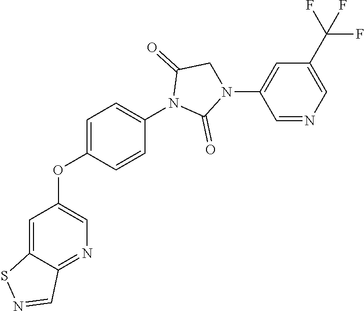 [SMILES: O=C1CN(c2cncc(C(F)(F)F)c2)C(=O)N1c1ccc(Oc2cnc3cnsc3c2)cc1]

A procedure for a purpose similar to that for Reference Example 6→Reference Example 7→Example 1 was carried out by using the compound prepared in Reference Example 46 instead of 2-aminopyrimidin-5-ol to give the title compound.

¹H NMR (400 MHz, DMSO-d₆) δ ppm: 9.26, 9.22-9.18, 8.78, 8.54, 8.34, 7.55, 7.38, 4.78;

LCMS: m/z 472 [M+H]+;

HPLC retention time: 1.489 min (method 5).

### Reference Example 47: 3-but-1-ynyl-4-nitrophenol

To a solution of 3-bromo-4-nitrophenol (500 mg) in THE (10 mL) was added TEA (0.95 mL), Pd(PPh₃)₄ (265 mg) and CuI (87 mg). Then but-1-yne (gas) was added to this mixture for 5 min at 0° C. The reaction mixture was stirred for 16 h at 70° C. The mixture was quenched with water and extracted with ethyl acetate and the organic layers were combined, washed with brine, dried over anhydrous sodium sulfate, filtered and concentrated under reduced pressure to afford crude product. The residue was purified by Prep-TLC (ethyl acetate/petroleum ether=1/3) to afford the title compound (270 mg).

### Reference Example 48: 4-amino-3-but-1-ynylphenol

To a solution of the compound prepared in Reference Example 47 (270 mg) in ethanol (7 mL) and water (3.5 mL) was added NH₄Cl (599 mg), Fe (631 mg) at room temperature. The reaction mixture was stirred for 2 h at 80° C. The mixture was filtered and washed with methanol. The solid was filtered out. The filtrate was concentrated under reduced pressure. The resulting solution was extracted with ethyl acetate and the organic layers were combined, washed with brine, dried over anhydrous sodium sulfate, filtered and concentrated under reduced pressure to afford crude product. The residue was purified by Prep-TLC to afford the title compound (100 mg).

LCMS: m/z 162 [M+H]+.

### Example 25: 3-{4-[(2-amino-4-pyrimidinyl)oxy]-2-(1-butyn-1-yl)phenyl}-1-[3-(methylsulfonyl)-5-(trifluoromethyl)phenyl]-2,4-imidazolidinedione trifluoroacetate (1:1)

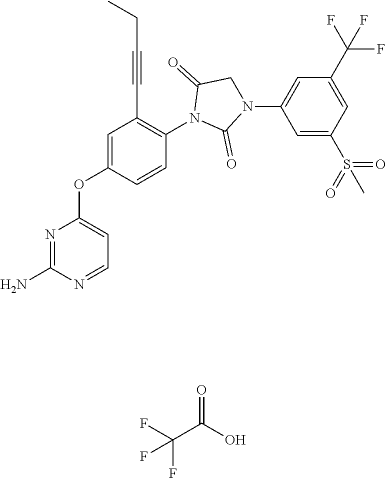 [SMILES: CCC#Cc1cc(Oc2ccnc(N)n2)ccc1N1C(=O)CN(c2cc(C(F)(F)F)cc(S(C)(=O)=O)c2)C1=O ; O=C(O)C(F)(F)F]

A procedure for a purpose similar to that for Reference Example 9→Example 3 was carried out by using the compound prepared in Reference Example 48 instead of 4-amino-2-ethylphenol, and a corresponding chlorocarbonyl compound instead of the compound prepared in Reference Example 5 to give the title compound; wherein a corresponding chlorocarbonyl compound was produced by operation in accordance with Reference Example 1→Reference Example 2→Reference Example 3→Reference Example 4→Reference Example 5 using 3-methanesulfonyl-5-(trifluoromethyl)aniline (CAS No. 1044271-86-4) instead of 5-(trifluoromethyl)pyridin-3-amine.

¹H NMR (400 MHz, DMSO-d₆) δ ppm: 8.55, 8.35, 8.23-8.22, 8.04, 7.54-7.52, 7.44-7.38, 6.43-6.41, 5.06-5.02, 4.92-4.87, 3.30, 2.39-2.33, 1.04-1.01;

LCMS: m/z 560 [M+H]+;

HPLC retention time: 1.339 min (method 88).

### Reference Example 49: 7,7-dimethyl-1,4-dioxaspiro[4.5]decan-8-one

To a stirred mixture of 1,4-dioxaspiro[4.5]decan-8-one (CAS No. 4746-97-8, 5.0 g) in THE (50 mL) were added CH₃I (6 mL) and t-BuOK (1M in THF; 70.5 mL) at 0° C. The resulting mixture was stirred for 15 h at room temperature under nitrogen atmosphere. The resulting solution was diluted with water and extracted with ethyl acetate. The organic layers were combined, washed with brine, dried over anhydrous sodium sulfate and concentrated under vacuum. The crude product was purified by column chromatography (SiO₂, Petroleum ether/EtOAc=4:1) to afford the title compound (1.0 g).

LCMS: m/z 185 [M+H]+.

### Reference Example 50: N-[(4-methoxyphenyl)methyl]-7,7-dimethyl-1,4-dioxaspiro[4.5]decan-8-amine

To a solution of the compound prepared in Reference Example 49 (1.0 g) in DCM (15 mL) were added (4-methoxyphenyl)methanamine (CAS No. 2393-23-9, 1 mL) at room temperature. The mixture was stirred at room temperature for 0.5 h. To the above mixture was added Na(OAc)₃BH (4 g). The resulting mixture was stirred for 14 h at room temperature. The reaction was concentrated to dryness and the residue was purified by Prep-TLC to afford the title compound (709 mg).

LCMS: m/z 306 [M+H]+.

### Reference Example 51: 4-[(4-methoxyphenyl)methylamino]-3,3-dimethyl-cyclohexanone

To a solution of the compound prepared in Reference Example 50 (709 mg) in acetone (6 mL) were added 2 M HCl (2 mL) at room temperature. The resulting mixture was stirred for 18 h at 25° C. The mixture was concentrated. The crude product was used in the next step directly without further purification.

### Reference Example 52: 4-[(4-methoxyphenyl)methylamino]-3,3-dimethyl-cyclohexanol

To a solution of the compound prepared in Reference Example 51 (171 mg) in methanol (2 mL) was added NaBH₄ (29.7 mg) at 0° C. The resulting mixture was stirred for 23 h at room temperature under nitrogen atmosphere. The filtrate was concentrated under reduced pressure. The crude product was used in the next step directly without further purification.

LCMS: m/z 306 [M+H]+.

### Reference Example 53: 4-amino-3,3-dimethyl-cyclohexanol

To a stirred mixture of the compound prepared in Reference Example 52 (176 mg) in methanol (2 mL) were added 20% Pd(OH)₂ on carbon (20 mg) at room temperature under Hz. The resulting mixture was stirred for 15 h at 20° C. The filtrate was concentrated under reduced pressure. The crude product was used in the next step directly without further purification.

### Example 26: 3-{4-[(7-methoxy-4-quinazolinyl)oxy]-2,2-dimethylcyclohexyl}-1-[5-(trifluoromethyl)-3-pyridinyl]-2,4-imidazolidinedione

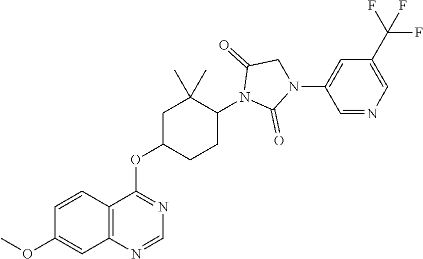 [SMILES: COc1ccc2c(OC3CCC(N4C(=O)CN(c5cncc(C(F)(F)F)c5)C4=O)C(C)(C)C3)ncnc2c1]

A procedure for a purpose similar to that for Reference Example 9→Example 3 was carried out by using the compound prepared in Reference Example 53 of 4-amino-2-ethylphenol, and 4-chloro-6-methoxyquinazoline instead of 2-amino-4-chloropyrimidine, to give the title compound.

¹H NMR (400 MHz, DMSO-d₆) δ 9.95, 8.74, 8.50, 8.03, 7.27, 5.52, 4.76-4.47, 4.13-3.88, 2.91-2.88, 2.39-2.33, 2.05, 1.80, 1.62, 1.08, 0.99;

LCMS: m/z 530 [M+H]+;

HPLC retention time: 1.395 min (method 84).

### Reference Example 54: 5-chloro-1H-pyrazolo[3,4-b]pyridin-3-ol

Into a 25-mL microwave tube, was placed methyl 2,5-dichloropyridine-3-carboxylate (CAS No. 67754-03-4, 500 mg), 1-Butanol (5 mL), N₂H4.H₂O (1.2 mL). The final reaction mixture was heated for 4 h at 120° C. The reaction mixture was concentrated under vacuum. The solid was collected by filtration. The solid was wash with n-BuOH. The solid was concentrated to dryness in vacuum to obtain the title compound (680 mg).

LCMS: m/z 170 [M+H]+.

### Reference Example 55: 5-chloro-1-[(4-methoxyphenyl)methyl]pyrazolo[3,4-b]pyridin-3-ol

To a stirred solution of the compound prepared in Reference Example 54 (2.5 g) in DMSO (25 mL) was added KOH (2481 mg), 1-(chloromethyl)-4-methoxybenzene (2.4 mL). The reaction was stirred at room temperature overnight. The resulting solution was extracted with ethyl acetate, washed with brine, dried over anhydrous Na₂SO₄. The reaction mixture was concentrated. The resulting solution was diluted with dichloromethane. The precipitation was filtered and washed with dichloromethane. The resulting mixture was concentrated under reduced pressure to obtain the title compound (1.2 g).

LCMS: m/z 290 [M+H]+.

### Reference Example 56: 5-chloro-3-(difluoromethoxy)-1-[(4-methoxyphenyl)methyl]pyrazolo[3,4-b]pyridine

To a solution of the compound prepared in Reference Example 55 (200 mg) in DMF (5 mL) was added K₂CO₃ (286 mg), (2-chloro-2,2-difluoro-acetyl)oxysodium (CAS No. 1895-39-2, 316 mg). The resulting mixture was stirred overnight at room temperature. The reaction was filtered and the filtrate was diluted with ethyl acetate, washed with brine, dried with Na₂SO₄ and concentration to dryness. The residue was purified by column chromatography to afford the title compound (110 mg).

LCMS: m/z 340 [M+H]+.

### Reference Example 57: 3-(difluoromethoxy)-1-[(4-methoxyphenyl)methyl]pyrazolo[3,4-b]pyridin-5-ol

To a solution of the compound prepared in Reference Example 56 (100 mg) in 1,4-dioxane (3 mL) and water (3 mL) was added t-Bubrettphos (57 mg), KOH (49 mg), Pd₂(dba)₃ (27 mg). The resulting mixture was stirred overnight at 90° C. The reaction was diluted with ethyl acetate, washed with brine, dried and concentration to dryness. The crude was purified by column chromatography to obtain the title compound (100 mg).

LCMS: m/z 322 [M+H]+.

### Reference Example 58: 3-(difluoromethoxy)-1-[(4-methoxyphenyl)methyl]-5-(4-nitrophenoxy)pyrazolo[3,4-b]pyridine

To a solution of the compound prepared in Reference Example 57 (90 mg) in DMF (1 mL) was added K₂CO₃ (116 mg), 1-fluoro-4-nitrobenzene (0.04 mL). The resulting mixture was stirred for 3 h at 60° C. The reaction was concentrated to dryness and the residue was diluted with ethyl acetate, washed with brine, dried and concentration to dryness. The crude was purified by column chromatography to obtain the title compound (120 mg).

LCMS: m/z 443 [M+H]+.

### Reference Example 59: 4-[3-(difluoromethoxy)-1-[(4-methoxyphenyl)methyl]pyrazolo[3,4-b]pyridin-5-yl]oxyaniline

To a solution of the compound prepared in Reference Example 58 (110 mg) in water (1 mL) and ethanol (3 mL) was added Fe (111 mg), NH₄Cl (106 mg). The resulting mixture was stirred for 2 h at 80° C. The mixture was allowed to cool down to room temperature, filtered and the filtrate was diluted with ethyl acetate, washed with brine, dried over anhydrous sodium sulfate and concentrated under vacuum to give the title compound (100 mg).

LCMS: m/z 413 [M+H]+.

### Reference Example 60: 3-[4-[3-(difluoromethoxy)-1-[(4-methoxyphenyl)methyl]pyrazolo[3,4-b]pyridin-5-yl]oxyphenyl]-1-[5-(trifluoromethyl)-3-pyridyl]imidazolidine-2,4-dione

To a solution of the compound prepared in Reference Example 59 (90 mg) in THE (1.00 mL) was added DIEA (0.18 mL), the compound prepared in Reference Example 5 (97 mg). The resulting mixture was stirred overnight at 80° C. The reaction was concentrated to dryness and the residue was diluted with ethyl acetate, washed with brine, dried over sodium sulfate, filtered and concentrated to obtain the title compound (110 mg).

LCMS: m/z 641 [M+H]+.

### Example 27: 3-(4-{[3-(difluoromethoxy)-1H-pyrazolo[3,4-b]pyridin-5-yl]oxy}phenyl)-1-[5-(trifluoromethyl)-3-pyridinyl]-2,4-imidazolidinedione

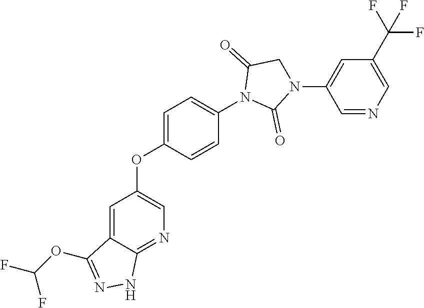 [SMILES: O=C1CN(c2cncc(C(F)(F)F)c2)C(=O)N1c1ccc(Oc2cnc3[nH]nc(OC(F)F)c3c2)cc1]

The compound prepared in Reference Example 60 (90 mg) was dissolved in TFA (1.00 mL) and stirred for 3 h at 70° C. The solvent was removed under vacuum. The residue was purified by Prep-HPLC to obtain the title compound (26 mg).

¹H NMR (400 MHz, DMSO-d₆) δ 13.49, 9.17, 8.77, 8.55-8.52, 7.98, 7.71-7.35, 7.17-7.15, 4.75;

LCMS: m/z 521 [M+H]+;

HPLC retention time: 1.566 min (method 85).

### Reference Example 61(1)˜61(2)

A procedure for a purpose similar to that for Reference Example 60→Example 27 was carried out by using a corresponding chlorocarbonyl compound instead of the compound prepared in Reference Example 5, to give the following compounds of Reference Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Reference Example 61(1): 3-(4-{[3-(difluoromethoxy)-1H-pyrazolo[3,4-b]pyridin-5-yl]oxy}phenyl)-1-[3-(2,2,2-trifluoro-1-hydroxyethyl)phenyl]-2,4-imidazolidinedione

¹H NMR (400 MHz, DMSO-d₆) δ13.48, 8.55, 7.97-7.93, 7.63-7.58, 7.52, 7.48-7.39, 7.30-7.25, 7.16-7.13, 5.20-5.10, 4.60;

LCMS: m/z 550 [M+H]+.

### Reference Example 61(2): 3-(4-{[3-(difluoromethoxy)-1H-pyrazolo[3,4-b]pyridin-5-yl]oxy}phenyl)-1-[5-(2,2,2-trifluoro-1-hydroxyethyl)-3-pyridinyl]-2,4-imidazolidinedione

¹H NMR (400 Hz, DMSO-d₆) δ 13.48, 8.79, 8.55, 8.46, 8.39, 7.97, 7.71-7.34, 7.17-7.13, 5.38-5.34, 4.68;

LCMS: m/z 551 [M+H]+.

### Example 28(1)˜28(2)

The racemate of the compound prepared in Reference Example 61(1) was splitted by Chiral-HPLC to afford the title compounds.

### Example 28(1): 3-(4-{[3-(difluoromethoxy)-1H-pyrazolo[3,4-b]pyridin-5-yl]oxy}phenyl)-1-[3-(2,2,2-trifluoro-1-hydroxyethyl)phenyl]-2,4-imidazolidinedione

¹H NMR (400 MHz, DMSO-d₆) δ ppm: 13.48, 8.55, 8.00-7.90, 7.70-7.58, 7.52-7.34, 7.30-7.25, 7.18-7.12, 6.91, 5.21-5.13, 4.61;

LCMS: m/z 550 [M+H]+;

HPLC retention time: 2.829 min (method 86).

Chiral HPLC retention time: 2.057 min (column: CHIRALPAK ID-3 (trademark), Mobile phase: MTBE (0.1% DEA)/MeOH=50/50, Flow: 1.0 mL/min, Temp: 25 degree).

### Example 28(2)

### 3-(4-{[3-(difluoromethoxy)-1H-pyrazolo[3,4-b]pyridin-5-yl]oxy}phenyl)-1-[3-(2,2,2-trifluoro-1-hydroxyethyl)phenyl]-2,4-imidazolidinedione

¹H NMR (400 MHz, DMSO-d₆) δ ppm: 13.48, 8.55, 7.98, 7.95, 7.72-7.60, 7.51-7.33, 7.31-7.25, 7.19-7.11, 6.91, 5.19-5.16, 4.61;

LCMS: m/z 550 [M+H]+;

HPLC retention time: 2.826 min (method 86).

Chiral HPLC retention time: 2.864 min (column: CHIRALPAK ID-3 (trademark), Mobile phase: MTBE (0.1% DEA)/MeOH=50/50, Flow: 1.0 mL/min, Temp: 25 degree).

### Example 29(1)˜29(2)

The racemate of the compound prepared in Reference Example 61(2) was splitted by Chiral-HPLC to afford the title compounds.

### Example 29(1): 3-(4-{[3-(difluoromethoxy)-1H-pyrazolo[3,4-b]pyridin-5-yl]oxy}phenyl)-1-[5-(2,2,2-trifluoro-1-hydroxyethyl)-3-pyridinyl]-2,4-imidazolidinedione

¹H NMR (400 Hz, DMSO-d₆) δ 13.48, 8.79, 8.55, 8.46, 8.39, 7.97, 7.71-7.34, 7.17-7.13, 5.38-5.34, 4.68;

LCMS: m/z 551 [M+H]+;

HPLC retention time: 1.443 min (method 88).

Chiral HPLC retention time: 1.712 min (column: CHIRALPAK IH-3 (trademark), Mobile phase: (Hexane:DCM=3:1)(0.1% DEA)/EtOH=70/30, Flow: 1.0 mL/min, Temp: 25 degree).

### Example 29(2): 3-(4-{[3-(difluoromethoxy)-1H-pyrazolo[3,4-b]pyridin-5-yl]oxy}phenyl)-1-[5-(2,2,2-trifluoro-1-hydroxyethyl)-3-pyridinyl]-2,4-imidazolidinedione

¹H NMR (400 Hz, DMSO-d₆) δ 13.48, 8.79, 8.55, 8.46, 8.39, 7.97, 7.71-7.34, 7.15-7.13, 5.38-5.34, 4.68;

LCMS: m/z 551 [M+H]+;

HPLC retention time: 1.448 min (method 88).

Chiral HPLC retention time: 1.213 min (column: CHIRALPAK IH-3 (trademark), Mobile phase: (Hexane:DCM=3:1)(0.1% DEA)/EtOH=70/30, Flow: 1.0 mL/min, Temp: 25 degree).

### Reference Example 62: 4-amino-6-chloro-pyridine-3-carboxylic acid

To a stirred mixture of methyl 4-amino-6-chloro-pyridine-3-carboxylate (CAS No. 1256785-40-6, 1.0 g) in THF (6 mL) and water (20 mL) was added LiOH (386 mg). The resulting mixture was stirred for 3 h at room temperature under nitrogen atmosphere. The residue was diluted with water, then adjusted to pH 6˜7 with 1M HCl. The residue was extracted with ethyl acetate The organics were concentrated to obtain the title compound (380 mg).

LCMS: m/z 173 [M+H]+.

### Reference Example 63: 7-chloro-1H-pyrido[4,3-d]pyrimidin-4-one

To a stirred solution of the compound prepared in Reference Example 62 (200 mg) in 2-Methoxyethanol (10 mL) was added Formamidine acetate (482 mg). The mixture was stirred for 3 h at 150° C. under microwave. The reaction was concentrated and the residue was diluted with water. The solid was collected by filtered and washed with water to afford the title compound (70 mg).

LCMS: m/z 182 [M+H]+.

### Reference Example 64: 7-methoxy-1H-pyrido[4,3-d]pyrimidin-4-one

A solution of the compound prepared in Reference Example 63 (70.0 mg) in 30% MeONa in MeOH (1.0 mL) was stirred for 4 h at 70° C. The residue was adjusted to pH 6˜7 with 1M HCL. The solid was collected to obtain the title compound (30 mg).

LCMS: m/z 178 [M+H]+.

### Reference Example 65: 4-chloro-7-methoxy-pyrido[4,3-d]pyrimidine

To a solution of the compound prepared in Reference Example 64 (120.0 mg) in chloroform (3 mL) was added POCl₃ (0.2 mL). The resulting mixture was stirred for 1 h at 70° C. under nitrogen atmosphere. Then to the mixture was added TEA (0.4 mL) at room temperature. The reaction was stirred at 70° C. for 4 h. The reaction was concentrated to dryness and the residue was taken up in dichloromethane and washed with sodium bicarbonate (sat.). The resulting solution was extracted with dichloromethane. The organics were concentrated and purified by flash column chromatography to obtain the title compound (70 mg).

LCMS: m/z 196 [M+NH₄]+.

### Example 30: 3-{trans-4-[(7-methoxypyrido[4,3-d]pyrimidin-4-yl)oxy]cyclohexyl}-1-[5-(trifluoromethyl)-3-pyridinyl]-2,4-imidazolidinedione

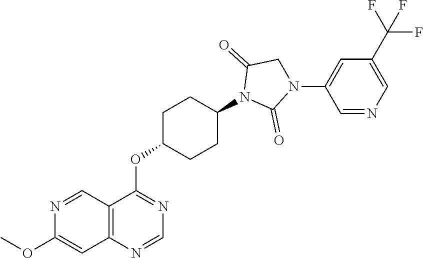 [SMILES: COc1cc2ncnc(O[C@H]3CC[C@H](N4C(=O)CN(c5cncc(C(F)(F)F)c5)C4=O)CC3)c2cn1]

A procedure for a purpose similar to that for Reference Example 40 was carried out by using the compound prepared in Reference Example 65 instead of 4-chloro-7-nitroquinazoline, to give the title compound.

¹H NMR (400 Hz, DMSO-d₆) δ 9.21, 9.10, 8.83, 8.75, 8.49, 7.11, 5.39-5.29, 4.59, 4.12-4.01, 2.37-2.27, 1.91-1.83, 1.81-1.69;

LCMS: m/z 503 [M+H]+;

HPLC retention time: 1.643 min (method 88).

### Example 31: 3-{4-[(7-methoxypyrido[4,3-d]pyrimidin-4-yl)oxy]phenyl}-1-[5-(trifluoromethyl)-3-pyridinyl]-2,4-imidazolidinedione

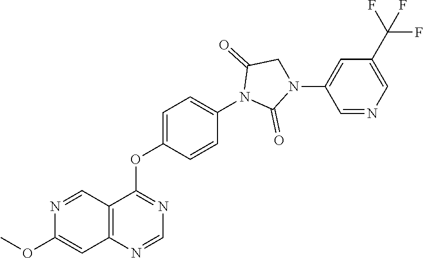 [SMILES: COc1cc2ncnc(Oc3ccc(N4C(=O)CN(c5cncc(C(F)(F)F)c5)C4=O)cc3)c2cn1]

A procedure for a purpose similar to that for Reference Example 9→Example 3 was carried out by using 4-aminophenol instead of 4-amino-2-ethylphenol, and the compound prepared in Reference Example 65 instead of 2-amino-4-chloropyrimidine, to give the title compound.

¹H NMR (400 Hz, DMSO-d₆) 9.53, 9.20, 8.79, 8.55, 7.58-7.57, 7.23, 4.81, 4.07;

LCMS: m/z 497 [M+H]+;

HPLC retention time: 1.562 min (method 88).

### Reference Example 66: 5-chloro-1-[(4-methoxyphenyl)methyl]pyrazolo[3,4-b]pyridin-3-amine

To a solution of KOH (1.66 g) in DMSO (20 mL) was added 5-chloro-1H-pyrazolo[3,4-b]pyridin-3-amine (CAS No. 1352909-30-8, 2.00 g). After 5 min, 1-(chloromethyl)-4-methoxybenzene (1.93 mL) was added to this mixture. The reaction mixture was stirred for 30 min at room temperature. The reaction mixture was quenched with water, extracted with chloroform, washed with brine, dried over anhydrous sodium sulfate, filtered and concentrated under reduced pressure. The crude product was purified by column chromatography to afford the title compound (2 g).

LCMS: m/z 289 [M+H]+.

### Reference Example 67: 3-amino-1-[(4-methoxyphenyl)methyl]pyrazolo[3,4-b]pyridin-5-ol

To a solution of the compound prepared in Reference Example 66 (500 mg) in dry 1,4-dioxane (10 mL) was added KOAc (509 mg), Pd₂(dba)₃CHCl₃ (179 mg), PCy₃HBF₄ (127 mg) and 4,4,4,4′,4′,5,5,5′,5′-octamethyl-2,2′-bi(1,3,2-dioxaborolane) (1319 mg) under nitrogen. The reaction mixture was stirred for 4 h at 100° C. The reaction was allowed to cool to room temperature. Then NaBO₃.4H₂O in water was added to this mixture. The reaction mixture was stirred for 1 h at room temperature. The reaction mixture was quenched with water, extracted with ethyl acetate, washed with brine, dried over anhydrous sodium sulfate, filtered and concentrated under reduced pressure. The crude product was purified by column chromatography to afford the title compound (200 mg).

LCMS: m/z 271 [M+H]+.

### Reference Example 68: 1-[(4-methoxyphenyl)methyl]-5-(4-nitrophenoxy)pyrazolo[3,4-b]pyridin-3-amine

To a solution of the compound prepared in Reference Example 67 (100 mg) in DMF (5 mL) was added K₂CO₃ (153 mg) and 1-fluoro-4-nitrobenzene (0.05 mL). The reaction mixture was stirred for 2 h at 60° C. The reaction mixture was quenched with water, extracted with ethyl acetate, washed with brine, dried over anhydrous sodium sulfate, filtered and concentrated under reduced pressure. The residue was purified by Prep-TLC to afford the title compound (110 mg).

LCMS: m/z 392 [M+H]+.

### Reference Example 69

### 3-chloro-1-[(4-methoxyphenyl)methyl]-5-(4-nitrophenoxy)pyrazolo[3,4-b]pyridine

To a stirred solution of the compound prepared in Reference Example 68 (300 mg) in MeCN (5 mL) was added CuCl₂ (124 mg) and amyl nitrate (CAS No. 463-04-7, 135 mg) dropwise at −10° C. under N₂. The resulting mixture was stirred for 12 h at room temperature. The resulting mixture was extracted with ethyl acetate, dried over anhydrous Na₂SO₄. After filtration, the filtrate was concentrated under reduced pressure. The residue was purified by column chromatography to afford the title compound (120 mg).

### Reference Example 70: 4-[3-chloro-1-[(4-methoxyphenyl)methyl]pyrazolo[3,4-b]pyridin-5-yl]oxyaniline

To a solution of the compound prepared in Reference Example 69 (140 mg) in ethanol (10 mL) was added a solution of NH₄Cl (92 mg) in water (10 mL). The mixture was heated to reflux, and then Fe (95 mg) was added. The mixture was stirred at reflux for 2 h. Solids were filtered out and the solvent was evaporated under vacuum, then the residue was extracted with EtOAc. The combined organic extracts were washed brine, dried over anhydrous sodium sulfate, and concentrated under vacuum. The crude product was purified by column chromatography to afford the title compound (120 mg).

### Reference Example 71: 3-[4-[3-chloro-1-[(4-methoxyphenyl)methyl]pyrazolo[3,4-b]pyridin-5-yl]oxyphenyl]-1-[5-(trifluoromethyl)-3-pyridyl]imidazolidine-2,4-dione

To a stirred solution of the compound prepared in Reference Example 70 (140 mg) and the compound prepared in Reference Example 5 (163 mg) in THE (5 mL) were added DIEA (5 mL). The resulting mixture was stirred for overnight at 60° C. under N₂ atmosphere.

The resulting mixture was concentrated under vacuum and concentrated under reduced pressure. The residue was purified by Prep-TLC to afford the title compound (150 mg).

LCMS: m/z 609 [M+H]+.

### Example 32: 3-{4-[(3-chloro-1H-pyrazolo[3,4-b]pyridin-5-yl)oxy]phenyl}-1-[5-(trifluoromethyl)-3-pyridinyl]-2,4-imidazolidinedione

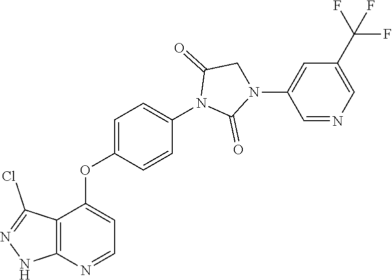 [SMILES: O=C1CN(c2cncc(C(F)(F)F)c2)C(=O)N1c1ccc(Oc2ccnc3[nH]nc(Cl)c23)cc1]

To a solution of the compound prepared in Reference Example 71 (70 mg) in DCM (1 mL) was added TFA (4 mL) stirred at room temperature. The reaction mixture was stirred for 20 h at 70° C. The reaction mixture was concentrated under reduced pressure to afford the crude product. The crude product was purified by Prep-HPLC to afford the title compound (11.6 mg).

¹H NMR (400 MHz, DMSO-d₆) δ ppm: 14.08, 9.20-9.16, 8.80-8.78, 8.60-8.49, 8.00-7.95, 7.48-7.40, 7.24-7.18, 4.76;

LCMS: m/z 489 [M+H]+;

HPLC retention time: 1.474 min (method 5).

### Reference Example 72: 1-(2,5-dichloro-3-pyridyl)-2,2,2-trifluoro-ethanol

To a stirred solution of 2,5-dichloronicotinaldehyde (2.0 g) in DMF (20 mL) was added K₂CO₃ (3.1 g), TMSCF₃ (2.4 g) under nitrogen atmosphere. The resulting mixture was stirred overnight at room temperature under nitrogen atmosphere. The reaction was diluted in ethyl acetate and washed with water and saturated brine solution. The organics phase was concentrated and the crude product was purified by column chromatography to obtain the title compound (2.0 g).

LCMS: m/z 246 [M+H]+.

### Reference Example 73: 1-(2,5-dichloro-3-pyridyl)-2,2,2-trifluoro-ethanone

To a stirred solution of the compound prepared in Reference Example 72 (2.0 g) in Ethyl acetate (20 mL) was added IBX (4.55 g) under nitrogen atmosphere. The resulting mixture was stirred overnight at 60° C. under nitrogen atmosphere. The resulting suspension was filtered, the filter cake was washed with ethyl acetate. The filtrate was concentrated under reduced pressure. The residue was purified by column chromatography to afford the title compound (850 mg).

### Reference Example 74: 5-chloro-3-(trifluoromethyl)-1H-pyrazolo[3,4-b]pyridine

A solution of the compound prepared in Reference Example 73 (250 mg) in NH₂NH₂.H₂O (2 mL) stirred for 4 hours at 80° C. under nitrogen atmosphere. The resulting mixture was diluted with ethyl acetate. The combined organic layers were washed with H₂O and brine, dried over anhydrous sodium sulfate. After filtration, the filtrate was concentrated under reduced pressure. The residue was purified by column chromatography, eluted with ethyl acetate/petroleum ether (1:5) to afford the title compound (170 mg).

### Reference Example 75: 5-chloro-1-tetrahydropyran-2-yl-3-(trifluoromethyl)pyrazolo[3,4-b]pyridine

To a stirred solution of the compound prepared in Reference Example 74 (170 mg) in DCM (5 mL) was added p-TSA (13 mg), 3,4-dihydro-2H-pyran (0.1 mL) at 0° C. under nitrogen atmosphere. The resulting mixture was stirred for 2 h at room temperature under nitrogen atmosphere. The reaction was concentrated to dryness and the residue was taken up in dichloromethane and the organics washed with water and saturated brine solution. The crude was purified by flash column chromatography to obtain the title compound (230 mg).

LCMS: m/z 306 [M+H]+.

### Reference Example 76: 1-tetrahydropyran-2-yl-3-(trifluoromethyl)pyrazolo[3,4-b]pyridin-5-ol

To a stirred solution of the compound prepared in Reference Example 75 (220 mg) in 1,4-dioxane (2 mL) and water (2 mL) were added KOH (81 mg), t-Bubrettphos (53 mg), Pd₂(dba)₃ (66 mg) under nitrogen atmosphere. The resulting mixture was stirred for 2 h at 80° C. under nitrogen atmosphere. The reaction was concentrated to dryness and the residue was taken up in ethyl acetate and the organics washed with brine. The organics were concentrated. The crude was then purified by flash column chromatography to obtain the title compound (200 mg).

LCMS: m/z 288 [M+H]+.

### Reference Example 77

### 5-(4-nitrophenoxy)-1-tetrahydropyran-2-yl-3-(trifluoromethyl)pyrazolo[3,4-b]pyridine

To a stirred solution of the compound prepared in Reference Example 76 (200 mg) in DMF (4 mL) was added K₂CO₃ (289 mg), 1-fluoro-4-nitrobenzene (0.11 mL) under nitrogen atmosphere. The resulting mixture was stirred for 2 h at 60° C. under nitrogen atmosphere. The reaction was concentrated to dryness and the residue was taken up in ethyl acetate and the organics washed with water and saturated brine solution. The organics were concentrated and purified by column chromatography to afford the title compound (200 mg).

LCMS: m/z 409 [M+H]+.

### Reference Example 78: 4-[1-tetrahydropyran-2-yl-3-(trifluoromethyl)pyrazolo[3,4-b]pyridin-5-yl]oxyaniline

To a stirred solution of the compound prepared in Reference Example 77 (200 mg) in ethanol (9 mL) and water (3 mL) were added Fe (219 mg) and NH₄Cl (209 mg) under nitrogen atmosphere. The resulting mixture was stirred for 2 h at 80° C. under nitrogen atmosphere. The resulting suspension was filtered, the filter cake was washed with ethyl acetate. The filtrate was concentrated under reduced pressure. The resulting mixture was extracted with ethyl acetate, washed with brine, dried over anhydrous sodium sulfate and concentrated under vacuum to afford the title compound (180 mg).

LCMS: m/z 379 [M+H]+.

### Reference Example 79: 3-[4-[1-tetrahydropyran-2-yl-3-(trifluoromethyl) pyrazolo[3,4-b]pyridin-5-yl]oxyphenyl]-1-[5-(trifluoromethyl)-3-pyridyl]imidazolidine-2,4-dione

To a stirred solution of the compound prepared in Reference Example 78 (90 mg) in THF (2.0 mL) was added DIEA (0.2 mL) and the compound prepared in Reference Example 5 (85 mg) at 0° C. under nitrogen atmosphere. The resulting mixture was stirred for 2 h at 60° C. under nitrogen atmosphere. The reaction was concentrated to dryness and the residue was diluted with water and extracted with ethyl acetate. The combined organic layer was washed with brine, dried over sodium sulfate, filtered, concentrated to obtain the title compound (100 mg).

LCMS: m/z 607 [M+H]+.

### Example 33: 3-(4-{[3-(trifluoromethyl)-1H-pyrazolo[3,4-b]pyridin-5-yl]oxy}phenyl)-1-[5-(trifluoromethyl)-3-pyridinyl]-2,4-imidazolidinedione

To a stirred solution of the compound prepared in Reference Example 79 (90 mg) in DCM (1 mL) was added TFA (3.0 mL). The resulting mixture was stirred for 2 h at room temperature. The reaction was concentrated to dryness. The residue was diluted with water, then adjusted to pH 8-9 with sodium bicarbonate and extracted with ethyl acetate. The combined organic layer was washed with brine, concentrated. The residue was purified by Prep-HPLC to afford the title compound (26.0 mg).

¹H NMR (400 MHz, DMSO-d₆) δ 14.82, 9.18, 8.78-8.67, 8.52, 8.03, 7.47-7.43, 7.24-7.20, 4.76;

LCMS: m/z 523 [M+H]+;

HPLC retention time: 1.670 min (method 17).

### Example 33(1)˜ 33(3)

A procedure for a purpose similar to that for Reference Example 75→Reference Example 76→Reference Example 77→Reference Example 78→Reference Example 79→Example 33 was carried out by using a corresponding chloroaryl compound instead of the compound prepared in Reference Example 74, to give the following compounds of Examples.

### Example 33(1): 3-{4-[(2-methyl-3H-imidazo[4,5-b]pyridin-6-yl)oxy]phenyl}-1-[5-(trifluoromethyl)-3-pyridinyl]-2,4-imidazolidinedione

¹H NMR (400 MHz, DMSO-d₆) δ ppm: 12.96-12.53, 9.17, 8.78, 8.52, 8.19-8.14, 7.75-7.67, 7.41-7.39, 7.13-7.08, 4.75, 2.58-2.54;

LCMS: m/z 469 [M+H]+;

HPLC retention time: 1.041 min (method 84).

### Example 33(2): 3-(4-1 [2-(trifluoromethyl)-3H-imidazo[4,5-b]pyridin-6-yl]oxy phenyl)-1-[5-(trifluoromethyl)-3-pyridinyl]-2,4-imidazolidinedione

¹H NMR (400 MHz, DMSO-d₆) δ ppm: 14.90-14.37, 9.18, 8.78, 8.52-8.51, 8.25-7.79, 7.45-7.43, 7.19, 4.76;

LCMS: m/z 523 [M+H]+;

HPLC retention time: 1.328 min (method 84).

### Example 33(3): 3-[4-(3H-[1,2,3]triazolo[4,5-b]pyridin-6-yloxy)phenyl]-1-[5-(trifluoromethyl)-3-pyridinyl]-2,4-imidazolidinedione

¹H NMR (400 MHz, DMSO-d₆) δ 9.18, 8.78, 8.64, 8.52, 8.41-7.89, 7.47, 7.27, 4.76;

LCMS: m/z 456 [M+H]+;

HPLC retention time: 1.173 min (method 84).

### Reference Example 80: 5-bromo-1H-pyrazolo[3,4-b]pyridin-3-ol

To a solution of methyl 5-bromo-2-chloronicotinate (CAS No. 78686-79-0, 1.50 g) in ethanol (30 mL) was added N₂114 (0.9 mL). The reaction mixture was stirred for 16 h at 80° C. The solids were collected by filtration to afford the title compound (1 g).

LCMS: m/z 213 [M+H]+.

### Reference Example 81: 5-bromo-1-[(4-methoxyphenyl)methyl]pyrazolo[3,4-b]pyridin-3-ol

To a solution of the compound prepared in Reference Example 80 (1.00 g) in DMSO (20 mL) was added KOH (655 mg). The reaction mixture was stirred for 10 min at room temperature. Then 1-(chloromethyl)-4-methoxybenzene (0.63 mL) was added to this mixture. The reaction mixture was stirred for 16 h at room temperature. The mixture was concentrated. The crude product was purified by preparative HPLC to provide the title compound (1 g).

LCMS: m/z 333 [M+H]+.

### Reference Example 82: 5-bromo-3-(difluoromethoxy)-1-[(4-methoxyphenyl)methyl]pyrazolo[3,4-b]pyridine

To a solution of the compound prepared in Reference Example 81 (400 mg) in DMF (5 mL) was added K₂CO₃ (495 mg) and (2-chloro-2,2-difluoro-acetyl)oxysodium (547 mg). The reaction mixture was stirred for 12 h at 60° C. The reaction mixture was quenched with water. The resulting solution was extracted with ethyl acetate and the organic layers were combined, washed with brine, dried over anhydrous sodium sulfate, and concentrated under reduced pressure to afford crude product. The crude product was purified by column chromatography (SiO₂, Petroleum ether/EtOAc=1:1) to afford the title compound (260 mg).

LCMS: m/z 384 [M+H]+.

### Reference Example 83: tert-butyl N—[trans-4-[[3-(difluoromethoxy)-1-[(4-methoxyphenyl)methyl]pyrazolo[3,4-b]pyridin-5-yl]amino] cyclohexyl] carbamate

To a solution of the compound prepared in Reference Example 82 (100 mg) in toluene (5 mL) was added K₂CO₃ (179 mg), tert-butyl ((1r,4r)-4-aminocyclohexyl)carbamate (278 mg) and XPhos Pd-2^{nd} (102 mg) stirred at room temperature. The reaction mixture was stirred for 20 h at 100° C. The resulting solution was diluted with water and extracted with ethyl acetate. The organic layers were combined, washed with brine, dried over anhydrous sodium sulfate and concentrated under vacuum. The crude product was purified by Prep-TLC (ethyl acetate/petroleum ether=1/1) to afford the title compound (40 mg).

LCMS: m/z 518 [M+H]+.

### Reference Example 84: trans-N₄-[3-(difluoromethoxy)-1-[(4-methoxyphenyl)methyl]pyrazolo[3,4-b]pyridin-5-yl]cyclohexane-1,4-diamine

The mixture of the compound prepared in Reference Example 83 (60 mg) in HCl (4 M in dioxane; 2 mL) was stirred for 1 h at room temperature. The reaction mixture was concentrated under reduced pressure to afford the crude product. The crude product was used in the next step directly without further purification.

LCMS: m/z 418 [M+H]+.

### Reference Example 85: 3-[trans-4-[[3-(difluoromethoxy)-1-[(4-methoxyphenyl)methyl]pyrazolo[3,4-b]pyridin-5-yl]amino] cyclohexyl]-1-[5-(trifluoromethyl)-3-pyridyl]imidazolidine-2,4-dione

To a solution of the compound prepared in Reference Example 84 (48 mg) in THF (0.50 mL) was added DIEA (2 mL) and the compound prepared in Reference Example 5 (51 mg). The reaction mixture was stirred for 16 h at 60° C. The reaction mixture was concentrated under reduced pressure to afford the crude product. The crude product was purified by Prep-TLC to afford the title compound (65 mg).

LCMS: m/z 646 [M+H]+.

### Example 34: 3-(trans-4-{[3-(difluoromethoxy)-1H-pyrazolo[3,4-b]pyridin-5-yl]amino}cyclohexyl)-1-[5-(trifluoromethyl)-3-pyridinyl]-2,4-imidazolidinedione

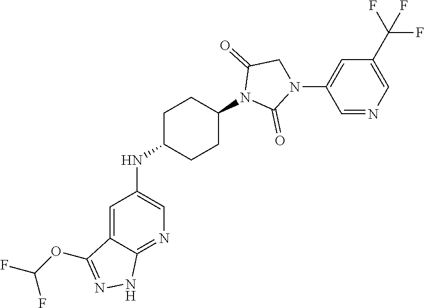 [SMILES: O=C1CN(c2cncc(C(F)(F)F)c2)C(=O)N1[C@H]1CC[C@H](Nc2cnc3[nH]nc(OC(F)F)c3c2)CC1]

To a solution of the compound prepared in Reference Example 85 (60 mg) in TFA (1.5 mL) was added trifluoromethanesulfonic acid (0.2 mL). The reaction mixture was stirred for 1 h at 60° C. The residue was diluted with water, then adjusted to pH=7 with sodium bicarbonate. The resulting solution was extracted with ethyl acetate and the organic layers were combined, washed with brine, dried over anhydrous sodium sulfate, filtered and concentrated under reduced pressure to afford crude product. The crude product was purified by Prep-TLC to afford the crude product. The crude product was purified by preparative HPLC to afford the title compound (7.2 mg).

¹H NMR (400 MHz, DMSO-d₆) δ ppm: 12.79, 9.09, 8.75, 8.49, 8.15, 7.60-7.30, 7.04, 5.69-5.67, 4.58, 3.98-3.92, 2.41-2.29, 2.19-2.08, 1.78-1.75, 1.32-1.19;

LCMS: m/z 526 [M+H]+;

HPLC retention time: 1.225 min (method 90).

### Example 34(1)˜34(4)

A procedure for a purpose similar to that for Reference Example 82→Reference Example 83→Reference Example 84→Reference Example 85→Example 34 was carried out by using a corresponding compound instead of (2-chloro-2,2-difluoro-acetyl)oxysodium, to give the following compounds of Examples.

### Example 34(1): 3-(trans-4-{[3-(2-methoxyethoxy)-1H-pyrazolo[3,4-b]pyridin-5-yl]amino}cyclohexyl)-1-[5-(trifluoromethyl)-3-pyridinyl]-2,4-imidazolidinedione

¹H NMR (400 MHz, DMSO-d₆) δ ppm: 12.02, 9.09, 8.75, 8.50, 8.10, 7.06, 5.55-5.30, 4.58, 4.45-4.36, 4.02-3.92, 3.78-3.70, 3.33-3.30, 2.39-2.23, 2.16-2.09, 1.82-1.70, 1.35-1.20;

LCMS: m/z 534 [M+H]+;

HPLC retention time: 1.332 min (method 88).

### Example 34(2): 3-(trans-4-{[3-(2-hydroxyethoxy)-1H-pyrazolo[3,4-b]pyridin-5-yl]amino}cyclohexyl)-1-[5-(trifluoromethyl)-3-pyridinyl]-2,4-imidazolidinedione

¹H NMR (400 MHz, DMSO-d₆) δ ppm: 11.96, 9.09, 8.74, 8.49, 8.07, 7.02, 5.45, 4.87, 4.58, 4.31-4.25, 4.01-3.89, 3.80-3.74, 3.23-3.10, 2.35-2.25, 2.18-2.09, 1.80-1.71, 1.30-1.20;

LCMS: m/z 520 [M+H]+;

HPLC retention time: 1.214 min (method 88).

### Example 34(3): 3-[trans-4-[[3-(3-morpholin-4-ylpropoxy)-1H-pyrazolo[3,4-b]pyridin-5-yl]amino]cyclohexyl]-1-[5-(trifluoromethyl)pyridin-3-yl]imidazolidine-2,4-dione;2,2,2-trifluoroacetic acid; trifluoromethanesulfonic acid

¹H NMR (400 MHz, DMSO-d₆) δ ppm: 12.26, 9.91, 9.09, 8.75, 8.49, 8.19, 7.21, 4.58, 4.39, 4.04-3.90, 3.72-3.65, 3.55-3.48, 3.38-3.25, 3.17-3.07, 2.32-2.18, 2.15-2.09, 1.82-1.72, 1.41-1.27;

LCMS: m/z 603 [M+H]+;

HPLC retention time: 2.071 min (method 89).

### Example 34(4): 3-[trans-4-({3-[3-(methylsulfonyl)propoxy]-1H-pyrazolo[3,4-b]pyridin-5-yl}amino)cyclohexyl]-1-[5-(trifluoromethyl)-3-pyridinyl]-2,4-imidazolidinedione

¹H NMR (300 MHz, DMSO-d₆) δ 12.03, 9.08, 8.74, 8.49, 8.08, 7.07, 5.43, 4.58, 4.41-4.36, 4.01-3.88, 3.31, 3.03, 2.35-2.21, 2.19-2.08, 1.83-1.71, 1.35-1.19;

LCMS: m/z 596 [M+H]+;

HPLC retention time: 1.308 min (method 88).

### Example 35(1)˜35(2)

A procedure for a purpose similar to that for Reference Example 80→Reference Example 81→Reference Example 83→Reference Example 84→Reference Example 85→Example 34 was carried out by using 5-bromo-2-chloronicotinonitrile instead of methyl 5-bromo-2-chloronicotinate, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 35(1): 3-{trans-4-[(3-amino-1H-pyrazolo[3,4-b]pyridin-5-yl)amino]cyclohexyl}-1-[5-(trifluoromethyl)-3-pyridinyl]-2,4-imidazolidinedione

¹H NMR (400 MHz, DMSO-d₆) δ 11.51, 9.09, 8.74, 8.48, 7.99, 7.29, 6.00-5.00, 4.57, 4.00-3.83, 3.20-3.13, 2.32-2.21, 2.28-2.11, 1.87-1.73, 1.32-1.21;

LCMS: m/z 475 [M+H]+;

HPLC retention time: 2.093 min (method 89).

### Example 35(2): 3-{4-[(3-amino-1H-pyrazolo[3,4-b]pyridin-5-yl)amino]cyclohexyl}-1-[3-fluoro-5-(trifluoromethyl)phenyl]-2,4-imidazolidinedione trifluoroacetate (1:1)

¹H NMR (400 MHz, DMSO-d₆) δ 8.27, 8.01, 7.74, 7.45, 7.27, 4.52, 3.98-3.95, 3.23-3.18, 2.29-2.20, 2.13-2.10, 1.80-1.75, 1.35-1.32;

LCMS: m/z 491 [M+H]+;

HPLC retention time: 1.559 min (method 94).

### Reference Example 86: tert-butyl N-[trans-4-[3-(difluoromethoxy)-1-[(4-methoxyphenyl)methyl]pyrazolo[3,4-b]pyridin-5-yl]oxycyclohexyl]carbamate

The mixture of the compound prepared in Reference Example 56 (1.1 g), tert-butyl ((1s,4s)-4-hydroxycyclohexyl)carbamate (1.5 g), DIAD (1.4 g) and PPh₃ (1.8 g) in THF (20 mL) was stirred at room temperature for 16 h. Then the mixture was diluted with water and extracted with ethyl acetate. The organic layers were combined, dried over anhydrous sodium sulfate and concentrated under vacuum. The residue was purified by column chromatography to afford the title compound (1.5 g).

LCMS: m/z 419 [M+H]+.

### Reference Example 87: 4-[trans-3-(difluoromethoxy)-1-[(4-methoxyphenyl)methyl]pyrazolo[3,4-b]pyridin-5-yl]oxycyclohexanamine

The mixture of the compound prepared in Reference Example 86 (150 mg) in HCl (4 M in dioxane; 5 mL) was stirred for 1 h at room temperature. The reaction mixture was concentrated under reduced pressure to afford the title compound (120 mg). The crude product was used in the next step directly without further purification.

### Reference Example 88: 3-[trans-4-[3-(difluoromethoxy)-1-[(4-methoxyphenyl)methyl]pyrazolo[3,4-b]pyridin-5-yl] oxycyclohexyl]-1-[5-(trifluoromethyl)-3-pyridyl]imidazolidine-2,4-dione

To a solution of the compound prepared in Reference Example 87 (100 mg) in THF (2 mL) was added diethyl acetate (2 mL) and the compound prepared in Reference Example 5 (85 mg). The reaction mixture was stirred for 3 h at 60° C. The reaction mixture was concentrated under reduced pressure to afford the crude product. The residue was purified by Prep-TLC to afford the title compound (90 mg).

LCMS: m/z 647 [M+H]+.

### Example 36: 3-(trans-4-{[3-(difluoromethoxy)-1H-pyrazolo[3,4-b]pyridin-5-yl]oxy}cyclohexyl)-1-[5-(trifluoromethyl)-3-pyridinyl]-2,4-imidazolidinedione trifluoroacetate (1:1)

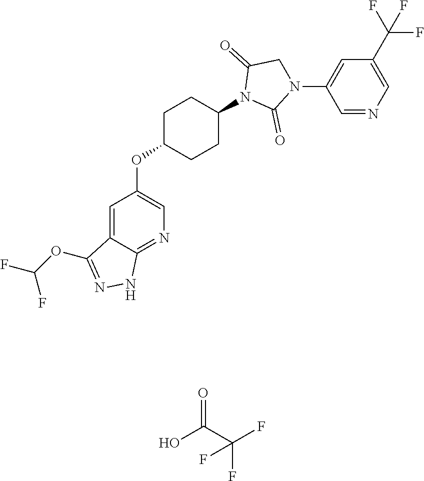 [SMILES: O=C1CN(c2cncc(C(F)(F)F)c2)C(=O)N1[C@H]1CC[C@H](Oc2cnc3[nH]nc(OC(F)F)c3c2)CC1 ; O=C(O)C(F)(F)F]

The mixture of the compound prepared in Reference Example 88 (80 mg) in TFA (3 mL) was stirred for 16 h at 70° C. The reaction mixture was concentrated under reduced pressure to afford the crude product. The crude product was purified by preparative HPLC to afford the title compound (12 mg).

¹H NMR (400 MHz, DMSO-d₆) δ ppm: 13.15, 9.09, 8.74, 8.49, 8.33-8.32, 7.83-7.82, 7.70-7.33, 4.58, 4.53-4.48, 4.03-3.97, 2.42-2.30, 2.28-2.18, 1.82-1.77, 1.58-1.48;

LCMS: m/z 527 [M+H]+;

HPLC retention time: 1.616 min (method 88).

### Example 36(1)˜ 36(7)

A procedure for a purpose similar to that for Reference Example 86→Reference Example 87→Reference Example 88→Example 36 was carried out by using a corresponding arylalcohol compound instead of the compound prepared in Reference Example 56, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 36(1)

### 3-{trans-4-[(3-amino-1H-pyrazolo[3,4-b]pyridin-5-yl)oxy]cyclohexyl}-1-[5-(trifluoromethyl)-3-pyridinyl]-2,4-imidazolidinedione trifluoroacetate (1:1)

¹H NMR (400 MHz, DMSO-d₆) δ 9.00, 8.69, 8.44, 8.18, 7.81, 4.51, 4.28-4.20, 4.04-3.95, 2.34-2.20, 1.87-1.78, 1.57-1.46;

LCMS: m/z 476 [M+H]+;

HPLC retention time: 1.291 min (method 57-1).

### Example 36(2): 3-{trans-4-[(2-amino-5-pyrimidinyl)oxy]cyclohexyl}-1-[3-(trifluoromethoxy)phenyl]-2,4-imidazolidinedione

¹H NMR (400 MHz, DMSO-d₆) δ ppm: 8.07, 7.83, 7.53, 7.18-7.09, 6.26, 4.47, 4.36, 4.05-3.94, 2.49-2.41, 2.06-1.96, 1.68-1.56, 1.54-1.48;

LCMS: m/z 452 [M+H]+;

HPLC retention time: 2.679 min (method 16-2).

### Example 36(3): 3-{cis-3-[(2-amino-5-pyrimidinyl)oxy]cyclopentyl}-1-[5-(trifluoromethyl)-3-pyridinyl]-2,4-imidazolidinedione

¹H NMR (400 MHz, DMSO-d₆) δ 9.08, 8.74, 8.47, 8.03, 6.21, 4.70-4.67, 4.55, 4.44-4.40, 2.38-2.32, 2.29-2.23, 1.99-1.92, 1.88-1.78;

LCMS: m/z 580 [M+H]+;

HPLC retention time: 1.027 min (method 84).

### Example 36(4): 3-{trans-3-[(2-amino-5-pyrimidinyl)oxy]cyclopentyl}-1-[5-(trifluoromethyl)-3-pyridinyl]-2,4-imidazolidinedione

¹H NMR (400 MHz, DMSO-d₆) δ 9.09, 8.74, 8.48, 8.05, 6.25, 4.89-4.87, 4.73-4.64, 4.56, 2.43-2.36, 2.27-2.19, 2.10-1.94, 1.88-1.71;

LCMS: m/z 423 [M+H]+;

HPLC retention time: 2.388 min (method 16-1).

### Example 36(5): 3-{trans-4-[(6,7-dimethoxy-3-quinolinyl)oxy]cyclohexyl}-1-[5-(trifluoromethyl)-3-pyridinyl]-2,4-imidazolidinedione

¹H NMR (400 MHz, DMSO-d₆) δ 9.10, 8.75, 8.49, 8.38, 7.82, 7.28, 4.58, 4.50-4.42, 4.10-3.95, 3.88, 2.38-2.32, 2.25-2.22, 1.82-1.79, 1.56-1.52;

LCMS: m/z 531 [M+H]+;

HPLC retention time: 1.176 min (method 84).

### Example 36(6): 3-{trans-3-[(3-amino-1H-pyrazolo[3,4-b]pyridin-5-yl)oxy]cyclobutyl}-1-[5-(trifluoromethyl)-3-pyridinyl]-2,4-imidazolidinedione trifluoroacetate (1:1)

¹H NMR (300 MHz, DMSO-d₆) δ 9.11, 8.75, 8.51, 8.22, 7.59, 5.07-5.02, 4.90-4.81, 4.60, 3.28-3.12, 2.73-2.59;

LCMS: m/z 448 [M+H]+;

HPLC retention time: 1.325 min (method 88).

### Example 36(7): 3-(trans-4-{[3-(2-hydroxyethoxy)-1H-pyrazolo[3,4-b]pyridin-5-yl]oxy}cyclohexyl)-1-[5-(trifluoromethyl)-3-pyridinyl]-2,4-imidazolidinedione

¹H NMR (400 MHz, DMSO-d₆) δ ppm 12.45-12.24, 9.12-9.04, 8.78-8.72, 8.53-8.44, 8.22, 7.70, 4.90-4.82, 4.59-4.53, 4.44-4.35, 4.35-4.29, 4.04-3.93, 3.83-3.72, 2.39-2.24, 2.24-2.13, 1.84-1.73, 1.58-1.41;

LCMS: m/z 521 [M+H]+;

HPLC retention time: 0.974 min (method 83).

### Reference Example 89: 5-nitro-1H-pyrazolo[3,4-b]pyridin-3-amine

A mixture of 2-chloro-5-nitro-pyridine-3-carbonitrile (CAS No. 31309-08-7, 1.00 g), N₂H4.H₂O (817 mg) in Methanol (25 mL) was stirred at 80° C. overnight. The mixture was diluted with water, extracted with ethyl acetate, dried over sodium sulfate, filtered, concentrated to obtain the title compound (800 mg).

LCMS: m/z 180 [M+H]+.

### Reference Example 90: N,N,1-tris[(4-methoxyphenyl)methyl]-5-nitro-pyrazolo[3,4-b]pyridin-3-amine

To a stirred solution of the compound prepared in Reference Example 89 (900 mg) in DMSO (10 mL) was added KOH (1127 mg) and 1-(chloromethyl)-4-methoxybenzene (2.38 mL). The resulting mixture was stirred overnight at room temperature under nitrogen atmosphere. The reaction was concentrated and the residue was diluted with water and extracted with ethyl acetate. The organics was washed with saturated sodium chloride solution. The organics were concentrated and the residue was purified by column chromatography to afford the title compound (600 mg).

LCMS: m/z 540 [M+H]+.

### Reference Example 91: N3,N3,1-tris[(4-methoxyphenyl)methyl]pyrazolo[3,4-b]pyridine-3,5-diamine

To a stirred mixture of the compound prepared in Reference Example 90 (800 mg) in ethanol (12 mL) and water (4 mL) were added Fe (662 mg), NH₄Cl (634 mg) under nitrogen atmosphere. The resulting mixture was stirred for 2 h at 80° C. under nitrogen atmosphere. The resulting suspension was filtered, the filter cake was washed with ethyl acetate. The filtrate was concentrated under reduced pressure. The resulting mixture was extracted with ethyl acetate, combined and washed with brine, dried over anhydrous sodium sulfate and concentrated under vacuum to give the title compound (300 mg).

LCMS: m/z 510 [M+H]+.

### Reference Example 92: N3,N3,1-tris[(4-methoxyphenyl)methyl]-N5-(4-nitrophenyl)pyrazolo[3,4-b]pyridine-3,5-diamine

To a stirred solution of the compound prepared in Reference Example 91 (300 mg) in DMF (5 mL) was added 1-bromo-4-nitrobenzene (0.07 mL), Pd₂(dba)₃ (54 mg), BINAP (73 mg), Cs₂CO₃ (192 mg) under nitrogen atmosphere. The resulting mixture was stirred overnight at 80° C. under nitrogen atmosphere. The reaction was filtered and purified by Prep-HPLC to obtain the title compound (280 mg).

LCMS: m/z 649 [M+H]+.

### Reference Example 93: N5-(4-aminophenyl)-N3,N3,1-tris[(4-methoxyphenyl)methyl]pyrazolo[3,4-b]pyridine-3,5-diamine

To a stirred solution of the compound prepared in Reference Example 92 (280 mg) in ethanol (6 mL) and water (2 mL) were added Fe (198 mg), NH₄Cl (24 mg) under nitrogen atmosphere. The resulting mixture was stirred for 2 h at 80° C. under nitrogen atmosphere. The resulting suspension was filtered, the filter cake was washed with ethyl acetate. The filtrate was concentrated under reduced pressure. The resulting mixture was extracted with ethyl acetate, combined and was washed with brine, dried over anhydrous sodium sulfate and concentrated under vacuum to give the title compound (260 mg).

LCMS: m/z 619 [M+H]+.

### Example 37: 3-{4-[(3-amino-1H-pyrazolo[3,4-b]pyridin-5-yl)amino]phenyl}-1-[5-(trifluoromethyl)-3-pyridinyl]-2,4-imidazolidinedione trifluoroacetate (1:1)

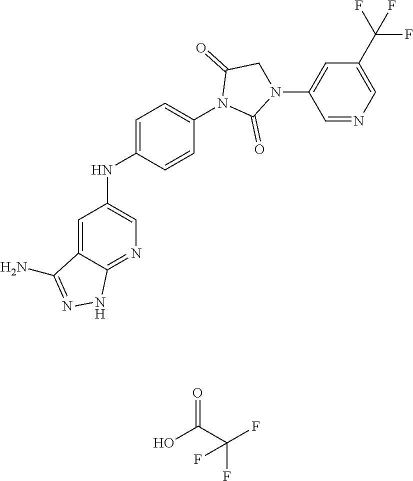 [SMILES: Nc1n[nH]c2ncc(Nc3ccc(N4C(=O)CN(c5cncc(C(F)(F)F)c5)C4=O)cc3)cc12 ; O=C(O)C(F)(F)F]

A procedure for a purpose similar to that for Reference Example 88→Example 36 was carried out by using the compound prepared in Reference Example 93 instead of the compound prepared in Reference Example 87, to give the title compound.

¹H NMR (400 MHz, DMSO-d₆) δ 11.89, 9.17, 8.76, 8.53, 8.26-8.21, 8.04, 7.19, 6.98, 6.22-4.91, 4.73;

LCMS: m/z 469 [M+H]+;

HPLC retention time: 1.245 min (method 67).

### Reference Example 94: 1-[3-(trifluoromethyl)phenyl]imidazolidine-2,4-dione

To a solution of [3-(trifluoromethyl)phenyl]urea (CAS No. 13114-87-9, 300 mg), ethyl 2-chloroacetate (0.17 mL) in DMF (15 mL) was added 60% NaH (105 mg). The resulting mixture was stirred at room temperature overnight. The filtrate was concentrated under reduced pressure. The reside was quenched with water and extracted with ethyl acetate. The organic phase was concentrated and the residue was purified by column chromatography to obtain the title compound (250 mg).

LCMS: m/z 245 [M+H]+.

### Reference Example 95: 3-(5-hydroxy-2-pyridyl)-1-[3-(trifluoromethyl)phenyl]imidazolidine-2,4-dione

To a mixture of the compound prepared in Reference Example 94 (244 mg) in pyridine (10 mL) was added 6-bromopyridin-3-ol (CAS No. 55717-45-8,869 mg) and Cu (220 mg) CuCl (19 mg) AcOK (294 mg). The resulting mixture was heated at 100° C. overnight, diluted with ethyl acetate, washed with brine and HCl (0.2 M), dried over Na₂SO₄, evaporated to give the title compound (70 mg).

LCMS: m/z 338 [M+H]+.

### Example 38: 3-{5-[(2-amino-4-pyrimidinyl)oxy]-2-pyridinyl}-1-[3-(trifluoromethyl)phenyl]-2,4-imidazolidinedione trifluoroacetate (1:1)

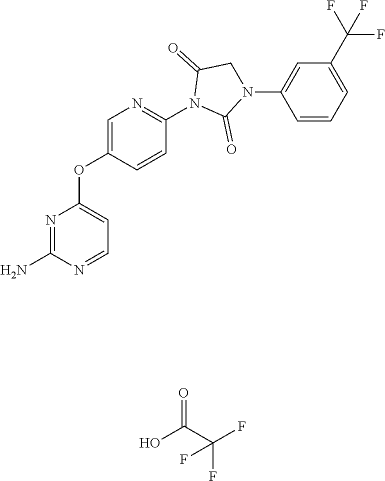 [SMILES: Nc1nccc(Oc2ccc(N3C(=O)CN(c4cccc(C(F)(F)F)c4)C3=O)nc2)n1 ; O=C(O)C(F)(F)F]

A solution of the compound prepared in Reference Example 95 (60 mg), 4-chloropyrimidin-2-amine (57 mg) and DMAP (113 mg) in chlorobenzene (5 mL) was stirred overnight at 130° C. under N₂ atmosphere. The filtrate was concentrated under reduced pressure. The reaction was diluted with water and extracted with ethyl acetate. The combined organic layer was washed with brine, dried over sodium sulfate, filtered and concentrated. The product was purified by Prep-HPLC to obtain the title compound (5.8 mg).

¹H NMR (400 MHz, DMSO-d₆) δ 8.61, 8.27, 8.16, 8.01, 7.95-7.87, 7.70, 7.64, 7.59-7.52, 7.48, 6.54, 4.80;

LCMS: m/z 431 [M+H]+;

HPLC retention time: 1.038 min (method 84).

### Reference Example 96: 3-(4-hydroxynorbornan-1-yl)-1-[5-(trifluoromethyl)-3-pyridyl]imidazolidine-2,4-dione

To a solution of 4-aminobicyclo[2.2.1]heptan-1-ol hydrochloride (CAS No. 1403865-39-3, 163 mg) and DIEA (645 mg) in THF (10 mL), was added the compound prepared in Reference Example 5 (297 mg). The resulting mixture was heated for overnight under reflux. The solvent was evaporated under vacuum, the crude product was purified by column chromatography to afford the title compound (250 mg).

### Reference Example 97: 2-[(6-bromo-4-chloro-pyrrolo[2,3-d]pyrimidin-7-yl)methoxy]ethyl-trimethyl-silane

The mixture of 6-bromo-4-chloro-7H-pyrrolo[2,3-d]pyrimidine (CAS No. 784150-41-0, 1.1 g), (2-(chloromethoxy)ethyl)trimethylsilane (1.13 mL) and NaH (60%; 0.17 g) in THF (40 mL) was stirred at room temperature for 4 h. The resulting solution was diluted with water and extracted with ethyl acetate. The organic layers were combined, washed with brine, dried over anhydrous sodium sulfate and concentrated under vacuum. The crude product was purified by column chromatography (SiO₂, Petroleum ether/EtOAc=1:10) to afford the title compound (1.47 g).

LCMS: m/z 364 [M+H]+.

### Reference Example 98: 1-[4-chloro-7-(2-trimethylsilylethoxymethyl)pyrrolo[2,3-d]pyrimidin-6-yl]ethanone

The mixture of the compound prepared in Reference Example 97 (1.35 g), tributyl(1-ethoxyvinyl)stannane (1.47 g) and Pd(PPh₃)₄ (430 mg) in toluene (10 mL) was stirred at 110° C. overnight. The resulting solution was diluted with water and extracted with ethyl acetate. The organic layers were combined, washed with brine, dried over anhydrous sodium sulfate and concentrated under vacuum. The crude product was purified by column chromatography (SiO₂, Petroleum ether/EtOAc=1:10) to afford the title compound (780 mg).

LCMS: m/z 326 [M+H]+.

### Reference Example 99: 2-[4-chloro-7-(2-trimethylsilylethoxymethyl)pyrrolo[2,3-d]pyrimidin-6-yl]propan-2-ol

To a solution of the compound prepared in Reference Example 98 (100 mg) in THF (3 mL) was added bromo(methyl)magnesium (0.21 mL) at 0° C. The reaction mixture was stirred for 16 h at room temperature. The reaction mixture was quenched with water, diluted with dichloromethane, washed with brine, dried over anhydrous sodium sulfate, filtered and concentrated under reduced pressure to afford crude product.

The residue was purified by Prep-TLC to afford the title compound (55 mg).

LCMS: m/z 342 [M+H]+.

### Reference Example 100: 3-[4-[6-(1-hydroxy-1-methyl-ethyl)-7-(2-trimethylsilylethoxymethyl)pyrrolo[2,3-d]pyrimidin-4-yl]oxynorbornan-1-yl]-1-[5-(trifluoromethyl)-3-pyridyl]imidazolidine-2,4-dione

To a solution of the compound prepared in Reference Example 99 (50 mg) in toluene (3 mL) was added K₂CO₃ (60 mg), BINAP (18 mg), Pd(PPh₃)₄ (17 mg) and the compound prepared in Reference Example 96 (62 mg). The reaction mixture was stirred for 20 h at 100° C. The reaction mixture was quenched with water, extracted with ethyl acetate, washed with brine, dried over anhydrous sodium sulfate, filtered and concentrated under reduced pressure to afford crude product. The residue was purified by Prep-TLC to afford the title compound (35 mg).

### Example 39: 3-(4-{[6-(2-hydroxy-2-propanyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione trifluoroacetate (1:1)

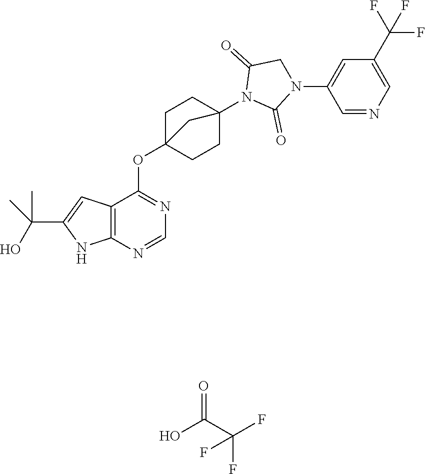 [SMILES: CC(C)(O)c1cc2c(OC34CCC(N5C(=O)CN(c6cncc(C(F)(F)F)c6)C5=O)(CC3)C4)ncnc2[nH]1 ; O=C(O)C(F)(F)F]

To a solution of the compound prepared in Reference Example 100 (32 mg) in DCM (2.7 mL) was added TFA (0.9 mL). The reaction mixture was stirred for 1 h at room temperature. The reaction mixture was concentrated under reduced pressure and then 7 M NH₃ in MeOH was added to this mixture. The reaction mixture was stirred for 30 min at room temperature. The reaction mixture was concentrated under reduced pressure to afford the crude product. The crude product was purified by Prep-HPLC to afford the title compound (7.3 mg).

¹H NMR (400 MHz, DMSO-d₆) δ ppm: 9.07-8.50, 8.28, 6.22, 4.55, 2.83, 2.41-2.30, 2.16-2.00, 1.51;

LCMS: m/z 531 [M+H]+;

HPLC retention time: 1.665 min (method 84-1).

### Reference Example 101: 2-[[4-chloro-6-(1,1-difluoroethyl)pyrrolo[2,3-d]pyrimidin-7-yl]methoxy]ethyl-trimethyl-silane

The compound prepared in Reference Example 98 (300 mg) was dissolved in DAST (CAS No. 38078-09-0, 10 mL). The reaction mixture was stirred for overnight at 70° C. The reaction mixture was quenched by ammonium chloride (aq) and diluted with ethyl acetate, washed with brine, dried over anhydrous sodium sulfate, filtered and concentrated under reduced pressure. The crude product was purified by column chromatography to afford the title compound (220 mg).

### Example 40: 3-(4-{[6-(1,1-difluoroethyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione

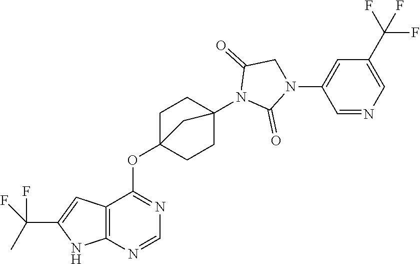 [SMILES: CC(F)(F)c1cc2c(OC34CCC(N5C(=O)CN(c6cncc(C(F)(F)F)c6)C5=O)(CC3)C4)ncnc2[nH]1]

A procedure for a purpose similar to that for Reference Example 100→Example 39 was carried out by using the compound prepared in Reference Example 101 instead of the compound prepared in Reference Example 99, to give the title compound.

¹H NMR (400 MHz, DMSO-d₆) δ ppm: 12.76, 9.07, 8.74, 8.50, 8.44, 6.79, 4.56, 2.84, 2.51-2.39, 2.16-2.06;

LCMS: m/z 537 [M+H]+;

HPLC retention time: 1.589 min (method 84).

### Reference Example 102: 3-[4-[6-acetyl-7-(2-trimethylsilylethoxymethyl)pyrrolo[2,3-d]pyrimidin-4-yl]oxynorbornan-1-yl]-1-[5-(trifluoromethyl)-3-pyridyl]imidazolidine-2,4-dione

The mixture of the compound prepared in Reference Example 98 (88 mg), the compound prepared in Reference Example 96 (80 mg), BINAP (56 mg), K₂CO₃ (155 mg) and Pd(PPh₃)₄ (52 mg) in toluene (3 mL) was stirred at 100° C. overnight. The reaction was quenched by water and extracted with ethyl acetate. The combined organic layers were washed with brine, dried over anhydrous sodium sulfate. After filtration, the filtrate was concentrated under reduced pressure. The crude product was purified by Prep-TLC (Petroleum ether:Ethyl acetate=1:10) to afford the title compound (20 mg).

### Reference Example 103: 3-[4-[6-(1-hydroxyethyl)-7-(2-trimethylsilylethoxymethyl)pyrrolo[2,3-d]pyrimidin-4-yl]oxynorbornan-1-yl]-1-[5-(trifluoromethyl)-3-pyridyl]imidazolidine-2,4-dione

The mixture of the compound prepared in Reference Example 102 (10 mg) and NaBH₄ (0.88 mg) in THF (1 mL) was stirred at 0° C. for 45 min. The reaction was quenched by water and extracted with ethyl acetate. The combined organic layers were washed with brine, dried over anhydrous sodium sulfate. After filtration, the filtrate was concentrated under reduced pressure. The crude product was purified by Prep-TLC (Petroleum ether:Ethyl acetate=1:10) to afford the title compound (10 mg).

LCMS: m/z 647 [M+H]+.

### Example 41: 3-(4-{[6-(1-hydroxyethyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione

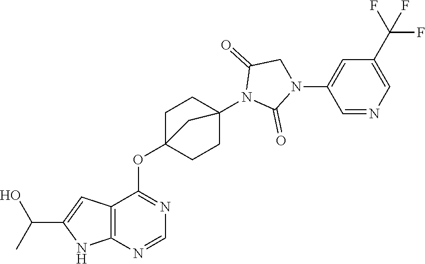 [SMILES: CC(O)c1cc2c(OC34CCC(N5C(=O)CN(c6cncc(C(F)(F)F)c6)C5=O)(CC3)C4)ncnc2[nH]1]

The mixture of the compound prepared in Reference Example 103 (27 mg) and 2,2,2-trifluoroacetaldehyde (2 mL) in DCM (2 mL) was stirred at room temperature for 2 h. The mixture was concentrate. To the above mixture was added 7M NH₃ in MeOH (2 mL) and stirred at room temperature for 1 h. The mixture was concentrated and the residue was purified by Prep-TLC to afford the title compound (1.4 mg).

¹H NMR (400 MHz, DMSO-d₆) δ 11.88, 9.06, 8.73, 8.50, 8.28, 6.25, 5.32, 4.82-4.79, 4.55, 2.82, 2.41-2.39, 2.36-2.33, 2.10-2.04, 1.45;

LCMS: m/z 517 [M+H]+

HPLC retention time: 1.538 min (method 92).

### Reference Example 104: 4-chloro-5-(trifluoromethyl)-7H-pyrrolo[2,3-d]pyrimidine

To the mixture of 4-chloro-7H-pyrrolo[2,3-d]pyrimidine (1.0 g) and sodium trifluoromethanesulfinate (3.1 g) in DCM (9 mL) and Water (3 mL) was added tert-Butyl hydroperoxide (2.9 g) at 0° C. The resulting mixture was stirred at r.t. for 48 h. The mixture was diluted with water, then adjusted to pH 8-9 with saturated sodium bicarbonate. The resulting solution was extracted with DCM. The organic layers were combined, washed with brine, dried over anhydrous sodium sulfate and concentrated under vacuum. The crude product was purified by column chromatography to afford the title compound (300 mg).

LCMS: m/z 222 [M+H]+.

### Reference Example 105: 2-[[4-chloro-5-(trifluoromethyl)pyrrolo[2,3-d]pyrimidin-7-yl]methoxy]ethyl-trimethyl-silane

To the solution of the compound prepared in Reference Example 104 (250 mg) in THF (5 mL) was added NaH in oil (60%; 68 mg) at 0° C. After stirring at 0° C. for 1 h, 2-(Trimethylsilyl)ethoxymethyl chloride (282 mg) was added to the mixture at 0° C. and stirred at r.t. for 3 h. The reaction was quenched by the addition of H₂O. The resulting mixture was extracted with ethyl acetate. The combined organic layers were washed with brine, dried over anhydrous sodium sulfate. After filtration, the filtrate was concentrated under reduced pressure. The crude product was purified by column chromatography to afford the title compound (270 mg).

LCMS: m/z 352 [M+H]+.

### Reference Example 106: 1-[5-(trifluoromethyl)-3-pyridyl]-3-[4-[5-(trifluoromethyl)-7-(2-trimethylsilylethoxymethyl)pyrrolo [2,3-d]pyrimidin-4-yl]oxynorbornan-1-yl]imidazolidine-2,4-dione

To the mixture of the compound prepared in Reference Example 105 (100 mg), the compound prepared in Reference Example 96 (151 mg), BINAP (35 mg) and Potassium carbonate (117 mg) in Toluene (5 mL) was added Tetrakis(triphenylphosphine)palladium (32 mg). The mixture was stirred at 100° C. overnight under nitrogen atmosphere. The reaction was quenched by the addition of H₂O. The resulting mixture was extracted with ethyl acetate. The combined organic layers were washed with brine, dried over anhydrous sodium sulfate. After filtration, the filtrate was concentrated under reduced pressure. The crude product was purified by column chromatography to afford the title compound (120 mg).

LCMS: m/z 671 [M+H]+.

### Example 42: 1-[5-(trifluoromethyl)-3-pyridinyl]-3-(4-{[5-(trifluoromethyl)-7H-pyrrolo[2,3-d]pyrimidin-4-yl]oxy}bicyclo[2.2.1]hept-1-yl)-2,4-imidazolidinedione

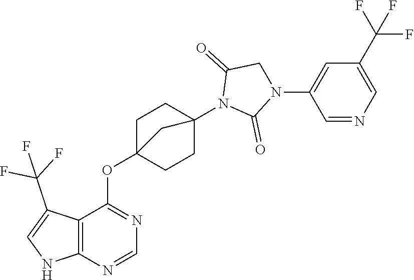 [SMILES: O=C1CN(c2cncc(C(F)(F)F)c2)C(=O)N1C12CCC(Oc3ncnc4[nH]cc(C(F)(F)F)c34)(CC1)C2]

To the solution of the compound prepared in Reference Example 106 (110 mg) in DCM (1 mL) was added TFA (1 mL). The mixture was stirred at room temperature for 3 h. The reaction was concentrated under vacuum and the crude product was purified by reversed phase chromatography to afford the title compound (45 mg).

¹H NMR (400 MHz, DMSO-d₆) δ 9.05, 8.74, 8.53-8.50, 7.10, 4.53, 2.83, 2.50-2.40, 2.14-1.94;

LCMS: m/z 541 [M+H]+;

HPLC retention time: 1.652 min (method 84).

### Example 42(1)˜ 42(11)

A procedure for a purpose similar to that for Reference Example 96→Reference Example 105→Reference Example 106→Example 42 was carried out by using a corresponding halide compound instead of the compound prepared in Reference Example 104, and a corresponding chlorocarbonyl compound instead of the compound prepared in Reference Example 5, to give the following compounds of Examples; wherein a corresponding chlorocarbonyl compound was produced by operation in accordance with Reference Example 1→Reference Example 2→Reference Example 3→Reference Example 4→Reference Example 5 using a corresponding amine compound instead of 5-(trifluoromethyl)pyridin-3-amine.

### Example 42(1): 3-{4-[(5-fluoro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione

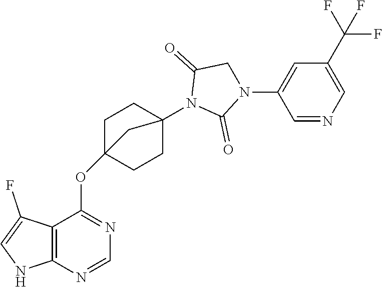 [SMILES: O=C1CN(c2cncc(C(F)(F)F)c2)C(=O)N1C12CCC(Oc3ncnc4[nH]cc(F)c34)(CC1)C2]

¹H NMR (400 MHz, CHLOROFORM-d) δ ppm 10.03-9.44, 8.91-8.77, 8.76-8.63, 8.55-8.46, 8.44-8.32, 6.90, 4.35, 2.97, 2.69-2.43, 2.29-2.17, 2.05;

LCMS: m/z 491 [M+H]+;

HPLC retention time: 1.137 min (method 83).

### Example 42(2): 3-{4-[(5-chloro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione

¹H NMR (400 MHz, CHLOROFORM-d) δ ppm 9.53-9.30, 8.89-8.78, 8.69-8.65, 8.54-8.47, 8.44-8.34, 7.12-7.05, 4.35, 2.99-2.93, 2.68-2.61, 2.59-2.48, 2.29-2.19, 2.15-2.05;

LCMS: m/z 507 [M+H]+;

HPLC retention time: 1.175 min (method 83).

### Example 42(3): 3-{4-[(5-bromo-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione trifluoroacetate (1:1)

¹H NMR (400 MHz, METHANOL-d4) δ ppm 9.07-9.00, 8.67-8.60, 8.58-8.50, 8.34-8.27, 7.28, 4.52-4.41, 2.96-2.91, 2.67-2.51, 2.27-2.1;

LCMS: m/z 551 [M+H]+;

HPLC retention time: 1.186 min (method 83).

### Example 42(4): 3-{4-[(5-iodo-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione trifluoroacetate (1:1)

¹H NMR (400 MHz, METHANOL-d4) δ ppm 9.10-9.03, 8.68-8.61, 8.58-8.50, 8.35-8.27, 7.37-7.31, 4.47, 2.96, 2.68-2.50, 2.26-2.10;

LCMS: m/z 599 [M+H]+;

HPLC retention time: 1.199 min (method 83).

### Example 42(5): 3-[4-(9H-purin-6-yloxy)bicyclo[2.2.1]hept-1-yl]-1-[5-(trifluoromethyl)-3-pyridinyl]-2,4-imidazolidinedione

¹H NMR (400 MHz, DMSO-d₆) δ ppm: 13.42-13.38, 9.08-9.07, 8.75, 8.51-8.47, 8.34, 4.56, 2.87, 2.49-2.31, 2.20-2.00;

LCMS: m/z 474 [M+H]+;

HPLC retention time: 1.315 min (method 9).

### Example 42(6): 1-[4-fluoro-3-(trifluoromethoxy)phenyl]-3-[4-(7H-pyrrolo[2,3-d]pyrimidin-4-yloxy)bicyclo[2.2.1]hept-1-yl]-2,4-imidazolidinedione

¹H NMR (400 MHz, DMSO-d₆) δ ppm 12.06-11.85, 8.33, 8.14-7.92, 7.69-7.43, 7.40-7.24, 6.56-6.39, 4.44, 2.92-2.73, 2.44-2.28, 2.18-2.02;

LCMS: m/z 506 [M+H]+;

HPLC retention time: 1.198 min (method 83).

### Example 42(7): 5-{2,4-dioxo-3-[4-(7H-pyrrolo[2,3-d]pyrimidin-4-yloxy)bicyclo[2.2.1]hept-1-yl]-1-imidazolidinyl}nicotinonitrile

¹H NMR (400 MHz, DMSO-d₆) δ ppm 12.07-11.91, 9.26-9.13, 8.86-8.74, 8.54-8.40, 8.33, 7.40-7.25, 6.53-6.33, 4.50, 2.89-2.79, 2.47-2.35, 2.21-2.02;

LCMS: m/z 430 [M+H]+;

HPLC retention time: 0.997 min (method 83).

### Example 42(8): 5-{2,4-dioxo-3-[4-(7H-pyrrolo[2,3-d]pyrimidin-4-yloxy)bicyclo[2.2.1]hept-1-yl]-1-imidazolidinyl}-2-fluorobenzonitrile

LCMS: m/z 447 [M+H]+;

HPLC retention time: 1.665 min (method 83).

### Example 42(9): 3-{2,4-dioxo-3-[4-(7H-pyrrolo[2,3-d]pyrimidin-4-yloxy)bicyclo[2.2.1]hept-1-yl]-1-imidazolidinyl}benzonitrile

¹H NMR (400 MHz, CHLOROFORM-d) δ ppm 9.13-8.99, 8.40, 7.99, 7.83-7.72, 7.56-7.41, 7.14, 6.56, 4.26, 2.99-2.90, 2.69-2.52, 2.25-2.06;

LCMS: m/z 429 [M+H]+;

HPLC retention time: 1.078 min (method 83).

### Example 42(10): 3-[4-(7H-pyrrolo[2,3-d]pyrimidin-4-yloxy)bicyclo[2.2.1]hept-1-yl]-1-[3-(trifluoromethoxy)phenyl]-2,4-imidazolidinedione

¹H NMR (400 MHz, CHLOROFORM-d) δ ppm 9.16-8.97, 8.49-8.33, 7.70-7.61, 7.39, 7.17-7.08, 7.06-6.97, 6.60-6.52, 4.25, 2.94, 2.72-2.46, 2.27-2.06;

LCMS: m/z 488 [M+H]+;

HPLC retention time: 1.192 min (method 83).

### Example 42(11): 1-[3-(methylsulfonyl)-5-(trifluoromethyl)phenyl]-3-[4-(7H-pyrrolo[2,3-d]pyrimidin-4-yloxy)bicyclo[2.2.1]hept-1-yl]-2,4-imidazolidinedione

¹H NMR (400 MHz, DMSO-d₆) δ 12.02, 8.50, 8.34, 8.26, 7.98, 7.36-7.34, 6.46-6.44, 4.59, 3.37, 2.85, 2.50-2.41, 2.15-2.02;

LCMS: m/z 550 [M+H]+;

HPLC retention time: 1.733 min (method 84-1).

### Reference Example 107: tert-butyl N—[4-[(2-fluoro-3-formyl-4-pyridyl)oxy]norbornan-1-yl]carbamate

To solution of tert-butyl N—{4-hydroxybicyclo[2.2.1]heptan-1-yl}carbamate (CAS No. 2231676-44-9, 500 mg), 4-bromo-2-fluoro-pyridine-3-carbaldehyde (CAS No. 128071-77-2, 583 mg) and K₂CO₃ (911 mg) in toluene (2 mL) was added BINAP (274 mg) and Pd(PPh₃)₄ (254 mg). The mixture was stirred at 100° C. for 1 h. The mixture was concentrated under vacuum and the residue was purified by column chromatography to afford the crude product.

LCMS: m/z 351 [M+H]+.

### Reference Example 108: tert-butyl N—[4-(1H-pyrazolo[3,4-b]pyridin-4-yloxy)norbornan-1-yl]carbamate

The mixture of the compound prepared in Reference Example 107 (85 mg) and NH₂NH₂.H₂O (36 mg) in ethanol (2 mL) was stirred at 60° C. for 8 h. The mixture was allowed to cool down to room temperature. The resulting solution was diluted with water and extracted with ethyl acetate. The organic layers were combined, washed with brine, dried over anhydrous sodium sulfate and concentrated under vacuum. The crude product was purified by column chromatography to afford the title compound (30 mg).

LCMS: m/z 345 [M+H]+.

### Reference Example 109: 4-(1H-pyrazolo[3,4-b]pyridin-4-yloxy)norbornan-1-amine

To solution of the compound prepared in Reference Example 108 (25 mg) in DCM (1 mL) was added TFA (0.3 mL) for 1 h at room temperature. The crude product was evaporated and used directly without purification.

LCMS: m/z 245 [M+H]+.

### Example 43: 3-[4-(1H-pyrazolo[3,4-b]pyridin-4-yloxy)bicyclo[2.2.1]hept-1-yl]-1-[5-(trifluoromethyl)-3-pyridinyl]-2,4-imidazolidinedione

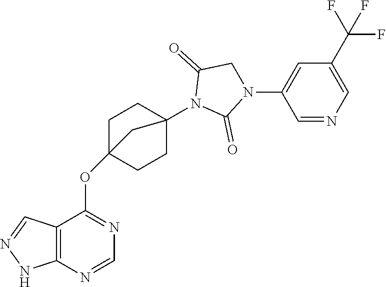 [SMILES: O=C1CN(c2cncc(C(F)(F)F)c2)C(=O)N1C12CCC(Oc3ncnc4[nH]ncc34)(CC1)C2]

To solution of the compound prepared in Reference Example 109 (30 mg) in THE (2 mL) was added DIEA (0.16 mL) and the compound prepared in Reference Example 5 (72 mg). The mixture was stirred at room temperature for 2 h. The mixture was concentrated. The residue was purified by HPLC to afford the title compound (4.9 mg).

¹H NMR (400 MHz, DMSO-d₆) δ 9.04, 8.72, 8.47, 8.30, 8.05, 6.85, 4.56, 2.73, 2.38-2.32, 2.15-2.10, 2.05-1.98;

LCMS: m/z 473 [M+H]+;

HPLC retention time: 2.661 min (method 16-1).

### Biological Experiment Example

Biological Experiment Examples are described below. Based on these experiment methods, the advantageous effect of the compound of the present invention was verified.

### Biological Experiment Example 1: Measurement of an DDR1 Inhibitory Activity (In Vitro Test)

An DDR1 enzyme inhibitory activity was measured by using LanthaScreen (registered trademark) system (Invitrogen) based on the attached instruction. The reagents used are shown below.

### Reagent Preparation

Assay buffer: 5× Kinase buffer A (Invitrogen) was diluted 5-fold with ddH2O.

1× Kinase Buffer A: 50 mmol/L HEPES pH 7.5, 10 mmol/L MgCl₂, 1 mmol/L EGTA, 0.01% Brij-35.

2×DDR1 Solution: a solution containing 2 mmol/L GST-DDR1 (Invitrogen) was prepared by using Assay buffer.

2× Tracer/antibody solution: a solution containing 20 nmol/L Kinase tracer 178 (Invitorgen) and 4 mmol/L Eu-Anti-GST Antibody (Invitorogen) was prepared by using Assay buffer. Test compound solution: a solution containing a test compound of 2-fold concentration with respect to the final concentration was prepared by 50-fold diluting a DMSO solution of test compound of each concentration with the Assay buffer.

### Experimental Steps

1 Add 5 μL 2×DDR1 Solution to proxiplate (PerkinElmer)
2 Add 5 μL test compound solution to proxiplate
3 Incubate for 15 min at RT
4 Add 5 μL 2× Tracer/Antibody solution to all well Incubate 1 h at RT
6 Read LANCE Eu on Envision (PerkinElmer) (Ex: 320 nm/Em 665 nm & 615 nm)

In the low control group, 5 μL Assay buffer was added instead of 5 μL 2×DDR1 Solution and the final 1% DMSO solution was added instead of test compound solution.

In the high control group, the final 1% DMSO solution was added instead of test compound solution.

### Data Analysis

The inhibition rate (%) in the test compound group was calculated based on the following mathematical formula.

% inhibition=100−100*(assay value of test compound−A)/(B−A) [Math. 1]

A: average value of low control group
B: average value of high control group

Values of 50% inhibition rate (IC50 values) of the test compound were calculated from the inhibition curve based on the inhibition rate of the test compounds in each concentration.

Results of IC50 values were shown in Table 2.

TABLE 2

| Example | IC50 (μmol/L) |
|---|---|
| 1 | 0.003 |
| 1(11) | 0.001 |
| 1(28) | 0.002 |
| 2 | 0.005 |
| 2(2) | 0.2 |
| 2(6) | 0.01 |
| 2(19) | 0.003 |
| 2(125) | 0.02 |
| 2(126) | 0.01 |
| 2(149) | 0.003 |
| 3(17) | 0.005 |
| 3(47) | 0.001 |
| 3(59) | 0.004 |
| 3(63) | 0.007 |
| 3(65) | 0.003 |
| 3(66) | 0.001 |
| 3(78) | 0.004 |
| 3(101) | 0.003 |
| 3(110) | 0.001 |
| 3(111) | 0.001 |
| 3(120) | 0.007 |
| 3(122) | 0.005 |
| 3(176) | 0.002 |
| 4(1) | 0.003 |
| 5(46) | 0.002 |
| 6(1) | 0.005 |
| 7(6) | 0.003 |
| 8(1) | 0.08 |
| 9(1) | 0.01 |
| 10 | 0.06 |
| 11(3) | 0.006 |
| 13 | 0.003 |

### Biological Example 2: Measurement of DDR1 Phosphorylation Suppression Rate by Using T-47D (ATCC, HTB-133) cells

### Cell Preparation

T-47D cells should be in culture for at least 7 days before using in the assay.

### Working Reagents Preparation

Cell culture medium: RPMI 1640 (HyClone) containing 10% Heat-FBS (HyClone) was prepared.

Assay medium: RPMI 1640 containing 0.1% BSA (solarbio) was prepared.

Collagen Type I (rat tail) solution: Assay medium containing 200 μg/mL Collagen I, rat tail (Gibco) was prepared.

Cell lysis buffer: a solution containing IX cell lysis buffer (CST) and 1× protease and phosphatase inhibitor cocktail (Thermofisher) was prepared by using ddH₂O.

### Assay Procedure

### 1 Cell Collection and Plating:

1. Collect cells into tubes and centrifuge at 1200 rpm for 5 min, at room temperature. Re-suspend the cells in cell culture medium pre-warmed to room temperature.
2. Count cells and suspend at a density of 2×10⁵/ml of cell culture medium. Plate the cells at 100 μL per well (Corning 3599 96-well plate, 2×10⁴ cells per well final), mixing the cells well between each draw of the pipettor.
3. Put lid on cell plate and place into the incubator at 37° C., 5% CO₂
4. Culture cell overnight

### 2 Change to Assay Medium (Contain 0.1% BSA)

1. Remove the plate from the incubator
2. Cell medium is replaced with the 90 μL Assay medium.

### 3 Compound Addition:

1. Compound plates are pre-titrated using DMSO (10-fold dilution for 6 points)
2. Dilute test compound using assay medium; Transfer 1 μL compound to other 96 well plate and then add 49 μL assay medium to each well (50 fold dilution).
3. Add 5 μL the diluted test compound solution to the appropriate wells by pipettor. Compound final concentration is start from 1000 mmol/L and DMSO final concentration is 0.1
4. Tap the plates gently to mix in compound. Reseal the plate with a lid and place in the incubator again, for 24 h.

### 4 Collagen Type I (Rat Tail) Addition:

1. Diluted collagen Type I in assay medium to the 200 μg/ml concentration
2. Using the pipettor adds 5 μL of 200 μg/mL collagen Type I solution to the cell plate (compound well and High control well, collagen final conc. is 10 μg/ml). Add assay medium buffer to the control wells (low control well)
3. Reseal the plate with a lid and place in the incubator. Incubate 24 h for collagen type I

### 5 Harvest Cell Lysis:

1. After incubation, add 100 L per well Cell lysis buffer.
2. Incubate 30 min on ice, then freezing to −80° C. to get the fully cell lysates.
3. Transfer cell lysates to new plate
4. Centrifuge for 30 min (×4000 rpm) at 4° C. Then transfer the supernatant to a new plate.

### ELISA Test Procedure

p-DDR1 (panTyr) ELISA kit (CST) was used in the study. The measurement was carried out according to the attached instruction.

### Data Analysis

The inhibition rate (%) in the test compound group was calculated based on the following mathematical formula.

% inhibition=100−100*(assay value of test compound−A)/(B−A) [Math. 2]

A: average value of low control group
B: average value of high control group
Values of 50% inhibition rate (IC50 values) of the test compound were calculated from the inhibition curve based on the inhibition rate of the test compounds in each concentration. Results of IC50 values were shown in Table 3.

TABLE 3

| Example | IC50 (μmol/L) |
|---|---|
| 1 | 0.002 |
| 1(11) | 0.001 |
| 1(28) | 0.002 |
| 2 | 0.003 |
| 2(2) | 0.05 |
| 2(6) | 0.006 |
| 2(19) | 0.001 |
| 2(125) | 0.005 |
| 2(126) | 0.006 |
| 2(149) | 0.003 |
| 2(163) | 0.006 |
| 3(17) | 0.009 |
| 3(47) | 0.001 |
| 3(59) | 0.0004 |
| 3(63) | 0.001 |
| 3(65) | 0.002 |
| 3(66) | 0.001 |
| 3(78) | 0.002 |
| 3(101) | 0.002 |
| 3(110) | 0.001 |
| 3(111) | 0.001 |
| 3(120) | 0.005 |
| 3(122) | 0.007 |
| 3(176) | 0.0001 |
| 4(1) | 0.001 |
| 6(1) | 0.002 |
| 7(6) | 0.002 |
| 8 | 0.07 |
| 9(1) | 0.02 |
| 10(1) | 0.01 |
| 11(3) | 0.001 |
| 13 | 0.003 |
| 36(7) | 0.0001 |
| 39 | 0.01 |
| 42(1) | 0.008 |
| 42(2) | 0.02 |
| 42(7) | 0.2 |
| 42(9) | 0.02 |
| 43 | 0.006 |

### Biological Example 3: Evaluation of Kinase Selectivity

% inhibition rate with respect to various kinases (for example KDR etc.) of 1 μmol/L the test compound is measured.

A general assay condition (substrates, cofactor, ATP concentrations and reaction time etc.) suitable for respective kinase enzyme inhibition activity test is used.

### Preparation of Compound

1 Prepare the test compounds into 1 mmol/L DMSO solution.
2 Transfer 20 nL of stock to 384 assay plate. Use DMSO as High and Low controls.

### Performing the Enzyme Assay

1 Prepare the 1.3× Enzyme solution containing enzyme, substrate and cofactors.
2 Add 15 μL of 1.3× Enzyme solutions to each well and incubate the plate at room temperature for 30 min.
3 Initiate the reaction by adding 5 μL of 4×ATP solution. The final volume of each well should be 20 μL.
4 Incubate the plate at room temperature for reaction and add 75 μL of stopping buffer (containing 0.5 mol/L EDTA) to terminate the reaction.

Analyze the sample of each well using EZ reader (PerkinElmer).

### Data Analysis

Calculate the % Inhibition using read conversion ratio (CR) following the equation as below.

Use well treated with DMSO as High Control
Use well without enzyme as Low Control

$$\% Inhibition = \frac{C {R}_{High} - C {R}_{Sample}}{C {R}_{High} - C {R}_{Low}}$$

Results of % inhibition rate were shown in Table 4.

TABLE 4

| Example(1 μmol/L) | KDR | Flt-3 | ROCK2 |
|---|---|---|---|
| 1(11) | 2 | 16 | −2 |
| 2 | 1 | 4 | −5 |
| 3(65) | −1 | 5 | 5 |
| 3(110) | 4 | 6 | −2 |
| 42(1) | −3 | −2 | −8 |

Results showed that the compound of the present invention had DDR1 selective inhibition effect on KDR, Flt-3, and ROCK. Therefore, it was suggested that the compounds of the present invention were excellent compounds capable of avoiding the side effect, which was caused by multikinase inhibition.

Biological Example 4: Measurement of DDR1 phosphorylation suppression rate by using UUO (unilateral ureteral obstruction) model (in vivo test).

Animal

Species & Strain: C57BL/6J mice

Age: 7 to 9 weeks

Sex: Male

Groups and Treatments

Mice were randomly assigned to respective groups based on the animal's body weight.

UUO Surgery

Surgery was performed in male C57BL/6J mice on Day 0. With the mice under anesthesia, operation area was disinfected with 75% alcohol after shaving. Mice in UUO group were exposed left ureter through a small incision on the left side of the abdomen and ligated with 3-0 silk at two locations about 0.5 cm below the renal hilum. Normal control mice were had their ureters manipulated but not ligated. Zoletil combined with Xylazine (20:1, v:v) were used in this study, the final dose of Zoletil was 25 mg/kg.

Dosing Procedure

Route: oral administration

Dosage: 1, 3 and 10 mg/kg

Volume: 10 mL/kg

Frequency: single dose

Body Weight and Clinical Observation

Body weights of all animals were measured prior surgery and prior dosing. Clinical observations of all animals were monitored and recorded daily throughout the study.

Blood Sample Collection

8 h after dosing, blood samples were collected via the cardiac puncture of the mice sacrificed by carbon dioxide into tube containing EDTA-2K as anticoagulant. The samples were immediately centrifuged at 4° C., 4000 g for 5 min and plasma was transferred into another sample tube. These plasma samples were kept at −80° C. and then is subjected to plasma concentrations analysis.

Tissue Collection

On Day 3, all animals were euthanized with carbon dioxide. The kidneys were collected from all animals at 8 h after dosing and snap frozen in liquid nitrogen immediately. Then the samples were analyzed by Elisa.

ELISA Analysis

DDR1 and p-DDR1 levels in ipsilateral kidney samples were analyzed by Elisa method. DDR1 and p-DDR1 (panTyr) ELISA kit (CST) were used in the study. The measurement was carried out according to the attached instruction.

In Vitro Analysis of Protein Levels in Kidney Samples

1 Kidney was placed in 2 mL tubes.
2 The tissue was completely crushed under low temperature.
3 800 L of Cell Lysis Buffer was added to each sample.
4 The samples were incubated at 4° C. for more than 30 min.
5 12000 rpm 15 min centrifuge at 4° C., and the supernatant was recovered.
6 Use BCA assay to determine protein concentration.
7 Kidney lysates were diluted with Cell Lysis Buffer to the concentration of 5.0 μg/μL.
8 Diluted kidney lysates (5.0 μg/L) were further diluted two fold in Sample Diluent (2.5 μg/μL).

Data Acquisition and Statistical Analysis

All statistical tests were conducted, and the level of significance was set at 5% or P<0.05. The group means and standard deviation were calculated for all measurement parameters as study designed. A one-way analysis of variance (ANOVA) was used among the groups with software GraphPad Prism 6.0 and T-test was used with EXCEL Software.

### Results

The compound described in Example 3(110) exhibited 50% DDR1 phosphorylation suppression at a dose of 10 mg/kg relative to the difference between the untreated group and control group.

### FORMULATION EXAMPLE

### Formulation example 1

The components indicated below are mixed by a standard method, followed by making the mixture into tablets to obtain 10,000 tablets each containing 10 mg of active ingredient.
3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione . . . 100 g
   calcium carboxymethyl cellulose (disintegrant) . . . 20 g
   magnesium stearate (lubricant) . . . 10 g
   microcrystalline cellulose . . . 870 g

### Formulation example 2

The components indicated below are mixed by a standard method, filtered through a dust-removing filter, filled into ampoules so that each ampule contains 5 ml, and thermally sterilized in an autoclave to obtain 10,000 ampoules each containing 20 mg active ingredient.
3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione . . . 200 g
   mannitol . . . 20 g
   distilled water . . . 50 L

### INDUSTRIAL APPLICABILITY

The compound of the present invention has a potent and selective DDR1 inhibitory activity. The compound of the present invention therefore, can be used for the prevention and/or treatment of a disease which is related to the DDR1 receptor. Example of the disease which is related to the DDR1 receptor include cancers, kidney diseases, cardiovascular diseases, central nervous system disease and fibrosis, and the like.

1. A compound of formula (I-a), or a salt thereof, 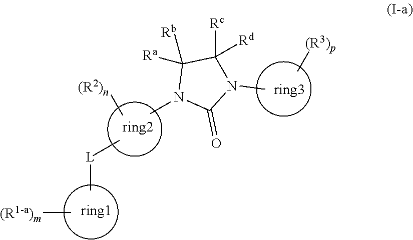
wherein:
ring 1 represents (1) a five- to seven-membered nitrogen-containing monocyclic heterocycle, or (2) an eight- to ten-membered nitrogen-containing bicyclic heterocycle,
R^{1-a} represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—(C1-6 alkylene)-NR⁴R⁵, (9) a five- to seven-membered monocyclic heterocyclyl, (10) NR⁴R⁵, (11) C(═O)NR⁴R⁵, (12) a cyano group, (13) a hydroxyl group, (14) an oxo group, (15) a C1-6 alkylsulfonyl group, (16) a Q-(C3-7 monocyclic carbocyclyl) group, or (17) a Q-(three- to seven-membered monocyclic heterocyclyl) group wherein:
Q represents any of NR⁶, O, and optionally oxidized S,
R⁴ and R⁵ each independently represent (1) a hydrogen atom, (2) a C1-6 alkyl group, (3) a C3-6 cycloalkyl group, (4) a three- to six-membered saturated heterocyclyl, (5) C(═O)R⁷, (6) (C1-6 alkylene)-NR⁸R⁹, (7) a phenyl group, or (8) a benzyl group, or R⁴ and R⁵ and the nitrogen atom they are attached thereto together form a five- to seven-membered nitrogen-containing monocyclic heterocycle, wherein, when R⁴ or R⁵ represents a C1-6 alkyl group, one of the carbon atoms in the C1-6 alkyl group may be replaced by NR¹⁰, O, or optionally oxidized S, and, when R⁴ or R⁵ represents (C1-6 alkylene)-NR⁸R⁹, the C1-6 alkylene may be substituted with an oxo group,
R⁶ represents (1) a hydrogen atom, (2) a C1-6 alkyl group, (3) a C3-6 cycloalkyl group, (4) a phenyl group, or (5) a benzyl group,
R⁷ represents (1) a hydrogen atom, (2) a C1-6 alkyl group, (3) a C3-6 cycloalkyl group, (4) a phenyl group, or (5) a benzyl group,
R⁸ and R⁹ each independently represent (1) a hydrogen atom, (2) a C1-6 alkyl group, (3) a C3-6 cycloalkyl group, (4) a phenyl group, or (5) a benzyl group,
R¹⁰ represents (1) a hydrogen atom, (2) a C1-6 alkyl group, (3) a C3-6 cycloalkyl group, (4) a phenyl group, or (5) a benzyl group,
when R^{1-a} represents any of (2) to (11), (15), (16) or (17), R^{1-a} may be substituted with one or more substituents selected from a halogen atom, a C1-6 alkyl group, a C1-6 haloalkyl group, a C1-6 hydroxyalkyl group, a hydroxyl group, a cyano group, and an oxo group,
when m is 2 or more, the plurality of R^{1-a} may be the same or different, and, two R^{1-a} may form a five- to seven-membered monocyclic ring with atoms they are attached thereto,
L represents (1) a bond, (2) CR¹¹R¹², (3) C(═O), (4) O, (5) NR¹³, or (6) optionally oxidized S, wherein R¹¹ and R¹² each independently represent (1) a hydrogen atom or (2) a C1-6 alkyl group, and R¹³ represents (1) a hydrogen atom or (2) a C1-6 alkyl group,
ring 2 represents (1) a C3-7 monocyclic carbocycle, (2) five- to seven-membered nitrogen-containing monocyclic heterocycle, (3) a C5-8 bridged carbocycle, 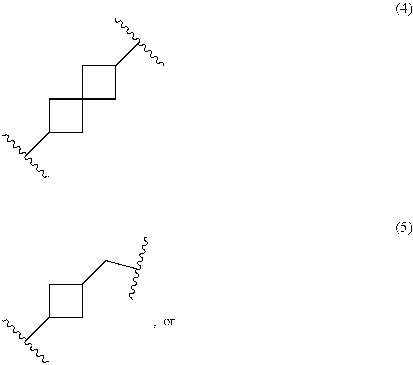 [SMILES: *C1CC2(C1)CC(*)C2 ; *CC1CC(*)C1]
R² represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) NR¹⁴R¹⁵, (8) a (C1-6 alkylene)-(five- to seven-membered monocyclic ring) group, (9) a cyano group, (10) a hydroxyl group, or (11) an oxo group, wherein:
R¹⁴ and R¹⁵ each independently represent (1) a hydrogen atom, (2) a C1-6 alkyl group, (3) a C3-6 cycloalkyl group, (4) a three- to six-membered saturated heterocyclyl, (5) acetyl group, (6) a phenyl group, or (7) a benzyl group, or R¹⁴ and R¹⁵ and the nitrogen atom they are attached thereto together form a five- to seven-membered nitrogen-containing monocyclic heterocycle,
when R¹⁴ or R¹⁵ represent a C1-6 alkyl group, one of the carbon atoms in the C1-6 alkyl group may be replaced by NR¹⁶, O, or optionally oxidized S, and
R¹⁶ represents (1) a hydrogen atom, (2) a C1-6 alkyl group, (3) a C3-6 cycloalkyl group, (4) a phenyl group, or (5) a benzyl group,
when R² represents any of (2) to (8), one of the carbon atoms in R² may be replaced by NR¹⁷, O, or optionally oxidized S, wherein R¹⁷ represents (1) a hydrogen atom, (2) a C1-6 alkyl group, or (3) C(═O)R¹⁸, wherein R¹⁸ represents (1) a NH—(C1-6 alkyl) group, or (2) an O(C1-6 alkyl) group,
when R² represents any of (2) to (8), R² may be substituted with one or more substituents selected from a halogen atom, a C1-6 alkyl group, a C1-6 haloalkyl group, a C1-6 hydroxyalkyl group, a hydroxyl group, a cyano group, and an oxo group,
when n is 2 or more, the plurality of R² may be the same or different, and, two R² may form a C3-7 monocyclic carbocycle with atoms they are attached thereto,
when L representing NR¹³, R^{1-a}, R¹³ and atoms they are attached thereto may form a five- to seven-membered nitrogen-containing monocyclic heterocycle, or when L representing NR¹³, R², R¹³ and atoms they are attached thereto may form a five- to seven-membered nitrogen-containing monocyclic heterocycle,
R^{a} and R^{b} each independently represent (1) a hydrogen atom, (2) a halogen atom, (3) a C1-6 alkyl group, (4) a C1-6 alkoxy group, or (5) a hydroxyl group, or (6) R^{a} and R^{b} together represent an oxo group,
R^{c} and R^{d} each independently represent (1) a hydrogen atom, (2) a halogen atom, (3) a C1-6 alkyl group, (4) a C1-6 alkoxy group, or (5) a hydroxyl group, or (6) R^{c} and R^{d} together represent an oxo group,
when R^{a} and R^{b} represent an oxo group, R^{c} and R^{d} do not represent an oxo group,
ring 3 represents (1) a C5-7 monocyclic carbocycle, or (2) a five- to seven-membered monocyclic heterocycle containing 1 to 4 heteroatoms selected from N, O, and optionally oxidized S,
R³ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) a C1-6 alkylsulfonyl group, (8) NR¹⁹R²⁰, (9) (C1-6 alkylene)-R²¹, (10) O—C1-6 alkylene)-R²¹, (11) O—R²², (12) a three- to seven-membered monocyclic heterocyclyl, (13) a cyano group, (14) a hydroxyl group, or (15) an oxo group, wherein:
R¹⁹ and R²⁰ each independently represent (1) a hydrogen atom or (2) a C1-6 alkyl group, or, R¹⁹ and R²⁰ and the nitrogen atom they are attached thereto may together form a five- to seven-membered nitrogen-containing monocyclic heterocycle, wherein one of the carbon atoms in the five- to seven-membered nitrogen-containing monocyclic heterocycle may be replaced by NR²³, O, or optionally oxidized S,
R²¹ represents (1) a C3-6 cycloalkyl group, (2) a C1-6 alkylsulfonyl group, or (3) NR¹⁹R²⁰,
R²² represents (1) a C3-6 cycloalkyl group, (2) a three- to six-membered saturated heterocyclyl, or (3) a C1-6 alkylsulfonyl group, wherein the C3-6 cycloalkyl group and the three- to six-membered saturated heterocyclyl may be substituted with C1-6 alkyl groups, and
R²³ represents (1) a hydrogen atom or (2) a C1-6 alkyl group,
when R³ represents any of (2) to (12), R³ may be substituted with one or more substituents selected from a halogen atom, a C1-6 alkyl group, a C1-6 haloalkyl group, a C1-6 hydroxyalkyl group, a hydroxyl group, a cyano group, and an oxo group,
when p is 2 or more, the plurality of R³ may be the same or different,
m represents an integer of 0 to 10,
n represents an integer of 0 to 10, and
p represents an integer of 0 to 10.

2. The compound or a salt thereof according to claim 1, wherein the compound is a compound of formula (I), 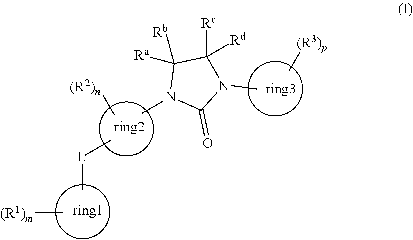
wherein:
ring 1 represents (1) a five- to seven-membered nitrogen-containing monocyclic heterocycle, or (2) an eight- to ten-membered nitrogen-containing bicyclic heterocycle,
R¹ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—C1-6 alkylene)-NR⁴R⁵, (9) a five- to seven-membered monocyclic heterocyclyl, (10) NR⁴R⁵, (11) C(═O)NR⁴R⁵, (12) a cyano group, (13) a hydroxyl group, or (14) an oxo group, wherein:
Q represents any of NR⁶, O, and optionally oxidized S,
R⁴ and R⁵ each independently represent (1) a hydrogen atom, (2) a C1-6 alkyl group, (3) a C3-6 cycloalkyl group, (4) a three- to six-membered saturated heterocyclyl, (5) C(═O)R⁷, (6) (C1-6 alkylene)-NR⁸R⁹, (7) a phenyl group, or (8) a benzyl group, wherein, when R⁴ or R⁵ represents a C1-6 alkyl group, one of the carbon atoms in the C1-6 alkyl group may be replaced by NR¹⁰, O, or optionally oxidized S, and, when R⁴ and R⁵ represent C1-6 alkyl groups, R⁴ and R⁵ and the nitrogen atom attached thereto may together form a five- to seven-membered nitrogen-containing monocyclic heterocycle, and, when R⁴ or R⁵ represents (C1-6 alkylene)-NR⁸R⁹, the C1-6 alkylene may be substituted with an oxo group,
R⁶ represents (1) a hydrogen atom, (2) a C1-6 alkyl group, (3) a C3-6 cycloalkyl group, (4) a phenyl group, or (5) a benzyl group,
R⁷ represents (1) a hydrogen atom, (2) a C1-6 alkyl group, (3) a C3-6 cycloalkyl group, (4) a phenyl group, or (5) a benzyl group,
R⁸ and R⁹ each independently represent (1) a hydrogen atom, (2) a C1-6 alkyl group, (3) a C3-6 cycloalkyl group, (4) a phenyl group, or (5) a benzyl group,
R¹⁰ represents (1) a hydrogen atom, (2) a C1-6 alkyl group, (3) a C3-6 cycloalkyl group, (4) a phenyl group, or (5) a benzyl group,
when R¹ represents any of (2) to (11), R¹ may be substituted with one or more substituents selected from a halogen atom, a C1-6 alkyl group, a C1-6 haloalkyl group, a C1-6 hydroxyalkyl group, a hydroxyl group, a cyano group, and an oxo group,
when m is 2 or more, the plurality of R¹ may be the same or different,
L represents (1) a bond, (2) CR¹¹R¹², (3) C(═O), (4) O, (5) NR¹³, or (6) optionally oxidized S, wherein R¹¹ and R¹² each independently represent (1) a hydrogen atom or (2) a C1-6 alkyl group, and R¹³ represents (1) a hydrogen atom or (2) a C1-6 alkyl group,
ring 2 represents (1) a C3-7 monocyclic carbocycle, (2) five- to seven-membered nitrogen-containing monocyclic heterocycle, (3) a C5-8 bridged carbocycle, 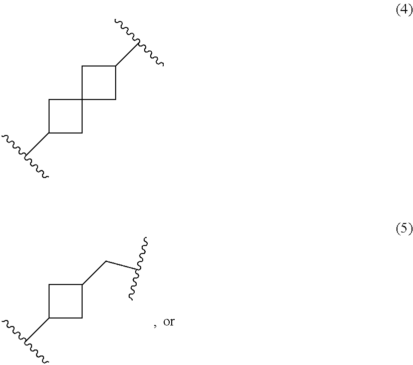 [SMILES: *C1CC2(C1)CC(*)C2 ; *CC1CC(*)C1]
R² represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) NR¹⁴R¹⁵, (8) a (C1-6 alkylene)-(five- to seven-membered monocyclic ring) group, (9) a cyano group, (10) a hydroxyl group, or (11) an oxo group, wherein:
R¹⁴ and R¹⁵ each independently represent (1) a hydrogen atom, (2) a C1-6 alkyl group, (3) a C3-6 cycloalkyl group, (4) a three- to six-membered saturated heterocyclyl, (5) acetyl group, (6) a phenyl group, or (7) a benzyl group,
when R¹⁴ or R¹⁵ represent a C1-6 alkyl group, one of the carbon atoms in the C1-6 alkyl group may be replaced by NR¹⁶, O, or optionally oxidized S,
when R¹⁴ and R¹⁵ represent C1-6 alkyl groups, R¹⁴ and R¹⁵ and the nitrogen atom attached thereto may together form a five- to seven-membered nitrogen-containing monocyclic heterocycle, and
R¹⁶ represents (1) a hydrogen atom, (2) a C1-6 alkyl group, (3) a C3-6 cycloalkyl group, (4) a phenyl group, or (5) a benzyl group,
when R² represents any of (2) to (8), one of the carbon atoms in R² may be replaced by NR¹⁷, O, or optionally oxidized S, wherein R¹⁷ represents (1) a hydrogen atom, (2) a C1-6 alkyl group, or (3) C(═O)R¹⁸, wherein R¹⁸ represents (1) a NH—(C1-6 alkyl) group, or (2) an O(C1-6 alkyl) group,
when R² represents any of (2) to (8), R² may be substituted with one or more substituents selected from a halogen atom, a C1-6 alkyl group, a C1-6 haloalkyl group, a C1-6 hydroxyalkyl group, a hydroxyl group, a cyano group, and an oxo group,
when n is 2 or more, the plurality of R² may be the same or different, and, when two R² represent C1-6 alkyl groups, the two R² may form a C3-7 monocyclic carbocycle with atoms constituting the ring 2,
when one of R¹ and R² represents a C1-6 alkyl group with L representing NR¹³ and R¹³ representing a C1-6 alkyl group, one of R¹ and R² may form a five- to seven-membered nitrogen-containing monocyclic heterocycle with NR¹³,
R^{a} and R^{b} each independently represent (1) a hydrogen atom, (2) a halogen atom, (3) a C1-6 alkyl group, (4) a C1-6 alkoxy group, or (5) a hydroxyl group, or (6) R^{a} and R^{b} together represent an oxo group,
R^{c} and R^{d} each independently represent (1) a hydrogen atom, (2) a halogen atom, (3) a C1-6 alkyl group, (4) a C1-6 alkoxy group, or (5) a hydroxyl group, or (6) R^{c} and R^{d} together represent an oxo group,
when R^{a} and R^{b} represent an oxo group, R^{c} and R^{d} do not represent an oxo group,
ring 3 represents a C5-7 monocyclic carbocycle, or a five- to seven-membered monocyclic heterocycle containing 1 to 4 heteroatoms selected from N, O, and optionally oxidized S,
R³ represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) a C1-6 alkylsulfonyl group, (8) NR¹⁹R²⁰, (9) (C1-6 alkylene)-R²¹, (10) O—C1-6 alkylene)-R²¹, (11) O—R²², (12) a three- to seven-membered monocyclic heterocyclyl, (13) a cyano group, (14) a hydroxyl group, or (15) an oxo group, wherein:
R¹⁹ and R²⁰ each independently represent (1) a hydrogen atom or (2) a C1-6 alkyl group, and when R¹⁹ and R²⁰ represent C1-6 alkyl groups, R¹⁹ and R²⁰ and the nitrogen atom attached thereto may together form a five- to seven-membered nitrogen-containing monocyclic heterocycle, wherein one of the carbon atoms in the five- to seven-membered nitrogen-containing monocyclic heterocycle may be replaced by NR²³, O, or optionally oxidized S,
R²¹ represents (1) a C3-6 cycloalkyl group, (2) a C1-6 alkylsulfonyl group, or (3) NR¹⁹R²⁰,
R²² represents (1) a C3-6 cycloalkyl group, (2) a three- to six-membered saturated heterocyclyl, or (3) a C1-6 alkylsulfonyl group, wherein the C3-6 cycloalkyl group and the three- to six-membered saturated heterocyclyl may be substituted with C1-6 alkyl groups, and
R²³ represents (1) a hydrogen atom or (2) a C1-6 alkyl group,
when R³ represents any of (2) to (12), R³ may be substituted with one or more substituents selected from a halogen atom, a C1-6 alkyl group, a C1-6 haloalkyl group, a C1-6 hydroxyalkyl group, a hydroxyl group, a cyano group, and an oxo group,
when p is 2 or more, the plurality of R³ may be the same or different,
m represents an integer of 0 to 10,
n represents an integer of 0 to 10, and
p represents an integer of 0 to 10.

3. The compound or a salt thereof according to claim 1, wherein the compound of formula (I-A), (I-B), or (I-C), 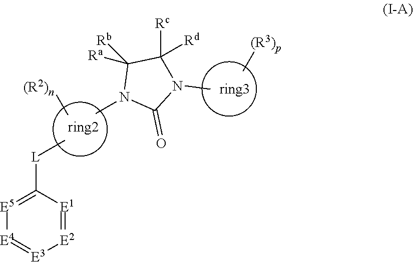
wherein:
E¹, E², E³, E⁴, and E⁵ each independently represent (1) CH, (2) CR¹, or (3) N, wherein at least one of E¹, E², E³, E⁴, and E⁵ represent N, and
other symbols represent the same meanings as described in claim 1, 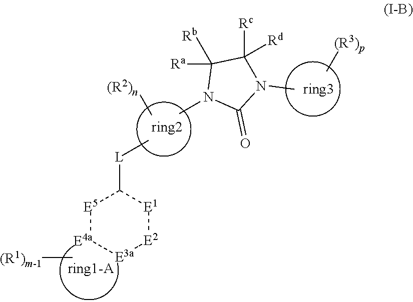
wherein:
E^{3a} and E^{4a} each independently represent (1) C or (2) N, wherein at least one of E¹, E², E^{3a}, E^{4a}, and E⁵ represent N,
ring 1-A, including E^{3a} and E^{4a}, represent a five- to seven-membered monocyclic ring group,
the broken-line bonds represent aromatic bonds,
m−1 represents an integer of 0 to 5, and
other symbols represent the same meanings as described in claim 1, 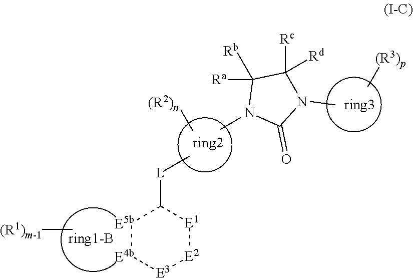
wherein:
E^{4b} and E^{5b} each independently represent (1) C or (2) N, wherein at least one of E¹, E², E³, E⁴b, and E⁵b represent N,
ring 1-B, including E^{4b} and E^{5b}, represents a five- to seven-membered monocyclic ring group, and
other symbols represent the same meanings as described in claim 1.

4. The compound or a salt thereof according to claim 1, wherein the moiety 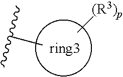
is 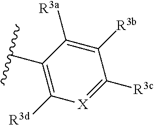
wherein:
X represents CR^{3e} or N,
R^{3a}, R^{3c}, R^{3d}, and R^{3e} each independently represent (1) a hydrogen atom, (2) a halogen atom, (3) a C1-6 alkyl group, (4) a C1-6 alkoxy group, (5) a cyano group, or (6) a hydroxyl group, and, when R^{3a}, R^{3c}, R^{3d}, or R^{3e} represents a C1-6 alkyl group or a C1-6 alkoxy group, the C1-6 alkyl group or a C1-6 alkoxy group may be substituted with 1 to 5 substituents selected from a halogen atom and a hydroxyl group,
R^{3b} represents (1) a C1-6 alkyl group, (2) a C1-6 alkoxy group, (3) a cyano group, or (4) a hydroxyl group, and, when R^{3b} represents a C1-6 alkyl group or a C1-6 alkoxy group, the C1-6 alkyl group or a C1-6 alkoxy group may be substituted with 1 to 5 substituents selected from a halogen atom and a hydroxyl group.

5. The compound or a salt thereof according to claim 1, wherein the moiety 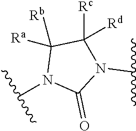
is

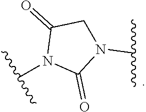 [SMILES: *N1CC(=O)N(*)C1=O]

6. The compound or a salt thereof according to claim 1, wherein the moiety 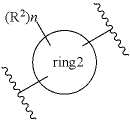
is selected from 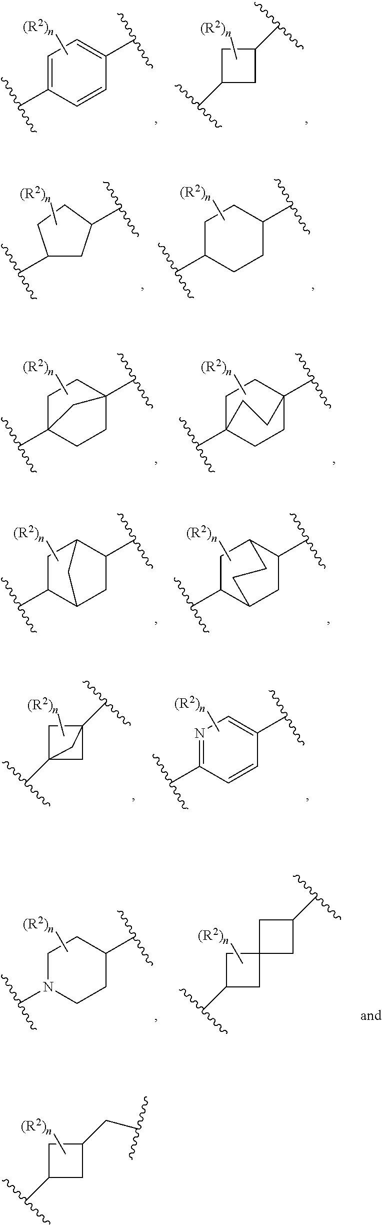
wherein all the symbols represent the same meanings as described in claim 1.

7. The compound or a salt thereof according to claim 3, wherein the compound is a compound of formula (I-1), 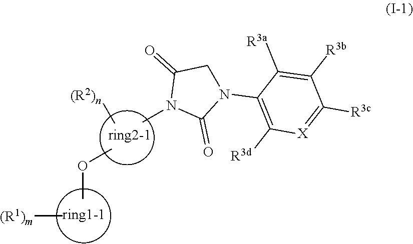
wherein the moiety 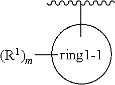
represents 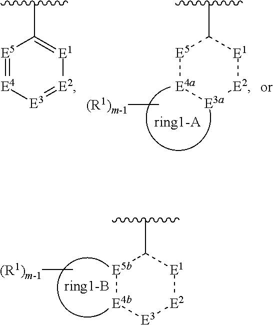
wherein all the symbols represent the same meanings as described in claim 3, and the moiety 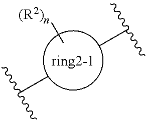
represents any one of the following 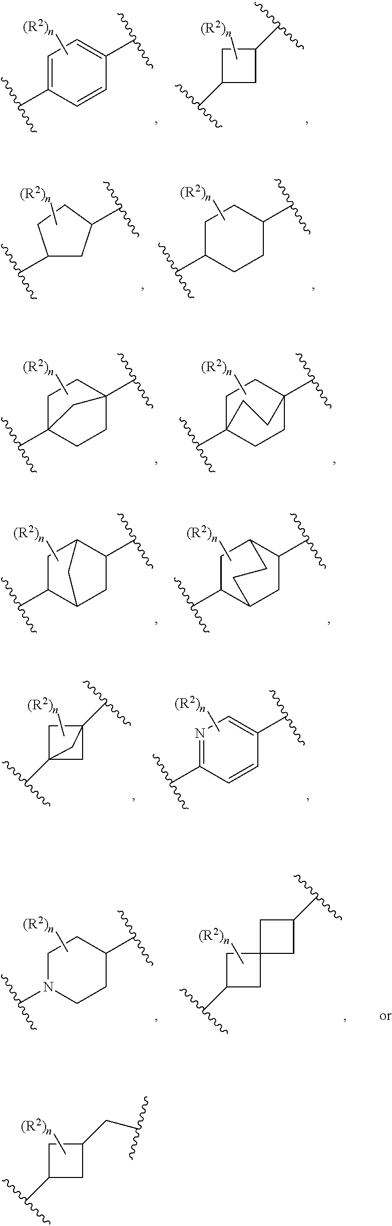
wherein all the symbols represent the same meanings as described in claim 3.

8. The compound or a salt thereof according to claim 1, wherein the compound is a compound of formula (I-3), 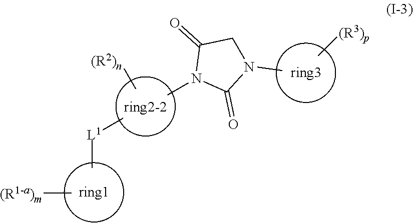
wherein L¹ represents (1) CR¹¹R¹², (2) C(═O), (3) O, (4) NR¹³, or (5) optionally oxidized S,
wherein R¹¹ and R¹² each independently represent (1) a hydrogen atom or (2) a C1-6 alkyl group, and R¹³ represents (1) a hydrogen atom or (2) a C1-6 alkyl group,
ring 2-2 represents (1) a C3-7 monocyclic carbocycle, (2) five- to seven-membered nitrogen-containing monocyclic heterocycle, (3) a C5-8 bridged carbocycle, or 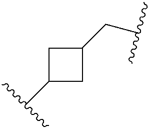 [SMILES: *CC1CC(*)C1]
and other symbols represent the same meanings as described in claim 1.

9. The compound or a salt thereof according to claim 8, wherein the moiety 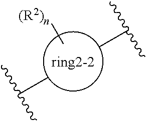
is selected from 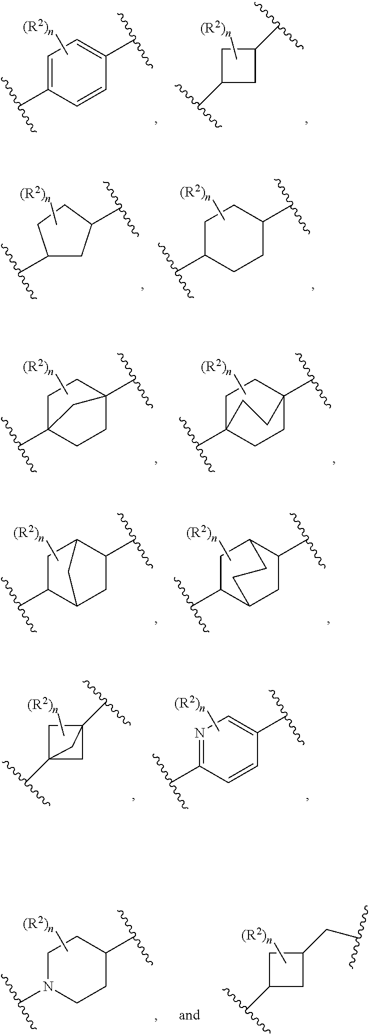
wherein all the symbols represent the same meanings as described in claim 1.

10. The compound or a salt thereof according to claim 1, wherein the compound is a compound of formula (I-4), 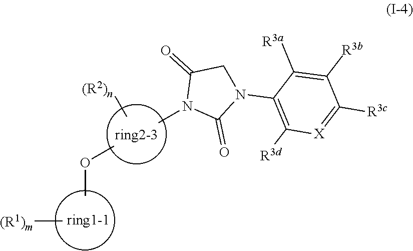
wherein the moiety 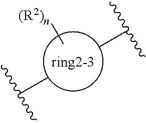
represents any one of the following 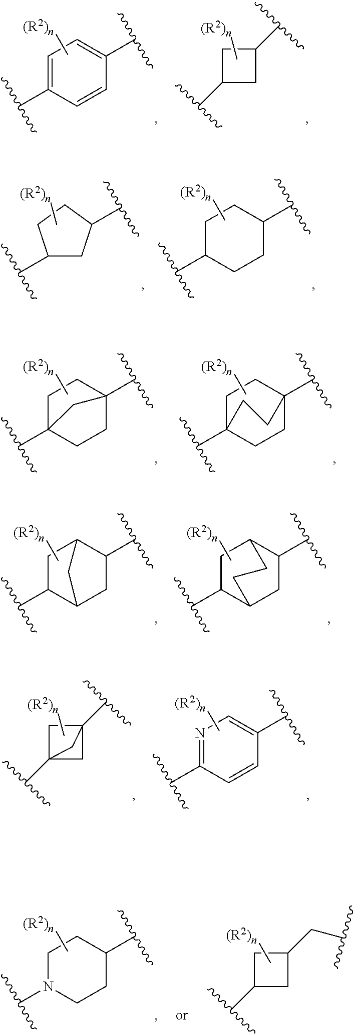
wherein all the symbols represent the same meanings as described in claim 1.

11. The compound or a salt thereof according to claim 1, wherein the compound is a compound of formula (I-5), 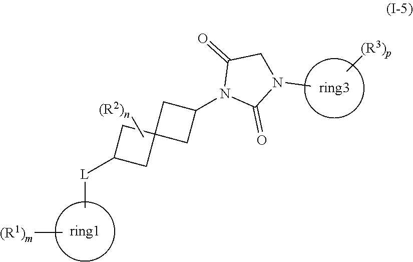
wherein R^{1-b} represents (1) a halogen atom, (2) a C1-6 alkyl group, (3) a C1-6 alkoxy group, (4) a C3-7 monocyclic carbocyclyl, (5) a C2-6 alkenyl group, (6) a C2-6 alkynyl group, (7) an O—(C1-6 alkylene)-Q-(C1-6 alkyl) group, (8) O—C1-6 alkylene)-NR⁴R⁵, (9) a five- to seven-membered monocyclic heterocyclyl, (10) NR⁴R⁵, (11) a cyano group, (12) a hydroxyl group, (13) an oxo group, (14) a C1-6 alkylsulfonyl group, (15) a Q-(C3-7 monocyclic carbocyclyl) group, or (16) a Q-(three- to seven-membered monocyclic heterocyclyl) group,
when R^{1-b} represents any of (2) to (10), (14), (15) or (16), R^{1-b} may be substituted with one or more substituents selected from a halogen atom, a C1-6 alkyl group, a C1-6 haloalkyl group, a C1-6 hydroxyalkyl group, a hydroxyl group, a cyano group, and an oxo group,
when m is 2 or more, the plurality of R^{1-b} may be the same or different, and, two R^{1-b} may form a five- to seven-membered monocyclic ring with atoms they are attached thereto, and other symbols represent the same meanings as described in claim 1.

12. The compound or a salt thereof according to claim 7, wherein the moiety 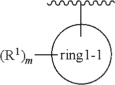
is 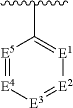
wherein all the symbols represent the same meanings as described in claim 7.

13. The compound or a salt thereof according to claim 7, wherein the moiety 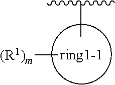
is 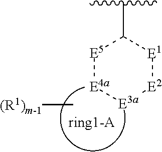
wherein all the symbols represent the same meanings as described in claim 7.

14. The compound or a salt thereof according to claim 7, wherein the moiety 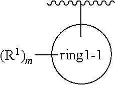
is 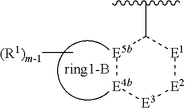
wherein all the symbols represent the same meanings as described in claim 7.

15. The compound or a salt thereof according to claim 7, wherein the compound is a compound of formula (I-1), 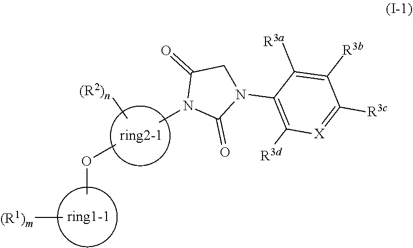
wherein the moiety 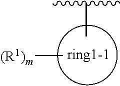
represents 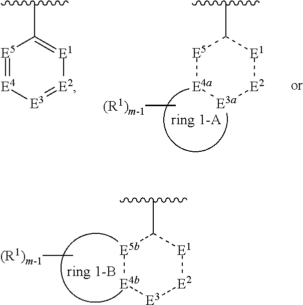
wherein
E¹, E², E³, E⁴, and E⁵ each independently represent (1) CH, (2) CR¹, or (3) N, wherein at least one of E¹, E², E³, E⁴, and E⁵ represent N, and
E^{3a} and E^{4a} each independently represent (1) C or (2) N, wherein at least one of E¹, E², E^{3a}, E^{4a}, and E⁵ represent N,
ring 1-A, including E^{3a} and E^{4a}, represent a five- to seven-membered monocyclic ring group,
E^{4b} and E^{5b} each independently represent (1) C or (2) N, wherein at least one of E¹, E², E³, E⁴b, and E⁵b represent N,
ring 1-B, including E^{4b} and E^{5b}, represents a five- to seven-membered monocyclic ring group, and
the broken-line bonds represent aromatic bonds,
R¹ represents (1) a halogen atom, (2) a C1-6 alkyl group, (3) a C1-6 alkoxy group, or (10) NR⁴R⁵,
when R¹ represents any of (2) to (3), R¹ may be substituted with one hydroxyl group,
R⁴ and R⁵ each independently represent (1) a hydrogen atom, or (2) a C1-6 alkyl group,
m−1 represents an integer of 0 to 5, and
when m is 2 or more, the plurality of R¹ may be the same or different,
the moiety 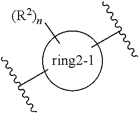
represents any one of the following 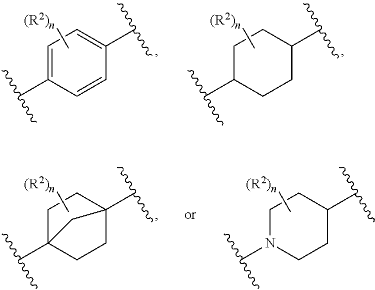
R² represents (2) a C1-6 alkyl group, or (4) a phenyl,
n represents an integer of 0 to 5,
when n is 2 or more, the plurality of R² may be the same or different,
X represents CR³, or N,
R^{3a}, R^{3c}, R^{3d}, and R³ each independently represent (1) a hydrogen atom, or (2) a halogen atom,
R^{3b} represents (1) a C1-6 alkyl group, (2) a C1-6 alkoxy group, or (7) a C1-6 alkylsulfonyl group, and, when R^{3b} represents a C1-6 alkyl group or a C1-6 alkoxy group, the C1-6 alkyl group or a C1-6 alkoxy group may be substituted with 1 to 5 halogen atom.

16. The compound or a salt thereof according to claim 7, wherein the compound is a compound of formula (I-1), 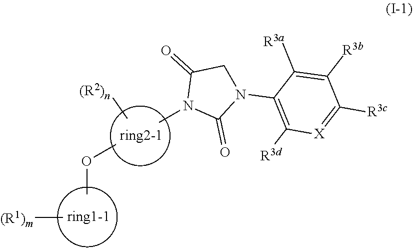
wherein the moiety 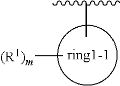
represents 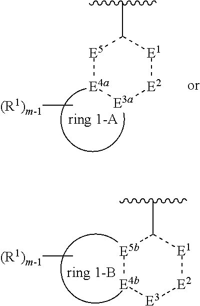
wherein
E¹, E², E³, and E⁵ each independently represent (1) CH, or (3) N, and
E^{3a} and E^{4a} each independently represent C, wherein at least one of E¹, E², and E⁵ represent N,
ring 1-A, including E^{3a} and E^{4a}, represent a five-membered monocyclic ring group,
E^{4b} and E^{5b} each independently represent C, wherein at least one of E¹, E², E³ represent N,
ring 1-B, including E^{4b} and E^{5b}, represents a five-membered monocyclic ring group, and
the broken-line bonds represent aromatic bonds,
R¹ represents (1) a halogen atom, (2) a C1-6 alkyl group, or (3) a C1-6 alkoxy group,
when R¹ represents any of (2) to (3), R¹ may be substituted with one hydroxyl group,
m−1 represents an integer of 0 to 5, and
when m is 2 or more, the plurality of R¹ may be the same or different,
the moiety 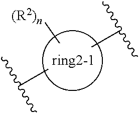
represents any one of the following 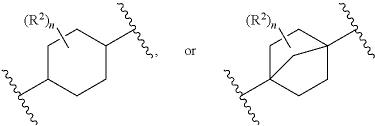
R² represents H,
n represents 0,
X represents N,
R^{3a}, R^{3c}, and R^{3d} each independently represent a hydrogen atom,
R^{3b} represents (1) a C1-6 alkyl group, and, the C1-6 alkyl group may be substituted with 1 to 5 halogen atom.

17. The compound or a salt thereof according to claim 7, wherein the compound is a compound of formula (I-1), 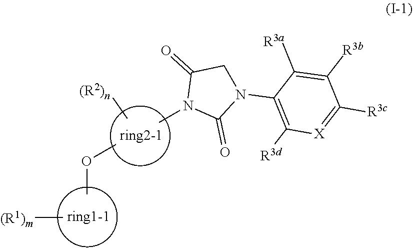
wherein the moiety 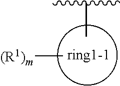
represents 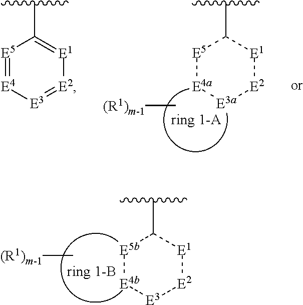
wherein
E¹, E², E³, E⁴, and E⁵ each independently represent (1) CH, (2) CR¹, or (3) N, wherein at least one of E¹, E², E³, E⁴, and E⁵ represent N, and
E^{3a} and E^{4a} each independently represent (1) C or (2) N, wherein at least one of E¹, E², E^{3a}, E^{4a}, and E⁵ represent N,
ring 1-A, including E^{3a} and E^{4a}, represent a five- to seven-membered monocyclic ring group,
E^{4b} and E^{5b} each independently represent (1) C or (2) N, wherein at least one of E¹, E², E³, E⁴b, and E⁵b represent N,
ring 1-B, including E^{4b} and E^{5b}, represents a five- to seven-membered monocyclic ring group, and
the broken-line bonds represent aromatic bonds,
R¹ represents (3) a C1-6 alkoxy group, or (10) NR⁴R⁵,
R⁴ and R⁵ each independently represent (1) a hydrogen atom, or (2) a C1-6 alkyl group,
m−1 represents an integer of 0 to 5, and
when m is 2 or more, the plurality of R¹ may be the same or different,
the moiety 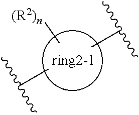
represents any one of the following 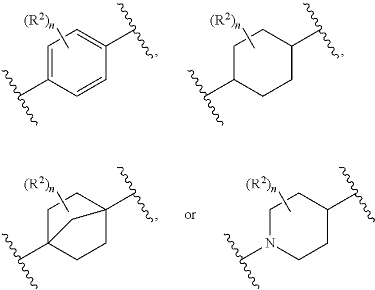
R² represents (2) a C1-6 alkyl group, or (4) a phenyl,
n represents an integer of 0 to 10,
when n is 2 or more, the plurality of R² may be the same or different,
X represents CR³, or N,
R^{3a}, R^{3c}, R^{3d}, and R^{3′} each independently represent (1) a hydrogen atom, or (2) a halogen atom,
R^{3b} represents (1) a C1-6 alkyl group, or (2) a C1-6 alkoxy group, and, when R^{3b} represents a C1-6 alkyl group or a C1-6 alkoxy group, the C1-6 alkyl group or a C1-6 alkoxy group may be substituted with 1 to 5 halogen atom.

18. The compound or a salt thereof according to claim 1, wherein the compound is
(1) 3-{4-[(2-amino-5-pyrimidinyl)oxy]phenyl}-1-[5-(trifluoromethyl)-3-pyridinyl]-2,4-imidazolidinedione,
(2) 3-[4-(1H-pyrazolo[3,4-b]pyridin-5-yloxy)phenyl]-1-[5-(trifluoromethyl)-3-pyridinyl]-2,4-imidazolidinedione,
(3) 1-[3-(methylsulfonyl)-5-(trifluoromethyl)phenyl]-3-[4-(1H-pyrazolo[3,4-b]pyridin-5-yloxy)phenyl]-2,4-imidazolidinedione,
(4) 3-{4-[(2-amino-4-pyrimidinyl)oxy]-2-methylphenyl}-1-[5-(trifluoromethyl)-3-pyridinyl]-2,4-imidazolidinedione,
(5) 3-{trans-4-[(7-methoxy-4-quinazolinyl)oxy]cyclohexyl}-1-[5-(trifluoromethyl)-3-pyridinyl]-2,4-imidazolidinedione,
(6) 1-[3-fluoro-5-(trifluoromethyl)phenyl]-3-[trans-4-(thieno[3,2-b]pyridin-6-yloxy)cyclohexyl]-2,4-imidazolidinedione,
(7) 3-{3-isopropyl-4-[(2-oxo-2,3-dihydro-1H-imidazo[4,5-b]pyridin-7-yl)oxy]phenyl}-1-[5-(trifluoromethyl)-3-pyridinyl]-2,4-imidazolidinedione,
(8) 3-[trans-4-(7H-pyrrolo[2,3-d]pyrimidin-4-yloxy)cyclohexyl]-1-[5-(trifluoromethyl)-3-pyridinyl]-2,4-imidazolidinedione,
(9) 3-[3-methyl-4-(pyrazolo[1,5-a]pyrimidin-7-yloxy)phenyl]-1-[5-(trifluoromethyl)-3-pyridinyl]-2,4-imidazolidinedione,
(10) 3-[4-(7H-pyrrolo[2,3-d]pyrimidin-4-yloxy)bicyclo[2.2.1]hept-1-yl]-1-[5-(trifluoromethyl)-3-pyridinyl]-2,4-imidazolidinedione,
(11) 3-{4-[(2-amino-4-pyrimidinyl)oxy]-2-methylphenyl}-1-[4-fluoro-3-(trifluoromethoxy)phenyl]-2,4-imidazolidinedione,
(12) 3-{4-[(2-amino-4-pyrimidinyl)oxy]-2-methylphenyl}-1-[3-fluoro-5-(trifluoromethyl)phenyl]-2,4-imidazolidinedione,
(13) 3-{5-[(2-amino-4-pyrimidinyl)oxy]-2-biphenylyl}-1-[5-(trifluoromethyl)-3-pyridinyl]-2,4-imidazolidinedione,
(14) 3-[3-ethyl-4-(1H-pyrrolo[2,3-b]pyridin-4-yloxy)phenyl]-1-[5-(trifluoromethyl)-3-pyridinyl]-2,4-imidazolidinedione,
(15) 1-{4-[(2-amino-4-pyrimidinyl)oxy]-3-ethylphenyl}-3-[3-(trifluoromethyl)phenyl]-2-imidazolidinone,
(16) 3-{1-[(7-methoxy-4-quinolinyl)methyl]-4-piperidinyl}-1-[5-(trifluoromethyl)-3-pyridinyl]-2,4-imidazolidinedione, or
(17) 3-[4-(1H-pyrazolo[3,4-b]pyridin-5-yloxy)phenyl]-1-{5-[2-(trifluoromethyl)-2-oxetanyl]-3-pyridinyl}-2,4-imidazolidinedione.

19. The compound or a salt thereof according to claim 1, wherein the compound is
(1) 3-(trans-4-{[3-(2-hydroxyethoxy)-1H-pyrazolo[3,4-b]pyridin-5-yl]oxy}cyclohexyl)-1-[5-(trifluoromethyl)-3-pyridinyl]-2,4-imidazolidinedione,
(2) 3-(4-{[6-(2-hydroxy-2-propanyl)-7H-pyrrolo[2,3-d]pyrimidin-4-yl]oxy}bicyclo[2.2.1]hept-1-yl)-1-[5-(trifluoromethyl)-3-pyridinyl]-2,4-imidazolidinedione,
(3) 3-{4-[(5-fluoro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(4) 3-{4-[(5-chloro-7H-pyrrolo[2,3-d]pyrimidin-4-yl)oxy]bicyclo[2.2.1]hept-1-yl}-1-[5-(trifluoromethyl)-3-pyridinyl]-2,4-imidazolidinedione,
(5) 5-{2,4-dioxo-3-[4-(7H-pyrrolo[2,3-d]pyrimidin-4-yloxy)bicyclo[2.2.1]hept-1-yl]-1-imidazolidinyl}nicotinonitrile,
(6) 3-{2,4-dioxo-3-[4-(7H-pyrrolo[2,3-d]pyrimidin-4-yloxy)bicyclo[2.2.1]hept-1-yl]-1-imidazolidinyl}benzonitrile, or
(7) 3-[4-(1H-pyrazolo[3,4-b]pyridin-4-yloxy)bicyclo[2.2.1]hept-1-yl]-1-[5-(trifluoromethyl)-3-pyridinyl]-2,4-imidazolidinedione.

20. A pharmaceutical composition comprising the compound of claim 1, or a salt thereof, and a pharmaceutically acceptable carrier.

21. The pharmaceutical composition according to claim 20, which is a DDR1 inhibitor.

22. The pharmaceutical composition according to claim 20, which is a preventive and/or therapeutic agent for a DDR1-related disease.

23. The pharmaceutical composition according to claim 22, wherein the DDR1-related disease is a cancer, a kidney disease, a cardiovascular disease, a central nervous system disease, or fibrosis.

24. The pharmaceutical composition according to claim 22, wherein the DDR1-related disease is a cancer, and the cancer is lung cancer, breast cancer, esophageal cancer, head and neck cancer, liver cancer, prostate cancer, lymphoma, leukemia, melanoma, pancreas cancer, neuroblastoma, glioma, colon cancer, renal cell carcinoma, thyroid cancer, stomach cancer, bladder cancer, ovarian cancer, endometrial cancer, brain tumor, or sarcoma.

25. The pharmaceutical composition according to claim 22, wherein the DDR1-related disease is a kidney disease, and the kidney disease is acute kidney failure, chronic kidney failure, renal fibrosis, diabetic nephropathy, membranoproliferative glomerulonephritis, mesangial proliferative nephritis, interstitial nephritis, lupus nephritis, amyloid kidney, pyelonephritis, crescentic glomerulonephritis, glomerular nephritis, membranous nephropathy, IgA nephropathy, focal glomerulosclerosis, hypertensive nephrosclerosis, Alport syndrome, Goodpasture's syndrome, or nephrotic nephritis.

26. A method for preventing and/or treating a DDR1-related disease, comprising administering to a mammal an effective amount of the compound of claim 1, a salt thereof, a solvate thereof, an N-oxide thereof, or a prodrug of any of these.

27. A compound of claim 1 or a salt thereof, for use in prevention and/or treatment of a DDR1-related disease.

28. (canceled)
